(19) United States
(12) Patent Application Publication
(10) Pub. No.: US 2020/0405832 A1
(43) Pub. Date: Dec. 31, 2020

(54) METHOD FOR ANTI-MUC1* CAR T CELL STIMULATION

(71) Applicant: MINERVA BIOTECHNOLOGIES CORPORATION, Waltham, MA (US)

(72) Inventors: Cynthia BAMDAD, Waltham, MA (US); Nelson GLENNIE, Waltham, MA (US)

(21) Appl. No.: 16/978,974

(22) PCT Filed: Mar. 11, 2019
(86) PCT No.: PCT/US2019/021556

§ 371 Date: Sep. 8, 2020

Related U.S. Application Data
(60) Provisional application No. 62/640,697, filed on Mar. 9, 2018.

(51) Int. Cl. A61K 39/00; C12N 5/0783; C07K 16/30; C07K 14/725; C07K 14/73; C07K 14/705

(52) U.S. Cl. CPC A61K 39/00117; C12N 5/0636; C07K 16/3092; C07K 14/7051; C07K 2317/622; C07K 14/70517; C07K 2317/34; A61K 2039/5158; C07K 14/70514

(57) ABSTRACT

The present application discloses a composition comprising a MUC1* extra cellular domain peptide attached to a surface and an immune cell that bears a receptor that binds to the peptide.

### BACKGROUND OF THE INVENTION

### 1. Field of the Invention

The present application relates to a method of activating, stimulating expansion of, or selecting for immune cells bearing a receptor for a specific antigen, involving contacting an immune cell expressing a cognate receptor with the antigen to which it binds. In one aspect the immune cell is an anti-MUC1*CAR T cell and the stimulating antigen is a peptide derived from the sequence of a cleaved MUC1 that is devoid of the tandem repeat domain.

### 2. General Background and State of the Art

We previously discovered that a cleaved form of the MUC1 (SEQ ID NO:1) transmembrane protein is a growth factor receptor that drives the growth of over 75% of all human cancers. The cleaved form of MUC1, which we called MUC1* (pronounced muk 1 star), is a powerful growth factor receptor. Cleavage and release of the bulk of the extracellular domain of MUC1 unmasks a binding site for activating ligands dimeric NME1, NME6, NME7, NME7_{AB}, NME7-X1 or NME8. It is an ideal target for cancer drugs as it is aberrantly expressed on over 75% of all cancers and is likely overexpressed on an even higher percentage of metastatic cancers (Mahanta et al. (2008) A Minimal Fragment of MUC1 Mediates Growth of Cancer Cells. PLoS ONE 3(4): e2054. doi:10.1371/journal.pone.0002054; Fessler et al. (2009), “MUC1* is a determinant of trastuzumab (Herceptin) resistance in breast cancer cells,” Breast Cancer Res Treat. 118(1):113-124). After MUC1 cleavage most of its extracellular domain is shed from the cell surface. The remaining portion has a truncated extracellular domain that comprises most or all of the primary growth factor receptor sequence called PSMGFR (SEQ ID NO:2).

Antibodies are increasingly used to treat human diseases. Antibodies generated in non-human species have historically been used as therapeutics in humans, such as horse antibodies. More recently, antibodies are engineered or selected so that they contain mostly human sequences in order to avoid a generalized rejection of the foreign antibody. The process of engineering recognition fragments of a non-human antibody into a human antibody is generally called ‘humanizing’. The amount of non-human sequences that are used to replace the human antibody sequences determines whether they are called chimeric, humanized or fully human.

Alternative technologies exist that enable generation of humanized or fully human antibodies. These strategies involve screening libraries of human antibodies or antibody fragments and identifying those that bind to the target antigen, rather than immunizing an animal with the antigen. Another approach is to engineer the variable region(s) of an antibody into an antibody-like molecule. Another approach involves immunizing a humanized animal. The present invention is intended to also encompass these approaches for use with recognition fragments of antibodies that the inventors have determined bind to the extracellular domain of MUC1*.

In addition to treating patients with an antibody, cancer immunotherapies have recently been shown to be effective in the treatment of blood cancers. One cancer immunotherapy, called CAR T (chimeric antigen receptor T cell) therapy, engineers a T cell so that it expresses a chimeric receptor having an extra cellular domain that recognizes a tumor antigen, and a transmembrane and cytoplasmic tail of a T cell (Dai H, Wang Y, Lu X, Han W. (2016) Chimeric Antigen Receptors Modified T-Cells for Cancer Therapy. J Natl Cancer Inst. 108(7): djv439). Such receptor is composed of an single chain antibody fragment (scFv) that recognizes a tumor antigen, linked to a T cell transmembrane and signaling domains. Upon binding of the receptor to a cancer associated antigen, a signal is transmitted resulting in T-cell activation, propagation and the targeted killing of the cancer cells. In practice, a patient's T cells are isolated and transduced with a CAR, expanded and then injected back into the patient. When the patient's CAR T cells bind to the antigen on a cancer cell, the CAR T cells expand and attack the cancer cells. A drawback of this method is the risk of activating the patient's immune system to destroy cells bearing the target antigen, when most cancer antigens are expressed on some healthy tissues, but overexpressed on cancerous tissues. To minimize the risk of off-tumor/on-target effects, the cancer antigen should be minimally expressed on healthy tissues.

Immune cells, other than T cells, can be engineered to express a receptor that recognizes a tumor antigen. Natural killer, NK, cells can also be engineered to express a receptor that recognizes a specific tumor antigen and methods of the invention can be used to activate, stimulate expansion or selecting for those immune cells that bear a receptor for a specific antigen, involving contacting the immune cell expressing the cognate receptor with the antigen to which it binds.

Another cancer immunotherapy involves BiTEs (Bi-specific T cell Engagers). The BiTE approach attempts to eliminate the CAR T associated risk of off-tumor/on-target effects. Unlike CAR T, BiTEs are bispecific antibodies that should not pose any greater risk than regular antibody-based therapies. However, unlike typical anti-cancer antibodies that bind to and block a cancer antigen, BiTEs are designed to bind to an antigen on the tumor cell and simultaneously bind to an antigen on an immune cell, such as a T cell. In this way, a BiTE recruits the T cell to the tumor. BiTEs are engineered proteins that simultaneously bind to a cancer associated antigen and a T-cell surface protein such as CD3-epsilon. BiTEs are antibodies made by genetically linking the scFv's of an antibody that binds to a T cell antigen, like anti-CD3-epsilon to a scFv of a therapeutic monoclonal antibody that binds to a cancer antigen (Patrick A. Baeuerle, and Carsten Reinhardt (2009) Bispecific T-cell engaging antibodies for cancer therapy. Cancer Res. 69(12):4941-4944).

### SUMMARY OF THE INVENTION

In one aspect, the present invention is directed to a non-human, human or humanized anti-MUC1* antibody or antibody fragment or antibody-like protein that binds to a region on extracellular domain of MUC1 isoform or cleavage product that is devoid of the tandem repeat domains. The non-human, human or humanized anti-MUC1* antibody or antibody fragment or antibody-like protein may specifically bind to

(i) PSMGFR region of MUC1;
(ii) PSMGFR peptide;
(iii) a peptide having amino acid sequence of
(SEQ ID NO: 620)
SNIKFRPGSVVVQLTLAFREGTINVHDVETQFNQYKTEAASRY;
(iv) a peptide having amino acid sequence of
(SEQ ID NO: 621)
SVVVQLTLAFREGTINVHDVETQFNQYKTEAASRY;
(v) a peptide having amino acid sequence of
(SEQ ID NO: 622)
VQLTLAFREGINVHDVETQFNQY;
or
(vi) a peptide having amino acid sequence of
(SEQ ID NO: 623)
SNIKFRPGSVVVQLTLAFREGTIN.

The non-human, human or humanized antibody may be IgG1, IgG2, IgG3, IgG4 or IgM. The human or humanized antibody fragment or antibody-like protein may be scFv or scFv-Fc.

The human or humanized antibody, antibody fragment or antibody-like protein as in above may comprise a heavy chain variable region and light chain variable region which is derived from mouse monoclonal MN-E6 antibody, and has at least 80%, 90% or 95% or 98% sequence identity to the mouse monoclonal MN-E6 antibody. The heavy chain variable region may have at least 90% or 95% or 98% sequence identity to SEQ ID NO:13 and the light chain variable region may have at least 90% or 95% or 98% sequence identity to SEQ ID NO:66.

The human or humanized antibody, antibody fragment or antibody-like protein according to above may include complementarity determining regions (CDRs) in the heavy chain variable region and light chain variable region having at least 90% or 95% or 98% sequence identity to CDR1, CDR2 or CDR3 regions having sequence as follows:

CDR1 heavy chain SEQ ID NO:17

CDR1 light chain SEQ ID NO:70,

CDR2 heavy chain SEQ ID NO:21

CDR2 light chain SEQ ID NO:74,

CDR3 heavy chain SEQ ID NO:25

CDR3 light chain SEQ ID NO:78.

The human or humanized antibody, antibody fragment or antibody-like protein described above may include a heavy chain variable region and light chain variable region which is derived from mouse monoclonal MN-C2 antibody, and has at least 80%, 90% or 95% or 98% sequence identity to the mouse monoclonal MN-C2 antibody. The heavy chain variable region may have at least 90% or 95% or 98% sequence identity to SEQ ID NO:119 and the light chain variable region has at least 90% or 95% or 98% sequence identity to SEQ ID NO:169. The complementarity determining regions (CDRs) in the heavy chain variable region and light chain variable region may have at least 90% or 95% or 98% sequence identity to CDR1, CDR2 or CDR3 regions having sequence as follows:

CDR1 heavy chain SEQ ID NO:123

CDR1 light chain SEQ ID NO:173,

CDR2 heavy chain SEQ ID NO:127

CDR2 light chain SEQ ID NO:177,

CDR3 heavy chain SEQ ID NO:131

CDR3 light chain SEQ ID NO:181.

The human or humanized antibody, antibody fragment or antibody-like protein as in above may include a heavy chain variable region and light chain variable region which is derived from mouse monoclonal MN-C3 antibody, and may have at least 80%, 90% or 95% or 98% sequence identity to the mouse monoclonal MN-C3 antibody. The heavy chain variable region may have at least 90% or 95% or 98% sequence identity to SEQ ID NO:414 and the light chain variable region may have at least 90% or 95% or 98% sequence identity to SEQ ID NO:459. The complementarity determining regions (CDRs) in the heavy chain variable region and light chain variable region may have at least 90% or 95% or 98% sequence identity to CDR1, CDR2 or CDR3 regions having sequence as follows:

CDR1 heavy chain SEQ ID NO:418

CDR1 light chain SEQ ID NO:463,

CDR2 heavy chain SEQ ID NO:422

CDR2 light chain SEQ ID NO:467,

CDR3 heavy chain SEQ ID NO:426,

CDR3 light chain SEQ ID NO:471.

The human or humanized antibody, antibody fragment or antibody-like protein described above may include a heavy chain variable region and light chain variable region which is derived from mouse monoclonal MN-C8 antibody, and has at least 80%, 90% or 95% or 98% sequence identity to the mouse monoclonal MN-C8 antibody. The heavy chain variable region may have at least 90% or 95% or 98% sequence identity to SEQ ID NO:506 and the light chain variable region may have at least 90% or 95% or 98% sequence identity to SEQ ID NO:544. The complementarity determining regions (CDRs) in the heavy chain variable region and light chain variable region may have at least 90% or 95% or 98% sequence identity to CDR1, CDR2 or CDR3 regions having sequence as follows:

CDR1 heavy chain SEQ ID NO:508

CDR1 light chain SEQ ID NO:546,

CDR2 heavy chain SEQ ID NO:510

CDR2 light chain SEQ ID NO:548,

CDR3 heavy chain SEQ ID NO:512,

CDR3 light chain SEQ ID NO:550.

In another aspect, the present invention is directed to an anti-MUC1* extracellular domain antibody comprised of sequences of a humanized MN-E6 represented by humanized IgG2 heavy chain, or humanized IgG1 heavy chain, paired with humanized Kappa light chain, or humanized Lambda light chain. The humanized IgG2 heavy chain may be SEQ ID NOS:53, humanized IgG1 heavy chain may be SEQ ID NO:57, humanized Kappa light chain may be SEQ ID NO:108, and humanized Lambda light chain may be SEQ ID NO:112, or a sequence having 90%, 95% or 98% sequence identity thereof.

In another aspect, the invention is directed to an anti-MUC1* extracellular domain antibody comprised of sequences of a humanized MN-C2 represented by humanized IgG1 heavy chain, humanized IgG2 heavy chain, paired with humanized Lambda light chain, and humanized Kappa light chain. The humanized IgG1 heavy chain MN-C2 may be SEQ ID NOS:159 or IgG2 heavy chain may be SEQ ID NOS:164 paired with Lambda light chain (SEQ ID NO:219) or Kappa light chain (SEQ ID NO:213), or a sequence having 90%, 95% or 98% sequence identity thereof.

In another aspect, the invention is directed to a human or humanized anti-MUC1* antibody or antibody fragment or antibody-like protein according to above, which inhibits the binding of NME protein to MUC1*. The NME may be NME1, NME6, NME7AB, NME7-X1, NME7 or NME8.

In yet another aspect, the invention is directed to a single chain variable fragment (scFv) comprising a heavy and light chain variable regions connected via a linker, further comprising CDRs of antibodies that bind to MUC1* extracellular domain. The CDRs may be derived from MN-E6, or MN-C2, antibodies or humanized antibodies thereof. The scFv may be one that possesses the SEQ ID NOS:233, 235 and 237 (E6); SEQ ID NOS:239, 241, and 243 (C2); SEQ ID NOS:245, 247, and 249 (C3); or SEQ ID NOS:251, 253, and 255 (C8).

In still another aspect, the invention is directed to a chimeric antigen receptor (CAR) comprising a scFv or a humanized variable region that binds to the extracellular domain of a MUC1 that is devoid of tandem repeats, a linker molecule, a transmembrane domain and a cytoplasmic domain. The single chain antibody fragment may bind to

(i) PSMGFR region of MUC1,
(ii) PSMGFR peptide,
(iii) a peptide having amino acid sequence
(SEQ ID NO: 620)
SNIKFRPGSVVVQLTLAFREGTINVHDVETQFNQYKTEAASRY;
(iv) a peptide having amino acid sequence of
(SEQ ID NO: 621)
SVVVQLTLAFREGTINVHDVETQFNQYKTEAASRY;
(v) a peptide having amino acid sequence of
(SEQ ID NO: 622)
VQLTLAFREGTINVHDVETQFNQY;
or
(vi) a peptide having amino acid sequence of
(SEQ ID NO: 623)
SNIKFRPGSVVVQLTLAFREGTIN.

In the CAR as described above, portions of any of the variable regions set forth and described above, or combination thereof may be used in the extracellular domain of the CAR. The CAR also comprises a transmembrane region and a cytoplasmic tail that comprises sequence motifs that signal immune system activation. The extracellular domain may be comprised of non-human, or humanized single chain antibody fragments of an MN-E6 scFv, MN-C2 scFv, MN-C3 scFv or MN-C8 scFv.

In the CAR as described above, the extracellular domain may include a non-human or humanized single chain antibody fragments of an MN-E6 scFv set forth as SEQ ID NOS: 233, 235, or 237), MN-C2 scFv (SEQ ID NOS:239, 241, or 243), MN-C3 scFv (SEQ ID NOS: 245, 247, or 249) or MN-C8 scFv (SEQ ID NOS:251, 253, or 255).

In any of the CARs described above, the cytoplasmic tail may be comprised of one or more of signaling sequence motifs CD3-zeta, CD27, CD28, 4-1BB, OX40, CD30, CD40, ICAm-1, LFA-1, ICOS, CD2, CD5, or CD7.

In any of the CARs described above, the sequence may be CARMN-E6 CD3z (SEQ ID NOS:295), CARMN-E6 CD28/CD3z (SEQ ID NOS:298); CARMN-E6 4-1BB/CD3z (SEQ ID NOS:301); CARMN-E6 OX40/CD3z (SEQ ID NOS:617); CARMN-E6 CD28/4-1BB/CD3z (SEQ ID NOS:304); CARMN-E6 CD28/OX40/CD3z (SEQ ID NOS:619); CAR MN-C2 CD3z (SEQ ID NOS:607); CAR MN-C2 CD28/CD3z (SEQ ID NOS:609); CAR MN-C2 4-1BB/CD3z (SEQ ID NOS:611 and SEQ ID NOS: 719); CAR MN-C2 OX40/CD3z (SEQ ID NOS:613); CAR MN-C2 CD28/4-1BB/CD3z (SEQ ID NOS: 307); CAR MN-C2 CD28/OX40/CD3z (SEQ ID NOS:615) or CAR MN-C3 4-1BB/CD3z (SEQ ID NOS: 601).

In another aspect, the CAR may have an extracellular domain unit that recognizes a peptide. The peptide may be PSMGFR (SEQ ID NO:2). The peptide may be a peptide derived from NME7. The peptide may be

| NME7A peptide 1 (A domain): | |
|---|---|
| MLSRKEALDFHVDHQS; | (SEQ ID NO: 7) |
| NME7A peptide 2 (A domain): | |
| SGVARTDASES; | (SEQ ID NO: 8) |
| NME7B peptide 1 (B domain): | |
| DAGFEISAMQMFNMDRVNVE; | (SEQ ID NO: 9) |
| NME7B peptide 2 (B domain): | |
| EVYKGVVTEYHDMVTE; | (SEQ ID NO: 10) |
| or | |
| NME7B peptide 3 (B domain): | |
| AIFGKTKIQNAVHCTDLPEDGLLEVQYFF. | (SEQ ID NO: 11) |

In another aspect, the invention is directed to a composition that includes at least two CARs with different extracellular domain units transfected into the same cell.

The at least two CARs may have one CAR that does not have a tumor antigen targeting recognition unit and the other CAR does have a tumor antigen targeting recognition unit. In another aspect of the invention, one of the extracellular domain recognition units may bind to MUC1* extracellular domain. In another aspect of the invention, one of the extracellular domain recognition units may be an antibody fragment and the other is a peptide, which may be devoid of transmembrane and signaling motifs; the peptide may be a single chain antibody fragment. In another aspect of the invention, one of the recognition units may bind PD-1 or PDL-1. In another aspect of the invention, one extra cellular domain recognition unit is an anti-MUC1* scFv chosen from the group consisting of scFv of MN-E6 antibody, scFv of MN-C2 antibody, scFv of MN-C3 antibody or scFv of MN-C8 antibody and the other is a peptide single chain antibody fragment that binds to the extra cellular domain of a cleaved MUC1 or binds to NME7 or is a peptide derived from NME7 or chosen from the group consisting of

| NME7A peptide 1 (A domain): | |
|---|---|
| MLSRKEALDFHVDHQS; | (SEQ ID NO: 7) |
| NME7A peptide 2 (A domain): | |
| SGVARTDASES; | (SEQ ID NO: 8) |
| NME7B peptide 1 (B domain): | |
| DAGFEISAMQMFNMDRVNVE; | (SEQ ID NO: 9) |
| NME7B peptide 2 (B domain): | |
| EVYKGVVTEYHDMVTE; | (SEQ ID NO: 10) |
| and | |
| NME7B peptide 3 (B domain): | |
| AIFGKTKIQNAVHCTDLPEDGLLEVQYFF. | (SEQ ID NO: 11) |

In another aspect, the invention is directed to a cell comprising a CAR with an extracellular domain that binds to the extra cellular domain of a MUC1 molecule that is devoid of tandem repeats. In another aspect, the invention is directed to a cell comprising a CAR with an extracellular domain that binds to a MUC1* transfected or transduced cell. The cell that includes the CAR may be an immune system cell, preferably a T cell, a natural killer cell (NK), a dendritic cell or mast cell.

In another aspect, the invention is directed to an engineered antibody-like protein.

In another aspect, the invention is directed to a method of screening a library of antibodies or antibody fragments that are human, for those that bind to

(i) PSMGFR peptide;
(ii) a peptide having amino acid sequence
(SEQ ID NO: 620)
SNIKFRPGSVVVQLTLAFREGTINVHDVETQFNQYKTEAASRY;
(iii) a peptide having amino acid sequence of
(SEQ ID NO: 621)
SVVVQLTLAFREGTINVHDVETQFNQYKTEAASRY;
(iv) a peptide having amino acid sequence of
(SEQ ID NO: 622)
VQLTLAFREGTINVHDVETQFNQY;
(v) a peptide having amino acid sequence of
(SEQ ID NO: 623)
SNIKFRPGSVVVQLTLAFREGTIN;

(vi) NME7 protein; or

(vii) a peptide fragment of NME7 protein.

In another aspect, the invention is directed to a method for treating a disease in a subject comprising administering an antibody according to any claim above, to a person suffering from the disease, wherein the subject expresses MUC1 aberrantly. The disease may be cancer, such as breast cancer, ovarian cancer, lung cancer, colon cancer, gastric cancer or esophageal cancer.

In another aspect, the invention is directed to a method for treating a disease in a subject comprising administering an NME peptide, to a person suffering from the disease, wherein the subject expresses MUC1 aberrantly.

In another aspect, the invention is directed to a scFv comprising variable domain fragments derived from an antibody that binds to an extracellular domain of MUC1 isoform or cleavage product that is devoid of the tandem repeat domains. The variable domain fragments may be derived from mouse monoclonal antibody MN-E6 (SEQ ID NO:13 and 66) or from the humanized MN-E6 (SEQ ID NO: 39 and 94), or from MN-E6 scFv (SEQ ID NO: 233, 235 and 237). Or, the variable domain fragments may be derived from mouse monoclonal antibody MN-C2 (SEQ ID NO: 119 and 169) or from the humanized MN-C2 (SEQ ID NO: 145 and 195), or from MN-C2 scFv (SEQ ID NO: 239, 241 and 243). Or, the variable domain fragments may be derived from mouse monoclonal antibody MN-C3 (SEQ ID NO: 414 and 459) or from the humanized MN-C3 (SEQ ID NO: 440 and 487), or from MN-C3 scFv (SEQ ID NO: 245, 247 and 249). Or, the variable domain fragments may be derived from mouse monoclonal antibody MN-C8 (SEQ ID NO: 505 and 544) or from the humanized MN-C8 (SEQ ID NO: 526 and 566), or from MN-C8 scFv (SEQ ID NO: 251, 253, 255).

In another aspect, the invention is directed to a method for the treatment of a person diagnosed with, suspected of having or at risk of developing a MUC1 or MUC1* positive cancer involving administering to the person an effective amount of the scFv described above.

In another aspect, the invention is directed to a polypeptide comprising at least two different scFv sequences, wherein one of the scFv sequences is a sequence that binds to extracellular domain of MUC1 isoform or cleavage product that is devoid of the tandem repeat domains. The polypeptide may bind to

(i) PSMGFR region of MUC1;
(ii) PSMGFR peptide;
(iii) a peptide having amino acid sequence of
(SEQ ID NO: 620)
SNIKFRPGSVVVQLTLAFREGTINVHDVETQFNQYKTEAASRY;
(iv) a peptide having amino acid sequence of
(SEQ ID NO: 621)
VQLTLAFREGTINVHDVETQFNQYKTEAASRY;
(v) a peptide having amino acid sequence of
(SEQ ID NO: 622)
VQLTLAFREGTINVHDVETQFNQY;
or
(vi) a peptide having amino acid sequence of
(SEQ ID NO: 623)
SNIKFRPGSVVVQLTLAFREGTIN.

The polypeptide may bind to a receptor on an immune cell, such as T cell, and in particular, CD3 on T-cell.

In another aspect, the invention is directed to a method of detecting presence of a cell that expresses MUC1* aberrantly, comprising contacting a sample of cells with the scFv-Fc described above and detecting for the presence of the binding of scFv-Fc to the cell. The cell may be cancer cell.

In another aspect, the invention is directed to a method for testing a subject's cancer for suitability of treatment with a composition comprising portions of the variable regions of MN-E6, MN-C2, MN-C3 or MN-C8, comprising the steps of contacting a bodily specimen from the patient with the corresponding MN-E6 scFv-Fc, MN-C2 scFv-Fc, MN-C3 scFv-Fc or MN-C8 scFv-Fc.

In another aspect, the invention is directed to a method of treating a subject suffering from a disease comprising, exposing T cells from the subject or from a donor to MUC1* peptides wherein through various rounds of maturation, T cells develop MUC1* specific receptors, creating adapted T cells, and expanding and administering the adapted T cells to the patient who is diagnosed with, suspected of having, or is at risk of developing a MUC1* positive cancer.

In one aspect, the invention may be directed to an immune cell transfected or transduced with a cleavage enzyme for the treatment of cancer. The cancer may be a MUC1 positive cancer. The immune cell may be a T cell. The immune cell may be derived from the patient to be treated. The cleavage enzyme may be an MMP or ADAM family member. The cleavage enzyme may be MMP2, MMP9, MMP3, MMP14, ADAM17, ADAM28, or ADAM TS16.

In another aspect of the invention, the cleavage enzyme is administered directly to the patient, alone or concurrent with an agent for the treatment of cancer, including but not limited to chemotherapy agents, targeted biologicals, CAR T cells, BiTEs or ADCs. In one aspect, the cleavage enzyme is MMP1, MMP2, MMP3, MMP7, MMP8, MMP9, MMP11, MMP12, MMP13, MMP14, MMP16, ADAMS, ADAM10, ADAM17, ADAM 19, ADAMTS16, ADAM28 or a catalytically active fragment thereof. In another aspect, the cleavage enzyme is MMP9 or MMP14 and the other agent for the treatment of cancer is an anti-MUC1* CAR T cell. In yet another aspect, the cleavage enzyme is MMP9 or MMP14 and the other agent for the treatment of cancer is an anti-MUC16 CAR T cell.

Another approach for the treatment of MUC1 positive cancers is to treat the patient, which may be by intra-tumor injection, with an agent that cleaves MUC1 to a form that is not recognized by the growth factors that activate MUC1*, especially growth factor NME7A_{B}. In one aspect of the invention, an immune cell is transduced with a cleavage enzyme that cleaves MUC1 at a position such that growth factors dimeric NME1 and monomeric NME7A_{B} or NME7-X1 do not bind to the remaining transmembrane portion, which is a variant of MUC1*. In some cases, the immune cell may be transduced with both a CAR and such cleavage enzyme. In yet another aspect, the cleavage enzyme that cleaves MUC1 at a position such that growth factors NME1, NME7A_{B} or NME7-X1 do not bind to the remaining transmembrane portion, is injected directly into or near the tumor site. In yet another aspect of the invention, an immune cell is transduced with a cleavage enzyme that cleaves MUC1 at a position such that growth factors NME7A_{B} or NME7-X1 do not bind to the remaining transmembrane portion, but dimeric or hexameric NME1 can bind to it. In some cases, the immune cell may be transduced with both a CAR and such cleavage enzyme. In yet another aspect, the cleavage enzyme that cleaves MUC1 at a position such that growth factors NME7A_{B} or NME7-X1 do not bind to the remaining transmembrane portion, but dimeric or hexameric NME1 can bind to it, is injected directly into or near the tumor site. In all of the instances of immune cells transduced described above, the cleavage enzyme may be expressed off of an inducible promoter. In one case its expression is induced by an NFAT protein.

One method of the invention is directed to taking a biopsy from patient tumor, identifying which MUC1 cleavage enzyme the tumor overexpresses, wherein the enzyme cleaves MUC1, and administering to the patient an immune cell transduced with a CAR wherein the antibody fragment, targeting head binds to MUC1 that has been cleaved by that enzyme. In another aspect of the invention, the patient is treated with an inhibitor of that cleavage enzyme. In one case, the immune cell, expressing a CAR that binds to the form of MUC1 that is created by cleavage by that specific enzyme, is also transduced with an inhibitor of the enzyme. In a one embodiment the inhibitor is a TIMP, which may be expressed off an inducible plasmid. In one case, it is expressed off of an NFAT inducible promoter. The cleavage enzyme inhibitor can also be an antibody that inhibits catalytic activity of the enzyme, for example an anti-MMP9 wherein the antibody is a single chain version of the antibody.

In another aspect, an immune cell is engineered to express a CAR and an inhibitor of an enzyme that cleaves MUC 1. In one aspect the immune cell is a T cell. In one aspect the inhibitor of the enzyme is a biological entity. In one case the biological inhibitor is a TIMP. In one embodiment an immune cell is transduced with a CAR whose antibody targeting head binds to a portion of the MUC 1 extracellular domain and is also transduced with an enzyme inhibitor that inhibits Cleavage of MUC1. In one case the antibody of the CAR binds to a portion on MUC1 that is N-terminal to the PSMGFR portion. In another case the antibody of the CAR binds to a region of the PSMGFR. In one case the enzyme that is inhibited is an MMP, which may be MMP9, MMP14, or MMP16 or other enzyme that cleaves MUC1 and the inhibitor is a TIMP that inhibits that enzyme. In a preferred embodiment the inhibitor is expressed off an inducible promoter. In one case it is expressed off an NFAT inducible plasmid.

In another aspect, the present invention is directed to methods of activating, stimulating expansion of, or selecting for a population of immune cells that express a receptor that recognizes a specific tumor antigen, involving contacting the immune cell expressing the cognate receptor with the antigen to which it binds. In one aspect the immune cell is an anti-MUC1* CAR T cell and the stimulating antigen is a peptide derived from the sequence of a cleaved MUC1 that is devoid of the tandem repeat domain. In one aspect, the antibody fragment that targets the CAR binds to a peptide and that peptide is used to activate, stimulate or select for the CAR T cells wherein the peptide has a sequence containing at least 15 contiguous amino acids, but can have up to 4 substitutions or deletions, wherein the peptide is derived from:

(i) PSMGFR region of MUC1;
(ii) PSMGFR peptide;
(iii) a peptide having amino acid sequence of
(SEQ ID NO: 620)
SNIKFRPGSVVVQLTLAFREGTINVHDVETQFNQYKTEAASRY;
(iv) a peptide having amino acid sequence of
(SEQ ID NO: 621)
SVVVQLTLAFREGTINVHDVETQFNQYKTEAASRY;
(v) a peptide having amino acid sequence of
(SEQ ID NO: 622)
VQLTLAFREGTINVHDVETQFNQY;
or
(vi) a peptide having amino acid sequence of
(SEQ ID NO: 623)
SNIKFRPGSVVVQLTLAFREGTIN.

In one aspect, the invention is directed to a method for training CAR T cell to be activated against a specific molecular target that is on a tumor, comprising incubating a CAR T cell, which has been transduced with nucleic acid encoding an antibody fragment that binds to the specific molecular target, with the specific molecular target. The molecular target may be any antigen specific to a tumor cell, which may be without limitation, a peptide derived from the MUC1 extra cellular domain, a peptide derived from the extra cellular domain of a transmembrane MUC1 cleavage product that is devoid of tandem repeats, a peptide derived from the extra cellular domain of MUC1*, wherein the extra cellular domain comprises PSMGFR, comprises all or part of PSMGFR plus 9 amino acids added at the N-terminus, or comprises all or part of PSMGFR plus 20 amino acids added at the N-terminus.

The specific molecular target, also known as antigen, may be in the form of:

a) antigen positive cancer cells;

b) an antigen-negative cancer cell line transfected with the antigen;

c) a substrate to which is attached a synthetic antigen or specifically reactive peptide fragment thereof;

d) free antigen or specifically reactive peptide fragment thereof; or

e) an antigen or specifically reactive peptide fragment thereof coupled to BSA but free in solution.

The incubation may be in vitro. The substrate may be magnetic bead, flask surface, cell culture dish or coated well. The antigen peptide may be attached to the substrate via bovine serum albumin or human serum albumin.

In one aspect, the antigen may be MUC1* and the specifically reactive peptide fragment may be MUC1* extra cellular domain peptide.

In one embodiment, the CAR T cells are co-cultured with the target antigen for a time period of 6 hours to 1 week. In one embodiment, the CAR T cells are co-cultured with the target antigen for a time period of 6 hours to 48 hours. In another embodiment, the CAR T cells are co-cultured with the target antigen for a time period of 12 hours to 24 hours. In another embodiment, the CAR T cells are co-cultured with the target antigen for a time period of 18 to 24 hours.

In one embodiment, the stimulated CAR T cells are administered to the patient within 4 days of the antigen stimulation. In another embodiment, the stimulated CAR T cells are administered to the patient within 2-3 days of the antigen stimulation. In still another embodiment, the stimulated CAR T cells are administered to the patient within 0-24 hours after the end of the antigen stimulation.

In one aspect, the stimulating antigen is removed from the CAR T cells prior to administration of the CAR T cell to the patient who has been diagnosed with or at risk of developing a cancer.

In one specific example, T cells are transduced with nucleic acids encoding a CAR and cultured in vitro for 8-11 days, after which the CAR T cells are cultured in the presence of the antigen peptide for a period of 18-24 hours, after which antigen peptide is removed from the CAR T cells, which are then administered to the patient within 0-48 hours after removal of the antigen peptide. In one case the patient has been diagnosed with, or at risk of developing, a cancer. In one case the cancer is a MUC1* positive cancer and the antibody fragment of the CAR binds to the peptide that is used to stimulate the CAR T cells. In a more specific example, the CAR T cells are huMNC2-CAR44 T cells and the antigen peptide is the PSMGFR peptide, or the N-10 peptide, wherein the peptide has been covalently couple to magnetic beads.

In another aspect, the invention is drawn to a method of culturing CAR T cells that produce desired memory phenotype and CD4:CD8 ratio of about 50/50, which results in persistence in vivo and low side effects when administered to a patient.

In another aspect, the invention is directed to a method of culturing a population of CAR T cells such that a greater portion of the CAR T cells are in the CD62L positive, CD45-RO negative (naïve) state and CD62L positive, CD45-RO positive (central memory) state, than CAR T cells in CD62L negative/CD45-RO positive (effector memory) and CD62L negative/CD45-RO negative effector states. The cells may be cultured in vitro for 9-12 days, 9-15 days, or 9-20 days, so as to yield a high percentage of the CAR T cells in the central memory and naïve state with far less in the effector memory and effector cell state.

In another aspect, the invention is directed to a method of treating cancer comprising administering to a cancer patient the CAR T cell prepared according to the above methods.

Thus, in one aspect, the invention is directed to a composition comprising a MUC1* extra cellular domain peptide attached to a surface and an immune cell that bears a receptor that binds to the peptide. The immune cell may be engineered to express a receptor that binds to the peptide. The MUC1* extra cellular domain peptide may be PSMGFR. The MUC1* extra cellular domain peptide may be PSMGFR with N-terminus extended by the next 9 amino acids or 20 amino acids from the MUC1* sequence. In yet another aspect, the invention is directed to a method of pre-stimulating an immune cell that bears a receptor to a cancer antigen comprising the steps of:

(i) contacting the immune cell with a peptide having a sequence derived from the cancer antigen; and

(ii) administering to a patient, diagnosed with cancer or at risk of developing a cancer, a therapeutic amount of the immune cells.

The immune cell is separated away from the peptide prior to administering to the patient. And the peptide may be free in solution, attached to a surface, attached to a bead or is expressed on a cell. The immune cell may be a CAR T cell bearing a chimeric antigen receptor (CAR) that comprises an antibody or antibody fragment that binds to the peptide. And the immune cell may be in a pool of naturally occurring T cells that may be obtained from a patient wherein the antigen is an antigen expressed by the patient's tumor, further comprising the step of expanding the patient T cells in the presence of the peptide antigen.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 28A-28C show schematics of CAR sequence components.

FIGS. 87A-87L show 4× magnification photographs of either untransduced T cells or huMNC2-scFv-CAR44 T cells subjected to either no stimulation, a 1-time bead bearing MUC1* peptide stimulation or a 2-times MUC1* positive cancer cell stimulation. FIGS. 87A-87F show the effect on untransduced T cells. FIGS. 87G-87L show the effect on huMNC2-scFv-CAR44 T cells. FIGS. 87A and 87G received no stimulation. FIGS. 87B and 87H were stimulated twice, 24 hours each stimulation with HCT-MUC1* cancer cells 24 hours prior to photography. FIGS. 87C-87F and FIGS. 87I-87L were stimulated once for 24 hours with 1 μm or 4.5 μm beads coated with the PSMGFR MUC1* extra cellular domain peptide 24 hours prior to photography.

FIGS. 88A-88D show FACS analysis of the sub-populations of human T cells transduced with huMNC2-scFv-CAR44 as a result of 1-time stimulation by co-culture with beads bearing MUC1* synthetic peptide or 3-time stimulation by co-culture with HCT-MUC1* cancer cells. FIG. 88A shows FACS scan of huMNC2-scFv-CAR44 transduced human T cells without stimulation. FIG. 88B shows FACS scan of huMNC2-scFv-CAR44 transduced human T cells with 1-time stimulation by co-culture with MUC1* peptide presenting beads. FIG. 88C shows FACS scan of huMNC2-scFv-CAR44 transduced human T cells stimulated 3-times by co-culture with HCT-MUC1* cancer cells. FIG. 88D shows graphical representation of the FACS data. FIGS. 88E-88J show graphs of FACS analysis of T cell activation markers after huMNC2-scFv-CAR44 transduced human T cells were subjected to 1-time MUC1* peptide presenting bead stimulation. FIGS. 88E-88F show FACS of activation marker CD25. FIGS. 88G-88H show FACS of activation marker CD69. FIGS. 88I-88J show FACS of activation marker Granzyme B. FIGS. 88E, 88G, 88I show FACS of huMNC2-scFv-CAR44 transduced human T cells without bead stimulation. FIGS. 88F, 88H, 88J show FACS of huMNC2-scFv-CAR44 transduced human T cells after bead stimulation.

FIGS. 89A-89C show graphs of real time CAR T induced cancer cell killing as measured on an xCELLigence instrument. The figures show the enhanced killing effect of huMNC2-scFv-CAR44 T cells after pre-stimulation by co-culture with MUC1* presenting beads. FIG. 89A shows the enhanced killing effect of peptide bead stimulated huMNC2-CAR44 T cells on SKOV-3 ovarian cancer cells wherein the ratio of T cells to cancer cells was 1:1. FIG. 89B shows the enhanced killing effect of peptide bead stimulated huMNC2-CAR44 T cells on BT-20 triple negative breast cancer cells wherein the ratio of T cells to cancer cells was 1:1. FIG. 89C shows the enhanced killing effect of peptide bead stimulated huMNC2-CAR44 T cells on HCT-MUC1* colon cancer cells wherein the ratio of T cells to cancer cells was 1:1.

FIGS. 90A-90D show graphs of real time cell growth versus cell death as measured on an xCELLigence instrument. Shown is the effect of MUC1* cancer cell stimulation of huMNC2-scFv-CAR44 transduced human T cells on a variety of cancer cells, some of which were previously resistant to CAR T cell killing. FIG. 90A shows an xCELLigence graph of the effect of huMNC2-scFv-CAR44 transduced human T cells that were pre-stimulated by co-culture with HCT-MUC1* cancer cells 24 hours prior to co-culture with the target T47D breast cancer cells. FIG. 90B shows an xCELLigence graph of the effect of huMNC2-scFv-CAR44 transduced human T cells that were pre-stimulated by co-culture with HCT-MUC1* cancer cells 24 hours prior to co-culture with the target BT-20 triple negative breast cancer cells. FIG. 90C shows an xCELLigence graph of the effect of huMNC2-scFv-CAR44 transduced human T cells that were pre-stimulated by co-culture with HCT-MUC1* cancer cells 24 hours prior to co-culture with the target SKOV-3 ovarian cancer cells. FIG. 90D shows an xCELLigence graph of the effect of huMNC2-scFv-CAR44 transduced human T cells that were pre-stimulated by co-culture with HCT-MUC1* cancer cells 24 hours prior to co-culture with the target HCT-MUC1* cancer cells that were effectively killed with or without pre-stimulation.

FIGS. 91A-91Y show fluorescent photographs of mice taken on an IVIS instrument. NSG (NOD/SCID/GAMMA) immune compromised mice that on Day 0 were sub-cutaneously implanted on the flank with 500,000 human MUC1* positive cancer cells that had been stably transfected with Luciferase. Tumors were allowed to engraft. On Day 5 after IVIS measurement and on Day 12, animals were injected with 10 million of either human T cells transduced with huMNC2-scFv-CAR44, untransduced T cells or PBS. 5 million T cells were injected intra-tumor and 5 million T cells were injected into the tail vein. 10 minutes prior to IVIS photographs, mice were injected intraperitoneally (IP) with Luciferin, which fluoresces after cleavage by Luciferase, thus making tumor cells fluoresce. FIGS. 91A, 91E, 91I, 91M, 91Q, 91U show photographs of mice that were treated with huMNC2-scFv-CAR44 T cells that had been pre-stimulated by co-culturing for 24 hours with 4 μm beads to which was attached a synthetic MUC1*, PSMGFR peptide 24 hours prior to administration: Protocol 1. FIGS. 91B, 91F, 91J, 91N, 91R, 91V show photographs of mice that were treated with huMNC2-scFv-CAR44 T cells that had been pre-stimulated by twice co-culturing for 24 hours with MUC1* positive cancer cells 24 hours prior to administration: Protocol 2. FIGS. 91C, 91G, 91K, 91O, 91S, 91W show photographs of mice that were treated with untransduced human T cells. FIGS. 91D, 91H, 91L, 91P, 91T, 91X show photographs of mice that were treated with PBS. FIGS. 91A-91D show IVIS photographs taken on Day 5 before T cell injection. FIGS. 91E-91H show IVIS photographs taken on Day 7. FIGS. 91I-91L show IVIS photographs taken on Day 11. FIGS. 91M-91P show IVIS photographs taken on Day 13. FIGS. 91Q-91T show IVIS photographs taken on Day 18. FIGS. 91U-91V show IVIS photographs taken on Day 21. Animals in untransduced T cell and PBS group had to be sacrificed on Day 20 due to excessive tumor volume. FIGS. 91W-91X show photographs of excised tumors. FIG. 91Y is a color scale relating fluorescence in photons/second to color.

FIGS. 92A-92J show fluorescent photographs of mice taken on an IVIS instrument. NSG (NOD/SCID/GAMMA) immune compromised mice that on Day 0 were sub-cutaneously injected into the flank with 500K human BT-20 cells which are a MUC1* positive triple negative breast cancer cell line. The cancer cells had been stably transfected with Luciferase. Tumors were allowed to engraft. On Day 6 after IVIS measurement, animals were given a one-time injection of 10 million of either human T cells transduced with huMNC2-scFv-CAR44 or untransduced T cells. 5 million T cells were injected intra-tumor and 5 million were injected into the tail vein. 10 minutes prior to IVIS photographs, mice were IP injected with Luciferin, which fluoresces after cleavage by Luciferase, thus making tumor cells fluoresce. FIGS. 92A, 92D, 92G show photographs of mice that were treated with huMNC2-scFv-CAR44 T cells that had been pre-stimulated by co-culturing for 24 hours with 4 μm beads to which was attached a synthetic MUC1*, PSMGFR peptide 24 hours prior to administration: Protocol 1. FIGS. 92B, 92E, 92H show photographs of mice that were treated with huMNC2-scFv-CAR44 T cells that had been pre-stimulated by twice co-culturing for 24 hours with MUC1* positive cancer cells 24 hours prior to administration: Protocol 2. FIGS. 92C, 92F, 92I show photographs of mice that were treated with untransduced human T cells. FIG. 92J is a color scale relating fluorescence in photons/second to color.

FIGS. 93A-93M show fluorescent photographs of mice taken on an IVIS instrument. NSG (NOD/SCID/GAMMA) immune compromised mice that on Day 0 were injected into the intraperitoneal cavity (IP) with 500K human SKOV-3 cells which are a MUC1* positive ovarian cancer cell line. The cancer cells had been stably transfected with Luciferase. Tumors were allowed to engraft. On Day 4, animals were injected into the intraperitoneal space with 10M either human T cells transduced with huMNC2-scFv-CAR44, untransduced T cells or PBS. On Day 11, animals were injected again except that half the cells were injected into the tail vein and the other half was IP injected. Animals were imaged by IVIS on Days 3, 7, 10 and 15. 10 minutes prior to IVIS photographs, mice were IP injected with Luciferin, which fluoresces after cleavage by Luciferase, thus making tumor cells fluoresce. FIGS. 93A, 93D, 93G, and 93J show photographs of mice that were treated with huMNC2-scFv-CAR44 T cells that had been pre-stimulated by co-culturing for 24 hours with 1 μm beads to which was attached a synthetic MUC1*, PSMGFR peptide 24 hours prior to administration. FIGS. 93B, 93E, 93H, and 93K show photographs of mice that were treated with untransduced human T cells. FIGS. 93C, 93F, 93I, and 93L show photographs of mice that were treated with PBS. FIGS. 93A, 93B and 93C are IVIS images taken Day 3 prior to CAR T, T cell or PBS administration. FIGS. 93D, 93E and 93F show IVIS images of animals on Day 7, just four (4) days after treatment. FIGS. 93G, 93H, and 93I show IVIS images of animals on Day 10. FIGS. 93J, 93K, and 93L show IVIS images of animals on Day 15 FIG. 93M is the IVIS color scale relating fluorescence in photons/second to color.

FIG. 112 shows a schematic of the timing of transducing T cells with the CAR, preferably on Day 2, expanding the CAR T cells until about Day 9, then incubating with the antigen for 24-48 hours, then either testing for killing effect or in practice, injecting into patient.

FIG. 113 shows a schematic of the variable methods for presenting antigen to CAR T cells for stimulating incubation.

FIG. 114 shows that incubating the CAR T cells, in this case anti-MUC1* CAR T cells, with a surface coated with the targeted antigen, in this case MUC1* peptide, causes the CAR T cells to bind and cluster around the antigen presenting entity, which is a visual sign of T cell activation.

FIG. 115 shows that presenting the antigen on a soluble particle, such as BSA or HSA molecules, or free in solution, has the same stimulating effect on the CAR T cells.

FIG. 116 shows that even at very low concentrations, presenting the antigen to the CAR T cells causes their activation.

FIG. 117 shows that analysis by FACS shows that incubating CAR T cells with their targeted antigen induces increased expression of T cell activation markers CD69 and CD25.

FIG. 118 shows that analysis by FACS shows that incubating CAR T cells with their targeted antigen induces increased expression of T cell activation markers CD69 and CD25.

FIG. 119 summarize visual and FACS evidence of exposure to surface-attached antigen inducing CAR T activation.

FIG. 120 shows a comparison of antigen stimulation of CAR T cells where the antigen, in this case MUC1* extra cellular domain peptide, is attached to a surface or a substrate, beads, soluble BSA or just peptide free in solution.

FIG. 121 shows that pre-stimulation of anti-MUC1* CAR T cells does not increase the killing of MUC1* negative HEK293 cells. The antigen stimulation does however increase the killing efficiency of anti-MUC1* CAR T cells killing MUC1* positive cancer cells. As can be seen in FIG. 121, the need for CAR T pre-stimulation by incubation with the target antigen is needed less when the target cancer cells express more of the target antigen.

FIG. 122 shows IVIS images of NOD/SCID/GAMMA mice implanted with human MUC1* positive tumors then treated with anti-MUC1* CAR T cells that have been pre-stimulated by incubation with either MUC1* extra cellular domain peptide attached to beads or by incubating with cancer cells of the same origin as the tumor.

FIG. 123 shows IVIS images of NOD/SCID/GAMMA mice implanted with human MUC1* positive tumors, then treated with anti-MUC1* CAR T cells. The CAR T cells were pre-stimulated in vitro by incubation with MUC1* extra cellular domain peptide attached to beads or in vivo by doping the tumor with the same tumor cells that have been modified to express even more of the target antigen, which in this case is the MUC1* transmembrane receptor.

FIG. 124 shows IVIS images of NOD/SCID/GAMMA mice implanted with human MUC1* positive tumors then treated with anti-MUC1* CAR T cells that have been pre-stimulated by incubation with MUC1* extra cellular domain peptide attached to surfaces or solid substrates, which in this case are either beads or the surface of a cell culture plate.

FIG. 125A-125D shows bar graphs that summarize FACS analysis of the CAR T cell memory sub-populations as the CAR T cells go through the in vitro manufacturing process. For the four (4) different donors shown, T cells were transduced with an anti-MUC1* CAR on Day 2 then expanded in culture until Day 9, stimulated by incubation with the target antigen for 24 hours, then washed and removed. Analysis was done by FACS on Day 10 and Day 12, since this would be the timing for patient injection. As can be seen, the protocol leaves large percentages of the T cells in the desirable Naïve and Central Memory state, which leads to persistence in vivo.

FIG. 126A-126G shows photographs and corresponding graphs that show the effect of antigen stimulation on CAR T cells in vivo. FIG. 126A-126E shows IVIS fluorescent photographs of female NOD/SCID/GAMMA mice that have been implanted with 90-day estrogen release pellets, then implanted with human MUC1* positive breast cancer cells, T47D wild type. The animals were injected on Day 5 and again on Day 12 with either PBS, untransduced human T cells, or human T cells transduced with huMNC2-CAR44 T cells that were either antigen stimulated or not. FIG. 126A shows mice injected with PBS. FIG. 126B shows mice injected with 10M untransduced human T cells. FIG. 126C shows mice injected with 7M huMNC2-CAR44 T cells that were not antigen stimulated. FIG. 126D shows mice injected with 7M huMNC2-CAR44 T cells that were previously antigen stimulated using 4.5 micron magnetic beads to which was attached synthetic PSMGFR, MUC1*, peptides at a bead to T cell ratio of 1:10. FIG. 126E shows mice injected with 7M huMNC2-CAR44 T cells that were previously antigen stimulated using 4.5 micron magnetic beads to which was attached synthetic PSMGFR, MUC1*, peptides at a bead to T cell ratio of 1:20. FIG. 126F shows a line graph summarizing tumor volume as measured by IVIS from Day 1 to Day 19 post tumor cell implantation. FIG. 126G shows a bar graph summarizing tumor volume as measured by IVIS from Day 1 to Day 19 post tumor cell implantation. Photographs were taken on an IVIS instrument that measures fluorescence emitted from the cancer cells which had been engineered to express Luciferase. Ten (10) minutes prior to IVIS analysis, the Luciferase substrate Luciferin was injected intra-peritonealy (i.p.) into the animal. As can be seen in the photos and graphs, pre-stimulation of CAR T cells with antigen greatly increased the killing efficiency of the CAR T cells. An increase in the ratio of beads to T cells resulted in increased killing.

FIG. 127A-127G shows photographs and corresponding graphs that show the effect of antigen stimulation on CAR T cells in vivo. FIG. 127A-127E shows IVIS fluorescent photographs of female NOD/SCID/GAMMA mice that have been implanted with 90-day estrogen release pellets, then implanted with human MUC1* positive breast cancer cells, T47D-30%. T47D-30% cells are a mixed population where 70% are T47D wild type breast cancer cells and 30% are T47D cells that have been stably transduced with even more MUC1*. The animals were injected on Day 5 and again on Day 12 with either PBS, untransduced human T cells, or human T cells transduced with huMNC2-CAR44 T cells that were either antigen stimulated or not. FIG. 127A shows mice injected with PBS. FIG. 127B shows mice injected with 10M untransduced human T cells. FIG. 127C shows mice injected with 7M huMNC2-CAR44 T cells that were not antigen stimulated. FIG. 127D shows mice injected with 7M huMNC2-CAR44 T cells that were previously antigen stimulated using 4.5 micron magnetic beads to which was attached synthetic PSMGFR, MUC1*, peptides at a bead to T cell ratio of 1:10. FIG. 127E shows mice injected with 7M huMNC2-CAR44 T cells that were previously antigen stimulated using 4.5 micron magnetic beads to which was attached synthetic PSMGFR, MUC1*, peptides at a bead to T cell ratio of 1:20. FIG. 127F shows a line graph summarizing tumor volume as measured by IVIS from Day 1 to Day 19 post tumor cell implantation. FIG. 127G shows a bar graph summarizing tumor volume as measured by IVIS from Day 1 to Day 19 post tumor cell implantation. Photographs were taken on an IVIS instrument that measures fluorescence emitted from the cancer cells which had been engineered to express Luciferase. Ten (10) minutes prior to IVIS analysis, the Luciferase substrate Luciferin was injected intra-peritonealy (i.p.) into the animal. As can be seen in the photos and graphs, the beneficial effect of antigen stimulation of CAR T cells with antigen is less dramatic when the cancer cells express high levels of MUC1*.

Table 1 shows details of many of the anti-MUC1* CARs that were generated and tested. For each construct shown, a number assigned to that CAR, promoter used, signal peptide, antibody species, sequences of scFv, hinge region, transmembrane domain, and signaling motifs used in each CAR, length of the insert in number of base pairs, its molecular weight and the length of the construct are displayed.

Table 2 shows cytokine release data for some of the CARs after transduction into human T cells and co-cultured with a variety of cancer cells.

### DETAILED DESCRIPTION OF THE PREFERRED EMBODIMENTS

In the present application, “a” and “an” are used to refer to both single and a plurality of objects.

As used herein, occasionally, in short hand, a polypeptide is indicated as being “transduced or transfected” into a cell. In these occurrences, it is understood that the nucleic acid encoding the polypeptide sequence is transduced or transfected into the cell, as it is an impossibility that a polypeptide could be transduced or transfected into a cell.

As used herein, occasionally when referring to number of cells injected into an animal or otherwise contextually wherein the number of cells is referred to, “M” refers to millions, and “K” refers to thousands.

As used herein, interchangeable designations for various monoclonal antibodies are used, such as, “MN-C2”, which is interchangeable with “C2”, “Min-C2” and “MNC2”; “MN-E6”, which is interchangeable with “E6”, “Min-E6” and “MNE6”; “MN-C3”, which is interchangeable with “C3”, “Min-C3” and “MNC3”; and “MN-C8”, which is interchangeable with “C8”, “Min-C8” and “MNC8”.

As used herein, “h” or “hu” placed before an antibody construct is short-hand for humanized.

As used herein, the term “antibody-like” means a molecule that may be engineered such that it contains portions of antibodies but is not an antibody that would naturally occur in nature. Examples include but are not limited to CAR (chimeric antigen receptor) T cell technology and the Ylanthia® technology. The CAR technology uses an antibody epitope fused to a portion of a T cell so that the body's immune system is directed to attack a specific target protein or cell. The Ylanthia® technology consists of an “antibody-like” library that is a collection of synthetic human Fabs that are then screened for binding to peptide epitopes from target proteins. The selected Fab regions can then be engineered into a scaffold or framework so that they resemble antibodies.

As used herein, “PSMGFR” is abbreviation for Primary Sequence of the MUC1 Growth Factor Receptor which is identified by SEQ ID NO:2, and thus is not to be confused with a six amino acid sequence. “PSMGFR peptide” or “PSMGFR region” refers to a peptide or region that incorporates the Primary Sequence of the MUC1 Growth Factor Receptor (SEQ ID NO:2).

As used herein, the “MUC1*” extra cellular domain is defined primarily by the PSMGFR sequence (GTINVHDVETQFNQYKTEAASRYNLTISDVSVSDVPFPFSAQSGA (SEQ ID NO:2)). Because the exact site of MUC1 cleavage depends on the enzyme that clips it, and that the cleavage enzyme varies depending on cell type, tissue type or the time in the evolution of the cell, the exact sequence of the MUC1* extra cellular domain may vary at the N-terminus.

Other clipped amino acid sequences may include SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621).

As used herein, the term “PSMGFR” is an acronym for Primary Sequence of MUC1 Growth Factor Receptor as set forth as GTINVHDVETQFNQYKTEAASRYNLTISDVSVSDVPFPFSAQSGA (SEQ ID NO:2). In this regard, the “N-number” as in “N-10 PSMGFR”, “N-15 PSMGFR”, or “N-20 PSMGFR” refers to the number of amino acid residues that have been deleted at the N-terminal end of PSMGFR. Likewise “C-number” as in “C-10 PSMGFR”, “C-15 PSMGFR”, or “C-20 PSMGFR” refers to the number of amino acid residues that have been deleted at the C-terminal end of PSMGFR.

As used herein, the “extracellular domain of MUC1*” refers to the extracellular portion of a MUC1 protein that is devoid of the tandem repeat domain. In most cases, MUC1* is a cleavage product wherein the MUC1* portion consists of a short extracellular domain devoid of tandem repeats, a transmembrane domain and a cytoplasmic tail. The precise location of cleavage of MUC1 is not known perhaps because it appears that it can be cleaved by more than one enzyme. The extracellular domain of MUC1* will include most of the PSMGFR sequence but may have an additional 10-20 N-terminal amino acids.

As used herein “sequence identity” means homology in sequence of a particular polypeptide or nucleic acid to a reference sequence of nucleic acid or amino acid such that the function of the homologous peptide is the same as the reference peptide or nucleic acid. Such homology can be so close with the reference peptide such that at times the two sequences may be 90%, 95% or 98% identical yet possess the same function in binding or other biological activities.

As used herein, “MUC1 positive” cell refers to a cell that expresses a gene for MUC1, MUC1-Y or MUC1-Z or other MUC1 variant.

As used herein, “MUC1 negative” cell refers to a cell that does not express a gene for MUC1.

As used herein, “MUC1* positive” cell refers to a cell that expresses a gene for MUC1, wherein that gene's expressed protein is a transmembrane protein that is devoid of tandem repeats, which may be a consequence of post-translational modification, cleavage, alternative splicing, or transfecting or transducing a cell with a MUC1 protein that is devoid of tandem repeats.

As used herein, “MUC1* negative” cell refers to a cell that may or may not express a gene for MUC1 but does not express a MUC1 transmembrane protein that is devoid of tandem repeats.

As used herein, “MUC1 positive” cancer cell refers to a cancer cell that overexpresses the gene for MUC1, expresses MUC1 in an aberrant pattern, wherein its expression is not restricted to the apical border and/or expresses a MUC1 that is devoid of tandem repeats.

As used herein, “MUC1 negative” cancer cell refers to a cancer cell that may or may not express a gene for MUC1 but does not overexpress MUC1 or does not overexpress a MUC1 transmembrane protein that is devoid of tandem repeats.

As used herein, “MUC1* positive” cancer cell refers to a cancer cell that overexpresses a MUC1 transmembrane protein that is devoid of tandem repeats.

As used herein, “MUC1* negative” cancer cell refers to a cancer cell that may or may not express a gene for MUC1 but does not overexpress a MUC1 transmembrane protein that is devoid of tandem repeats.

As used herein, “activation”, “stimulation” and “pre-stimulation” of CAR T cells have similar meaning and its use may vary according to context. Activation, stimulation or pre-stimulation of CAR T cell with its cognate antigen means presenting or incubating the CAR T cell with the antigen, which by the resultant activation, stimulation or pre-stimulation of the CAR T cell results in greater overall cancer cell destroying capability of the CAR T than if such presenting or incubating of the antigen had not occurred.

As used herein, a “surface” in the context of the CAR T cell being contacted with an antigen on a surface, the surface can be a solid substrate, porous substrate or a non-solid substrate. Such surface may include without limitation glass, plastic or any suitable substrate to which can be attached a peptide. Coated wells and bottom of flask bottles are some examples of such a substrate.

MUC1* Antibodies (Anti-PSMGFR) for Treatment or Prevention of Cancers

We discovered that a cleaved form of the MUC1 (SEQ ID NO:1) transmembrane protein is a growth factor receptor that drives the growth of over 75% of all human cancers. The cleaved form of MUC1, which we called MUC1* (pronounced muk 1 star), is a powerful growth factor receptor. Enzymatic cleavage releases the bulk of the MUC1 extracellular domain. It is the remaining portion comprising a truncated extracellular domain, transmembrane domain and cytoplasmic tail that is called MUC1*. Cleavage and release of the bulk of the extracellular domain of MUC1 unmasks a binding site for activating ligands dimeric NME1, NME6, NME8, NME7-AB, NME7-X1 or NME7. Cell growth assays show that it is ligand-induced dimerization of the MUC1* extracellular domain that promotes growth. MUC1* positive cells treated with either bivalent ‘by’ anti-MUC1* antibody, monovalent ‘my’ or Fab, NM23-H1 dimers or NME7-AB. Bivalent anti-MUC1* antibodies stimulate growth of cancer cells whereas the monovalent Fab inhibits growth. Classic bell-shaped curve indicates ligand induced dimerization stimulates growth. Dimeric NM23-H1, aka NME1, stimulates growth of MUC1* positive cancer cells but siRNA to suppress MUC1 expression eliminate its effect. NME7-AB also stimulates the growth of MUC1* positive cells.

MUC1* is an excellent target for cancer drugs as it is aberrantly expressed on over 75% of all cancers and is likely overexpressed on an even higher percentage of metastatic cancers. After MUC1 cleavage, most of its extracellular domain is shed from the cell surface. The remaining portion has a truncated extracellular domain that at least comprises the primary growth factor receptor sequence, PSMGFR (SEQ ID NO:2). Antibodies that bind to the PSMGFR sequence and especially those that competitively inhibit the binding of activating ligands such as NME proteins, including NME1, NME6, NME8, NME7AB, NME7-X1 and NME7, are ideal therapeutics and can be used to treat or prevent MUC1 positive or MUC1* positive cancers, as stand-alone antibodies, antibody fragments or variable region fragments thereof incorporated into bispecific antibodies, or chimeric antigen receptors also called CARs, which are then transfected or transduced into immune cells, then administered to a patient.

Therapeutic anti-MUC1* antibodies can be monoclonal, polyclonal, antibody mimics, engineered antibody-like molecules, full antibodies or antibody fragments. Examples of antibody fragments include but are not limited to Fabs, scFv, and scFv-Fc. Human or humanized antibodies are preferred for use in the treatment or prevention of cancers. In any of these antibody-like molecules, mutations can be introduced to prevent or minimize dimer formation. Anti-MUC1* antibodies that are monovalent or bispecific are preferred because MUC1* function is activated by ligand induced dimerization. Typical binding assays show that NME1 and NME7-AB bind to the PSMGFR peptide portion of MUC1*. Further, they show that these activating growth factors bind to the membrane proximal portion of MUC1*, as they do not bind to the PSMGFR peptide if the 10 C-terminal amino acids are missing. Similarly, anti-MUC1* antibodies MN-C2 and MN-E6 bind to the PSMGFR peptide if an only if the 10 C-terminal amino acids are present. Antibodies MN-C3 and MN-C8 bind to epitopes that are different from MN-C2 and MN-E6, as they do not depend on the presence of the 10 C-terminal amino acids of the PSMGFR peptide. Antibodies MN-C2, MN-E6, MN-C3 or MN-C8, or fragments derived from them, can be administered to a patient for the treatment or prevention of cancers, as stand-alone antibodies or incorporated into bispecific antibodies, BiTEs or chimeric antigen receptors also called CARs that have been transduced into immune cells. MNC2 and MNE6 and other anti-MUC1* antibodies that competitively inhibit the binding of NME1 and NME7-AB are preferred for use as stand alone antibody therapeutics.

Therapeutic anti-MUC1* antibodies for use as a stand alone antibody therapeutic or for integration into a BiTE or a CAR can be selected based on specific criteria. The parent antibody can be generated using typical methods for generating monoclonal antibodies in animals. Alternatively, they can be selected by screening antibody and antibody fragment libraries for their ability to bind to a MUC1* peptide, which can be the PSMGFR peptide (SEQ ID NO:2), SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621).

Resultant antibodies or antibody fragments generated or selected in this way can then be further selected by passing additional screens. For example, antibodies or antibody fragments become more preferred based on their ability to bind to MUC1* positive cancer cells or tissues but not to MUC1 negative cancer cells or to normal tissues. Further, anti-MUC1* antibodies or antibody fragments may be de-selected as anti-cancer therapeutics if they bind to stem or progenitor cells. Anti-MUC1* antibodies or antibody fragments become more preferred if they have the ability to competitively inhibit the binding of activating ligands to MUC1*. MN-E6 and MN-C2 competitively inhibit the binding of activating ligands NME1 and NME7 to MUC1*.

A human or humanized MN-E6 antibody or antibody fragment, Fab, MN-E6 scFv or hu MN-E6 scFv-Fcₘᵤₜ are effective anti-cancer agents that can be administered to a person diagnosed with a MUC1 or MUC1* positive cancer, suspected of having a MUC1 or MUC1* positive cancer or is at risk of developing a MUC1 or MUC1* positive cancer.

Antibodies that bind to PSMGFR (SEQ ID NO:2) peptide of the extracellular domain of the MUC1* receptor are potent anti-cancer therapeutics that are effective for the treatment or prevention of MUC1* positive cancers. They have been shown to inhibit the binding of activating ligands dimeric NME1 (SEQ ID NOS: 3 and 4) and NME7 (SEQ ID NOS: 5 and 6) to the extracellular domain of MUC1*. Anti-MUC1* antibodies that bind to the PSMGFR sequence inhibit the growth of MUC1*-positive cancer cells, specifically if they inhibit ligand-induced receptor dimerization. Fabs of anti-MUC1* antibodies have been demonstrated to block tumor growth in animals. Thus, antibodies or antibody fragments that bind to the extracellular domain of MUC1* would be beneficial for the treatment of cancers wherein the cancerous tissues express MUC1*.

CAR T and Cancer Immunotherapy Techniques

In another aspect of the invention, some or all of the single chain portions of anti-MUC1* antibody fragments are biochemically fused onto immune system molecules, using several different chimeric antigen receptor, ‘CAR’ strategies. The idea is to fuse the recognition portion of an antibody, typically as a single chain variable fragment, to an immune system molecule that has a transmembrane domain and a cytoplasmic tail that is able to transmit signals that activate the immune system. The recognition unit can be an antibody fragment, a single chain variable fragment, scFv, or a peptide. In one aspect, the recognition portion of the extracellular domain of the CAR is comprised of sequences from the humanized variable region of MN-E6 (SEQ ID NOS: 38-39 and 93-94), MN-C2 (SEQ ID NOS: 144-145 and 194-195), MN-C3 (SEQ ID NOS: 439-440 and 486-487) and MN-C8 (SEQ ID NOS: 525-526 and 565-566). In another aspect, it is comprised of sequences from a single chain variable fragment. Examples of single chain constructs are given. Several humanized MN-E6 single chain proteins, scFv, were generated (SEQ ID NOS: 232-237). Several humanized MN-C2 single chain proteins, scFv, were generated (SEQ ID NOS: 238-243). Several humanized MN-C3 single chain proteins, scFv, were generated (SEQ ID NOS: 244-249). Several humanized MN-C8 single chain proteins, scFv, were generated (SEQ ID NOS: 250-255). The transmembrane region of the CAR can be derived from CD8, CD4, antibody domains or other transmembrane region, including the transmembrane region of the proximal cytoplasmic co-stimulatory domain, such as CD28, 4-1BB or other. The cytoplasmic tail of the CAR can be comprised of one or more motifs that signal immune system activation. This group of cytoplasmic signaling motifs, sometimes referred to as, co-stimulatory cytoplasmic domains, includes but is not limited to CD3-zeta, CD27, CD28, 4-1BB, OX40, CD30, CD40, ICAm-1, LFA-1, ICOS, CD2, CD5, CD7 and Fc receptor gamma domain. A minimal CAR may have the CD3-zeta or an Fc receptor gamma domain then one or two of the above domains in tandem on the cytoplasmic tail. In one aspect, the cytoplasmic tail comprises CD3-zeta, CD28, 4-1BB and/or OX40.

Table 1 lists many of the anti-MUC1* CARs that we generated and tested. Several examples of MN-E6 CARs were generated: CAR MN-E6 CD3z (SEQ ID NOS: 294-295); CAR MN-E6 CD28/CD3z (SEQ ID NOS: 297-298); CAR MN-E6 4-1BB/CD3z (SEQ ID NOS: 300-301); CAR MN-E6 OX40/CD3z (SEQ ID NOS: 616-617); CAR MN-E6 CD28/OX40/CD3z (SEQ ID NOS: 618-619); CAR MN-E6 CD28/4-1BB/CD3z (SEQ ID NOS: 303-304). Several examples of humanized MN-C2 CARs were generated: CAR MN-C2 CD3z (SEQ ID NOS: 606-607); CAR MN-C2 CD28/CD3z (SEQ ID NOS: 608-609); CAR MN-C2 4-1BB/CD3z (SEQ ID NOS: 610-611); CAR MN-C2 OX40/CD3z (SEQ ID NOS: 612-613); CAR MN-C2 CD28/4-1BB/CD3z (SEQ ID NOS: 306-307); CAR MN-C2 CD28/OX40/CD3z (SEQ ID NOS: 614-615). Humanized MN-C3 CAR was generated: CAR MN-C3 4-1BB/CD3z (SEQ ID NOS: 600-601).

Several examples of humanized MN-E6 CARs with different hinge regions (SEQ ID NOS:345-360) were generated: CAR MN-E6-Fc/8/41BB/CD3z (SEQ ID NOS:310-311); CAR MN-E6 FcH/8/41BB/CD3z (SEQ ID NOS:315-316); CAR MN-E6 Fc/4/41BB/CD3z (SEQ ID NOS:318-319); CAR MN-E6 FcH/4/41BB/CD3z (SEQ ID NOS:321-322); CAR MN-E6 IgD/8/41BB/CD3z (SEQ ID NOS:323-324); CAR MN-E6 IgD/4/41BB/CD3z (SEQ ID NOS:327-328); CAR MN-E6 X4/8/41BB/CD3z (SEQ ID NOS:330-331); CAR MN-E6 X4/4/41BB/CD3z (SEQ ID NOS:333-334); CAR MN-E6 8+4/4/41BB/CD3z (SEQ ID NOS:336-337). In addition, several humanized MN-C3 single chain variable fragment and humanized MN-C8 single chain variable fragments were also generated.

Several CARs were also generated and tested wherein the targeting head of the CAR was derived from the anti-MUC1* antibody MNC2. CAR MN-C2-Fc/41BB/CD3z (SEQ ID NOS:732-733); CAR-MN-C2 IgD/Fc/4-1BB/CD3z (SEQ ID NOS:734-735); CAR MN-C2 FcH/41BB/CD3z (SEQ ID NOS:736-737); CAR-MN-C2 IgD/FcH/4-1BB/CD3z (SEQ ID NOS:738-739); CAR MN-C2 IgD/41BB/CD3z (SEQ ID NOS:740-741); CAR MN-C2 X4/41BB/CD3z (SEQ ID NOS:742-743).

The extracellular domain recognition unit of a MUC1* targeting CAR can comprise variable regions of any non-human, humanized or human antibody that is able to bind to at least 12 contiguous amino acids of the PSMGFR peptide (SEQ ID NO:2). In one aspect, the MUC1* targeting portion of the CAR comprises variable regions from non-human, humanized or human MN-E6, MN-C2, MN-C3 or MN-C8. In one aspect, the extracellular domain recognition unit of a CAR is comprised essentially of a humanized MN-E6, MN-C2, MN-C3 or MN-C8 single chain variable fragment scFv. The transmembrane region of the CAR can be derived from CD8 (SEQ ID NOS:363-364), or can be the transmembrane domain of CD3-zeta, CD28, 41bb, OX40 or other transmembrane region (SEQ ID NOS:361-372) and the cytoplasmic domain of a CAR with antibody fragment targeting MUC1* extracellular domain can be comprised of one or more selected from the group comprising an immune system co-stimulatory cytoplasmic domain. The group of immune system co-stimulatory domains includes but is not limited to CD3-zeta, CD27, CD28, 4-1BB, OX40, CD30, CD40, ICAm-1, LFA-1, ICOS, CD2, CD5, CD7 and Fc receptor gamma domain (SEQ ID NOS:373-382). Alternatively, the recognition unit portion of a CAR can comprise a peptide wherein the peptide binds to the target. NME7 binds to and activates MUC1*. In one aspect of the invention, the recognition unit of a CAR is a peptide derived from NME7 (SEQ ID NOS: 5-6) or a peptide derived from NME7, including but not limited to NME7 peptide A1 (SEQ ID NO: 7), NME7 peptide A2 (SEQ ID NO: 8), NME7 peptide B1 (SEQ ID NO: 9), NME7 peptide B2 (SEQ ID NO: 10) and NME7 peptide B3 (SEQ ID NO: 11).

Some strategies for generating CARs include a portion of the molecule that dimerizes with itself. In some cases, dimerization of the target is not desirable. Therefore, CARs can be constructed such that they heterodimerize. In one case the recognition unit of the first CAR binds to a first target while the recognition unit of the second CAR binds to a second target. Both recognition units can be antibody fragments, both can be peptides or one can be an antibody fragment and the other a peptide. A first target of the CAR can be the extracellular domain of MUC1*. The recognition unit of the CAR would be comprised of an antibody fragment that binds to MUC1* extracellular domain or to a PSMGFR peptide. Alternatively, the recognition unit of the CAR would be comprised of a peptide that binds to MUC1* extracellular domain, such peptides include peptides derived from an NME protein such as NME1 or NME7, more particularly NME7 derived peptides listed as SEQ ID NOS: 7-11. A second target of a heterodimeric CAR may be a peptide or antibody fragment that binds to NME7. Alternatively, a second target of a heterodimeric CAR may be a peptide or antibody fragment that binds to PD1 or its cognate ligand PDL-1 or other target ligand of the target cancer cell. A second target may be a peptide or antibody fragment that binds to NME1 or NME7-AB. Because it is desirable to prevent dimerization of MUC1 induced by a CAR, heterodimeric CARs can be constructed so that only the extracellular domain of one molecule has an extracellular recognition unit that binds to a target (SEQ ID NOS:584-587). The other molecule can have a truncated extracellular domain that is devoid of a target recognition unit or antibody fragment (SEQ ID NOS:588-599).

The CARs described can be transfected or transduced into a cell of the immune system. In a preferred embodiment, a MUC1* targeting CAR is transfected or transduced into a T cell. In one aspect, the T cell is a CD3+/CD28+ T cell. In another case it is a dendritic cell. In another case it is a B cell. In another case it is a mast cell. The recipient cell can be from a patient or from a donor. If from a donor, it can be engineered to remove molecules that would trigger rejection. Cells transfected or transduced with a CAR of the invention can be expanded ex vivo or in vitro then administered to a patient. Administrative routes are chosen from a group containing but not limited to bone marrow transplant, intravenous injection, in situ injection or transplant. In a preferred embodiment, the MUC1* targeting CAR is administered to a person diagnosed with or at risk of developing a MUC1-positive cancer.

There are many possible anti-MUC1* CAR constructs that can be transduced into T cells or other immune cells for the treatment or prevention of MUC1* positive cancers. CARs are made up of modules and the identity of some of the modules is relatively unimportant, while the identity of other modules is critically important.

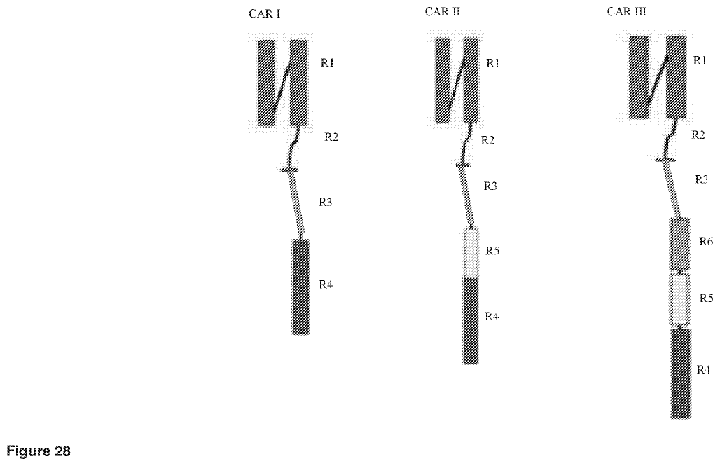

Our experiments demonstrate that the antibody recognition fragment at the outermost portion of the CAR is critically important because it targets the immune cell bearing the CAR to the tumor site. The intracellular signaling motifs are also very important but can be interchanged. FIG. 28 shows a schematic of the components of CAR and the various sequences that may be included in a CAR. Referring to FIG. 28,

R1 is: nothing; or

a ligand or a fragment of a ligand of a cancer associated antigen; or

a ligand or a fragment of a ligand of MUC1 or MUC1*; or

an antibody or antibody fragment wherein the antibody or antibody fragment binds to MUC1 or MUC1*; or an antibody or antibody fragment wherein the antibody or antibody fragment binds to PSMGFR*, wherein the antibody may be human or humanized; or an antibody or antibody fragment of MN-E6, MN-C2, MN-C3 or MN-C8 or humanized MN-E6, MN-C2, MN-C3 or MN-C8; or a single chain variable fragment of an antibody, scFv, that binds to a cleaved MUC1 or MUC1*; or a scFv of MN-E6, MN-C2, MN-C3 or MN-C8, which may be humanized; or a peptide that binds to MUC1* or PSMGFR peptide; or is an antibody fragment, a scFv, or a peptide that binds the PSMGFR portion of MUC1*; or is comprised of sequence from the humanized variable region of MN-E6 (SEQ ID NOS: 38-39 and 93-94), MN-C2 (SEQ ID NOS: 144-145 and 194-195), MN-C3 (SEQ ID NOS: 439-440 and 486-487) and MN-C8 (SEQ ID NOS: 525-526 and 565-566). In one aspect, R1 is a scFv that binds the PSMGFR portion of MUC1* comprised of sequence from humanized MN-E6 scFv (SEQ ID NOS: 232-237), humanized MN-C2 scFv (SEQ ID NOS: 238-243), humanized MN-C3 scFv (SEQ ID NOS: 244-249) or humanized MN-C8 scFv (SEQ ID NOS: 250-255). In another aspect, R1 is a scFv that binds the PSMGFR portion of MUC1* comprised of sequence from humanized MN-E6 scFv (SEQ ID NOS: 232-237) or humanized MN-C2 scFv (SEQ ID NOS: 238-243). In one example R1 is a scFv that binds the PSMGFR portion of MUC1* comprised of sequence from humanized MN-E6 scFv (SEQ ID NOS: 232-237)

R2 is a polypeptide flexible linker that connects the recognition portion to the transmembrane domain of the CAR. In one aspect, R2 can be a polypeptide linker of different length from 5 to 250 amino acids. In another aspect, R2 is a polypeptide linker of human origin. In one aspect, R2 can be made of or a modification of the Fc region of a human immunoglobulin (IgG, IgA, IgE, IgM or IgD). I another aspect, R2 can be the hinge region or a modification of the hinge region of a human immunoglobulin (IgG, IgA, IgE, IgM or IgD). In one aspect, R2 can be the hinge region or a modification of the hinge region of a T-cell receptor (CD8a, CD28 or CD4). In one example, R2 is the hinge region of CD8a, the hinge region of human IgD or the Fc domain of human IgG1.

R3 is a transmembrane domain. In one aspect, R3 can be a transmembrane domain or a modification of a transmembrane domain of any transmembrane human proteins. In another aspect, R3 can be a transmembrane domain or a modification of a transmembrane domain from human cell receptor. In one aspect, R3 can be a transmembrane domain or a modification of a transmembrane domain of a T-cell receptor (CD8a, CD4, CD28, CD3z, OX40 or 41-BB). In another aspect, R3 is a transmembrane domain from the first cytoplasmic co-stimulatory domain of the CAR. In one aspect, R3 can be a transmembrane domain or a modification of a transmembrane domain of a T-cell receptor extended with 1, 2, 3, 4 or 5 amino acids of the cytoplasmic domain associated to the transmembrane domain. In another aspect, R3 can be a transmembrane domain or a modification of a transmembrane domain of a T-cell receptor extended with 1, 2, 3, 4 or five amino acids of the cytoplasmic domain associated to the transmembrane domain followed by a cystein for disulfide bond formation. In one example, R3 is the transmembrane domain of CD8a or CD4.

R4 is a signaling domain from a T-cell receptor. In one aspect, R4 can be the cytoplasmic signaling domain of CD3-zeta, CD27, CD28, 4-1BB, OX40, CD30, CD40, ICAm-1, LFA-1, ICOS, CD2, CD5, CD7 and Fc receptor gamma domain. In one example, R4 is the cytoplasmic domain of CD3-zeta. Several examples of humanized CAR with single signaling domain (CAR I) were regenerated: CAR MN-E6 CD3z (SEQ ID NOS: 294-295); CAR MN-C2 CD3z (SEQ ID NOS: 606-607)

R5 is a co-stimulatory domain from a T-cell receptor. In one aspect, R5 can be the cytoplasmic signaling domain of CD27, CD28, 4-1BB, OX40, CD30, CD40, ICAm-1, LFA-1, ICOS, CD2, CD5, CD7 and Fc receptor gamma domain. R5 will be different from R4 and R6. In one example, R5 is the cytoplasmic domain of CD28, 4-1BB or OX40. Several examples of humanized CAR with two signaling domain (CAR II) were regenerated: CAR MN-E6 CD28/CD3z (SEQ ID NOS: 297-298); CAR MN-E6 4-1BB/CD3z (SEQ ID NOS: 300-301); CAR MN-E6 OX40/CD3z (SEQ ID NOS: 616-617); CAR MN-C2 CD28/CD3z (SEQ ID NOS: 608-609); CAR MN-C2 4-1BB/CD3z (SEQ ID NOS: 610-611); CAR MN-C2 OX40/CD3z (SEQ ID NOS: 612-613); MN-C3 4-1BB/CD3z (SEQ ID NOS: 600-601); CAR MN-E6-Fc/8/41BB/CD3z (SEQ ID NOS:310-311); CAR MN-E6 FcH/8/41BB/CD3z (SEQ ID NOS:315-316); CAR MN-E6 Fc/4/41BB/CD3z (SEQ ID NOS:318-319); CAR MN-E6 FcH/4/41BB/CD3z (SEQ ID NOS:321-322); CAR MN-E6 IgD/8/41BB/CD3z (SEQ ID NOS:323-324); CAR MN-E6 IgD/4/41BB/CD3z (SEQ ID NOS:327-328); CAR MN-E6 X4/8/41BB/CD3z (SEQ ID NOS:330-331); CAR MN-E6 X4/4/41BB/CD3z (SEQ ID NOS:333-334); CAR MN-E6 8+4/4/41BB/CD3z (SEQ ID NOS:336-337).

R6 is a co-stimulatory domain from a T-cell receptor. In one aspect, R6 can be the cytoplasmic signaling domain of CD27, CD28, 4-1BB, OX40, CD30, CD40, ICAm-1, LFA-1, ICOS, CD2, CD5, CD7 and Fc receptor gamma domain. R6 will be different from R4 and R5. In one example, R5 is the cytoplasmic domain of CD28. Several examples of humanized CAR with two signaling domain (CAR III) were regenerated: CAR MN-E6 CD28/OX40/CD3z (SEQ ID NOS: 618-619); CAR MN-E6 CD28/4-1BB/CD3z (SEQ ID NOS: 303-304); CAR MN-C2 CD28/4-1BB/CD3z (SEQ ID NOS: 306-307); CAR MN-C2 CD28/OX40/CD3z (SEQ ID NOS: 614-615)

We and others have shown that intracellular signaling modules, such as CD3-zeta (SEQ ID NOS: 373-376), CD28 (SEQ ID NOS: 377-378) and 41BB (SEQ ID NOS: 379-380), alone or in combinations stimulate immune cell expansion, cytokine secretion and immune cell mediated killing of the targeted tumor cells (Pule M A, Straathof K C, Dotti G, Heslop H E, Rooney C M and Brenner M K (2005) A chimeric T cell antigen receptor that augments cytokine release and supports clonal expansion of primary human T cells. Mol Ther. 12(5):933-941; Hombach A A, Heiders J, Foppe M, Chmielewski M and Abken H. (2012) OX40 costimulation by a chimeric antigen receptor abrogates CD28 and IL-2 induced IL-10 secretion by redirected CD4(+) T cells. Oncoimmunology. 1(4):458-466; Kowolik C M, Topp M S, Gonzalez S, Pfeiffer T, Olivares S, Gonzalez N, Smith D D, Forman S J, Jensen M C and Cooper L J. (2006) CD28 costimulation provided through a CD19-specific chimeric antigen receptor enhances in vivo persistence and antitumor efficacy of adoptively transferred T cells. Cancer Res. 66(22):10995-11004; Loskog A, Giandomenico V, Rossig C, Pule M, Dotti G and Brenner M K. (2006) Addition of the CD28 signaling domain to chimeric T-cell receptors enhances chimeric T-cell resistance to T regulatory cells. Leukemia. 20(10):1819-1828; Milone M C, Fish J D, Carpenito C, Carroll R G, Binder G K, Teachey D, Samanta M, Lakhal M, Gloss B, Danet-Desnoyers G, Campana D, Riley J L, Grupp S A and June C H. (2009) Chimeric receptors containing CD137 signal transduction domains mediate enhanced survival of T cells and increased antileukemic efficacy in vivo. Mol Ther. 17(8):1453-1464; Song D G, Ye Q, Carpenito C, Poussin M, Wang L P, Ji C, Figini M, June C H, Coukos G, Powell D J Jr. (2011) In vivo persistence, tumor localization, and antitumor activity of CAR-engineered T cells is enhanced by costimulatory signaling through CD137 (4-1BB). Cancer Res. 71(13):4617-4627). Less important is the identity of the short extracellular piece that presents the antibody fragment, the transmembrane domain, and the short cytoplasmic tail that comes before the intracellular signaling motifs.

The identity of the recognition antibody fragment that targets the CAR to a tumor is critically important. For the treatment of MUC1 positive or MUC1* positive cancers, that antibody recognition fragment must bind to the extracellular domain of portion of MUC1 that remains after cleavage and shedding of the bulk of the extracellular domain, which contains the tandem repeat domains. In one aspect of the invention, the portion that remains comprises the PSMGFR sequence. In another aspect of the invention, the portion of MUC1 that remains after cleavage and shedding contains the PSMGFR sequence plus up to nine (9) more amino acids extended at the N-terminus. In another aspect of the invention, the portion of MUC1 that remains after cleavage and shedding contains the PSMGFR sequence plus up to twenty one (21) more amino acids extended at the N-terminus. In one aspect, the antibody recognition fragment binds to at least twelve contiguous amino acids of a PSMGFR peptide. In another aspect of the invention, the antibody recognition fragment binds to a peptide comprising the sequence SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621).

As a demonstration, a single chain antibody fragment that included the variable domain of the monoclonal anti-MUC1* antibodies called MN-E6 or MN-C2 were engineered into a panel of CARs (Table 1). The MUC1* targeting CARs were then transduced, separately or in combinations, into immune cells. When challenged with surfaces presenting a MUC1* peptide, an antigen presenting cell transfected with MUC1*, or MUC1* positive cancer cells, the immune cells that were transduced with MUC1* targeting CARs elicited immune responses, including cytokine release, killing of the targeted cells and expansion of the immune cells (Table 2).

The identity of molecules that make up the non-targeting portions of the CAR such as the extracellular domain, transmembrane domain and membrane proximal portion of the cytoplasmic domain, are not essential to the function of a MUC1*-targeting CAR. For example, the extracellular domain, transmembrane domain and membrane proximal portion of the cytoplasmic domain can be comprised of portions of CD8, CD4, CD28, or generic antibody domains such as Fc, CH2CH3, or CH3. Further, the non-targeting portions of a CAR can be a composite of portions of one or more of these molecules or other family members.

One aspect of the invention is a method for treating a patient diagnosed with, suspected of having, or at risk of developing a MUC1 positive or MUC1* positive cancer, wherein the patient is administered an effective amount of immune cells that have been transduced with a MUC1* targeting CAR. In another aspect of the invention, the immune cells are T cells isolated from a patient, which are then transduced with CARs wherein the targeting head of the CAR binds to MUC1*, and after expansion of transduced T cells, the CAR T cells are administered in an effective amount to the patient. In yet another aspect of the invention, the immune cells are T cells isolated from a patient or a donor, which are then transduced with CARs wherein the targeting head of the CAR comprises portions of huMN-E6, huMN-C2, huMN-C3 or huMN-C8, and after optional expansion of transduced T cells, the CAR T cells are administered in an effective amount to the patient. In yet another aspect of the invention, the CAR that is transduced into the immune cell and administered to the patient diagnosed with a MUC1 or MUC1* positive cancer is chosen from the list of CARs in Table 1 or Table 2.

Specifics of CARs Made and Tested

One aspect of the invention is a method for treating a patient diagnosed with, suspected of having, or at risk of developing a MUC1 positive or MUC1* positive cancer, wherein a specimen is obtained from the patient's cancer and is tested for reactivity with an antibody that binds to PSMGFR SEQ ID NO:2, SNIKFRPGSVVVQLTLAFREGTINVHDVETQFNQYKTEAASRY (SEQ ID NO:620) or SVVVQLTLAFREGTINVHDVETQFNQYKTEAASRY (SEQ ID NO:621). The patient is then treated with an scFv, scFv-Fc or CAR T that comprises antibody variable fragments from the antibody that reacted with their cancer specimen. Another aspect of the invention is a method for treating a patient diagnosed with, suspected of having, or at risk of developing a MUC1 positive or MUC1* positive cancer, wherein a specimen is obtained from the patient's cancer and is tested for reactivity with MN-E6-scFv, MN-C2-scFv, MN-C3-scFv or MN-C8-scFv; the patient is then treated with the scFv, scFv-Fc-mut or CAR T that comprises portions of the antibody that reacted with their cancer specimen.

We discovered that MUC1 can be cleaved to MUC1* by more than one cleavage enzyme and that the site of cleavage affects its fold and consequently affects which monoclonal antibody is able to recognize that form of MUC1*. Different cancer cells or cancerous tissues express different cleavage enzymes. We tested various cleavage enzyme inhibitors on different cancer cell lines and found that an inhibitor that inhibits cleavage of MUC1 in one cancer cell line did not inhibit its cleavage in another cancer cell line. Similarly, PCR experiments showed that cleavage enzymes are expressed at different levels in different cells or cell lines. For example, hematopoietic stem cells of the bone marrow express a MUC1* that is recognized by monoclonal antibody MNC3 but not MNE6 or MNC2. The growth of DU145 prostate cancer cells and T47D breast cancer cells is inhibited by the Fabs of MNC2 and MNE6 but not by the Fabs of MNC3 or MNC8, indicating that the cancer cell lines express a MUC1* that is recognized by MNE6 and MNC2 but not by MNC3 or MNC8. PCR experiments show that CD34 positive cells of the bone marrow express about 2,500-times more MMP2 and about 350-times more ADAM28 than T47D breast cancer cells, while DU145 prostate cancer cells express about 2,000-times more ADAM TS16, about 400-times more MMP14 and about 100-times more MMP1 than T47D breast cancer cells. Conversely, T47D breast cancer cells express about 80-times more MMP9 than the bone marrow cells and about twice as much as DU145 prostate cancer cells. Various cleavage enzyme inhibitors were tested for their ability to inhibit cleavage in different kinds of cancer cells. TAPI-1 that inhibits MMP2, MMP9, and ADAM17 and MMP2/9 V inhibitor that inhibits MMP2, MMP9, MMP14, inhibited the cleavage of MUC1 in T47D breast cancer cells, but none of the cleavage enzyme inhibitors tested had an effect in DU145 prostate cancer cells.

BiTEs

Divalent (or bivalent) single-chain variable fragments (di-scFvs, bi-scFvs) can be engineered by linking two scFvs. This can be done by producing a single peptide chain with two V_{H} and two V_{L} regions, yielding tandem scFvs. Another possibility is the creation of scFvs with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Consequently, diabody drugs could be dosed much lower than other therapeutic antibodies and are capable of highly specific targeting of tumors in vivo. Still shorter linkers (one or two amino acids) lead to the formation of trimers, so-called triabodies or tribodies. Tetrabodies have also been produced. They exhibit an even higher affinity to their targets than diabodies.

All of these formats can be composed from variable fragments with specificity for two different antigens, in which case they are types of bispecific antibodies. The furthest developed of these are bispecific tandem di-scFvs, known as hi-specific T-cell engagers (BiTE antibody, constructs). BiTEs are fusion proteins consisting of two says of different antibodies, on a single peptide chain of about 55 kilodaltons. One of the scFvs may bind to T cells such as via the CD3 receptor, and the other to a tumor cell via a tumor specific molecule, such aberrantly expressed MUC1*.

Another aspect of the invention is a method for treating a patient diagnosed with, suspected of having, or at risk of developing a MUC1 positive or MUC1* positive cancer, wherein the patient is administered an effective amount of a BiTE wherein one antibody variable fragment of the BiTE binds to a T cell surface antigen and the other antibody variable fragment of the BiTE binds to PSMGFR SEQ ID NO:2, SNIKFRPGSVVVQLTLAFREGTINVHDVETQFNQYKTEAASRY (SEQ ID NO:620) or SVVVQLTLAFREGTINVHDVETQFNQYKTEAASRY (SEQ ID NO:621). In one case, the antibody variable fragment of the BiTE that binds to MUC1* comprises portions of huMN-E6, huMN-C2, huMN-C3, or huMN-C8.

In another aspect of the invention, MUC1* peptides including PSMGFR SEQ ID NO:2, most or all of SNIKFRPGSVVVQLTLAFREGTINVHDVETQFNQYKTEAASRY (SEQ ID NO:620) or SVVVQLTLAFREGTINVHDVETQFNQYKTEAASRY (SEQ ID NO:621) are used in adoptive T cell approaches. In this case, a patient's T cells are exposed to the MUC1* peptides and through various rounds of maturation, the T cells develop MUC1* specific receptors. The adapted T cells are then expanded and administered to the donor patient who is diagnosed with, suspected of having, or is at risk of developing a MUC1* positive cancer.

A series of CARs were also made that had MNC2 and humanized MNC2 as the extra cellular, targeting head of the CAR. The constructs for these CARs were inserted into a plasmid that was then inserted into a Lenti viral vector. Human T cells were then transduced with the lenti viral vector carrying the MNC2 CARs and huMNC2 CARs. MNC2-scFv-CARs that were mouse sequence or humanized were generated. CARs comprising MNC2-scFv and a variety of transmembrane and intracellular co-stimulatory domains were generated including constructs listed in Table 1. In one aspect of the invention, the CAR comprised huMNC2-scFv-short hinge region-transmembrane domain derived from CD8-short intracellular piece-4-1BB-3zeta. In another aspect, the transmembrane domain was derived from CD4 transmembrane sequence. In another aspect, the intracellular co-stimulatory domain was CD28-3zeta. In yet another aspect, the intracellular co-stimulatory domain was CD28-4-1BB-3zeta.

In addition to FACS analysis, many researchers now use an xCELLigence instrument to measure CAR T killing of cancer cells. FACS is not the best method for tracking T cell induced cell killing because the T cells lyse the target cell. By FACS it is difficult to measure dead cells because they are excluded as cell debris, so one must infer an amount of cell killing and by various methods determine if the missing cells are T cells or cancer cells.

The xCELLigence instrument uses electrode arrays upon which cancer cells are plated. The adherent cancer cells insulate the electrode and so cause an increase in impedance as they grow. Conversely, T cells are not adherent and remain in suspension so do not contribute to insulation of the electrode which would increase impedance. However, if the T cells or CAR T cells kill the cancer cells on the electrode plate, the cancer cells ball up and float off as they die, which causes the impedance to decrease. The xCELLigence instrument measures impedance as a function of time, which is correlated to cancer cell killing. In addition, the electrode plates also have a viewing window. When CAR T cells effectively kill the adsorbed target cancer cells, there is a decrease in impedance but also one can see that there are no cancer cells left on the plate surface.

Pre-Activation of CAR T Cells

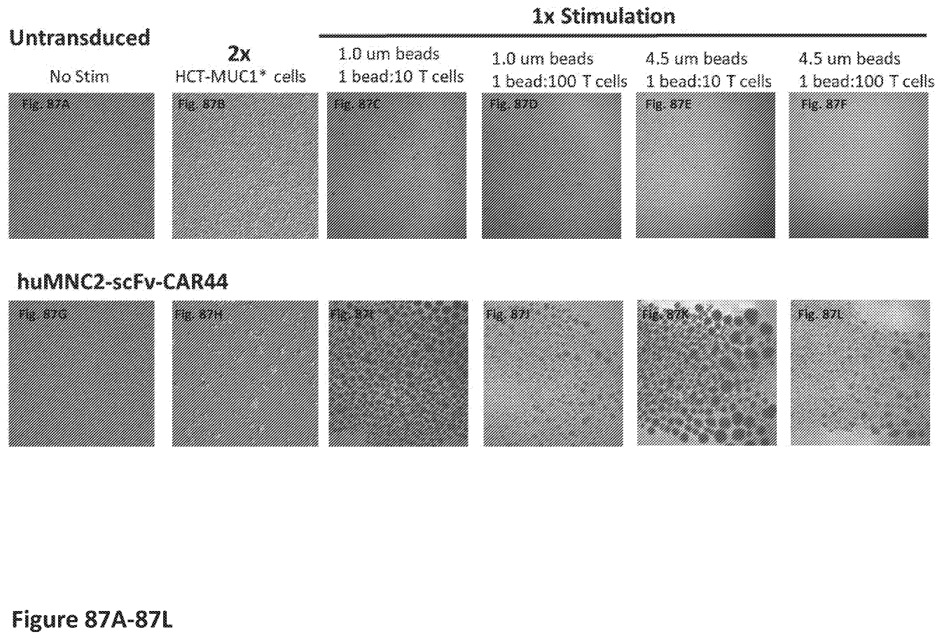

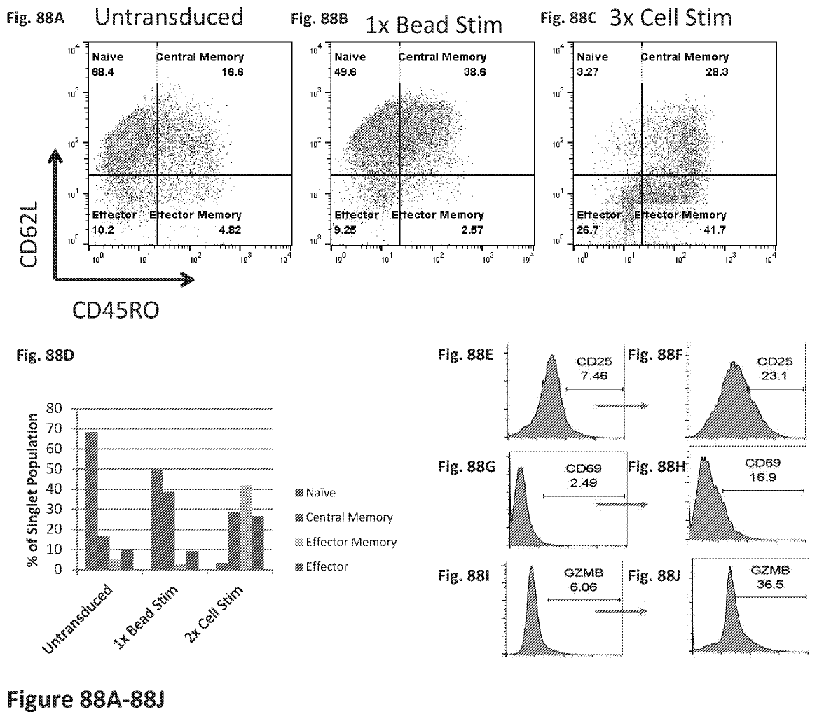

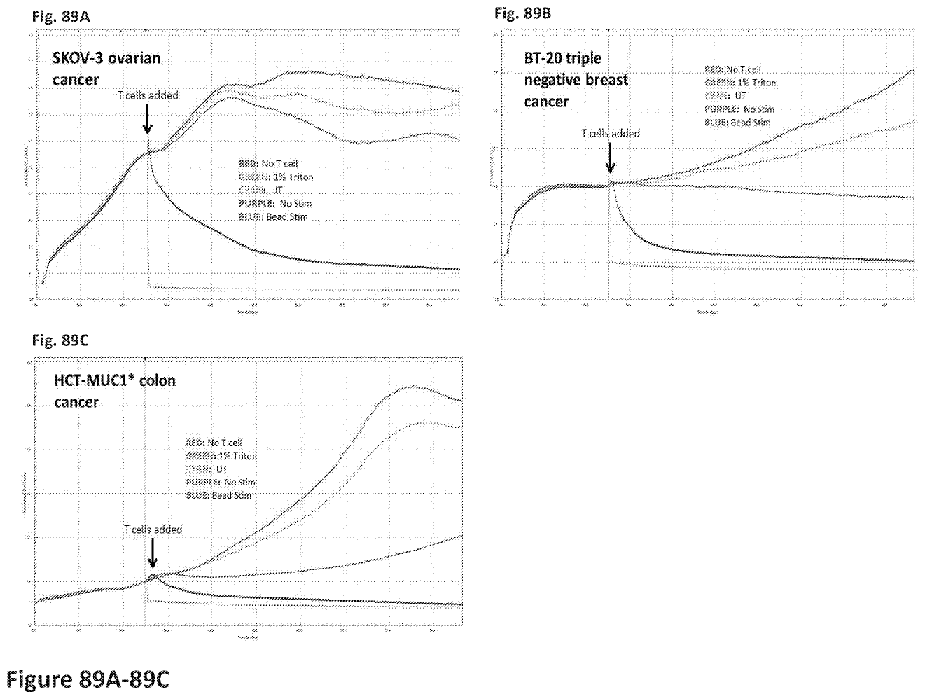

Experiments were also performed exploring methods of pre-activating the CAR T cells to more effectively kill the target cancer cells. We first tested pre-stimulation of the CAR T cells using beads presenting anti-CD3 and anti-CD28 antibodies. This pre-stimulation increased the amount of cell killing but the increase was not specific for the target of the CAR. Rather, the CD3-CD28 stimulated CAR T cells non-specifically killed MUC1* positive and negative cells. We next tried pre-stimulating the CAR T cells with either beads or cancer cells that expressed the target of the antibody portion of the CAR. A synthetic MUC1*extra cellular domain peptide was attached to either 1 μm or 4.5 μm beads. Anti-MUC1* CAR T cells were incubated with the peptide presenting beads for 12-24 hours. FIGS. 87A-87L show the untransduced T cells or the CAR T cells after 24 hour incubation with MUC1* peptide presenting beads. As can be seen, only the CAR transduced T cells show activation-induced clustering. The CAR T cells were separated from the beads by centrifugation, then analyzed by FACS to measure expression of T cell activation markers CD25, CD69 and granzyme B. As can be seen in FIGS. 88A-88D, T cell activation markers increase after incubation with MUC1* presenting beads if and only if the T cell had been transduced with a CAR whose extra cellular domain comprised an anti-MUC1* antibody fragment. In sharp contrast to pre-activation with CD3-CD28 beads, stimulation with MUC1* peptide beads only increased specific killing. There was no increase in the killing of MUC1* negative cells. FIGS. 89A-89C show xCELLigence scans that show the enhanced killing of bead-stimulated anti-MUC1* CAR T cells on human ovarian cancer cells, triple negative breast cancer cells and a MUC1 negative colon cancer cell line that was stably transfected with MUC1*. The enhanced killing capability of MUC1* peptide bead stimulated CAR T cells enabled the CAR T cells to effectively kill target cancer cells for longer periods of time and at much lower T cell to cancer cell ratios. In one aspect of the invention, CAR T cells are pre-stimulated by incubation with beads or surfaces that present a peptide derived from the MUC1* extra cellular domain, before administering to a patient diagnosed with or at risk of developing a MUC1* positive cancer.

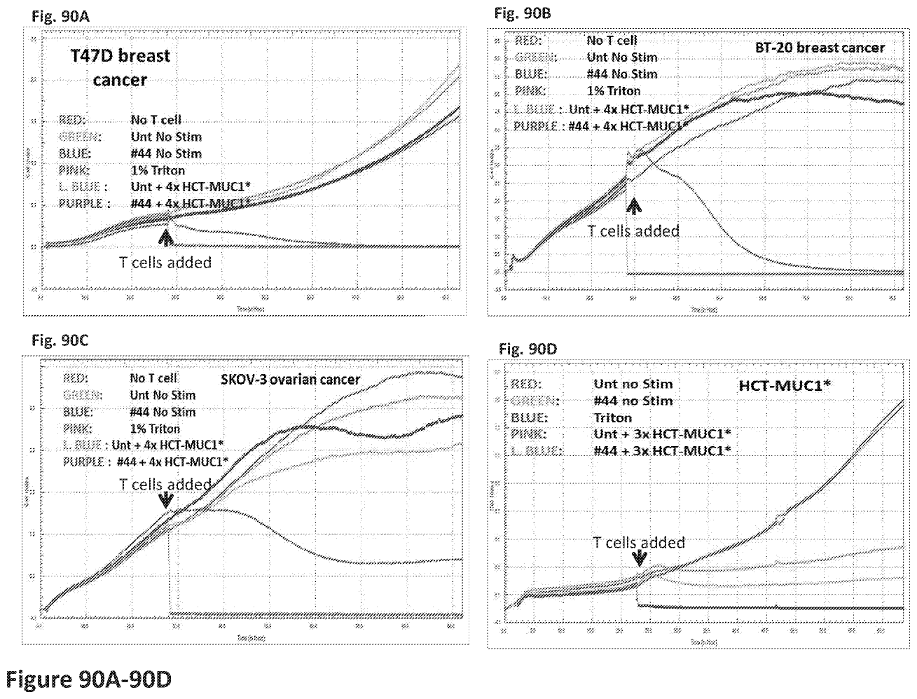

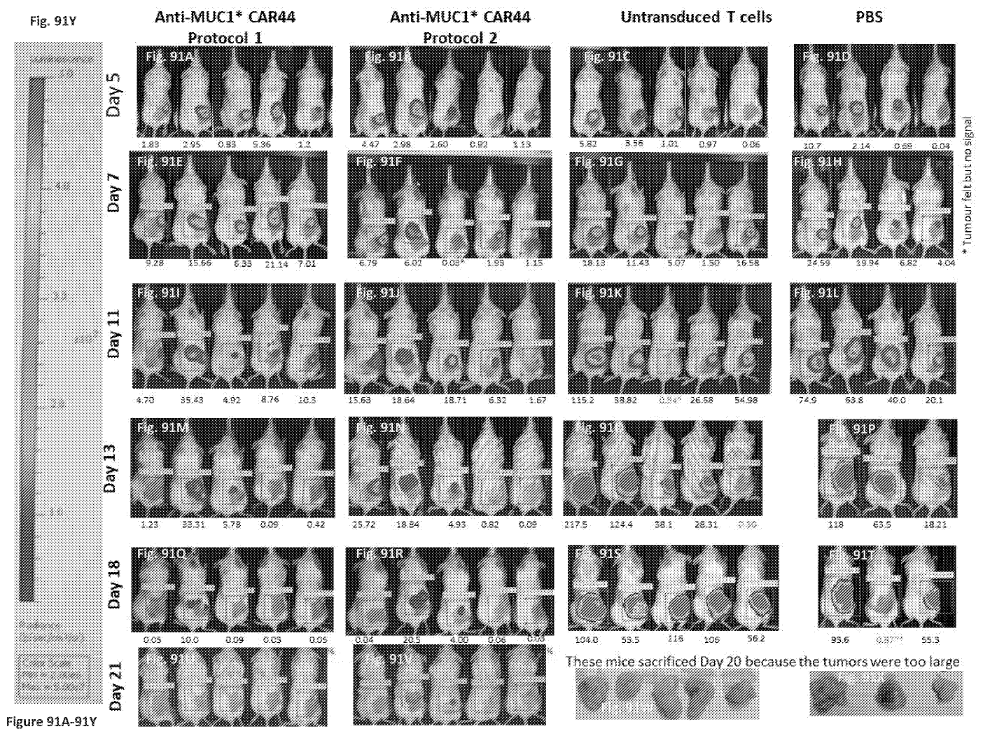

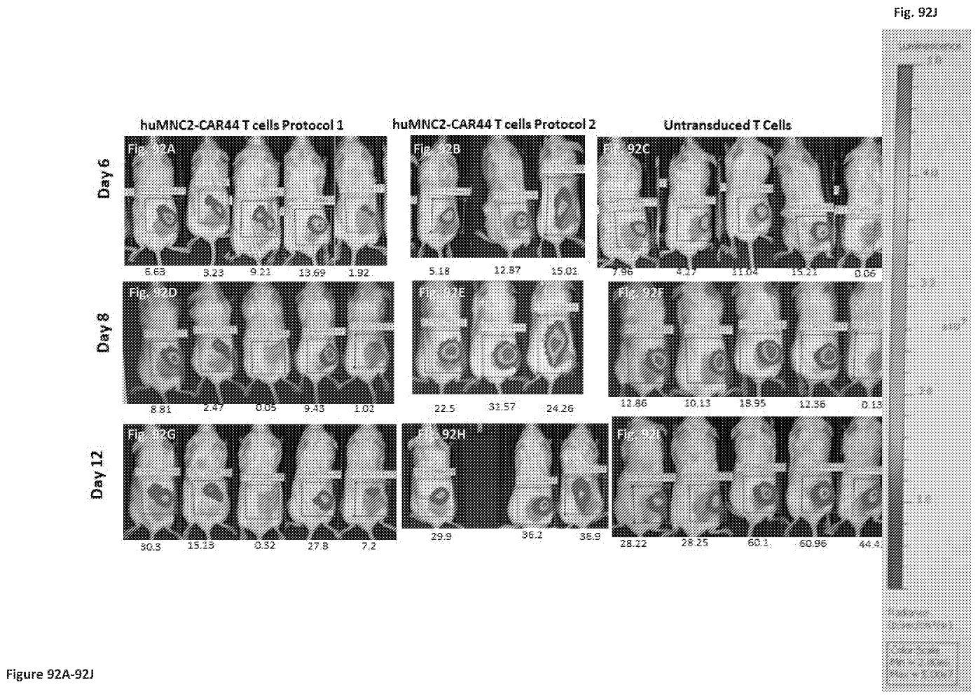

We also tested pre-activating CAR T cells by incubating them with cancer cells that present the target antigen. We incubated huMNC2-CAR44 T cells with HCT-MUC1* cells for 12-24 hours. This pre-stimulation was done once, twice, three or four times. Target cell pre-stimulation also greatly enhanced the specific killing of CAR T cells. As can be seen in FIGS. 90A-90D, specific cell killing by the cancer cell stimulated CAR T cells increased their killing potential even at low CAR T to cancer cell ratios and for longer periods of time. FIGS. 90A-90D show cancer cell stimulated huMNC2-scFv-CAR44 transduced human T cells effectively kill T47D breast cancer cells, BT-20 triple negative breast cancer cells, SKOV-3 ovarian cancer cells and HCT-MUC1* cancer cells. In one aspect of the invention, CAR T cells are pre-stimulated by incubation with MUC1* expressing cells, which may be cancer cells, before administering to a patient diagnosed with or at risk of developing a MUC1* positive cancer. In a preferred embodiment, the MUC1* stimulation cells are UV or chemically inactivated before co-culture with the CAR T cells.

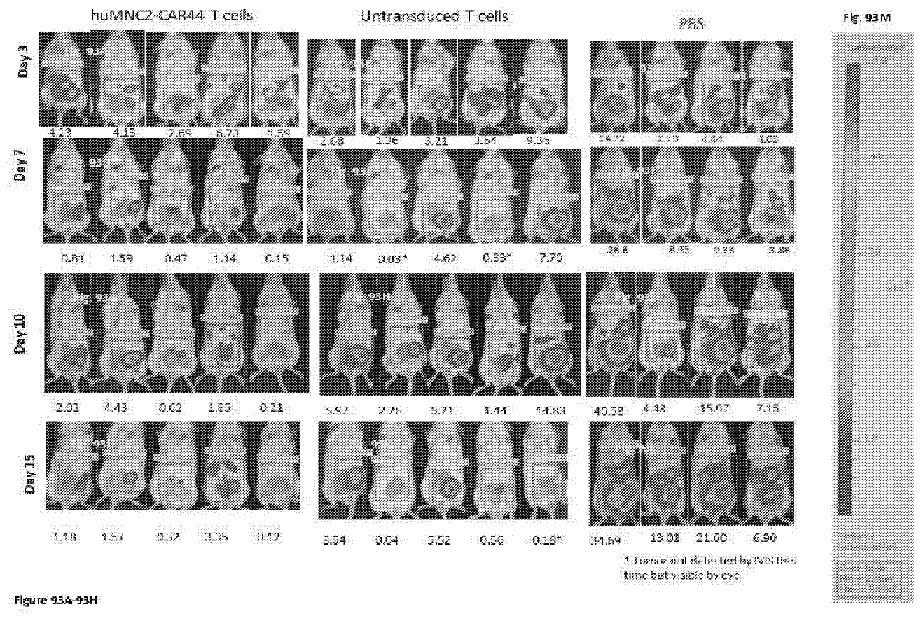

huMNC2-scFv-CAR44 transduced human T cell that were bead stimulated (Protocol 1) or cancer cell stimulated (Protocol 2) were tested for their ability to inhibit tumor growth in animals. Human cancer cells that had been stably transfected with Luciferase were injected into female NOD/SCID/GAMMA (NSG) mice between 11 and 15 weeks of age. In one experiment, 500,000 HCT-MUC1* cancer cells were injected sub-cutaneously into a rear flank. Tumor engraftment was verified by injecting the animals with Luciferin and then imaging the fluorescent cancer cells using an IVIS instrument. IVIS images taken Day 5 post implantation showed the presence of tumor cells. On Day 6 and on Day 12, 10M huMNC2-scFv-CAR44 T cells were administered to the animals. 5M of the CAR T cells were administered by intratumor injection and the other 5M were administered by tail vein injection. Control groups were injected by same administration routes with either the same number of untransduced T cells or same volume of PBS. IVIS measurements of tumor burden were taken on Days 7, 11, 13, and 21. As can be seen in FIGS. 91A-91Y, both groups of control mice had tumors that continuously grew, whereas the mice treated with bead-stimulated huMNC2-scFv-CAR44 T cells have no detectable cancer cells by Day 21. Three (3) of the five (5) mice treated with cancer cell-stimulated huMNC2-scFv-CAR44 T cells have no detectable cancer cells by Day 21. The other two (2) mice have a barely detectable number of cancer cells remaining by Day 21.

huMNC2-scFv-CAR44 transduced human T cell that were bead stimulated (Protocol 1) or cancer cell stimulated (Protocol 2) were also tested for their ability to inhibit tumor growth in animals. Human cancer cells that had been stably transfected with Luciferase were injected into female NOD/SCID/GAMMA (NSG) mice between 11 and 15 weeks of age. In another experiment, 500,000 BT-20 MUC1* positive triple negative breast cancer cells were injected sub-cutaneously into a rear flank. Tumor engraftment was verified by injecting the animals with Luciferin and then imaging the fluorescent cancer cells using an IVIS instrument. IVIS images taken Day 6 post implantation showed the presence of tumor cells. On Day 6, after IVIS imaging, 10M huMNC2-scFv-CAR44 T cells were administered to the animals. 5M of the CAR T cells were administered by intratumor injection and the other 5M were administered by tail vein injection. Control group was injected by same administration routes with the same number of untransduced T cells. IVIS measurements of tumor burden were taken on Days 6, 8, and 12. As can be seen in FIGS. 92A-92J, both groups of mice treated with huMNC2-CAR44 T cells showed a decrease in tumor burden compared to the control group.

huMNC2-scFv-CAR44 transduced human T cell that were bead stimulated (Protocol 1) were also tested for their ability to inhibit ovarian cancer growth in animals. Human SKOV-3 MUC1* positive ovarian cancer cells that had been stably transfected with Luciferase were injected into female NOD/SCID/GAMMA (NSG) mice between 11 and 15 weeks of age. In one experiment, 500,000 SKOV-3 cancer cells were injected into the intraperitoneal cavity to mimic metastatic ovarian cancer in humans. Tumor engraftment was verified by injecting the animals with Luciferin and then imaging the fluorescent cancer cells using an IVIS instrument. IVIS images taken Day 3 post implantation showed the presence of tumor cells. On Day 4 and Day 11, post tumor implantation, 10M huMNC2-scFv-CAR44 T cells were IP administered to the animals. On Day 4, CAR T cells were IP injected. On Day 11 half the CAR T cells were injected into the intraperitoneal space and the other half was injected into the tail vein. Control groups were injected by same administration routes with either the same number of untransduced T cells or same volume of PBS. Subsequent IVIS measurements of tumor burden were taken on Day 7, Day 10 and Day 15. As can be seen in FIGS. 93A-93L, control mice have tumors that are growing at a much faster rate than the huMNC2-CAR44 T cell treated mice. FIG. 93M shows the IVIS color bar correlating photons/second to color.

One aspect of the invention is a method for treating a patient diagnosed with, suspected of having, or at risk of developing a MUC1 positive or MUC1* positive cancer, wherein the patient is administered an effective amount of immune cells that have been transduced with a MUC1* targeting CAR, wherein the CAR is chosen from among the group consisting of MN-E6-CD8-3z (SEQ ID NOS:294-295); MN-E6-CD4-3z (SEQ ID NOS:746-747); MN-E6-CD8-CD28-3z (SEQ ID NOS:297-298); MN-E6-CD4-CD28-3z (SEQ ID NOS:748-749); MN-E6-CD8-41BB-3z (SEQ ID NOS:300-301); MN-E6-CD4-41BB-3z (SEQ ID NOS:750-751); MN-E6-CD8-CD28-41BB-3z (SEQ ID NOS:303-304); MN-E6-CD4-CD28-41BB-3z (SEQ ID NOS:754-755); MN-E6scFv-Fc-8-41BB-CD3z (SEQ ID NOS:310-311); MN-E6scFv-IgD-Fc-8-41BB-CD3z (SEQ ID NOS:770-771); MN-E6scFv-FcH-8-41BB-CD3z (SEQ ID NOS:315-316); MN-E6scFv-IgD-FcH-8-41BB-CD3z (SEQ ID NOS:772-773); MN-E6scFv-Fc-4-41BB-CD3z (SEQ ID NOS:318-319); MN-E6scFv-FcH-4-41BB-CD3z (SEQ ID NOS:321-322); MN-E6scFv-IgD-8-41BB-CD3z (SEQ ID NOS:323-324); MN-E6scFv-IgD-4-41BB-CD3z (SEQ ID NOS:327-328); MN-E6scFv-X4-8-41BB-CD3z (SEQ ID NOS:330-331); MN-E6scFv-X4-4-41BB-CD3z (SEQ ID NOS:333-334); MN-E6scFv-8-4-41BB-CD3z (SEQ ID NOS:336-337), or any of the aforementioned CARs wherein the MN-E6 is replaced by MN-C2, MN-C3 or MN-C8; MN-C2-CD8-3z (SEQ ID NOS:606-607); MN-C2-CD4-3z (SEQ ID NOS:758-759); MN-C2-CD8-CD28-3z (SEQ ID NOS:608-609); MN-C2-CD4-CD28-3z (SEQ ID NOS:760-761); MN-C2-CD8-41BB-3z (SEQ ID NOS:610-611 and SEQ ID NOS:718-719); MN-C2-CD4-41BB-3z (SEQ ID NOS:762-763); MN-C2-CD8-CD28-41BB-3z (SEQ ID NOS:306-307); MN-C2-CD4-CD28-41BB-3z (SEQ ID NOS:766-767); MN-C2-Fc-8-41BB-CD3z (SEQ ID NOS:732-733); MN-C2-IgD-Fc-8-41BB-CD3z (SEQ ID NOS:734-735); MN-C2-FcH-8-41BB-CD3z (SEQ ID NOS:736-737); MN-C2-IgD-FcH-8-41BB-CD3z (SEQ ID NOS:738-739); MN-C2-IgD-8-41BB-CD3z (SEQ ID NOS:740-741); MN-C2-X4-8-41BB-CD3z (SEQ ID NOS:742-743). Another aspect of the invention is a method for treating a patient diagnosed with, suspected of having, or at risk of developing a cancer, wherein the patient is administered an effective amount of immune cells that have been transduced with one of the aforementioned CARs wherein the MN-E6 is replaced by a peptide comprising antibody variable domain fragments that are specific for a cancer antigen. In any of the above methods, the immune cell may be a T cell and may further be isolated from the patient to be treated.

Other MUC1 Cleavage Sites

It is known that MUC1 is cleaved to the growth factor receptor form, MUC1*, on some healthy cells in addition to cancer cells. For example, MUC1 is cleaved to MUC1* on healthy stem and progenitor cells. A large percentage of bone marrow cells are MUC1* positive. Portions of the intestine are MUC1* positive.

The inventors have discovered that MUC1 can be cleaved at different positions that are relatively close to each other but the location of cleavage changes the fold of the remaining portion of the extracellular domain. As a result, monoclonal antibodies can be identified that bind to MUC1* cleaved at a first position but do not bind to MUC1* that has been cleaved at a second position. This discovery is disclosed in WO2014/028668, filed Aug. 14, 2013, the contents of which are incorporated by reference herein its entirety. We identified a set of anti-MUC1* monoclonal antibodies that bind to MUC1* as it appears on cancer cells but do not bind to MUC1* as it appears on stem and progenitor cells. Conversely, we identified a second set of monoclonal antibodies that bind to stem and progenitor cells but do not bind to cancer cells. One method used to identify stem specific antibodies is as follows: supernatants from monoclonal hybridomas were separately adsorbed onto 2 multi-well plates. Stem cells, which are non-adherent cells, were put into one plate and cancer cells which are adherent were put into an identical plate. After an incubation period, the plates were rinsed and inverted. If the non-adherent stem cells stuck to the plate, then the monoclonal antibody in that particular well recognizes stem cells and will not recognize cancer cells. Antibodies that did not capture stem cells or antibodies that captured cancer cells were identified as cancer specific antibodies. FACS analysis has confirmed this method works.

Antibodies MN-E6 and MN-C2 are examples of cancer-specific antibodies. Antibodies MN-C3 and MN-C8 are examples of stem-specific antibodies. Although both sets of antibodies are able to bind to a peptide having the PSMGFR sequence, FACS analysis shows that the anti-MUC1* polyclonal antibody and MN-C3 bind to MUC1* positive bone marrow cells but MN-E6 does not. The MUC1* polyclonal antibody was generated by immunizing a rabbit with the PSMGFR peptide. Similarly, MN-C3 binds to stem cells of the intestinal crypts but MN-E6 does not. Conversely, MN-E6 antibody binds to cancerous tissue while the stem-specific MN-C3 does not. Competition ELISA experiments indicate that the C-terminal 10 amino acids of the PSMGFR peptide are required for MN-E6 and MN-C2 binding, but not for MN-C3 and MN-C8. Therefore, another method for identifying antibodies that are cancer specific is to immunize with a peptide having the sequence of the PSMGFR peptide minus the 10 N-terminal amino acids or use that peptide to screen for antibodies or antibody fragments that will be cancer specific. Antibodies that bind to a peptide with a sequence of PSMGFR peptide minus the N-terminal 10 amino acids but do not bind to a peptide with a sequence of PSMGFR peptide minus the C-terminal 10 amino acids are cancer specific antibodies for use in the treatment or prevention of cancers.

The extracellular domain of MUC1 is also cleaved on stem cells and some progenitor cells, where activation of cleaved MUC1 by ligands NME1 in dimer form or NME7 promotes growth and pluripotency and inhibits differentiation. The transmembrane portion of MUC1 that remains after cleavage is called MUC1* and the extracellular domain is comprised essentially of the Primary Sequence of MUC1 Growth Factor Receptor (PSMGFR) sequence. However, the exact site of cleavage can vary depending on cell type, tissue type, or which cleavage enzyme a particular person expresses or overexpresses. In addition to the cleavage site that we previously identified which leaves the transmembrane portion of MUC1* comprising most or all of the PSMGFR SEQ ID NO:2, other cleavage sites result in an extended MUC1* comprised of most or all of SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621). The site of MUC1 cleavage affects how the remaining extracellular domain folds. We have identified monoclonal antibodies that bind to cleaved MUC1* on cancer cells but do not bind to cleaved MUC1* as it exists on healthy stem and progenitor cells.

Whereas an anti-MUC1* antibody or antibody-like molecule may be most effective if it competitively inhibits the binding of NME1, NME6, NME8 or NME7 or NME7-AB to MUC1*, for example an antibody that binds to the PSMGFR sequence especially if said antibody is unable to bind to a PSMGFR peptide if the 10 C-terminal amino acids are missing, antibodies or antibody-like molecules that carry a payload need not competitively inhibit the binding of MUC1* ligands to be effective as anti-cancer agents. For example antibodies or antibody-like molecules that are conjugated to a toxin could be effective at killing target cancer cells without necessarily inhibiting binding of the activating ligands. For example, antibodies or antibody-like molecules incorporated into CAR Ts or BiTEs which recruit the patient's immune system to the tumor can be effective as anti-cancer agents even if the antibody fragment targets a portion of MUC1* such that antibody fragment binding does not competitively inhibit the binding of NME1, NME6, NME8, NME7-AB or NME7. In a preferred embodiment the antibody fragment incorporated into a CAR, an adaptive T cell receptor or a BiTE competitively inhibits the binding of NME1, NME6, NME8, NME7-AB or NME7 to MUC1*.

Antibodies that are able to bind to the extracellular domain of the remaining transmembrane portion block the interaction between the MUC1* extracellular domain and activating ligands and in this way can be used as therapeutic agents, for example for the treatment of cancers. Anti-MUC1* antibodies are also useful for the growth, delivery, identification or isolation of stem cells both in vitro and in vivo.

General Strategy for Using Antibodies, Antibody Fragments and CARs that Target the Extracellular Domain of MUC1*

Monoclonal antibodies MN-C3 and MN-C8 have a greater binding affinity for blood cells than solid tumor cancer cells. Humanized antibodies and antibody fragments containing sequences derived from the variable regions of MN-C3 and MN-C8 can be used as a stand alone therapy or integrated into CAR Ts, BiTEs, ADCs for the treatment of blood cancers.

Alternatively, humanized antibodies and antibody fragments containing sequences derived from the variable regions of MN-C3 and MN-C8 can be used to deliver stem cells to a specific location such as for in situ human therapeutics. In one case, a substrate coated with humanized MN-C3 or MN-C8 derived antibodies or antibody fragments is loaded with stem cells then inserted into a patient. In another case, a substrate coated with humanized MN-C3 or MN-C8 derived antibodies or antibody fragments is inserted into a patient in order to recruit the patient's own stem cells to a specific area for therapy. Human therapies in which antibodies that bind to human stem cells will be of therapeutic use include spinal cord repair. Substrates coated with humanized MN-C3 or MN-C8 derived antibodies or antibody fragments are also used to identify or isolate human antibodies. Humanized MN-C3 or MN-C8 derived antibodies can also be used to stimulate the growth of stem cells.

CARs and Cleavage Enzymes

Many applications of CAR T therapy are limited by the length or flexibility of the extracellular domain between the T cell membrane and the antibody fragment that will direct the T cell to the desired location. For example, the surface of solid tumor cancer cells is populated with a myriad of cell surface proteins and growth factor receptors. Many of these cell surface proteins have bulky extracellular domains that limit the access of immune cells, such as T cells or CAR T cells, to the tumor cell surface. In one example, MUC1 and the cleaved growth factor receptor form MUC1* are overexpressed on over 75% of solid tumor cancers and on some blood cancers. The extracellular domain of MUC1 full-length contains between about 1,500 and 2,500 amino acids while the extracellular domain of MUC1* contains only about 45 to 65 amino acids. Variability in the length of MUC1 full-length is due to variability in the number of tandem repeat units that are expressed. Variability in the length of MUC1* is due to different cleavage sites when MUC1 is cleaved by different cleavage enzymes. Whereas it is most desirable to get the T cell close to the surface of the cancer cell, access can be sterically hindered by neighboring proteins, including full-length MUC1, that have large and bulky extracellular domains. This is especially true for early stage cancers. Tissue studies show that early stage cancers have more full-length MUC1 than late stage cancers that can be devoid of any full-length MUC 1. This problem can in some cases severely limit the efficacy of cancer immunotherapies, including CAR T, adaptive T cell therapy, BiTEs and other T cell engagers.

One solution to this problem is to express or activate cleavage enzymes in the area of the targeted tumor cells to cleave the bulky proteins that restrict access of T cells to the tumor.

In one aspect of the invention, the cleavage enzyme and the CAR are transduced into the same T cell. In another aspect of the invention, the cleavage enzyme is on an inducible promoter such that its expression is activated when the CAR engages the targeted cancer cells. In some cases, the expression of the cleavage enzyme is controlled by an inducible promoter. In one aspect of the invention, expression of the cleavage enzyme is induced when the immune cell is activated, for example when it recognizes or engages its target. In one example, a T cell is transfected or transduced with a cleavage enzyme whose expression is induced when the T cell recognizes a target cancer cell. One way to do this is to induce expression of the cleavage enzyme when, or shortly after, an NFAT protein is expressed or translocated to the nucleus. For example, a sequence derived from an NFAT promoter region is put upstream of the gene for the cleavage enzyme. In this way, when the transcription factors that bind to the promoter of the NFAT protein are present in sufficient concentration to bind to and induce transcription of the NFAT protein, they will also bind to that same promoter that is engineered in front of the sequence for transcription of the cleavage enzyme. The NFAT protein may be NFAT1 also known as NFATc2, NFAT2 also known as NFATc or NFATc1, NFAT3 also known as NFATc4, NFAT4 also known as NFATc3, or NFAT5. In one aspect of the invention, the NFAT is NFATc1, NFATc3 or NFATc2. In one aspect of the invention, the NFAT is NFAT2 also known as NFATc1. SEQ ID NO:646 shows nucleic acid sequence of the upstream transcriptional regulatory region for NFAT2. The promoter sequence for NFAT gene may include the nucleic acid sequence of SEQ ID NO:781-783 or SEQ ID NO:815 as examples, but it can be seen that the optimal sequence or minimal sequence for expression of the cleavage enzyme may be obtained by making fragments, extensions or mutations of the promoter and testing for the strength of the promoter with respect to expression of the cleavage enzyme. In one aspect of the invention, the transcriptional regulatory region for NFAT2 is engineered upstream of the gene encoding the cleavage enzyme MMP9 (SEQ ID NO:647) or the catalytic sub-unit of MMP9 (SEQ ID NO:648). In one aspect of the invention, the NFAT is NFATc3 and the promoter sequence of NFATc3 includes nucleic acid sequences from SEQ ID NO:816. In one aspect of the invention, the transcriptional regulatory region for NFATc3 is engineered upstream of the gene encoding the cleavage enzyme MMP9 (SEQ ID NO:647) or the catalytic sub-unit of MMP9 (SEQ ID NO:648). In another aspect of the invention, the NFAT is NFATc2. SEQ ID NO:817-818 shows nucleic acid sequence of the upstream transcriptional regulatory region for NFATc2. In one aspect of the invention, the transcriptional regulatory region for NFATc2 is engineered upstream of the gene encoding the cleavage enzyme MMP9 (SEQ ID NO:647) or the catalytic sub-unit of MMP9 (SEQ ID NO:648).

Another method for having the expression of the cleavage enzyme induced when the T cell or CAR T cell is activated is to have the gene for the cleavage enzyme on an inducible promoter where the NFAT protein itself binds to and induces transcription of the cleavage enzyme. In this case, an NFAT response element (NFAT RE) may be positioned upstream of the gene for the cleavage enzyme or fragment of the cleavage enzyme. The NFAT may bind to its responsive element upstream of the cleavage enzyme alone or as part of a complex. The NFAT protein may be NFATc1, NFATc2, NFATc3, NFATc4, or NFAT5. In a preferred embodiment, the NFAT protein is NFAT2 aka NFATc1, aka NFATc. The gene of the cleavage enzyme or fragment thereof is cloned downstream of an NFAT-response element (SEQ ID NO:649), which may be repeats of the response element (SEQ ID NO:650) and CMV minimal promoter (mCMV) (SEQ ID NO:651) to induce expression of cleavage enzyme by NFAT protein. The NFAT response element may include nucleic acid sequence of NFAT consensus sequence (SEQ ID NO:804). The NFAT response element may include the nucleic acid sequence of SEQ ID NOS:805-814 as examples, but it can be seen that the optimal sequence or minimal sequence for expression of the cleavage enzyme may be obtained by making fragments, extensions or mutations of the responsive element nucleic acid and testing for the strength of the responsive element with respect to expression of the cleavage enzyme. The enhancer region of Foxp3 also contains NFAT response elements within the 120-bp from 2079 to 2098 (SEQ ID NO:821). The NFAT response element may include nucleic acid NFAT consensus sequence of (5′-cattttttccat-3′) (SEQ ID NO:819) or (5′-tttttcca-3′) (SEQ ID NO:820), which NFATc1 specifically binds to (Xu et al., Closely related T-memory stem cells correlate with in vivo expansion of CAR. CD19-T cells and are preserved by IL-7 and IL-15, Blood 2014 123:3750-3759), or repeats thereof. The NFAT response elements may also be separated by nucleic acid spacer sequences. Other NFAT responsive elements may exist and may further be discovered, and a skilled artisan in the art when directed to determine NFAT responsive element may do so by carrying out molecular biological assays to obtain it given the guidance of at least the responsive elements as set forth as SEQ ID NOS: 804-814 albeit as only mere examples. In one aspect of the invention, the cleavage enzyme that is downstream of the NFAT-response element and CMV minimal promoter is MMP9 (SEQ ID NO:652). In another aspect of the invention, the cleavage enzyme is a catalytic sub-unit of MMP9 (SEQ ID NO:653).

Because NFATs 1-4 are regulated by the calcineurin pathway, potential toxicities that may arise in a patient can be stopped by treatment with an immunosuppressive agent such as FK506, Cyclosporin, Cyclosporin A, or Tacrolimus that block calcineurin activity and inhibit NFAT translocation to the nucleus. The T cell transduced or transfected with a cleavage enzyme on an inducible promoter may also be transfected or transduced with a CAR that recognizes a protein or molecule on the cancer cell. In a specific example, the cleavage enzyme is one that is able to cleave MUC1 full-length and the CAR bears an antibody fragment that directs it to MUC1* on the surface of cancer cells.

To determine which cleavage enzymes cleave MUC1 on cancer cells, we tested a series of MMP and ADAM enzyme inhibitors. These experiments pointed to MMP9 as being an important cleavage enzyme in cancer cells. To confirm that MMP9 cleaves MUC1 on cancer cells, we transfected HCT-116 MUC1 negative colon cancer cells with a mimic of full-length MUC1 having 41 tandem repeat domains: HCT-MUC1-41TR. Through single cell cloning we were able to establish this cell line wherein MUC1 only minimally gets cleaved to MUC1*. In one aspect of the invention, an immune cell is transduced with both a CAR to target the immune cell to the tumor, and a cleavage enzyme. The CAR and the cleavage enzyme can be encoded on the same plasmid or on two different plasmids. In one aspect, the cleavage enzyme is on an inducible promoter. In another aspect, expression of the cleavage enzyme is induced by a protein that is expressed when the immune cell is activated. In one case, expression of the cleavage enzyme is induced by an NFAT protein. In another aspect, expression of the cleavage enzyme is induced by NFATc1. In another aspect, expression of the cleavage enzyme is induced when one of the NFAT proteins binds to an NFAT response element that is inserted upstream of the gene for the cleavage enzyme or a catalytically active fragment thereof. In one aspect, the cleavage enzyme is MMP9 or a fragment of MMP9 that is catalytically active.

In one aspect of the invention, the cleavage enzyme is MMP9 (SEQ ID NO:643). In one case, the cleavage enzyme is an MMP9 fragment that is catalytically active. One example of an MMP9 catalytic fragment is given as SEQ ID NO:645.

In one example, T47D MUC1 positive tumor cells were incubated with a recombinant catalytic domain of MMP9 (Enzo Life Sciences, Inc., Farmingdale, N.Y.) at either 100 ng/mL or 500 ng/mL. Western blot analysis showed that the MUC1/MUC1* positive cancer cells underwent extensive cleavage of MUC1 to MUC1*. In another example, T47D breast cancer cells were pre-incubated with a human recombinant MMP9 catalytic domain protein then co-cultured with anti-MUC1* CAR44 T cells. The specific killing of the T47D cells by CAR44 T cells was monitored in real-time on an xCelligence instrument that measures impedance as a function of time. This analysis uses electrode arrays upon which cancer cells are plated. The adherent cancer cells insulate the electrode and cause an increase in impedance as they grow. Conversely, T cells are not adherent and remain in suspension so do not increase or decrease impedance. However, if the T cells or CAR T cells kill the cancer cells on the electrode plate, the cancer cells ball up and float as they die, which causes the impedance to decrease. The addition of MMP9 catalytic domain dramatically increased the killing of T47D cancer cells. In one aspect of the invention, an immune cell is transfected or transduced with both a CAR comprising an antibody fragment that targets a tumor antigen and a cleavage enzyme. In another aspect of the invention, an immune cell is transfected or transduced with both a CAR comprising an antibody fragment that targets a tumor antigen and a cleavage enzyme that cleaves a tumor antigen to a form recognized by the antibody fragment of the CAR. In one aspect, an immune cell is transfected or transduced with both a CAR comprising an antibody fragment that targets a tumor antigen and a cleavage enzyme that cleaves a tumor antigen to a form recognized by the antibody fragment of the CAR, wherein the antibody fragment of the CAR recognizes MUC1* extra cellular domain and the cleavage enzyme cleaves MUC1 to MUC1*. In one aspect, an immune cell, which may be a T cell or an NK cell, is transfected or transduced with a CAR comprising an antibody fragment derived from MNC2, MNE6, MNC3 or MNC8 and a cleavage enzyme chosen from the group comprising MMP1, MMP2, MMP3, MMP7, MMP8, MMP9, MMP11, MMP12, MMP13, MMP14, MMP16, ADAMS, ADAM10, ADAM17, ADAM 19, ADAMTS16, ADAM28 or a catalytically active fragment thereof.

A method for studying activation of the NFAT pathway is by chemically activating the pathway using PMA with lonomycin (Lyakh et al., Expression of NFAT-Family proteins in normal human T cells, MOLECULAR AND CELLULAR BIOLOGY, Vol. 17, No. 5, May 1997, p. 2475-2484; Rao et al., Transcription factors of the NFAT family—Regulation and function, Annu. Rev. Immunol. 1997. 15:707-47; Macian, NFAT proteins—Key regulators of T-cell development and function, Nature Reviews Immunology, Vol. 5, pp 472-484 June (2005)). It has been demonstrated that PMA and lonomycin induce expression of NFAT proteins. The above-cited references show a scheme of the regulation of NFAT activation. lonomycin increases calcium which activates the Calcineurin/Calmodulin complex. Calcineurin/Calmodulin dephosphorylate NFAT, which causes NFATs, especially NFATc1, to be translocated to the nucleus where it binds to DNA to stimulate transcription of target genes. NFATc1 is one of the first NFAT proteins to be translocated to the nucleus upon T cell activation and it is only there transiently before it exits the nucleus. Therefore, PMA plus lonomycin activation of cells we transfected or transduced with NFAT inducible cleavage enzymes is physiologically relevant and mimics in vivo T cell activation turning on expression of the NFAT inducible cleavage enzymes described herein.

A plasmid was constructed then transfected into HEK293T cells, wherein the gene for MMP9 catalytic domain was inserted downstream of either 3 or 4 NFAT response elements. The NFAT pathway was activated by the addition of PMA at 10 ng/mL and lonomycin at either luM or 2 uM. Lysate from cells transfected with the plasmid containing 3 or 4 repeats of a NFAT Response element, or the conditioned media from the cells, were assayed for the presence of MMP9 in a Western blot assay.

In one aspect of the invention, a person diagnosed with cancer or at risk of developing cancer is administered a sufficient amount of an immune cell transduced with both a CAR and a cleavage enzyme. In another aspect of the invention, a person diagnosed with cancer or at risk of developing cancer is administered a sufficient amount of an immune cell transduced with both a CAR and a cleavage enzyme, wherein the cleavage enzyme is on an inducible promoter that is activated by proteins that are expressed when the immune cell becomes activated. In another aspect of the invention, a person diagnosed with cancer or at risk of developing cancer is administered a sufficient amount of an immune cell transduced with both a CAR and a cleavage enzyme, wherein the cleavage enzyme is on an inducible promoter that is activated by one or more NFAT. In one case the NFAT is NFATc1. In another aspect, the NFAT is NFATc3. In another aspect, the NFAT is NFATc2. In any of the instances above, the extra cellular domain of the CAR comprises a fragment of an anti-MUC1* antibody. In one aspect, the anti-MUC1* antibody is MNC2scFv or a humanized form of MNC2scFv. In another aspect, the anti-MUC1* antibody is MNE6scFv or a humanized form of MNE6scFv. In any of the instances above, the immune cell can be a T cell, an NK cell, a mast cell, or a dendritic cell.

It is not intended that the present invention be limited to one or two specific methods of having expression of a cleavage enzyme induced by an activated T cell. We have demonstrated specific expression of a cleavage enzyme only upon T cell activation by constructing a plasmid with the cleavage enzyme gene downstream of an NFAT promoter sequence or downstream of one or more repeats of NFAT response elements. In another aspect of the invention, expression of the cleavage enzyme is induced by constructing a plasmid where the cleavage enzyme gene is inserted downstream of an IL-2 promoter sequence or downstream of an IL-2 response element, then inserting the plasmid into an immune cell. In another aspect of the invention, expression of the cleavage enzyme is induced by constructing a plasmid where the cleavage enzyme gene is inserted downstream of a Calcineurin promoter sequence or downstream of a Calcineurin response element, then inserting the plasmid into an immune cell and then administering to a patient for the treatment or prevention of cancers. There are also drug-inducible plasmids that can be used to induce expression of the cleavage enzyme or used to stop expression induced by an element of an activated T cell. These drug inducible systems may include tetracycline-inducible systems, Tet-on, Tet-off, tetracycline response elements, doxycycline, tamoxifen inducible systems, ecdysone inducible systems and the like.

It is not intended that the present invention be limited to one or two specific promoters used in the plasmids encoding the CARs or inducible cleavage enzymes. As is known by those skilled in the art, many promoters can be interchanged including SV40, PGK1, Ubc, CAG, TRE, UAS, Ac5, polyhedron, CaMKIIa, GAL1, GAL10, TEF1, GDS, ADH1, CaMV35S, Ubi, H1 and U6.

Table 2 shows cytokine release data for human T cells transfected with some of the long linker CARs.

In another aspect, the invention is directed to a composition that includes at least two different plasmids transfected into the same immune cell, wherein the first encodes a CAR comprising an antibody fragment, scFv, or peptide that binds to a tumor antigen and the other encodes a gene that is not a CAR, wherein the gene that is not a CAR is expressed from an inducible promoter that is activated by elements of an activated immune cell. In one aspect, the immune cell is a T cell or an NK cell. In one aspect the CAR comprises an antibody fragment, scFv or peptide that binds to the extra cellular domain of MUC1*. In one aspect the CAR comprises an scFv derived from MNC2, MNE6, MNC3 or MNC8. In one aspect the non-CAR species is a cleavage enzyme. In one aspect the cleavage enzyme is MMP2, MMP3, MMP9, MMP13, MMP14, MMP16, ADAM10, ADAM17, ADAM28 or catalytically active fragments thereof. In another aspect the non-CAR species is a cytokine. In one aspect, the Cytokine is IL-7. In one aspect the cytokine is IL-15. In another aspect the cytokine is IL-7 and IL-15. In one case expression of the non-CAR species is induced by elements of an activated immune cell. In one aspect the element of an activated immune cell is an NFAT. In one aspect the NFAT is NFATc1, NFATc3 or NFATc2. Cytokines IL-7 and IL-15 are known to promote T cell persistence. In one aspect of the invention an immune cell described above is administered to a patient for the treatment or prevention of cancer. In one aspect of the invention, the cancer is a MUC1 positive cancer or a MUC1* positive cancer.

In another aspect, the invention is directed to a composition that includes at least two different plasmids transfected into the same immune cell, wherein the first encodes a CAR comprising an antibody fragment, scFv or peptide that binds to the extra cellular domain of an antigen on the surface of a B cell and the other encodes a gene that is not a CAR, wherein the gene that is not a CAR is expressed from an inducible promoter that is activated by elements of an activated immune cell. In one aspect, the immune cell is a T cell or an NK cell. In one aspect the CAR comprises an antibody fragment, scFv or peptide that binds to CD19. In one aspect the CAR comprises sequences derived from SEQ ID NO:830-831. In another aspect the antibody fragment, scFv or peptide binds to a surface antigen of a B cell or a B cell prescursor, or binds to CD19, CD20, CD22, BCMA, CD30, CD138, CD123, CD33 or LeY antigen. In one aspect the non-CAR species is a cleavage enzyme. In another aspect the non-CAR species is a cytokine. In one aspect, the Cytokine is IL-7. In one aspect the cytokine is IL-15. In another aspect the cytokine is IL-7 and IL-15. In one case expression of the non-CAR species is induced by elements of an activated immune cell. In one aspect the element of an activated immune cell is an NFAT. In one aspect the NFAT is NFATc1, NFATc3 or NFATc2. that is not a CAR, wherein the gene that is not a CAR is expressed from an inducible promoter wherein expression is induced by elements of an activated immune cell. In one aspect the immune cell transfected or transduced with the composition is administered to a patient for the treatment or prevention of cancer. In one case the cancer is a leukemia, lymphoma or blood cancer.

It is not intended for the invention to be limited by a specific method or technology for inserting the gene or plasmid comprising a sequence encoding a CAR or activated T cell inducible protein or peptide there encoded. For example, the gene encoding the CARs and activated T cell induced genes described herein can be virally transduced into an immune cell using viruses, which may or may not result in the CAR gene being integrated into the genome of the recipient cell. Virus delivery systems and viral vectors include but are not limited to retroviruses, including gamma-retroviruses, lentivirus, adenoviruses, adeno-associated viruses, baculoviruses, poxvirus, herpes simplex viruses, oncolytic viruses, HF10, T-Vec and the like. In addition to viral transduction, CARs and activated T cell induced genes decribed herein can be directly spliced into the genome of the recipient cell using methods such as CRISPR technology, CRISPR-Cas9 and -CPF1, TALEN, Sleeping Beauty transposon system, and SB 100X.

Bulky cell surface proteins such as MUC1-FL can also cause a steric hindrance problem for BiTEs. A BiTE is a two-headed bi-specific antibody wherein one head binds to a T cell and the other head binds to a tumor-associated antigen. In this way, the BiTE links together the T cell and the tumor cells. The antibody that binds to the T cell should be an antibody that activates the T cell, such as an antibody against CD3 such as OKT3 scFv (SEQ ID NO:687) or CD28. To solve the steric hindrance problem, the linker between the T cell specific antibody and the tumor specific antibody is lengthened. Examples of BiTEs with extended linkers Anti-CD3-linker-anti-MUC1*, are shown as SEQ ID NOS:689, 691, 693, 695, 697, and 699.

In another aspect of the invention, an anti-MUC1* single chain molecule is fused to a cleavage enzyme or a catalytically active fragment of a cleavage enzyme. In one aspect of the invention, the cleavage enzyme is MMP9 (SEQ ID NO:701). In another aspect of the invention, the enzyme is a catalytically active fragment of MMP9 (SEQ ID NO:703), In some cases, the antibody fragment of the CAR is chosen for its ability to recognize MUC1* when cleaved by that specific cleavage enzyme. In one embodiment, the cleavage enzyme is MMP9, MMP3, MMP14, MMP2, ADAM17, ADAM TS16, and/or ADAM28. In one embodiment, the antibody or antibody fragment binds to a peptide having the sequence of SEQ ID NO:2 (PSMGFR) GTINVHDVETQFNQYKTEAASRYNLTISDVSVSDVPFPFSAQSGA, PSMGFR N-10, QFNQYKTEAASRYNLTISDVSVSDVPFPFSAQS GA, or PSMGFR N+18 SNIKFRPGSVVVQLTLAFREGTINVHDVETQFNQYKTEAASRYNLTISDVSVSDVPFPFSAQS GA. “PSMGFR N+18” refers to a fragment of MUC1 receptor in which 18 amino acid residues have been added at the N-terminal end of PSMGFR segment within the MUC1 receptor of SEQ ID NO:1. In another embodiment, cleavage enzymes MMP9 and MMP3 are transduced into a T cell that is also transduced with a CAR with an antibody fragment that is a fragment of MNC2.

In many cases it is desirable to have the cleavage enzyme expressed only after an immune cell recognizes the tumor-associated target on a solid tumor. In this way, the cleavage enzyme will not freely move throughout the body, cleaving MUC1, MUC16 or other proteins, wherein their cleavage could actually promote cancer. However, there are cancers that are physically accessible to direct application of chemotherapy agents, CAR T cells and other anti-cancer agents. For example, types of brain cancers, prostate cancer and ovarian cancers have all shown the benefit of direct application of anti-cancer agents into the local vicinity of the cancer. CAR T cells have been injected directly into the brain and/or cerebral spinal fluid of glioblastoma patients. Radiation has been directed to the prostate area for the treatment of prostate cancers, including those that have metastasized. Hot chemo therapy agents have been directly injected into the intraperitoneal cavity for the treatment of ovarian cancers. In these and other cases, where the cancers that are physically accessible to direct application of chemotherapy agents, a cleavage enzyme is administered in the presence or absence of another anti-cancer agent, which could be a CAR T cell, an immune cell engineered to recognize a tumor-associated antigen, a BiTE, an ADC, a biological or a standard chemotherapy agent. Although ovarian cancer can metastasize to anywhere in the body, it usually stays in the abdomen as it spreads to adjacent organs, such as the intestines, liver and stomach. This makes ovarian cancer an ideal test case for improving the effect of anti-cancer agents by administering a cleavage enzyme in combination with other anti-cancer agents, including a platinum-based drug such as carboplatin (Paraplatin) or cisplatin, and/or a taxane such as paclitaxel (Taxol) or docetaxel (Taxotere). Alkeran (Melphalan), Avastin (Bevacizumab), Carboplatin, Clafen (Cyclophosphamide), and Cytoxan have all been approved for the treatment of ovarian cancer. Other treatments that are being tested for the treatment of ovarian cancers include agents that target MUC1, MUC16 and as described herein, MUC1*.

Stimulation of CAR T Cell with Antigen

Researchers have been trying to train or pre-stimulate CAR T cells so that they are more effective killing cells when they are injected into the patient. In some cases, they incubate the CAR T cells with anti-CD3/CD28 magnetic beads, which activates the CAR T cells but results in two negative outcomes. First, stimulating CAR T cells with anti-CD3/CD28 beads increases killing of target as well as non-target cells, which would end up killing normal cells and tissues in the patient. One researcher attempted to train anti-CD19 CAR T cells to better recognize and kill target blood cancer cells, by taking some of the patient's cancer cells and incubating the CAR T cells with them, then injecting the CAR T cells plus cancer cells back into the patient. One potential problem with this approach is that it introduces risk by injecting cancer cells back into the patient. The other risk with all of these pre-stimulation approaches is that they extend the amount of time that the CAR T cells are in culture which makes them mature from the universal naïve state to the Effector Memory and Effector state. Research now shows that CAR T cells comprising sub-populations that include a significant percentage in the Naïve state and Central Memory state increase persistence in vivo, leading to a permanent cure. In contrast, CAR T cells where most of the sub-populations are Effector Memory or Effector state gives a transient therapeutic response.

In another aspect, the present invention is directed to methods of activating, stimulating expansion of, or selecting for a population of immune cells that express a receptor that recognizes a specific tumor antigen, involving contacting the immune cell expressing the cognate receptor with the antigen to which it binds. In one aspect the immune cell is an anti-MUC1* CAR T cell and the stimulating antigen is a peptide derived from the sequence of a cleaved MUC1 that is devoid of the tandem repeat domain. In one aspect, the antibody fragment that targets the CAR binds to a peptide and that peptide is used to activate, stimulate or select for the CAR T cells wherein the peptide has a sequence containing at least 15 contiguous amino acids, but can have up to 4 substitutions or deletions, wherein the peptide is derived from:

(i) PSMGFR region of MUC1;
(ii) PSMGFR peptide;
(iii) a peptide having amino acid sequence of
(SEQ ID NO: 620)
SNIKFRPGSVVVQLTLAFREGTINVHDVETQFNQYKTEAASRY;
(iv) a peptide having amino acid sequence of
(SEQ ID NO: 621)
SVVVQLTLAFREGTINVHDVETQFNQYKTEAASRY;
(v) a peptide having amino acid sequence of
(SEQ ID NO: 622)
VQLTLAFREGTINVHDVETQFNQY;
or
(vi) a peptide having amino acid sequence of
(SEQ ID NO: 623)
SNIKFRPGSVVVQLTLAFREGTIN.

Here, we describe novel methods of pre-stimulating CAR T cells that increases specific killing of target cells without increasing non-specific killing and does so in a short time period that keeps the CAR T cells, mostly in the Naïve and Central Memory state, with lower populations in Effector Memory and Effector state. An improvement over previous training or stimulation methods is that instead of incubating CAR T cells with the patient cancer cells, we incubated the CAR T cells with patient cancer cells that had been transfected with even more antigen, which led to an increased specific stimulation. In another case, CAR T cells are pre-stimulated by quick incubation with a synthetic mimic of the antigen attached to a surface which can be a cell culture plate. Although these examples were performed with anti-MUC1* CAR T cells and MUC1* positive cancer cells, the methods can be extended to increase the specificity and efficiency of killing for any cancer immunotherapy.

In one aspect of the invention, immune cells bearing a chimeric antigen receptor (CAR), are incubated with an entity that presents the antigen to which the antibody portion of the CAR binds, before the CAR presenting therapeutic cells are administered to a patient. In this way, the immune cells are pre-stimulated or trained to attack cells that present that antigen. In one case the entity that presents the antigen is a cell. In another case the entity that presents the antigen is a bead. In yet another case, the entity that presents the antigen is a surface or a substrate. In still another case, the entity that presents the antigen is a particle, which may be a soluble protein, to which the antigen is attached. In another case, the immune cell bearing a CAR is incubated with the free antigen.

In one aspect of the invention, the immune cells bearing a CAR are incubated with the antigen in vitro for a period of time, then the most or all of the antigen is removed before the CAR bearing immune cells are administered to the patient. In another aspect of the invention, the immune cells bearing a CAR are mixed with the antigen in vitro and administered together to the patient. In yet another aspect of the invention, the immune cells bearing a CAR are incubated with patient cells that have been transfected or transduced with the antigen and the CAR-bearing immune cells together with the antigen modified patient cells are administered to the patient. In one aspect of the invention, the patient cells are the targeted cancer cells and they are transfected or transduced with an even greater amount of the target antigen.

Here we show the effect of incubating the CAR-bearing immune cells with engineered antigen presenting entities wherein the immune cells are T cells, the CAR comprises an antibody fragment that binds to MUC1* and the antigen presented by the engineered cell, bead, surface, particle or free in solution is the MUC1* peptide SEQ ID NO:2. However, this is an example, whereas the invention can be used with virtually any CAR that comprises an antibody that binds to any target antigen.

We showed that the specific killing potential of anti-MUC1* CAR T cells was increased by incubating the CAR T cells with any entity that presented the MUC1* antigen. In one case, we incubated huMNC2-CAR44 T cells with MUC1* positive T47D breast cancer cells. In another case we incubated huMNC2-CAR44 T cells with a MUC1 negative cell line, HCT-116, that had been transfected with the transmembrane receptor MUC1*. In another case, we incubated huMNC2-CAR44 T cells with synthetic beads to which was attached a synthetic peptide having the sequence of the extra cellular domain of the MUC1* receptor, referred to herein as MUC1* peptide or MUC1*ecd peptide. In yet another case, we incubated huMNC2-CAR44 T cells with a surface that had been coated with bovine serum albumin (BSA) or human serum albumin (HSA) to which was attached the MUC1* peptide. In another case, we incubated huMNC2-CAR44 T cells with a soluble BSA or HAS to which was attached the MUC1* peptide. In another case, we incubated huMNC2-CAR44 T cells with the free MUC1* peptide. In still another case, we mixed huMNC2-CAR44 T cells with the target cancer cells that had been transfected with even more MUC1* transmembrane receptor.

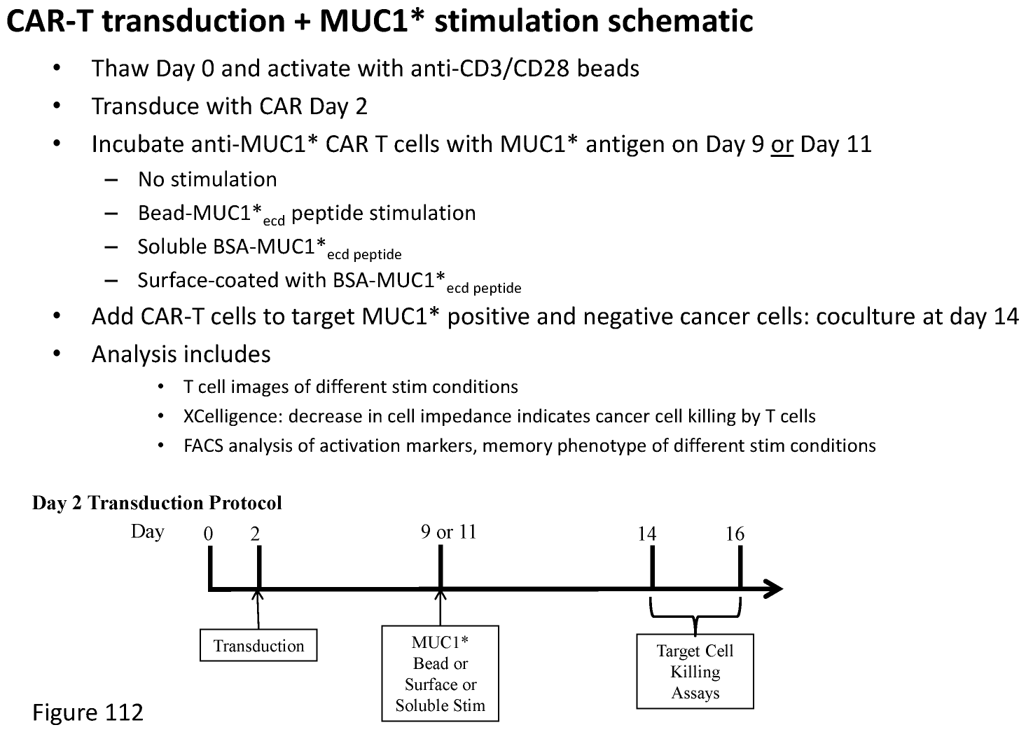

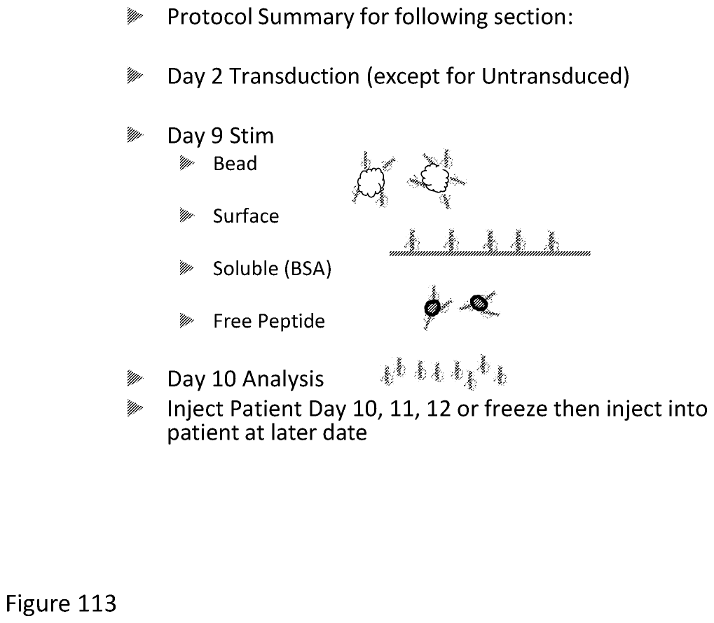

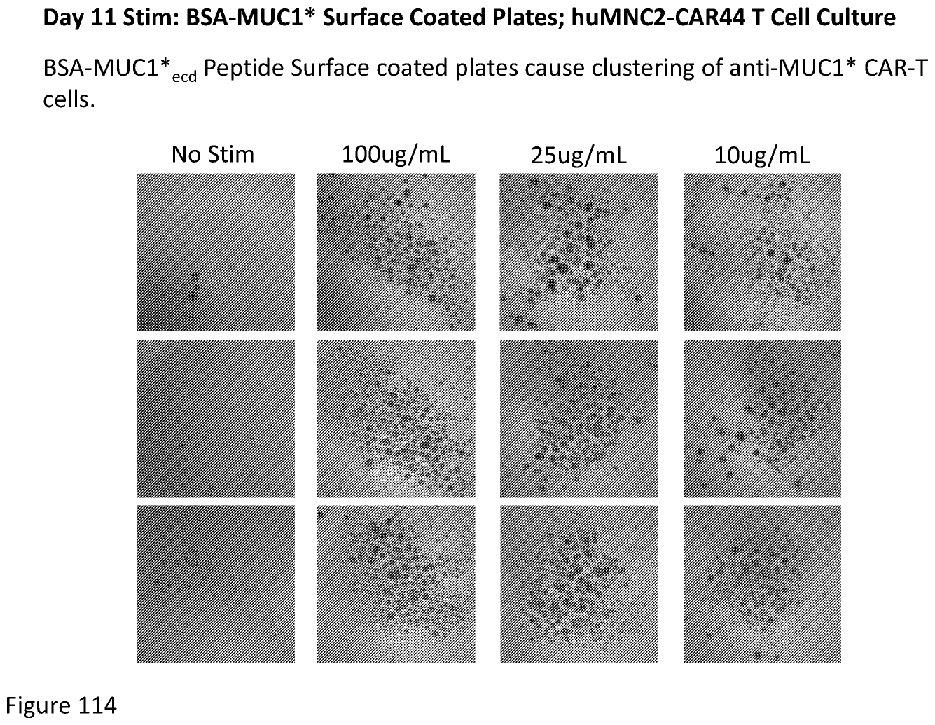

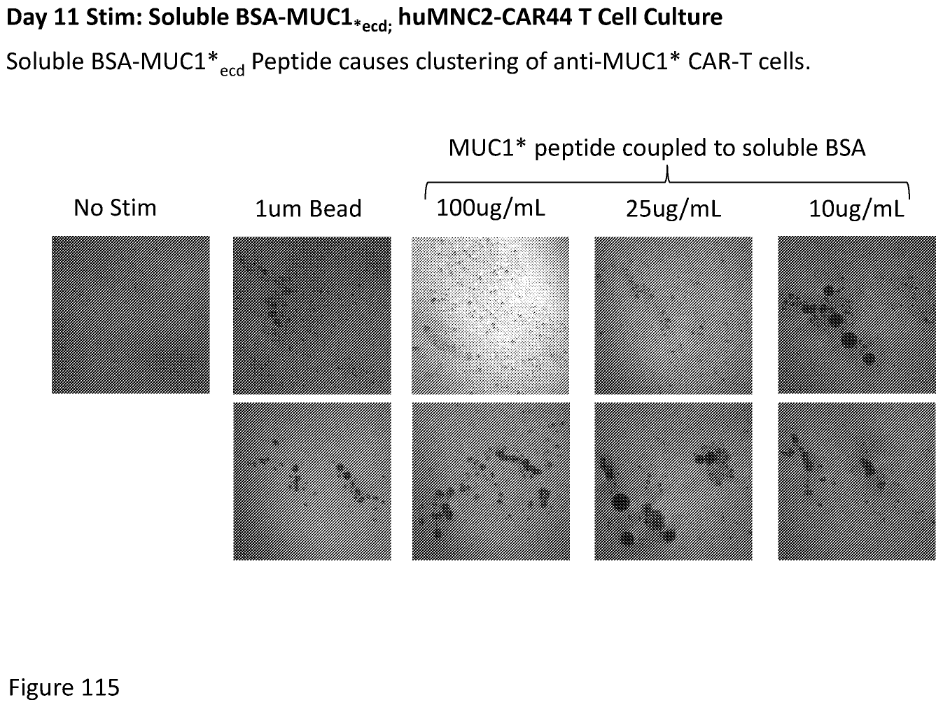

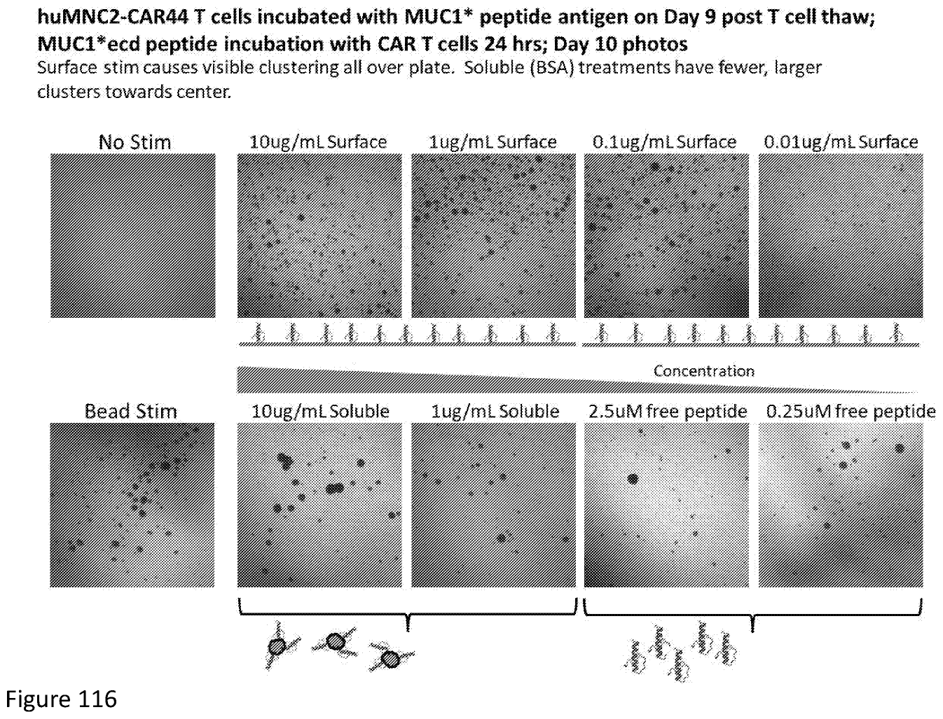

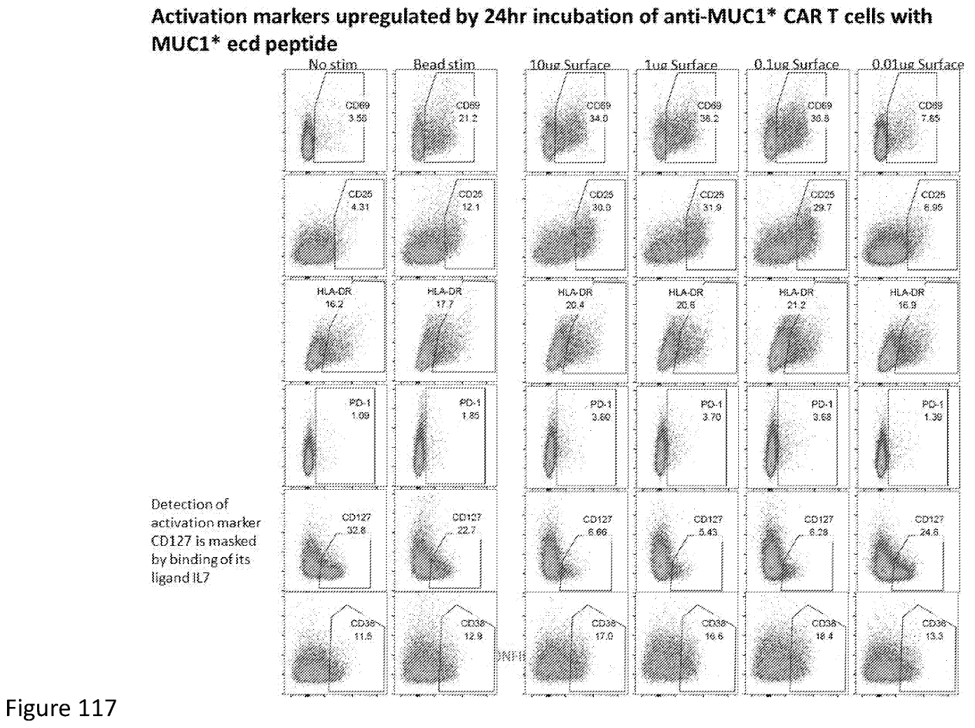

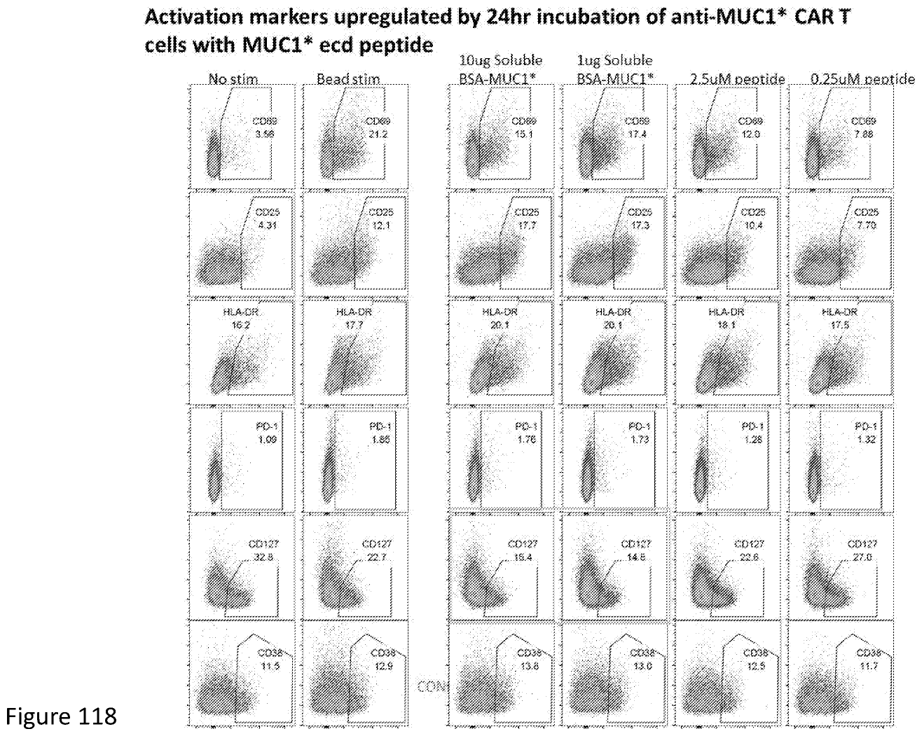

FIG. 112 shows a schematic of the timing of transducing T cells with the CAR, preferably on Day 2, expanding the CAR T cells until about Day 9, then incubating with the antigen for 24-48 hours, then either testing for killing effect or in practice, injecting into patient. FIG. 113 shows the schematic of the variable methods for presenting antigen to CAR T cells for stimulating incubation. FIG. 114 shows that incubating the CAR T cells, in this case anti-MUC1* CAR T cells, with a surface coated with the targeted antigen, in this case MUC1* peptide, causes the CAR T cells to bind and cluster around the antigen presenting entity, which is a visual sign of T cell activation. FIG. 115 shows that presenting the antigen on a soluble particle, such as BSA or HSA molecules, or free in solution, has the same stimulating effect on the CAR T cells. FIG. 116 shows that even at very low concentrations, presenting the antigen to the CAR T cells causes their activation. FIG. 117 and FIG. 118 show that analysis by FACS shows that incubating CAR T cells with their targeted antigen induces increased expression of T cell activation markers CD69 and CD25.

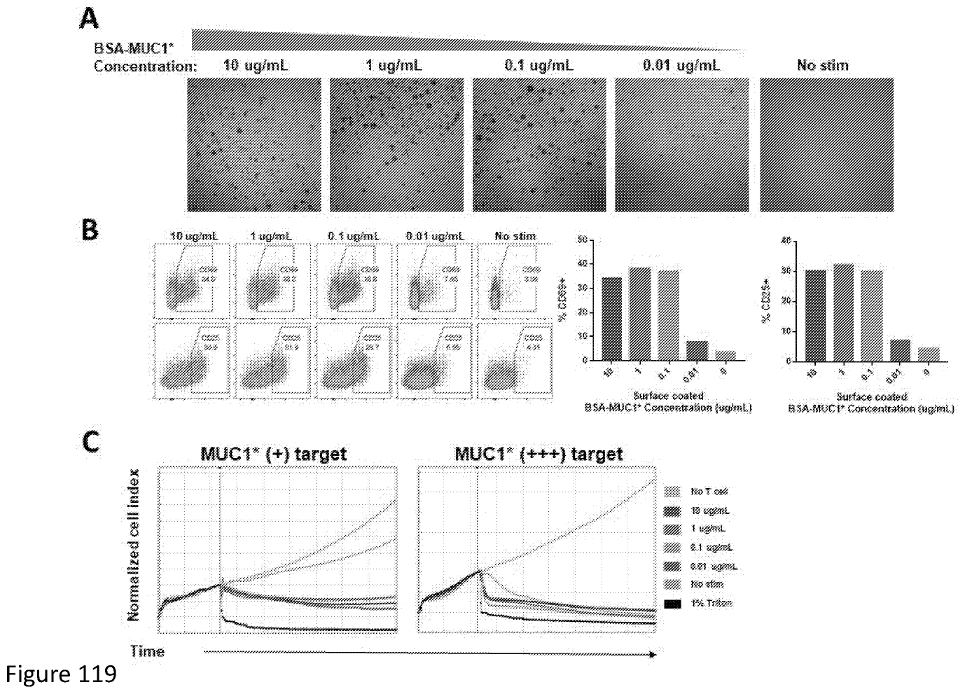

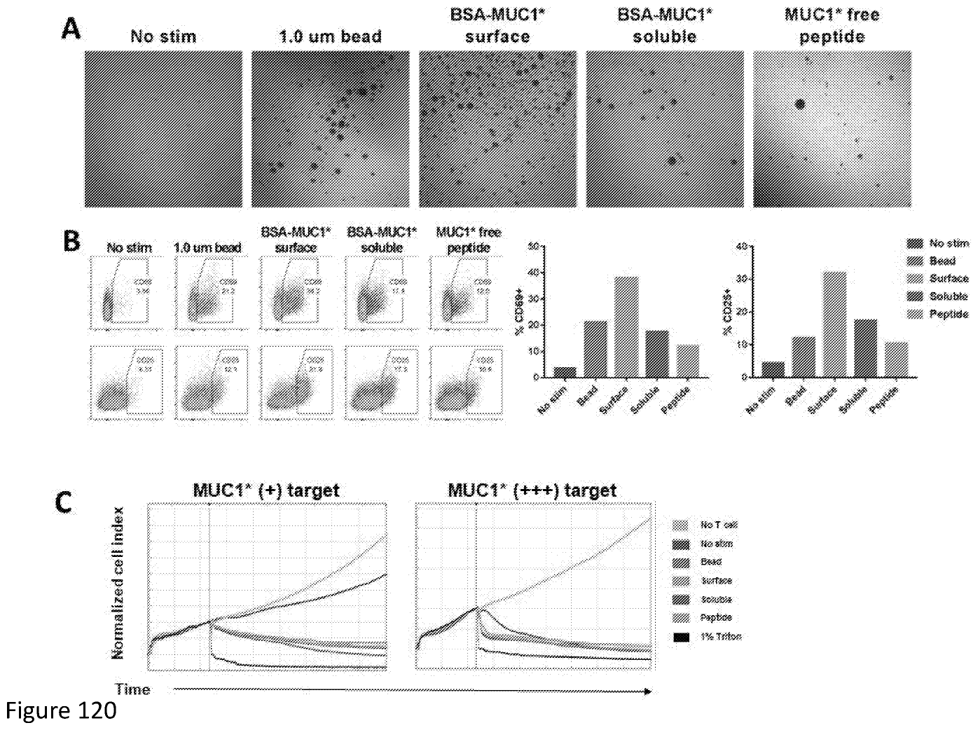

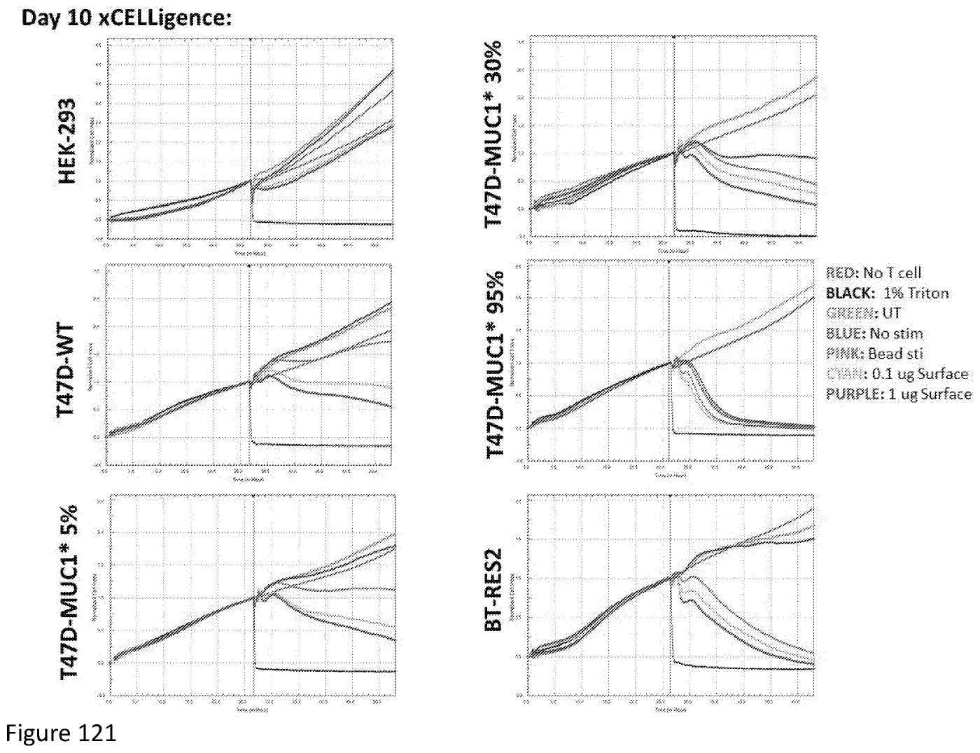

FIGS. 119A-119B summarize visual and FACS evidence of exposure to surface-attached antigen inducing CAR T activation. The photographs of FIG. 119A show that exposure to the targeted antigen induces CAR T cell clustering which is a sign of T cell activation. FIG. 119B shows FACS scans that verify that exposure to antigen induced upregulation of the T cell activation markers CD69 and CD25. The bar graph summarizes the FACS analysis of activation markers CD69 and CD25 in response to incubation with antigen. Some cell killing assays were done using an xCELLigence instrument. The instrument measures impedance as a function of time. Solid tumor cancer cells are adherent, so as they grow they increasingly insulate the electrode. T cells, and CAR T cells, are non-adherent so do not contribute to insulating the electrode. As the CAR T cells attack and kill the cancer cells, impedance falls. Normalized Cell Index, which is a measure of impedance, is plotted on the Y-axis over time. FIG. 119C is a trace produced by an xCELLigence instrument, which measures CAR T cell killing of cancer cells. As can be seen in FIG. 119C, T47D (MUC1+ breast cancer cells), cultured in the absence of T cells continue to grow, which increases impedance and increases the measure of Cell Index on the Y-axis. However, if the MUC1* positive cancer cells are co-cultured with human T cells transduced with an anti-MUC1* CAR, then there is CAR T cell mediated cancer cell killing that is reflected in a reduced slope of the impedance trace. Still referring to FIG. 119C, the graph on the left shows that anti-MUC1* CAR T cells that have not been pre-stimulated by culturing with the MUC1* antigen are not as effective at killing the cancer cells as the surface-attached MUC1* peptide stimulated anti-MUC1* CAR T cells. Anti-MUC1* CAR T cells that were first incubated with the antigen, MUC1* extra cellular domain peptide, are much more effective at killing the target cancer cells. The graph on the right of FIG. 119C shows an xCELLigence experiment where T47D MUC1* positive breast cancer cells have been transfected with even more MUC1*. As can be seen, anti-MUC1* CAR T cells can effectively kill the target cancer cells, even without antigen stimulation, when the target cancer cells express a lot of the antigen, in this case MUC1*. FIG. 120 shows a comparison of antigen stimulation of CAR T cells where the antigen, in this case MUC1* extra cellular domain peptide, is attached to a surface, beads, soluble BSA or just peptide free in solution. After a pre-stimulation period of 24 hours, the anti-MUC1* CAR T cells were removed from the surface or the beads, or the BSA-MUC1*-peptide or free MUC1* peptide was washed away from the CAR T cells. The experiments shown in FIG. 120A-120C shows that pre-incubation with antigen induces CAR T cell activation and increases the killing efficiency of the CAR T cells. Other methods of CAR T cell activation, such as exposure to anti-CD3/CD28 beads, increases the killing potential of CAR T cells but the killing is not specific. In FIG. 121, we show that pre-stimulation of anti-MUC1* CAR T cells does not increase the killing of MUC1* negative HEK293 cells. The antigen stimulation does however increase the killing efficiency of anti-MUC1* CAR T cells killing MUC1* positive cancer cells. As can be seen in FIG. 121, the need for CAR T pre-stimulation by incubation with the target antigen is needed less when the target cancer cells express more of the target antigen.

Various methods of pre-stimulating CAR T cells by incubating with the target antigen were also tested in vivo. Female NOD/SCID/GAMMA (NSG) mice that had been implanted with 90-day release estrogen pellets were subcutaneously implanted with 500,000 HCT-MUC1* cancer cells. HCT-MUC1* are HCT-116 cells, a MUC1* negative colon cancer cell line, that we transfected with the MUC1* transmembrane receptor. HCT-MUC1* cancer cells were also stably transfected with Luciferase so that tumors could be visualized on an IVIS instrument after animals are injected with the substrate Luciferin. After tumor engraftment was verified by IVIS, mice were injected with 10M anti-MUC1* CAR T cells: huMNC2-CAR44 transduced into human T cells. Half of the CAR T cells were injected near the tumor and the other half were injected into the circulation via the tail vein. As controls, animals were injected with untransduced T cells or an equal volume of PBS. The anti-MUC1* CAR T cells were stimulated by two different methods.

In Protocol 1, CAR T cells were stimulated by incubation with synthetic beads to which was attached the MUC1* extra cellular domain peptide. CAR T cells were separated from beads and washed before injecting into animals.

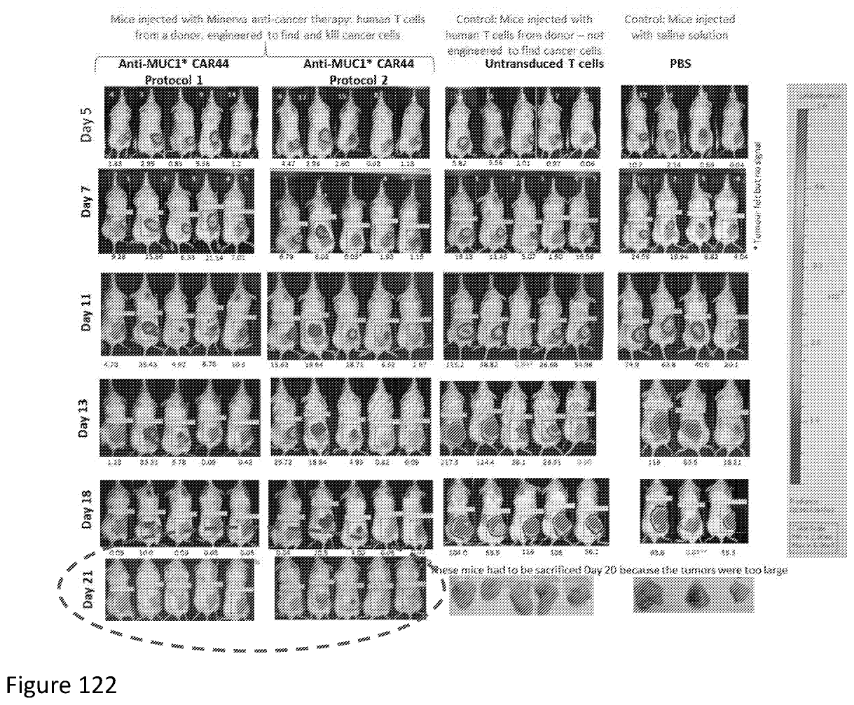

In Protocol 2, CAR T cells were stimulated by incubation with the same cancer cells that had been implanted into the mice. CAR T cells were separated from the stimulating cancer cells and washed before injecting into animals. IVIS measurements documenting the size of the tumors and response to treatment were taken about twice a week (See FIG. 122). The control mice grew tumors that were so large that the animals had to be sacrificed on Day 20. In comparison, of the 10 mice that were treated with anti-MUC1* CAR T cells, only one animal had a barely measurable tumor left by Day 21.

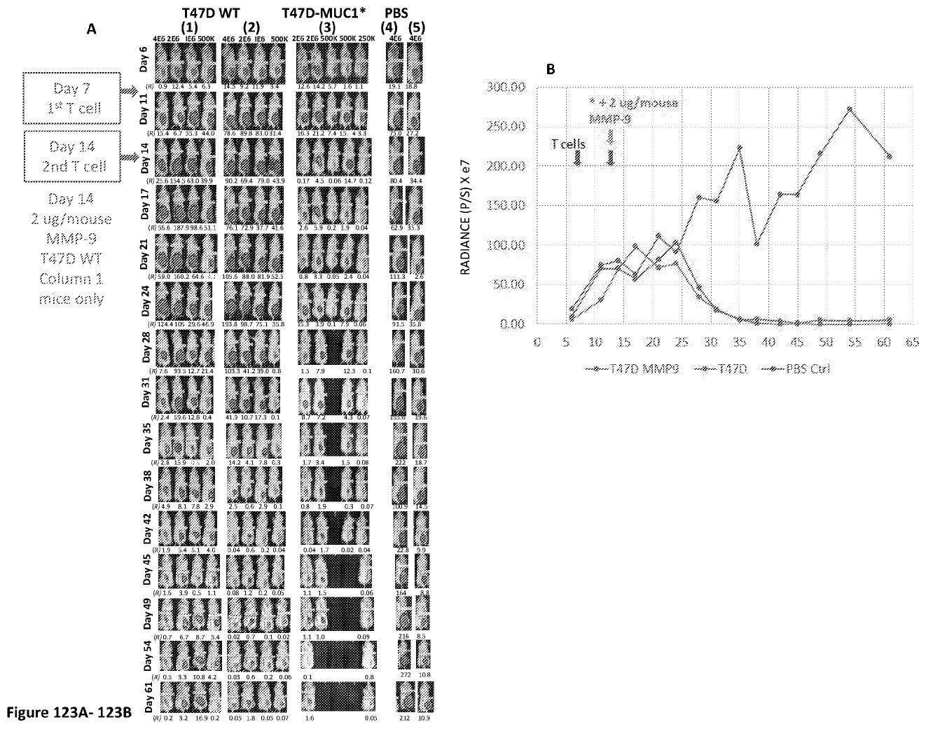

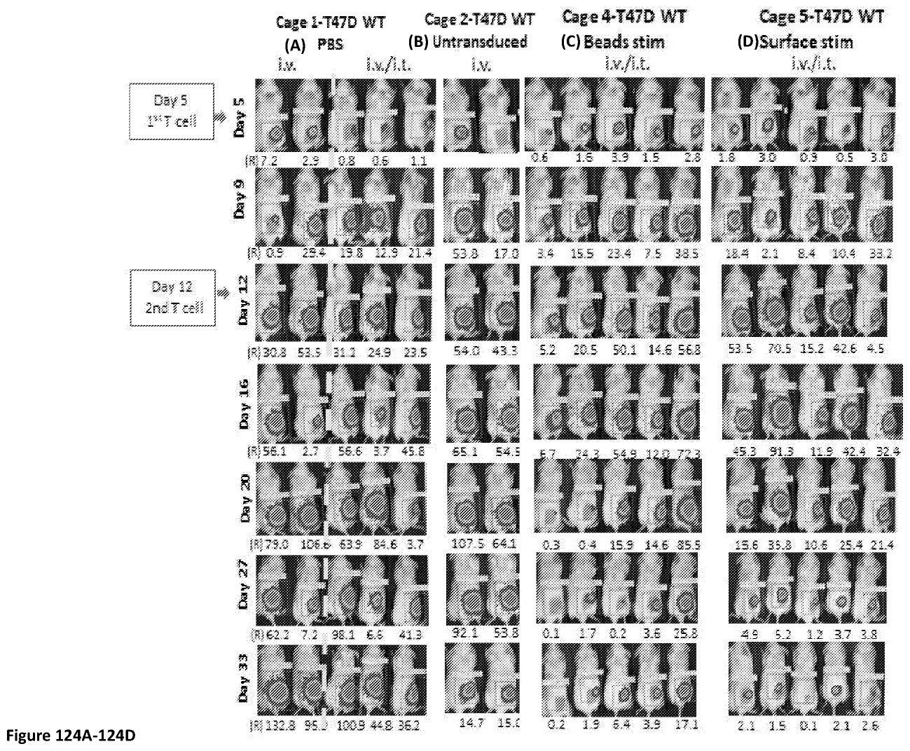

In another in vivo experiment, we compared pre-stimulating CAR T cells with antigen in vitro to stimulating the CAR T cells in vivo. In this experiment, female NSG mice were implanted with T47D breast cancer cells (FIG. 123). 90-day release estrogen pellets had been implanted into all the animals, except one control animal, 1 week before tumor engraftment. Mice were implanted with either 4 million, 2 million, 1 million or 500K cancer cells. Mice shown in FIG. 123A, Column 1 and Column 2 were injected with anti-MUC1* CAR T cells that had been incubated for 24 hours with beads to which was attached the MUC1* extra cellular domain peptide. The CAR T cells were separated from the beads and washed before injecting into the animals. CAR T cells were separated from the peptide beads and washed, then injected into the animals. An in vivo CAR T stimulation strategy was used for the mice shown in FIG. 123A, Column 3. The mice were injected with the same total number of cancer cells as the other mice. However, 40% of the T47D breast cancer that were injected, had been transfected with even more MUC1* transmembrane receptor. This approach simulates taking patient tumor cells, transfecting them with more of the target antigen and then injecting them back into the patient to stimulate the CAR T cells in vivo.

As can be seen, this condition had the most dramatic tumor reduction, showing that in vivo CAR T stimulation also works and that patient cells can be harvested, transfected with more of the target antigen and then injected back into the patient. In the animals, we injected the antigen-enhanced tumor cells near the tumor site. In a patient with localized cancer, the antigen-enhanced tumor cells would be injected back into the patient at or near the tumor site. In a metastatic patient, the antigen-enhanced tumor cells can be injected into the patient systemically.

In this experiment, animals shown in FIG. 123A, Column 1 were injected with 2 ug of a catalytically active MMP9 on Day 14, at the same time as the anti-MUC1* CAR T injection. The strategy was another method of in vivo CAR T stimulation by making the target tumor cells express more of the target antigen. T47D breast cancer cells express both full-length MUC1 and the cleaved growth factor receptor form, MUC1*. MMP9 cleaves MUC1 to the growth factor receptor form MUC1*. In this way, we made at least some of the tumor cells express more of the antigen MUC1*. The mice shown in FIG. 123A, Column 4 and Column 5 were control mice injected only with PBS. The mouse shown in Column 5 was not implanted with an estrogen pellet, which stimulates breast tumor growth.

Mice that were injected with huMNC2-CAR44 T, anti-MUC1* CAR T cells, were injected on Day 7 and on Day 14. Half of the 10 million T cells were injected into the tail vein and the other half were injected near the tumor site. The number of tumor cells implanted did not affect tumor clearance. Six (6) of the ten (10) mice treated with huMNC2-CAR44 T cells were tumor free at Day 61, which was the end of the study. Three (3) of the four (4) mice with residual tumors at the end of the study had tumors that were barely detectable with IVIS readings of ˜2e7 photons/sec/mm\hat{ }3. The fourth mouse had a tumor that measured 16e7 photons/sec/mm\hat{ }3. However, the control without MMP9 treatment and no anti-MUC1* CAR T treatment had a tumor of 212e7 photons/sec/mm\hat{ }3, which is more than 10-times the size of the largest residual tumor in the treated group. FIG. 123B shows a graph of IVIS measurements for all the animals in the study. The mice shown in Column 3 that died, did not die from tumor or treatment. One animal was attacked by cage-mates and the two others died upon injection, which autopsy showed was due to hemorrhage.

In yet another experiment, female NSG mice were subcutaneously implanted with 500,000 T47D naturally MUC1* positive breast cancer cells (FIG. 124). After tumor engraftment was verified, animals were injected with either PBS (FIG. 124A), untransduced human T cells (FIG. 124B), anti-MUC1* CAR T cells that were pre-stimulated in vitro by incubation with beads to which was attached the MUC1* extra cellular domain peptide (FIG. 124C), or anti-MUC1* CAR T cells that were pre-stimulated in vitro by incubation with the MUC1* extra cellular domain peptide which was attached to the bottom of a cell culture flask (FIG. 124D). In this particular experiment, the surface attached peptide was MUC1* extra cellular domain peptide that had been coupled to BSA then coated onto a cell culture flask at 1 ug/mL.

In these examples, anti-MUC1* CAR T cells, huMNC2-CAR44 T cells, are stimulated by incubating them for 24 hours with the MUC1* extra cellular domain peptide attached to a bead, to BSA then coated onto a surface, to BSA then added in solution, to a surface or free in solution. Most or all the stimulating antigen is washed away and the anti-MUC1* CAR T cells are co-cultured with MUC1* positive cancer cells. However, this strategy can be used to pre-stimulate any CAR T cell, or other immune cell bearing a CAR, just by incubating the CAR T cells with the antigen their antibody head binds to, for a period of time that can be as little as 8 hours and as much as days. The trade-off is that as CAR T cells continue to be activated, as evidenced by an increase in expression of CD69 and CD25, they continue to mature, which research has shown makes them less persistent when injected into a patient. Current research indicates that it is important for persistence in vivo to have a significant percentage of the CAR T cells in the naïve state and central memory state, as well as in the effector memory and effector state.

Described here is a method of culturing CAR T cells that produces desired memory phenotype and CD4 vs CD8 ratio, which result in persistence and low side effects when administered to patient. Previous work showed that a roughly 50/50-30/70 ratio of CD4:CD8 increased persistence of CAR T cells in vivo. It has also been shown that it is desirable to have a good portion of the CAR T cells in the CD62L positive CD45-RO negative (naïve) state and CD62L positive CD45-RO positive (central memory) state, with much smaller portion in the CD62L negative/CD45-RO positive (effector memory) and CD62L negative/CD45-RO negative effector state. In general, we found that the longer the T cells are in culture in vitro, the more differentiated they become and the less cells are in the desired naïve and central memory state. Using our protocol, the patient's T cells are only in culture for 9-12 days but the yield is great enough for at least two CAR T cell injections and the protocol produces a high percentage of the CAR T cells in the Central memory and naïve state with far less in the effector memory and effector cell state.

In brief, our protocol takes patient T cells (Day 0), activates T cells with anti-CD3/anti-CD28 beads or surfaces for 48 hrs, followed on Day 2 by transduction (can be viral, lenti virus, Sleeping Beauty, Crispr etc.) of the T cells with plasmid bearing the CAR, expansion in media containing IL2, IL15, and/or IL17 until Day 9, on Day 9 pre-stimulate CAR T cells by incubating with target antigen attached to bead or surface for 18-24 hours, inject into patient, preferably within 24-72 hours after removal from the peptide activating surface. The timing of each of these steps can be varied without too much of an effect on outcome. In general, CAR T cells should be kept in culture for as short a time as possible and administered to patient, preferably within 24-48 hours of antigen stimulation.

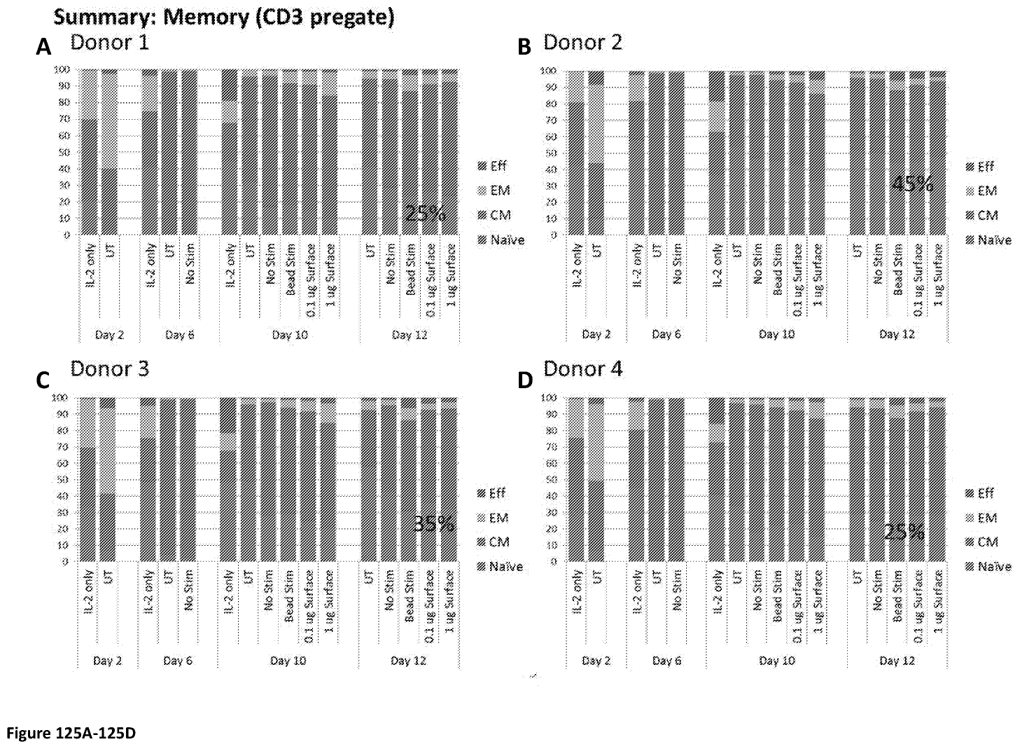

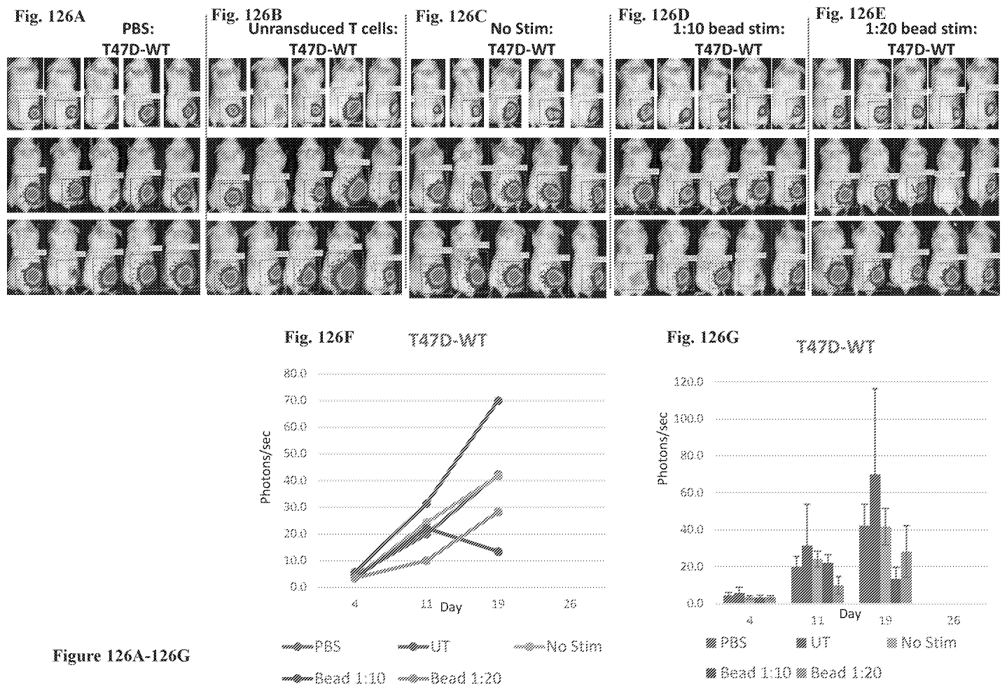

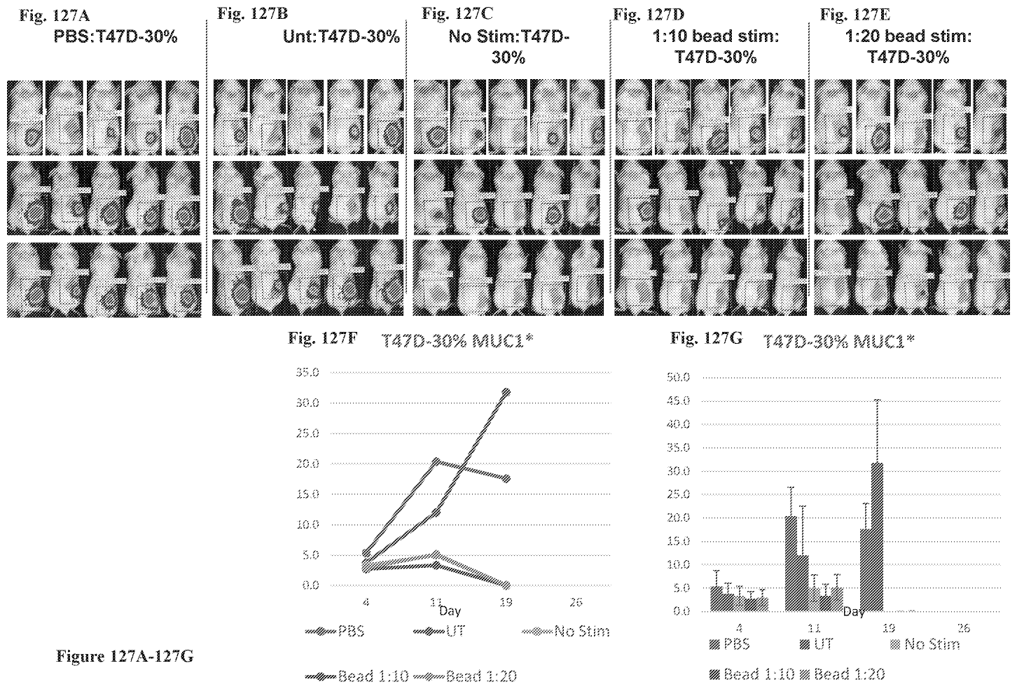

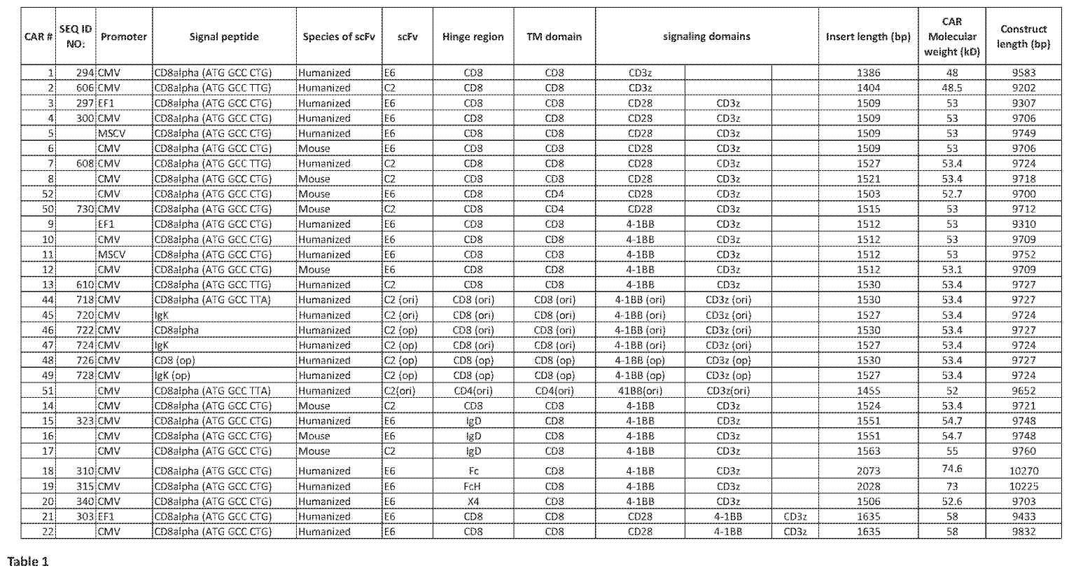

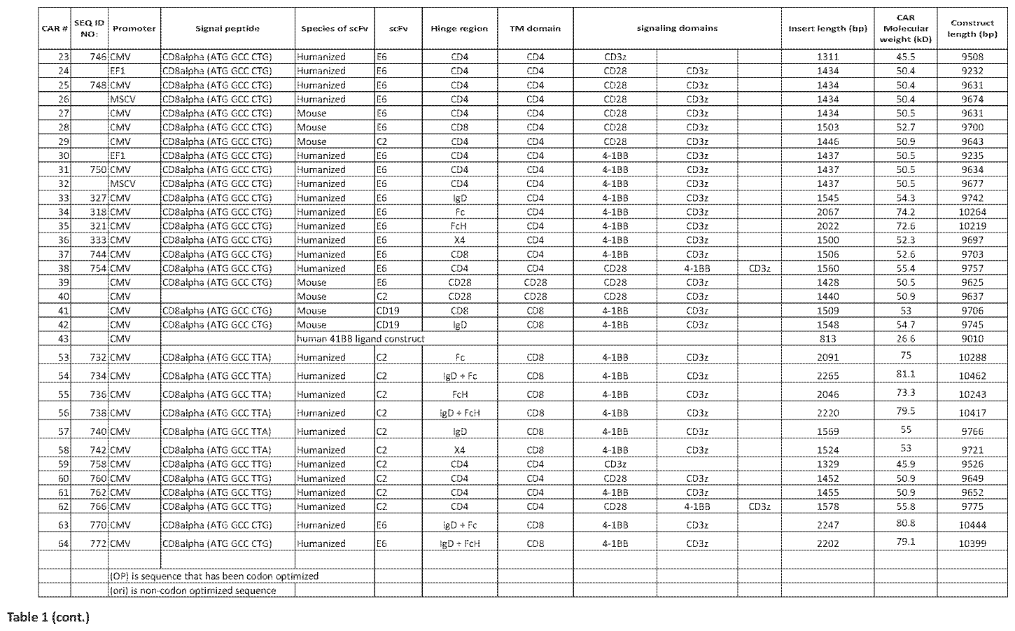

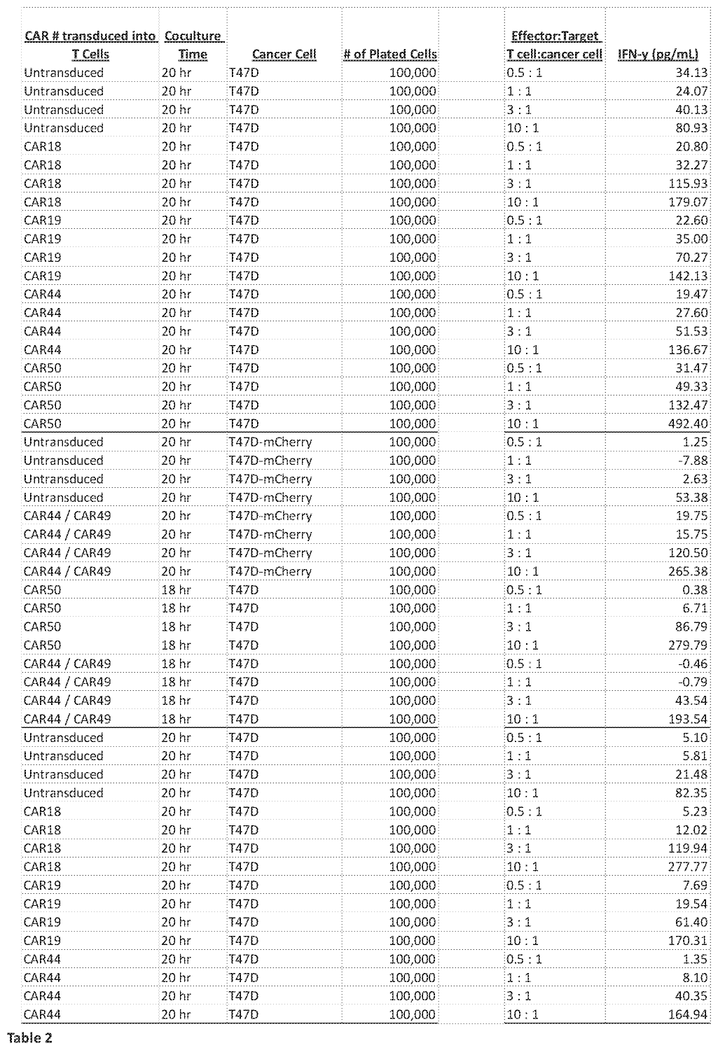

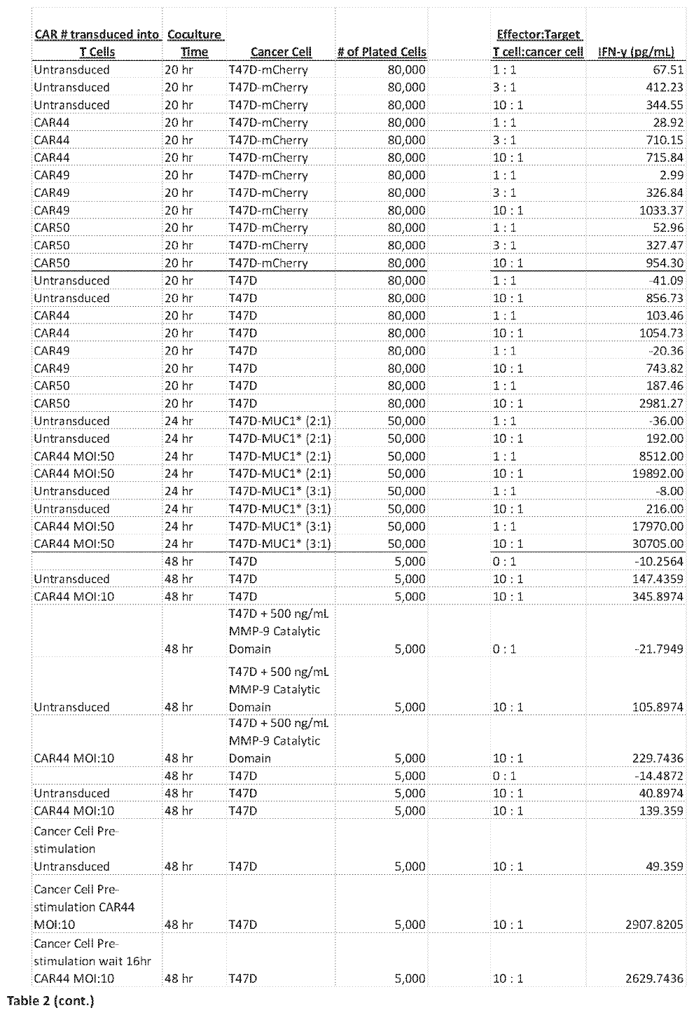

FIGS. 125A-125D show bar graphs that summarize FACS analysis of the CAR T cell memory sub-populations as the CAR T cells go through the in vitro manufacturing process. For the four (4) different donors shown, T cells were transduced with an anti-MUC1* CAR on Day 2 then expanded in culture until Day 9, stimulated by incubation with the target antigen for 24 hours, then washed and removed. Analysis was done by FACS on Day 10 and Day 12, since this would be the timing for patient injection. As can be seen, the protocol leaves large percentages of the T cells in the desirable Naïve and Central Memory state, which leads to persistence in vivo. The effect in vivo of antigen stimulation of CAR T cells was directly compared to that of CAR T cells that had not been antigen stimulated. Immune compromised female NOD/SCID/GAMMA mice were implanted with 90-day estrogen release pellets, then implanted sub-cutaneously with 500,000 MUC1* positive breast cancer, T47D wild type, cells. Another group was implanted with 500,000 cancer cells, 70% of which were T47D wild type and 30% were T47D wild type that had been transduced with even more MUC1*. These tumor cells expressing more MUC1* are referred to herein as T47D-30%. All cancer cells had been previously transduced with Luciferase to facilitate visualization of the tumor using an IVIS instrument. Ten minutes before IVIS measurements, animals were i.p. injected with the Luciferase substrate Luciferin, which causes the tumor cells to emit light. Tumor engraftment was verified by IVIS measurement 5 days post tumor cell implantation. Animals were injected into the tail vein with either mock treatment or anti-MUC1* CAR T cells, with or without prior antigen stimulation. FIG. 126A-126G shows photographs and corresponding graphs that show the effect of antigen stimulation on CAR T cells in vivo. FIG. 126A-126E shows IVIS fluorescent photographs of female NOD/SCID/GAMMA mice that have been implanted with 90-day estrogen release pellets, then implanted with human MUC1* positive breast cancer cells, T47D wild type. The animals were injected on Day 5 and again on Day 12 with either PBS, untransduced human T cells, or human T cells transduced with huMNC2-CAR44 T cells that were either antigen stimulated or not. FIG. 126A shows mice injected with PBS. FIG. 126B shows mice injected with 10M untransduced human T cells. FIG. 126C shows mice injected with 7M huMNC2-CAR44 T cells that were not antigen stimulated. FIG. 126D shows mice injected with 7M huMNC2-CAR44 T cells that were previously antigen stimulated using 4.5 micron magnetic beads to which was attached synthetic PSMGFR, MUC1*, peptides at a bead to T cell ratio of 1:10. FIG. 126E shows mice injected with 7M huMNC2-CAR44 T cells that were previously antigen stimulated using 4.5 micron magnetic beads to which was attached synthetic PSMGFR, MUC1*, peptides at a bead to T cell ratio of 1:20. FIG. 126F shows a line graph summarizing tumor volume as measured by IVIS from Day 1 to Day 19 post tumor cell implantation. FIG. 126G shows a bar graph summarizing tumor volume as measured by IVIS from Day 1 to Day 19 post tumor cell implantation. Photographs were taken on an IVIS instrument that measures fluorescence emitted from the cancer cells which had been engineered to express Luciferase. Ten (10) minutes prior to IVIS analysis, the Luciferase substrate Luciferin was injected intra-peritonealy (i.p.) into the animal. As can be seen in the photos and graphs, pre-stimulation of CAR T cells with antigen greatly increased the killing efficiency of the CAR T cells. An increase in the ratio of beads to T cells resulted in increased killing. FIG. 127A-127G shows photographs and corresponding graphs that show the effect of antigen stimulation on CAR T cells in vivo. FIG. 127A-127E shows IVIS fluorescent photographs of female NOD/SCID/GAMMA mice that have been implanted with 90-day estrogen release pellets, then implanted with human MUC1* positive breast cancer cells, T47D wild type. The animals were injected on Day 5 and again on Day 12 with either PBS, untransduced human T cells, or human T cells transduced with huMNC2-CAR44 T cells that were either antigen stimulated or not. FIG. 127A shows mice injected with PBS. FIG. 127B shows mice injected with 10M untransduced human T cells. FIG. 127C shows mice injected with 7M huMNC2-CAR44 T cells that were not antigen stimulated. FIG. 127D shows mice injected with 7M huMNC2-CAR44 T cells that were previously antigen stimulated using 4.5 micron magnetic beads to which was attached synthetic PSMGFR, MUC1*, peptides at a bead to T cell ratio of 1:10. FIG. 127E shows mice injected with 7M huMNC2-CAR44 T cells that were previously antigen stimulated using 4.5 micron magnetic beads to which was attached synthetic PSMGFR, MUC1*, peptides at a bead to T cell ratio of 1:20. FIG. 127F shows a line graph summarizing tumor volume as measured by IVIS from Day 1 to Day 19 post tumor cell implantation. FIG. 127G shows a bar graph summarizing tumor volume as measured by IVIS from Day 1 to Day 19 post tumor cell implantation. Photographs were taken on an IVIS instrument that measures fluorescence emitted from the cancer cells which had been engineered to express Luciferase. Ten (10) minutes prior to IVIS analysis, the Luciferase substrate Luciferin was injected intra-peritonealy (i.p.) into the animal. As can be seen in the photos and graphs, pre-stimulation of CAR T cells with antigen greatly increased the killing efficiency of the CAR T cells. An increase in the ratio of beads to T cells resulted in increased killing.

Peptide coupled to Mono-Mag Maleimide Activated Magnetic Beads (Bead Stim)

Commercially available mono-mag maleimide activated magnetic beads (Ocean Nanontech catalog #MM1001) were conjugated through the thiol of C-terminal cysteine of FLR-Cys. To 2.5 mg of 1 μm Mono-Mag beads (50 mg of powder) in coupling buffer (1 mL, 100 mM PBS with 5 mM EDTA, 0.01% tween, pH 7.4) was added 25 nanomoles of peptide in DMSO (250 uL). The coupling reaction was rocked overnight at 4 C. Quenching buffer (100 uL) composed of 10 mg/mL mercaptosuccinic acid in 100 mM PBS was added and the incubated for an addition 4 hours at room temperature. The beads were then separated from the supernatant in a magnetic separator and washed three times with storage buffer composed of 10 mM PBS with 0.02% NaN3, 0.01% tween 20, 0.1% BSA, pH 7.4. The resulting beads were stored at +4 C until ready for use.

Peptide Coupled to Epoxy Dynabeads Magentic Beads (Bead Stim)

Commerically available epoxy dynabeads (Invitrogen/Thermo H-450, 4.5 um, catalog #14011) (1 mL) were washed with water in a magnetic separator, then conc. ammonium hydroxide (1 mL) was added. The beads were rocked at 15 C over the weekend (36 h). The resulting amine beads were washed with water (4×1 mL) in a magnetic separator until the pH was 7 (litmus paper). The amine beads were then suspended in coupling buffer composed of 100 mM PBS, 5 mM EDTA, 0.01% tween, pH 7.4 and to that was added sulfo-SMCC (10 mg) dissolved in DMSO (200 uL). The amide formation reaction was rocked at 15 C for 3 hours. Then more sulfo-SMCC (10 mg) dissolved in DMSO (200 uL) was added and the reaction mixture rocked overnight at room temperature. The maleimide-activated beads were then separated and washed with coupling buffer (3×1 mL). The maleimide-activated beads were then suspended in 0.75 mL of coupling buffer (100 mM PBS, 5 mM EDTA, 0.01% tween, pH 7.4). FLR-cysteine peptide (25 nmoles) in 250 uL of 5% DMSO in coupling buffer. The beads were rocked at 25 C overnight. The beads were then magnetically separated and washed with PBS containing 0.1% BSA at pH 7.4. The beads were then separated and suspended in storage buffer composed of 10 mM PBS with 0.02% NaN3, 0.01% tween 20, 0.1% BSA, pH 7.4. The resulting beads were stored at +4 C until ready for use.

Methods Used in Carrying Out Experimentation in Relation to the Present Invention

Lentivirus Production and Viral Transduction of Immune Cells

HEK293 or HEK293T cells (ATCC) were used to produce lentivirus. The day prior transfection plates (6well plate) were coated with poly-D-lysine and cells seeded so that cell density reaches 90-95% at the time of transfection and cultures in a 5% CO2 atmosphere. The next day cells were transfected with Lipofectamine 3000 (life technologies) and Opti-MEM® I Reduced Serum Medium according to the manufacturer instructions (0.75 ug of lentiviral expression vector and 2.25 ug of pPACKH1 packaging mix was used). After 6 h incubation, the media was changed and media containing lentivirus was harvested after 24 and 48 hours. Lentivirus was concentrated with Lenti-X concentrator (Clontech) and titer was calculated using the Lenti-X p@4 Rapid Titer Kit (Clontech). Lentivirus was store at −80 C in single-use aliquots.

Transduction of Immune Cells with Constructs Including CARs

Human T cells, if frozen, were thawed and pre-warmed in 100-200 units IL-2 and TexMACS medium, 20 ml, and pelleted by centrifugation. Cells were resuspended in 10 ml of medium and cultured at 37° C., 5% CO₂ at 1×10⁶ cells/ml in complete medium with anti-CD3/anti-CD28 beads (TransAct kit).

After 4 days in culture, cells were counted and 450 ul of cell suspension was placed in single well of a 24-well plate at a density of approximately 1×10⁶ cells/ml. Cells were allowed to settle. 150 ul was carefully removed from the top of each well. To each well was added an appropriate dilution of lentiviral vector, diluted in plain TexMACS medium, along with protamine sulfate to a final concentration of 10 ug/ml, in a 150 ul volume, for a final total volume of 450 ul per well and incubated for 24 hrs. Transduced cells were removed, pelleted by centrifugation, and resuspended in fresh medium, adjusting cell density, not to exceed 1.0×10⁶ cells/ml. Transduced T cells can be expanded and frozen or used directly. Typically transduced T cells are used or frozen between Day 7 and Day 20 post activation with IL-2 and TransAct media.

Analysis of CAR T Cell Induced Killing of MUC1* Positive Cancer Cells by xCELLigence

In addition to FACS analysis, many researchers now use an xCELLigence instrument to measure CAR T killing of cancer cells. The xCELLigence instrument uses electrode arrays upon which cancer cells are plated. The adherent cancer cells insulate the electrode and so cause an increase in impedance as they grow. Conversely, T cells are not adherent and remain in suspension so do not contribute to insulation of the electrode which would increase impedance. However, if the T cells or CAR T cells kill the cancer cells on the electrode plate, the cancer cells ball up and float off as they die, which causes the impedance to decrease. The xCELLigence instrument measures impedance as a function of time, which is correlated to cancer cell killing. In addition, the electrode plates also have a viewing window. When CAR T cells effectively kill the adsorbed target cancer cells, there is a decrease in impedance but also one can see that there are no cancer cells left on the plate surface.

In most of the XCELLigence experiments, 5,000 cancer cells were plated per well of a 96-well electrode array plate. Cells were allowed to adhere and grow for 24 hours. CAR T cells were then added at an Effector to Target ratio (E:T) of 0.5:1, 1:1, 2:1, 5:1, 10:1 and sometimes 20:1. The E:T ratio assumes 100% transduction of the CAR into the T cells, when the actual transduction efficiency is 40%.

The xCELLigence instrument records impedance as a function of time and experiments can go on for up to 7 days.

FIGS. 89A-89C show results of CAR T and cancer cell experiments performed on an xCELLigence instrument.

Anti-MUC1* CAR T Cell Therapy in Mice Bearing Human Tumors

Female NOD/SCID/GAMMA (NSG) mice between 8-12 weeks of age were implanted with 500,000 human cancer cells, wherein the cancer cells had previously been stably transfected with Luciferase. Mice bearing Luciferase positive cells can be injected with the enzyme's substrate Luciferin just prior to imaging, which makes the cancer cells fluoresce. The cancer cells are imaged in live mice within 10-15 minutes after injection with Luciferin on an IVIS instrument. The readout is flux or photons per second. Tumors were allowed to engraft until tumors were clearly visible by IVIS.

FIGS. 91A-91Y show fluorescent photographs of mice taken on an IVIS instrument. NSG (NOD/SCID/GAMMA) immune compromised mice that on Day 0 were subcutaneously implanted on the flank with 500,000 human MUC1* positive cancer cells that had been stably transfected with Luciferase. Tumors were allowed to engraft. On Day 5 after IVIS measurement and on Day 12, animals were injected with 10 million of either human T cells transduced with huMNC2-scFv-CAR44, untransduced T cells or PBS. 5 million T cells were injected intra-tumor and 5 million T cells were injected into the tail vein. 10 minutes prior to IVIS photographs, mice were injected intraperitoneally (IP) with Luciferin, which fluoresces after cleavage by Luciferase, thus making tumor cells fluoresce.

FIGS. 92A-92J show fluorescent photographs of mice taken on an IVIS instrument. NSG (NOD/SCID/GAMMA) immune compromised mice that on Day 0 were subcutaneously injected into the flank with 500K human BT-20 cells which are a MUC1* positive triple negative breast cancer cell line. The cancer cells had been stably transfected with Luciferase. Tumors were allowed to engraft. On Day 6 after IVIS measurement, animals were given a one-time injection of 10 million of either human T cells transduced with huMNC2-scFv-CAR44 or untransduced T cells. 5 million T cells were injected intra-tumor and 5 million were injected into the tail vein. 10 minutes prior to IVIS photographs, mice were IP injected with Luciferin.

FIGS. 93A-93H show fluorescent photographs of mice taken on an IVIS instrument.

NSG (NOD/SCID/GAMMA) immune compromised mice that on Day 0 were injected into the intraperitoneal cavity (IP) with 500K human SKOV-3 cells which are a MUC1* positive ovarian cancer cell line. The cancer cells had been stably transfected with Luciferase. Tumors were allowed to engraft. On Day 3 after IVIS measurement, animals were IP injected with 10M either human T cells transduced with huMNC2-scFv-CAR44, untransduced T cells or PBS. Animals were IVIS imaged again on Day 7. 10 minutes prior to IVIS photographs, mice were IP injected with Luciferin.

Antigen Peptide Coupled to BSA (Bovine Serum Albumin)

A MUC1* extra cellular domain peptide, PSMGFR sequence with Cys modification at C-terminus was coupled to maleimide activated bovine serum albumin (ThermoFisher, Cat #77115) through thiol coupling of the terminal cysteine. Peptide was serially diluted in 0.1M bicarbonate buffer (pH 9.6) to concentrations of 10, 2, 1, 0.2, 0.1, 0.01, 0.001 ug/mL. 1 mL of each dilution was added to each well of a 6well Nunc plate. A bicarbonate buffer only well was also coated as a negative control. Plates were wrapped in parafilm and stored at 4 degrees C. overnight. The following day coating solution was aspirated and then wells were washed with 2 mL of PBS. Day 9 T Cells were then added at 1 million cells per mL. 24 hrs later, wells were inspected for changes in T Cell morphology and clumping behavior that denotes activation; images were taken. A portion of the T Cells were removed from the coated well at 24, 48, 72, and 96 hours, washed and re-plated in normal uncoated culture conditions. Analysis of activation and memory markers was done at multiple time points. T Cells were also subjected to co-culture assays with various cancer cell lines to assess cytotoxicity.

Antigen Peptide Coupled to HSA (Human Serum Albumin)

Peptide was coupled to maleimide activated human serum albumin (Nanoc, Cat # HS1-ML-1) (Minerva Biotechnologies, STM258158) through thiol coupling of the terminal cysteine. Peptide was serially diluted in 0.1M bicarbonate buffer (pH 9.6) to concentrations of 10, 2, 1, 0.2, 0.1, 0.01, 0.001 ug/mL. 1 mL of each dilution was added to each well of a 6well Nunc plate. A bicarbonate buffer only well was also coated as a negative control. Plates were wrapped in parafilm and stored at 4 degrees C. overnight. The following day coating solution was aspirated and then wells were washed with 2 mL of PBS. Day 9 T Cells were then added at 1 million cells per mL. 24 hrs later wells were inspected for changes in T Cell morphology and clumping behavior that denotes activation; images were taken. T Cells were then removed from coated wells and analyzed by flow cytometry for activation/memory markers and subjected to co-culture assays with various cancer cell lines.

Antigen Peptide Attached to Amine Derivatized Surface

Amine derivatized 6-well plates from Corning were coupled to SMCC by reaction with sulfo-SMCC. MUC1* peptide was conjugated to maleimide group on SMCC through thiol of terminal cysteine. Reaction was performed at different concentrations of peptide in the presence of capping agent—mercaptosuccinic acid to achieve different amounts of peptide conjugated to the plate. At the conclusion of the reaction, excess mercaptosuccinic acid was used to cap remaining maleimide. Plates were then washed 3 times with PBS and stored at 4 degrees C. overnight. The following day PBS was aspirated and then wells were washed again with 2 mL of PBS. Day 9 T Cells were then added at 1 million cells per mL. 24 hrs later wells were inspected for changes in T Cell morphology and clumping behavior indicative of activation; images were taken. T Cells were then removed from coated wells and analyzed by flow cytometry for activation/memory markers and subjected to co-culture assays with various cancer cell lines.

Antigen Peptide-BSA Free in Solution (Soluble Stim)

Peptide was coupled to maleimide activated bovine serum albumin (ThermoFisher, Cat #77115) through thiol coupling of the terminal cysteine. Day 9 T Cells were plated at 1 million cells per mL. Peptide-BSA was pre-diluted in 1 mL of media and then added to respective wells of a 6 well plate. Day 9 T Cells were added to each well for a final concentration of 1 million cells per mL and to the effect of 10, 1, 0.1, 0.01, 0.001 ug/mL of peptide-BSA. 24 hrs later wells were inspected for changes in T Cell morphology and clumping behavior indicative of activation; images were taken. A portion of the T Cells were removed from the well at 24, 48, 72, and 96 hours, washed and re-plated in normal uncoated culture conditions. Analysis of activation and memory markers was done at multiple time points. T Cells were also subjected to co-culture assays with various cancer cell lines to assess cytotoxicity.

Antigen Peptide Free in Solution

Peptide was coupled to maleimide activated bovine serum albumin (ThermoFisher, Cat #77115) through thiol coupling of the terminal cysteine. Day 9 T Cells were plated at 1 million cells per mL. Peptide-BSA was prediluted in 1 mL of media and then added to respective wells of a 6 well plate. Day 9 T Cells were added to each well for a final concentration of 1 million cells per mL and to the effect of 1 ug/mL of MUC1* peptide. 24 hrs later wells were inspected for changes in T Cell morphology and clumping behavior indicative of activation; images were taken. A portion of the T Cells were removed from the well at 24 hours, washed and re-plated in normal uncoated culture conditions. Analysis of activation and memory markers was done at multiple time points. T Cells were also subjected to co-culture assays with various cancer cell lines to assess cytotoxicity.

Antigen Peptide Non-Specifically Adsorbed to a Plate

Peptide was serially diluted in 0.1M bicarbonate buffer (pH 9.6) to concentrations of 10, 1, 0.1, 0.01, 0.001, 0.0001 ug/mL. 1 mL of each dilution was added to respective well of a 6well Nunc plate. A bicarbonate buffer only well was also coated as a negative control. Plates were wrapped in parafilm and stored at 4 degrees C. overnight. The following day coating solution was aspirated and then wells were washed with 2 mL of PBS. Day 9 T Cells were then added at 1 million cells per mL. 24 hrs later wells were inspected for changes in T Cell morphology and clumping behavior that denotes activation; images were taken. A portion of the T Cells were removed from the coated well at 24 hours, washed and re-plated in normal uncoated culture conditions. Analysis of activation and memory markers was done at multiple time points. T Cells were also subjected to co-culture assays with various cancer cell lines to assess cytotoxicity.

All of the references cited herein are incorporated by reference in their entirety.

Sequence Listing Free Text
As regards the use of nucleotide symbols other than a, g, c, t, they
follow the convention set forth in WIPO Standard ST.25, Appendix 2,
Table 1, wherein k represents t or g; n represents a, c, t or g; m
represents a or c; r represents a or g; s represents c or g; w
represents a or t and y represents c or t.
MUC1 Receptor
(Mucin 1 precursor, Genbank Accession number: P15941)
(SEQ ID NO: 1)
MTPGTQSPFFLLLLLTVLTVVTGSGHASSTPGGEKETSATQRSSVPSSTEKNAVSMTSSVLSSHSPGSGSSTTQGQDVTLA
PATEPASGSAATWGQDVTSVPVTRPALGSTTPPAHDVTSAPDNKPAPGSTAPPAHGVTSAPDTRPAPGSTAPPAHGVTSAP
DTRPAPGSTAPPAHGVTSAPDTRPAPGSTAPPAHGVTSAPDTRPAPGSTAPPAHGVTSAPDTRPAPGSTAPPAHGVTSAPD
TRPAPGSTAPPAHGVTSAPDTRPAPGSTAPPAHGVTSAPDTRPAPGSTAPPAHGVTSAPDTRPAPGSTAPPAHGVTSAPDT
RPAPGSTAPPAHGVTSAPDTRPAPGSTAPPAHGVTSAPDTRPAPGSTAPPAHGVTSAPDTRPAPGSTAPPAHGVTSAPDTR
PAPGSTAPPAHGVTSAPDTRPAPGSTAPPAHGVTSAPDTRPAPGSTAPPAHGVTSAPDTRPAPGSTAPPAHGVTSAPDTRP
APGSTAPPAHGVTSAPDTRPAPGSTAPPAHGVTSAPDTRPAPGSTAPPAHGVTSAPDTRPAPGSTAPPAHGVTSAPDTRPA
PGSTAPPAHGVTSAPDTRPAPGSTAPPAHGVTSAPDTRPAPGSTAPPAHGVTSAPDTRPAPGSTAPPAHGVTSAPDTRPAP
GSTAPPAHGVTSAPDTRPAPGSTAPPAHGVTSAPDTRPAPGSTAPPAHGVTSAPDTRPAPGSTAPPAHGVTSAPDTRPAPG
STAPPAHGVTSAPDTRPAPGSTAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGSTAPPAHGVTSAPDTRPAPGST
APPAHGVTSAPDTRPAPGSTAPPAHGVTSAPDTRPAPGSTAPPAHGVTSAPDNRPALGSTAPPVHNVTSASGSASGSASTL
VHNGTSARATTTPASKSTPFSIPSHHSDTPTTLASHSTKTDASSTHHSSVPPLTSSNHSTSPQLSTGVSFFFLSFHISNLQ
FNSSLEDPSTDYYQELQRDISEMFLQIYKQGGFLGLSNIKFRPGSVVVQLTLAFREGTINVHDVETQFNQYKTEAASRYNL
TISDVSVSDVPFPFSAQSGAGVPGWGIALLVLVCVLVALAIVYLIALAVCQCRRKNYGQLDIFPARDTYHPMSEYPTYHTH
GRYVPPSSTDRSPYEKVSAGNGGSSLSYTNPAVAAASANL
PSMGFR
(SEQ ID NO: 2)
GTINVHDVETQFNQYKTEAASRYNLTISDVSVSDVPFPFSAQSGA
Human NME1
(DNA)
(SEQ ID NO: 3)
atggccaactgtgagcgtaccttcattgcgatcaaaccagatggggtccagcggggtcttgtgggagagattatcaagcg
ttttgagcagaaaggattccgccttgttggtctgaaattcatgcaagcttccgaagatcttctcaaggaacactacgttg
acctgaaggaccgtccattctttgccggcctggtgaaatacatgcactcagggccggtagttgccatggtctgggagggg
ctgaatgtggtgaagacgggccgagtcatgctcggggagaccaaccctgcagactccaagcctgggaccatccgtggaga
cttctgcatacaagttggcaggaacattatacatggcagtgattctgtggagagtgcagagaaggagatcggcttgtggt
ttcaccctgaggaactggtagattacacgagctgtgctcagaactggatctatgaatga
(amino acids)
(SEQ ID NO: 4)
MANCERTFIAIKPDGVQRGLVGEIIKRFEQKGFRLVGLKFMQASEDLLKEHYVDLKDRPFFAGLVKYMHSGPVVAMVWEG
LNVVKTGRVMLGETNPADSKPGTIRGDFCIQVGRNIIHGSDSVESAEKEIGLWFHPEELVDYTSCAQNWIYE-
Human NME7
(DNA)
(SEQ ID NO: 5)
atgaatcatagtgaaagattcgttttcattgcagagtggtatgatccaaatgcttcacttcttcgacgttatgagctttt
attttacccaggggatggatctgttgaaatgcatgatgtaaagaatcatcgcacctttttaaagcggaccaaatatgata
acctgcacttggaagatttatttataggcaacaaagtgaatgtcttttctcgacaactggtattaattgactatggggat
caatatacagctcgccagctgggcagtaggaaagaaaaaacgctagccctaattaaaccagatgcaatatcaaaggctgg
agaaataattgaaataataaacaaagctggatttactataaccaaactcaaaatgatgatgctttcaaggaaagaagcat
tggattttcatgtagatcaccagtcaagaccctttttcaatgagctgatccagtttattacaactggtcctattattgcc
atggagattttaagagatgatgctatatgtgaatggaaaagactgctgggacctgcaaactctggagtggcacgcacaga
tgcttctgaaagcattagagccctctttggaacagatggcataagaaatgcagcgcatggccctgattcttttgcttctg
cggccagagaaatggagttgttttttccttcaagtggaggttgtgggccggcaaacactgctaaatttactaattgtacc
tgttgcattgttaaaccccatgctgtcagtgaaggactgttgggaaagatcctgatggctatccgagatgcaggttttga
aatctcagctatgcagatgttcaatatggatcgggttaatgttgaggaattctatgaagtttataaaggagtagtgaccg
aatatcatgacatggtgacagaaatgtattctggcccttgtgtagcaatggagattcaacagaataatgctacaaagaca
tttcgagaattttgtggacctgctgatcctgaaattgcccggcatttacgccctggaactctcagagcaatctttggtaa
aactaagatccagaatgctgttcactgtactgatctgccagaggatggcctattagaggttcaatacttcttcaagatct
tggataattag
(amino acids)
(SEQ ID NO: 6)
MNHSERFVFIAEWYDPNASLLRRYELLFYPGDGSVEMHDVKNHRTFLKRTKYDNLHLEDLFIGNKVNVFSRQLVLIDYGD
QYTARQLGSRKEKTLALIKPDAISKAGEIIEIINKAGFTITKLKMMMLSRKEALDFHVDHQSRPFFNELIQFITTGPIIA
MEILRDDAICEWKRLLGPANSGVARTDASESIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKGVVTEYHDMVTEMYSGPCVAMEIQQNNATKT
FREFCGPADPEIARHLRPGTLRAIFGKTKIQNAVHCTDLPEDGLLEVQYFFKILDN-
NME7 peptides
NME7A peptide 1 (A domain):
(SEQ ID NO: 7)
MLSRKEALDFHVDHQS
NME7A peptide 2 (A domain):
(SEQ ID NO: 8)
SGVARTDASES
NME7B peptide 1 (B domain):
(SEQ ID NO: 9)
DAGFEISAMQMFNMDRVNVE
NME7B peptide 2 (B domain):
(SEQ ID NO: 10)
EVYKGVVTEYHDMVTE
NME7B peptide 3 (B domain):
(SEQ ID NO: 11)
AIFGKTKIQNAVHCTDLPEDGLLEVQYFF
Mouse E6 Heavy chain variable region sequence:
(DNA)
(SEQ ID NO: 12)
gaggtgaaggtggtggagtctgggggagacttagtgaagcctggagggtccctgaaactctcctgtgtagtctctggattc
actttcagtagatatggcatgtcttgggttcgccagactccaggcaagaggctggagtgggtcgcaaccattagtggtggc
ggtacttacatctactatccagacagtgtgaaggggcgattcaccatctccagagacaatgccaagaacaccctgtacctg
caaatgagcagtctgaagtctgaggacacagccatgtatcactgtacaagggataactacggtaggaactacgactacggt
atggactactggggtcaaggaacctcagtcaccgtctcctca
(amino acids)
(SEQ ID NO: 13)
EVKVVESGODLVKPGGSLKLSCVVSGFTFSRYGMSWVRQTPGKRLEWVATISGGGTYIYYPDSVKGRFTISRDNAKNTLYL
QMSSLKSEDTAMYHCTRDNYGRNYDYGMDYWGQGTSVTVSS
Mouse E6 heavy chain variable framework region 1 (FWR1) sequence:
(DNA)
(SEQ ID NO: 14)
gaggtgaaggtggtggagtctgggggagacttagtgaagcctggagggtccctgaaactctcctgtgtagtctct
(amino acids)
(SEQ ID NO: 15)
EVKVVESGGDLVKPGGSLKLSCVVSGFTFS
Mouse E6 heavy chain variable complementarity determining regions 1 (CDR1)
sequence:
(DNA)
(SEQ ID NO: 16)
ggattcactttcagtagatatggcatgtct
(amino acids)
(SEQ ID NO: 17)
RYGMS
Mouse E6 heavy chain variable framework region 2 (FWR2) sequence:
(DNA)
(SEQ ID NO: 18)
tgggttcgccagactccaggcaagaggctggagtgggtcgca
(amino acids)
(SEQ ID NO: 19)
WVRQTPGKRLEWVA
Mouse E6 heavy chain variable complementarity determining regions 2 (CDR2)
sequence:
(DNA)
(SEQ ID NO: 20)
accattagtggtggcggtacttacatctactatccagacagtgtgaagggg
(amino acids)
(SEQ ID NO: 21)
TISGGGTYIYYPDSVKG
Mouse E6 heavy chain variable framework region 3 (FWR3) acid sequence:
(DNA)
(SEQ ID NO: 22)
cgattcaccatctccagagacaatgccaagaacaccctgtacctgcaaatgagcagtctgaagtctgaggacacagccat
gtatcactgtacaagg
(amino acids)
(SEQ ID NO: 23)
RFTISRDNAKNTLYLQMSSLKSEDTAMYHCTR
Mouse E6 heavy chain variable complementarity determining regions 3 (CDR3)
sequence:
(DNA)
(SEQ ID NO: 24)
gataactacggtaggaactacgactacggtatggactac
(amino acids)
(SEQ ID NO: 25)
DNYGRNYDYGMDY
Humanized E6 heavy chain variable region sequence:
(DNA)
(SEQ ID NO: 38)
gaggtgcagctggtggagtctgggggaggcctggtcaagcctggggggtccctgagactctcctgtgcagcctctggatt
caccttcagtaggtatggcatgagctgggtccgccaggctccagggaagaggctggagtgggtctcaaccattagtggcg
gaggcacctacatatactacccagactcagtgaagggccgattcaccatctccagagacaacgccaagaacaccctgtat
ctgcaaatgaacagcctgagagccgaggacacggctgtgtattactgtaccagagataactatggccgcaactatgatta
tggcatggattattggggccagggcaccctggtgaccgtgagcagc
(amino acids)
(SEQ ID NO: 39)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSS
Humanized E6 heavy chain variable framework region 1 (FWR1) acid sequence:
(DNA)
(SEQ ID NO: 40)
gaggtgcagctggtggagtctgggggaggcctggtcaagcctggggggtccctgagactctcctgtgcagcctctggatt
caccttcagt
(amino acids)
(SEQ ID NO: 41)
EVQLVESGGGLVKPGGSLRLSCAASGFTFS
Humanized E6 heavy chain variable complementarity determining regions 1 (CDR1)
sequence:
(DNA)
(SEQ ID NO: 42)
aggtatggcatgagc
(amino acids)
(SEQ ID NO: 43)
RYGMS
Humanized E6 heavy chain variable framework region 2 (FWR2) acid sequence:
(DNA)
(SEQ ID NO: 44)
tgggtccgccaggctccagggaagaggctggagtgggtctca
(amino acids)
(SEQ ID NO: 45)
WVRQAPGKRLEWVS
Humanized E6 heavy chain variable complementarity determining regions 2 (CDR2)
sequence:
(DNA)
(SEQ ID NO: 46)
accattagtggcggaggcacctacatatactacccagactcagtgaagggc
(amino acids)
(SEQ ID NO: 47)
TISGGGTYIYYPDSVKG
Humanized E6 heavy chain variable framework region 3 (FWR3) acid sequence:
(DNA)
(SEQ ID NO: 48)
cgattcaccatctccagagacaacgccaagaacaccctgtatctgcaaatgaacagcctgagagccgaggacacggctgtg
tattactgtaccaga
(amino acids)
(SEQ ID NO: 49)
RFTISRDNAKNTLYLQMNSLRAEDTAVYYCTR
Humanized E6 heavy chain variable complementarity determining regions 3 (CDR3)
sequence:
(DNA)
(SEQ ID NO: 50)
gataactatggccgcaactatgattatggcatggattat
(amino acids)
(SEQ ID NO: 51)
DNYGRNYDYGMDY
Humanized E6 IgG2 heavy chain synthesized by Genescript:
(DNA)
(SEQ ID NO: 52)
gaattctaagcttgggccaccatggaactggggctccgctgggttttccttgttgctattttagaaggtgtccagtgtgag
gtgcagctggtggagtctgggggaggcctggtcaagcctggggggtccctgagactctcctgtgcagcctctggattcacc
ttcagtaggtatggcatgagctgggtccgccaggctccagggaagaggctggagtgggtctcaaccattagtggcggaggc
acctacatatactacccagactcagtgaagggccgattcaccatctccagagacaacgccaagaacaccctgtatctgcaa
atgaacagcctgagagccgaggacacggctgtgtattactgtaccagagataactatggccgcaactatgattatggcatg
gattattggggccagggcaccctggtgaccgtgagcagcgcctccaccaagggcccatcggtcttccccctggcgccctgc
tccaggagcacctccgagagcacagccgccctgggctgcctggtcaaggactacttccccgaaccggtgacggtgtcgtgg
aactcaggcgctctgaccagcggcgtgcacaccttcccagctgtcctacagtcctcaggactctactccctcagcagcgtg
gtgaccgtgccctccagcaacttcggcacccagacctacacctgcaacgtagatcacaagcccagcaacaccaaggtggac
aagacagttgagcgcaaatgttgtgtcgagtgcccaccgtgcccagcaccacctgtggcaggaccgtcagtcttcctcttc
cccccaaaacccaaggacaccctcatgatctcccggacccctgaggtcacgtgcgtggtggtggacgtgagccacgaagac
cccgaggtccagttcaactggtacgtggacggcgtggaggtgcataatgccaagacaaagccacgggaggagcagttcaac
agcacgttccgtgtggtcagcgtcctcaccgttgtgcaccaggactggctgaacggcaaggagtacaagtgcaaggtctcc
aacaaaggcctcccagcccccatcgagaaaaccatctccaaaaccaaagggcagccccgagaaccacaggtgtacaccctg
cccccatcccgggaggagatgaccaagaaccaggtcagcctgacctgcctggtcaaaggcttctaccccagcgacatcgcc
gtggagtgggagagcaatgggcagccggagaacaactacaagaccacacctcccatgctggactccgacggctccttcttc
ctctacagcaagctcaccgtggacaagagcaggtggcagcaggggaacgtcttctcatgctccgtgatgcatgaggctctg
cacaaccactacacgcagaagagcctctccctgtctccgggtaaatagtaagtttaaactctaga
(amino acids)
(SEQ ID NO: 53)
EF*AWATMELGLRWVFLVAILEGVQCEVQLVESGGGLVKPGGSLRLSCAASGFTFSRYGMSWVRQAPGKRLEWVSTISGGG
TYIYYPDSVKGRFTISRDNAKNTLYLQMNSLRAEDTAVYYCTRDNYGRNYDYGMDYWGQGTLVTVSSASTKGPSVFPLAPC
SRSTSESTAALGCLVKDYFPEPVTVSWNSGALTSGVHTFPAVLQSSGLYSLSSVVTVPSSNFGTQTYTCNVDHKPSNTKVD
KTVERKCCVECPPCPAPPVAGPSVFLFPPKPKDTLMISRTPEVTCVVVDVSHEDPEVQFNWYVDGVEVHNAKTKPREEQFN
STFRVVSVLTVVHQDWLNGKEYKCKVSNKGLPAPIEKTISKTKGQPREPQVYTLPPSREEMTKNQVSLTCLVKGFYPSDIA
VEWESNGQPENNYKTTPPMLDSDGSFFLYSKLTVDKSRWQQGNVFSCSVMHEALHNHYTQKSLSLSPGK**V*TLX
Human IgG2 heavy chain constant region sequence:
(DNA)
(SEQ ID NO: 54)
gcctccaccaagggcccatcggtcttccccctggcgccctgctccaggagcacctccgagagcacagccgccctgggctgc
ctggtcaaggactacttccccgaaccggtgacggtgtcgtggaactcaggcgctctgaccagcggcgtgcacaccttccca
gctgtcctacagtcctcaggactctactccctcagcagcgtggtgaccgtgccctccagcaacttcggcacccagacctac
acctgcaacgtagatcacaagcccagcaacaccaaggtggacaagacagttgagcgcaaatgttgtgtcgagtgcccaccg
tgcccagcaccacctgtggcaggaccgtcagtcttcctcttccccccaaaacccaaggacaccctcatgatctcccggacc
cctgaggtcacgtgcgtggtggtggacgtgagccacgaagaccccgaggtccagttcaactggtacgtggacggcgtggag
gtgcataatgccaagacaaagccacgggaggagcagttcaacagcacgttccgtgtggtcagcgtcctcaccgttgtgcac
caggactggctgaacggcaaggagtacaagtgcaaggtctccaacaaaggcctcccagcccccatcgagaaaaccatctcc
aaaaccaaagggcagccccgagaaccacaggtgtacaccctgcccccatcccgggaggagatgaccaagaaccaggtcagc
ctgacctgcctggtcaaaggcttctaccccagcgacatcgccgtggagtgggagagcaatgggcagccggagaacaactac
aagaccacacctcccatgctggactccgacggctccttcttcctctacagcaagctcaccgtggacaagagcaggtggcag
caggggaacgtcttctcatgctccgtgatgcatgaggctctgcacaaccactacacgcagaagagcctctccctgtctccg
ggtaaatag
(amino acids)
(SEQ ID NO: 55)
ASTKGPSVFPLAPCSRSTSESTAALGCLVKDYFPEPVTVSWNSGALTSGVHTFPAVLQSSGLYSLSSVVTVPSSNFGTQTY
TCNVDHKPSNTKVDKTVERKCCVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMHEALHNHYTQKSLSLSP
GK
Humanized E6 IgG1 heavy chain sequence:
(DNA)
(SEQ ID NO: 56)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacccactgtatctg
caaatgaacagcctgagagccgaggacacggctgtgtattactgtcccagagataactatggccgcaactatgattatggc
atggattattggggccagggcaccctggtgaccgtgagcagcgctagcaccaagggcccatcggtcttccccctggcaccc
tcctccaagagcacctctgggggcacagcggccctgggctgcctggtcaaggactacttccccgaaccggtgacggtgtcg
tggaactcaggcgccctgaccagcggcgtgcacaccttcccggctgtcctacagtcctcaggactctactccctcagcagc
gtggtgacagtgccctccagcagcttgggcacccagacctacatctgcaacgtgaatcacaagcccagcaacaccaaggtg
gacaagaaagttgagcccaaatcttgtgacaaaactcacacatgcccaccgtgcccagcacctgaactcctggggggaccg
tcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgg
gaggagcagtacaacagcacgtaccgtgtggtcagcgtcctcaccgtcctgcaccaggactggctgaatggcaaggagtac
aagtgcaaggtctccaacaaagccctcccagcccccatcgagaaaaccatctccaaagccaaagggcagccccgagaacca
caggtgtacaccctgcccccatcccgggaggagatgaccaagaaccaggtcagcctgacctgcctggtcaaaggcttctat
cccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtg
atgcatgaggctctgcacaaccactacacgcagaagagcctctccctgtctccgggtaaatgataa
(amino acids)
(SEQ ID NO: 57)
EVQLVESGGGLVKPGGSLRLSCAASGFTFSRYGMSWVRQAPGKRLEWVSTISGGGTYIYYPDSVKGRFTISRDNAKNPLYL
QMNSLRAEDTAVYYCPRDNYGRNYDYGMDYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK**
Human IgG1 heavy chain constant region sequence:
(DNA)
(SEQ ID NO: 58)
gctagcaccaagggcccatcggtcttccccctggcaccctcctccaagagcacctctgggggcacagcggccctgggctgc
ctggtcaaggactacttccccgaaccggtgacggtgtcgtggaactcaggcgccctgaccagcggcgtgcacaccttcccg
gctgtcctacagtcctcaggactctactccctcagcagcgtggtgacagtgccctccagcagcttgggcacccagacctac
atctgcaacgtgaatcacaagcccagcaacaccaaggtggacaagaaagttgagcccaaatcttgtgacaaaactcacaca
tgcccaccgtgcccagcacctgaactcctggggggaccgtcagtcttcctcttccccccaaaacccaaggacaccctcatg
atctcccggacccctgaggtcacatgcgtggtggtggacgtgagccacgaagaccctgaggtcaagttcaactggtacgtg
gacggcgtggaggtgcataatgccaagacaaagccgcgggaggagcagtacaacagcacgtaccgtgtggtcagcgtcctc
accgtcctgcaccaggactggctgaatggcaaggagtacaagtgcaaggtctccaacaaagccctcccagcccccatcgag
aaaaccatctccaaagccaaagggcagccccgagaaccacaggtgtacaccctgcccccatcccgggaggagatgaccaag
aaccaggtcagcctgacctgcctggtcaaaggcttctatcccagcgacatcgccgtggagtgggagagcaatgggcagccg
gagaacaactacaagaccacgcctcccgtgctggactccgacggctccttcttcctctacagcaagctcaccgtggacaag
agcaggtggcagcaggggaacgtcttctcatgctccgtgatgcatgaggctctgcacaaccactacacgcagaagagcctc
tccctgtctccgggtaaatgataa
(amino acids)
(SEQ ID NO: 59)
ASTKGPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHEALHNHYTQKSL
SLSPGK**
E6 heavy chain variable region overlapping sequence:
(DNA)
(SEQ ID NO: 62)
atggcatggattattggggccagggcaccct
IgG1 heavy chain constant region overlapping region sequence:
(DNA)
(SEQ ID NO: 63)
tacgtggacggcgtggaggtgcataatgccaag
pCDNA3.1 V5 and pSECTag2 overlapping sequence:
(DNA)
(SEQ ID NO: 64)
ccgctgatcagcctcgactgtgccttctagttg
Mouse E6 Light Chain variable region sequence:
(DNA)
(SEQ ID NO: 65)
caaattgttctcacccagtctccagcaatcatgtctgcatctccaggggaggaggtcaccctaacctgcagtgccacctca
agtgtaagttacatacactggttccagcagaggccaggcacttctcccaaactctggatttatagcacatccaacctggct
tctggagtccctgttcgcttcagtggcagtggatatgggacctcttactctctcacaatcagccgaatggaggctgaagat
gctgccacttattactgccagcaaaggagtagttccccattcacgttcggctcggggacaaagttggaaataaaa
(amino acids)
(SEQ ID NO: 66)
QIVLTQSPAIMSASPGEEVTLTCSATSSVSYIHWFQQRPGTSPKLWIYSTSNLASGVPVRFSGSGYGTSYSLTISRMEAED
AATYYCQQRSSSPFTFGSGTKLEIK
Mouse E6 light chain variable framework region 1 (FWR1) sequence:
(DNA)
(SEQ ID NO: 67)
caaattgttctcacccagtctccagcaatcatgtctgcatctccaggggaggaggtcaccctaacctgc
(amino acids)
(SEQ ID NO: 68)
QIVLTQSPAIMSASPGEEVTLTC
Mouse E6 light chain variable complementarity determining regions 1 (CDR1)
sequence:
(DNA)
(SEQ ID NO: 69)
AGTGCCACCTCAAGTGTAAGTTACATACAC
(amino acids)
(SEQ ID NO: 70)
SATSSVSYIH
Mouse E6 light chain variable framework region 2 (FWR2) sequence:
(DNA)
(SEQ ID NO: 71)
tggttccagcagaggccaggcacttctcccaaactctggatttat
(amino acids)
(SEQ ID NO: 72)
WFQQRPGTSPKLWIY
Mouse E6 light chain variable complementarity determining regions 2 (CDR2)
sequence:
(DNA)
(SEQ ID NO: 73)
agcacatccaacctggcttct
(amino acids)
(SEQ ID NO: 74)
STSNLAS
Mouse E6 light chain variable framework region 3 (FWR3) sequence:
(DNA)
(SEQ ID NO: 75)
ggagtccctgttcgcttcagtggcagtggatatgggacctcttactctctcacaatcagccgaatggaggctgaagatgc
tgccacttattactgc
(amino acids)
(SEQ ID NO: 76)
GVPVRFSGSGYGTSYSLTISRMEAEDAATYYC
Mouse E6 light chain variable complementarity determining regions 3 (CDR3)
sequence:
(DNA)
(SEQ ID NO: 77)
cagcaaaggagtagttccccattcacg
(amino acids)
(SEQ ID NO: 78)
QQRSSSPFT
Humanized E6 light chain variable region sequence:
(DNA)
(SEQ ID NO: 93)
gaaattgtgttgacacagtctccagccaccctgtctttgtctccaggggaaagagccaccctcacctgcagcgccaccagc
agtgttagctacatccactggtaccaacagaggcctggccagagccccaggctcctcatctatagcacctccaacctggcc
agcggcatcccagccaggttcagtggcagtgggtctgggagcgactacactctcaccatcagcagcctagagcctgaagat
tttgcagtttattactgtcagcagcgtagcagctcccctttcacctttggcagcggcaccaaagtggaaattaaa
(amino acids)
(SEQ ID NO: 94)
EIVLTQSPATLSLSPGERATLTCSATSSVSYIHWYQQRPGQSPRLLIYSTSNLASGIPARFSGSGSGSDYTLTISSLEPED
FAVYYCQQRSSSPFTFGSGTKVEIK
Humanized E6 light chain variable framework region 1 (FWR1) acid sequence:
(DNA)
(SEQ ID NO: 95)
gaaattgtgttgacacagtctccagccaccctgtctttgtctccaggggaaagagccaccctcacctgc
(amino acids)
(SEQ ID NO: 96)
EIVLTQSPATLSLSPGERATLTC
Humanized E6 light chain variable complementarity determining regions 1 (CDR1)
sequence:
(DNA)
(SEQ ID NO: 97)
agcgccaccagcagtgttagctacatccac
(amino acids)
(SEQ ID NO: 98)
SATSSVSYIH
Humanized E6 heavy light variable framework region 2 (FWR2) acid sequence:
(DNA)
(SEQ ID NO: 99)
tggtaccaacagaggcctggccagagccccaggctcctcatctat
(amino acids)
(SEQ ID NO: 100)
WYQQRPGQSPRLLIY
Humanized E6 light chain variable complementarity determining regions 2 (CDR2)
sequence:
(DNA)
(SEQ ID NO: 101)
agcacctccaacctggccagc
(amino acids)
(SEQ ID NO: 102)
STSNLAS
Humanized E6 light chain variable framework region 3 (FWR3) acid sequence:
(DNA)
(SEQ ID NO: 103)
ggcatcccagccaggttcagtggcagtgggtctgggagcgactacactctcaccatcagcagcctagagcctgaagatttt
gcagtttattactgt
(amino acids)
(SEQ ID NO: 104)
GIPARFSGSGSGSDYTLTISSLEPEDFAVYYC
Humanized E6 light chain variable complementarity determining regions 3 (CDR3)
sequence:
(DNA)
(SEQ ID NO: 105)
cagcagcgtagcagctcccctttcacc
(amino acids)
(SEQ ID NO: 106)
QQRSSSPFT
Humanized E6 Kappa light chain synthesized by Genescript:
(DNA)
(SEQ ID NO: 107)
gaattctaagcttgggccaccatggaagccccagcgcagcttctcttcctcctgctactctggctcccagataccactgga
gaaattgtgttgacacagtctccagccaccctgtctttgtctccaggggaaagagccaccctcacctgcagcgccaccagc
agtgttagctacatccactggtaccaacagaggcctggccagagccccaggctcctcatctatagcacctccaacctggcc
agcggcatcccagccaggttcagtggcagtgggtctgggagcgactacactctcaccatcagcagcctagagcctgaagat
tttgcagtttattactgtcagcagcgtagcagctcccctttcacctttggcagcggcaccaaagtggaaattaaaaggacg
gtggctgcaccatctgtcttcatcttcccgccatctgatgagcagttgaaatctggaactgcctctgttgtgtgcctgctg
aataacttctatcccagagaggccaaagtacagtggaaggtggataacgccctccaatcgggtaactcccaggagagtgtc
acagagcaggacagcaaggacagcacctacagcctcagcagcaccctgacgctgagcaaagcagactacgagaaacacaaa
gtctacgcctgcgaagtcacccatcagggcctgagctcgcccgtcacaaagagcttcaacaggggagagtgttagtaagtt
taaactctaga
(amino acids)
(SEQ ID NO: 108)
EF*AWATMEAPAQLLFLLLLWLPDTTGEIVLTQSPATLSLSPGERATLTCSATSSVSYIHWYQQRPGQSPRLLIYSTSNLA
SGIPARFSGSGSGSDYTLTISSLEPEDFAVYYCQQRSSSPFTFGSGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSPVTKSFNRGEC**V
*TLX
Human Kappa light chain constant region sequence:
(DNA)
(SEQ ID NO: 109)
aggacggtggctgcaccatctgtcttcatcttcccgccatctgatgagcagttgaaatctggaactgcctctgttgtgtgc
ctgctgaataacttctatcccagagaggccaaagtacagtggaaggtggataacgccctccaatcgggtaactcccaggag
agtgtcacagagcaggacagcaaggacagcacctacagcctcagcagcaccctgacgctgagcaaagcagactacgagaaa
cacaaagtctacgcctgcgaagtcacccatcagggcctgagctcgcccgtcacaaagagcttcaacaggggagagtgttag
(amino acids)
(SEQ ID NO: 110)
RTVAAPSVFIFPPSDEQLKSGTASVVCLLNNFYPREAKVQWKVDNALQSGNSQESVTEQDSKDSTYSLSSTLTLSKADYEK
HKVYACEVTHQGLSSPVTKSFNRGEC
Humanized E6 lambda light chain sequence:
(DNA)
(SEQ ID NO: 111)
gaaattgtgttgacacagtctccagccaccctgtctttgtctccaggggaaagagccaccctcacctgcagcgccaccagc
agtgttagctacatccactggtaccaacagaggcctggccagagccccaggctcctcatctatagcacctccaacctggcc
agcggcatcccagccaggttcagtggcagtgggtctgggagcgactacactctcaccatcagcagcctagagcctgaagat
tttgcagtttattactgtcagcagcgtagcagctcccctttcacctttggcagcggcaccaaagtggaaattaaaggtcag
cccaaggctgccccctcggtcactctgttcccgccctcctctgaggagcttcaagccaacaaggccacactggtgtgtctc
ataagtgacttctacccgggagccgtgacagtggcctggaaggcagatagcagccccgtcaaggcgggagtggagaccacc
acaccctccaaacaaagcaacaacaagtacgcggccagcagctatctgagcctgacgcctgagcagtggaagtcccacaga
agctacagctgccaggtcacgcatgaagggagcaccgtggagaagacagtggcccctacagaatgttcatagtaa
(amino acids)
(SEQ ID NO: 112)
EIVLTQSPATLSLSPGERATLTCSATSSVSYIHWYQQRPGQSPRLLIYSTSNLASGIPARFSGSGSGSDYTLTISSLEPED
FAVYYCQQRSSSPFTFGSGTKVEIKGQPKAAPSVTLFPPSSEELQANKATLVCLISDFYPGAVTVAWKADSSPVKAGVETT
TPSKQSNNKYAASSYLSLTPEQWKSHRSYSCQVTHEGSTVEKTVAPTECS**
Humanized lambda light chain constant region sequence:
(DNA)
(SEQ ID NO: 113)
ggtcagcccaaggctgccccctcggtcactctgttcccgccctcctctgaggagcttcaagccaacaaggccacactggtg
tgtctcataagtgacttctacccgggagccgtgacagtggcctggaaggcagatagcagccccgtcaaggcgggagtggag
accaccacaccctccaaacaaagcaacaacaagtacgcggccagcagctatctgagcctgacgcctgagcagtggaagtcc
cacagaagctacagctgccaggtcacgcatgaagggagcaccgtggagaagacagtggcccctacagaatgttcatagtaa
(amino acids)
(SEQ ID NO: 114)
GQPKAAPSVTLFPPSSEELQANKATLVCLISDFYPGAVTVAWKADSSPVKAGVETTTPSKQSNNKYAASSYLSLTPEQWKS
HRSYSCQVTHEGSTVEKTVAPTECS**
E6 light chain variable region overlapping sequence:
(DNA)
(SEQ ID NO: 116)
agcgccaccagcagtgttagctacatccact
pCDNA3.1 V5 and pSECTag2 overlapping sequence:
(DNA)
(SEQ ID NO: 117)
ccgctgatcagcctcgactgtgccttctagttg
Mouse C2 heavy chain variable region sequence:
(DNA)
(SEQ ID NO: 118)
gaggtccagctggaggagtcagggggaggcttagtgaagcctggagggtccctgaaactctcctgtgcagcctctggatt
cactttcagtggctatgccatgtcttgggttcgccagactccggagaagaggctggagtgggtcgcaaccattagtagtg
gtggtacttatatctactatccagacagtgtgaaggggcgattcaccatctccagagacaatgccaagaacaccctgtac
ctgcaaatgagcagtctgaggtctgaggacacggccatgtattactgtgcaagacttgggggggataattactacgaata
cttcgatgtctggggcgcagggaccacggtcaccgtctcctccgccaaaacgacacccccatctgtctat
(amino acids)
(SEQ ID NO: 119)
EVQLEESGGGLVKPGGSLKLSCAASGFTFSGYAMSWVRQTPEKRLEWVATISSGGTYIYYPDSVKGRFTISRDNAKNTLY
LQMSSLRSEDTAMYYCARLGGDNYYEYFDVWGAGTTVTVSSAKTTPPSVY
Mouse C2 heavy chain variable framework region 1 (FWR1) sequence:
(DNA)
(SEQ ID NO: 120)
gaggtccagctggaggagtcagggggaggcttagtgaagcctggagggtccctgaaactctcctgtgcagcctctggatt
cactttcagt
(amino acids)
(SEQ ID NO: 121)
EVQLEESGGGLVKPGGSLKLSCAASGFTFS
Mouse C2 heavy chain variable complementarity determining regions 1 (CDR1)
sequence:
(DNA)
(SEQ ID NO: 122)
ggctatgccatgtct
(amino acids)
(SEQ ID NO: 123)
GYAMS
Mouse C2 heavy chain variable framework region 2 (FWR2) sequence:
(DNA)
(SEQ ID NO: 124)
tgggttcgccagactccggagaagaggctggagtgggtcgca
(amino acids)
(SEQ ID NO: 125)
WVRQTPEKRLEWVA
Mouse C2 heavy chain variable complementarity determining regions 2 (CDR2)
sequence:
(DNA)
(SEQ ID NO: 126)
accattagtagtggtggtacttatatctactatccagacagtgtgaagggg
(amino acids)
(SEQ ID NO: 127)
TISSGGTYIYYPDSVKG
Mouse C2 heavy chain variable framework region 3 (FWR3) sequence:
(DNA)
(SEQ ID NO: 128)
cgattcaccatctccagagacaatgccaagaacaccctgtacctgcaaatgagcagtctgaggtctgaggacacggccatg
tattactgtgcaaga
(amino acids)
(SEQ ID NO: 129)
RFTISRDNAKNTLYLQMSSLRSEDTAMYYCAR
Mouse C2 heavy chain variable complementarity determining regions 3 (CDR3)
sequence:
(DNA)
(SEQ ID NO: 130)
cttgggggggataattactacgaatacttcgatgtc
(amino acids)
(SEQ ID NO: 131)
LGGDNYYEYFDV
Humanized C2 heavy chain variable region sequence:
(DNA)
(SEQ ID NO: 144)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctcc
(amino acids)
(SEQ ID NO: 145)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSS
Humanized C2 heavy chain variable framework region 1 (FWR1) sequence:
(DNA)
(SEQ ID NO: 146)
gaggtgcagctggtggagtctgggggaggcctggtcaagcctggggggtccctgagactctcctgtgcagcctctggattc
accttcagt
(amino acids)
(SEQ ID NO: 147)
EVQLVESGGGLVKPGGSLRLSCAASGFTFS
Humanized C2 heavy chain variable complementarity determining regions 1 (CDR1)
sequence:
(DNA)
(SEQ ID NO: 148)
ggctatgccatgagc
(amino acids)
(SEQ ID NO: 149)
GYAMS
Humanized C2 heavy chain variable framework region 2 (FWR2) sequence:
(DNA)
(SEQ ID NO: 150)
tgggtccgccaggctccagggaaggggctggagtgggtctcaa
(amino acids)
(SEQ ID NO: 151)
WVRQAPGKGLEWVS
Humanized C2 heavy chain variable complementarity determining regions 2 (CDR2)
sequence:
(DNA)
(SEQ ID NO: 152)
accattagtagtggcggaacctacatatactaccccgactcagtgaagggc
(amino acids)
(SEQ ID NO: 153)
TISSGGTYIYYPDSVKG
Humanized C2 heavy chain variable framework region 3 (FWR3) sequence:
(DNA)
(SEQ ID NO: 154)
cgattcaccatctccagagacaacgccaagaactcactgtatctgcaaatgaacagcctgagagccgaggacacggccgtg
tattactgtgcgaga
(amino acids)
(SEQ ID NO: 155)
RFTISRDNAKNSLYLQMNSLRAEDTAVYYCAR
Humanized C2 heavy chain variable complementarity determining regions 3 (CDR3)
sequence:
(DNA)
(SEQ ID NO: 156)
cttgggggggataattactacgaatacttcgatgtc
(amino acids)
(SEQ ID NO: 157)
LGGDNYYEYFDV
Humanized C2 IgG1 heavy chain sequence
(DNA)
(SEQ ID NO: 157)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccgctagcaccaagggcccatcggtcttccccctggcaccctcc
tccaagagcacctctgggggcacagcggccctgggctgcctggtcaaggactacttccccgaaccggtgacggtgtcgtgg
aactcaggcgccctgaccagcggcgtgcacaccttcccggctgtcctacagtcctcaggactctactccctcagcagcgtg
gtgacagtgccctccagcagcttgggcacccagacctacatctgcaacgtgaatcacaagcccagcaacaccaaggtggac
aagaaagttgagcccaaatcttgtgacaaaactcacacatgcccaccgtgcccagcacctgaactcctggggggaccgtca
gtcttcctcttccccccaaaacccaaggacaccctcatgatctcccggacccctgaggtcacatgcgtggtggtggacgtg
agccacgaagaccctgaggtcaagttcaactggtacgtggacggcgtggaggtgcataatgccaagacaaagccgcgggag
gagcagtacaacagcacgtaccgtgtggtcagcgtcctcaccgtcctgcaccaggactggctgaatggcaaggagtacaag
tgcaaggtctccaacaaagccctcccagcccccatcgagaaaaccatctccaaagccaaagggcagccccgagaaccacag
gtgtacaccctgcccccatcccgggaggagatgaccaagaaccaggtcagcctgacctgcctggtcaaaggcttctatccc
agcgacatcgccgtggagtgggagagcaatgggcagccggagaacaactacaagaccacgcctcccgtgctggactccgac
ggctccttcttcctctacagcaagctcaccgtggacaagagcaggtggcagcaggggaacgtcttctcatgctccgtgatg
catgaggctctgcacaaccactacacgcagaagagcctctccctgtctccgggtaaatgataa
(amino acids)
(SEQ ID NO: 158)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK**
pCDNA3.1 V5 overlapping sequence:
(DNA)
(SEQ ID NO: 161)
actcactatagggagacccaagctggctagtt
Human IgG1 constant region overlapping sequence:
(DNA)
(SEQ ID NO: 162)
gacggtgtcgtggaactcaggcgccctgaccagc
Humanized C2 IgG2 heavy chain sequence
(DNA)
(SEQ ID NO: 163)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccgcctccaccaagggcccatcggtcttccccctggcgccctgc
tccaggagcacctccgagagcacagccgccctgggctgcctggtcaaggactacttccccgaaccggtgacggtgtcgtgg
aactcaggcgctctgaccagcggcgtgcacaccttcccagctgtcctacagtcctcaggactctactccctcagcagcgtg
gtgaccgtgccctccagcaacttcggcacccagacctacacctgcaacgtagatcacaagcccagcaacaccaaggtggac
aagacagttgagcgcaaatgttgtgtcgagtgcccaccgtgcccagcaccacctgtggcaggaccgtcagtcttcctcttc
cccccaaaacccaaggacaccctcatgatctcccggacccctgaggtcacgtgcgtggtggtggacgtgagccacgaagac
cccgaggtccagttcaactggtacgtggacggcgtggaggtgcataatgccaagacaaagccacgggaggagcagttcaac
agcacgttccgtgtggtcagcgtcctcaccgttgtgcaccaggactggctgaacggcaaggagtacaagtgcaaggtctcc
aacaaaggcctcccagcccccatcgagaaaaccatctccaaaaccaaagggcagccccgagaaccacaggtgtacaccctg
cccccatcccgggaggagatgaccaagaaccaggtcagcctgacctgcctggtcaaaggcttctaccccagcgacatcgcc
gtggagtgggagagcaatgggcagccggagaacaactacaagaccacacctcccatgctggactccgacggctccttcttc
ctctacagcaagctcaccgtggacaagagcaggtggcagcaggggaacgtcttctcatgctccgtgatgcatgaggctctg
cacaaccactacacgcagaagagcctctccctgtctccgggtaaatagtaa
(amino acids)
(SEQ ID NO: 164)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSSASTKGPSVFPLAPCSRSTSESTAALGCLVKDYFPEPVTVSW
NSGALTSGVHTFPAVLQSSGLYSLSSVVTVPSSNFGTQTYTCNVDHKPSNTKVDKTVERKCCVECPPCPAPPVAGPSVFLF
PPKPKDTLMISRTPEVTCVVVDVSHEDPEVQFNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYPSDIAVEWESNGQPENNYKTTPPMLDSDGSFF
LYSKLTVDKSRWQQGNVFSCSVMHEALHNHYTQKSLSLSPGK**
Mouse C2 light chain variable region sequence:
(DNA)
(SEQ ID NO: 168)
gacattgtgatcacacagtctacagcttccttaggtgtatctctggggcagagggccaccatctcatgcagggccagcaaa
agtgtcagtacatctggctatagttatatgcactggtaccaacagagaccaggacagccacccaaactcctcatctatctt
gcatccaacctagaatctggggtccctgccaggttcagtggcagtgggtctgggacagacttcaccctcaacatccatcct
gtggaggaggaggatgctgcaacctattactgtcagcacagtagggagcttccgttcacgttcggaggggggaccaagctg
gagataaaacgggctgatgctgcaccaactgtatcc
(amino acids)
(SEQ ID NO: 169)
DIVITQSTASLGVSLGQRATISCRASKSVSTSGYSYMHWYQQRPGQPPKLLIYLASNLESGVPARFSGSGSGTDFTLNIHP
VEEEDAATYYCQHSRELPFTFGGGTKLEIKRADAAPTVS
Mouse C2 light chain variable framework region 1 (FWR1) sequence:
(DNA)
(SEQ ID NO: 170)
gacattgtgatcacacagtctacagcttccttaggtgtatctctggggcagagggccaccatctcatgc
(amino acids)
(SEQ ID NO: 171)
DIVITQSTASLGVSLGQRATISC
Mouse C2 light chain variable complementarity determining regions 1 (CDR1)
sequence:
(DNA)
(SEQ ID NO: 172)
agggccagcaaaagtgtcagtacatctggctatagttatatgcac
(amino acids)
(SEQ ID NO: 173)
RASKSVSTSGYSYMH
Mouse C2 light chain variable framework region 2 (FWR2) sequence:
(DNA)
(SEQ ID NO: 174)
tggtaccaacagagaccaggacagccacccaaactcctcatctat
(amino acids)
(SEQ ID NO: 175)
WYQQRPGQPPKLLIY
Mouse C2 light chain variable complementarity determining regions 2 (CDR2)
sequence:
(DNA)
(SEQ ID NO: 176)
cttgcatccaacctagaatc
(amino acids)
(SEQ ID NO: 177)
LASNLES
Mouse C2 light chain variable framework region 3 (FWR3) sequence:
(DNA)
(SEQ ID NO: 178)
tggggtccctgccaggttcagtggcagtgggtctgggacagacttcaccctcaacatccatcctgtggaggaggaggatgc
tgcaacctattactgt
(amino acids)
(SEQ ID NO: 179)
GVPARFSGSGSGTDFTLNIHPVEEEDAATYYC
Mouse C2 light chain variable complementarity determining regions 3 (CDR3)
sequence:
(DNA)
(SEQ ID NO: 180)
cagcacagtagggagcttccgttcacg
(amino acids)
(SEQ ID NO: 181)
QHSRELPFT
Humanized C2 light chain variable region sequence:
(DNA)
(SEQ ID NO: 194)
gacattgtgctgacccagtctccagcctccttggccgtgtctccaggacagagggccaccatcacctgcagagccagtaag
agtgtcagtaccagcggatactcctacatgcactggtatcagcagaaaccaggacaacctcctaaactcctgatttacctg
gcatccaatctggagagcggggtcccagccaggttcagcggcagtgggtctgggaccgatttcaccctcacaattaatcct
gtggaagctaatgatactgcaaattattactgtcagcacagtagggagctgcctttcacattcggcggagggaccaaggtg
gagatcaaacgaact
(amino acids)
(SEQ ID NO: 195)
DIVLTQSPASLAVSPGQRATITCRASKSVSTSGYSYMHWYQQKPGQPPKLLIYLASNLESGVPARFSGSGSGTDFTLTINP
VEANDTANYYCQHSRELPFTFGGGTKVEIKRT
Humanized C2 light chain variable framework region 1 (FWR1) acid sequence:
(DNA)
(SEQ ID NO: 196)
gacattgtgctgacccagtctccagcctccttggccgtgtctccaggacagagggccaccatcacctgc
(amino acids)
(SEQ ID NO: 197)
DIVLTQSPASLAVSPGQRATITC
Humanized C2 light chain variable complementarity determining regions 1 (CDR1)
sequence:
(DNA)
(SEQ ID NO: 198)
agagccagtaagagtgtcagtaccagcggatactcctacatgcac
(amino acids)
(SEQ ID NO: 199)
RASKSVSTSGYSYMH
Humanized C2 heavy light variable framework region 2 (FWR2) acid sequence:
(DNA)
(SEQ ID NO: 200)
tggtatcagcagaaaccaggacaacctcctaaactcctgatttac
(amino acids)
(SEQ ID NO: 201)
WYQQKPGQPPKLLIY
Humanized C2 light chain variable complementarity determining regions 2 (CDR2)
sequence:
(DNA)
(SEQ ID NO: 202)
ctggcatccaatctggagagc
(amino acids)
(SEQ ID NO: 203)
LASNLES
Humanized C2 light chain variable framework region 3 (FWR3) acid sequence:
(DNA)
(SEQ ID NO: 204)
ggggtcccagccaggttcagcggcagtgggtctgggaccgatttcaccctcacaattaatcctgtggaagctaatgatact
gcaaattattactgt
(amino acids)
(SEQ ID NO: 205)
GVPARFSGSGSGTDFTLTINPVEANDTANYYC
Humanized C2 light chain variable complementarity determining regions 3 (CDR3)
sequence:
(DNA)
(SEQ ID NO: 206)
cagcacagtagggagctgcctttcaca
(amino acids)
(SEQ ID NO: 207)
QHSRELPFT
Humanized C2 light chain variable complementarity determining regions 3 (CDR3)
sequence:
(DNA)
(SEQ ID NO: 208)
ctgcagagtaagaattttcctcccaca
(amino acids)
(SEQ ID NO: 209)
LQSKNFPPT
Murine Ig kappa chain leader sequence
(DNA)
(SEQ ID NO: 222)
atggagacagacacactcctgctatgggtactgctgctctgggttccaggttccactggtgac
(amino acids)
(SEQ ID NO: 223)
METDTLLLWVLLLWVPGSTGD
Interleukin-2 (IL-2) leader sequence
(DNA)
(SEQ ID NO: 224)
atgtacaggatgcaactcctgtcttgcattgcactaagtcttgcacttgtcacaaacagt
(amino acids)
(SEQ ID NO: 225)
MYRMQLLSCIALSLALVTNS
CD33 leader sequence
(DNA)
(SEQ ID NO: 226)
atgcctcttctgcttctgcttcctctgctttgggctggagctcttgct
(amino acids)
(SEQ ID NO: 227)
MPLLLLLPLLWAGALA
IGHV3-21*03 leader sequence
(DNA)
(SEQ ID NO: 228)
atggaactggggctccgctgggttttccttgttgctattttagaaggtgtccagtgt
(amino acids)
(SEQ ID NO: 229)
MELGLRWVFLVAILEGVQC
IGHV3-11*02 leader sequence
(DNA)
(SEQ ID NO: 230)
atggaagccccagcgcagcttctcttcctcctgctactctggctcccagataccactgga
(amino acids)
(SEQ ID NO: 231)
MEAPAQLLFLLLLWLPDTTG
Humanized E6 single chain GS3
(DNA)
(SEQ ID NO: 232)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacaccctgtatctg
caaatgaacagcctgagagccgaggacacggctgtgtattactgtaccagagataactatggccgcaactatgattatggc
atggattattggggccagggcaccctggtgaccgtgagcagcggcggtggcggatccggcggtggcggatccggcggtggc
ggatccgaaattgtgttgacacagtctccagccaccctgtctttgtctccaggggaaagagccaccctcacctgcagcgcc
accagcagtgttagctacatccactggtaccaacagaggcctggccagagccccaggctcctcatctatagcacctccaac
ctggccagcggcatcccagccaggttcagtggcagtgggtctgggagcgactacactctcaccatcagcagcctagagcct
gaagattttgcagtttattactgtcagcagcgtagcagctcccctttcacctttggcagcggcaccaaagtggaaattaaa
(amino acids)
(SEQ ID NO: 233)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSGGGGSGGGGSGGGGSEIVLTQSPATLSLSPGERATLTC
SATSSVSYIHWYQQRPGQSPRLLIYSTSNLASGIPARFSGSGSGSDYTLTISSLEPEDFAVYYCQQRSSSPFTFGSGTKV
EIK
Humanized E6 single chain IgGlnoC
(DNA)
(SEQ ID NO: 234)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacaccctgtatctg
caaatgaacagcctgagagccgaggacacggctgtgtattactgtaccagagataactatggccgcaactatgattatggc
atggattattggggccagggcaccctggtgaccgtgagcagcgataaaacccatactaaaccgccaaaaccggcgccggaa
ctgctgggtggtcctggtaccggtgaaattgtgttgacacagtctccagccaccctgtctttgtctccaggggaaagagcc
accctcacctgcagcgccaccagcagtgttagctacatccactggtaccaacagaggcctggccagagccccaggctcctc
atctatagcacctccaacctggccagcggcatcccagccaggttcagtggcagtgggtctgggagcgactacactctcacc
atcagcagcctagagcctgaagattttgcagtttattactgtcagcagcgtagcagctcccctttcacctttggcagcggc
accaaagtggaaattaaa
(amino acids)
(SEQ ID NO: 235)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSDKTHTKPPKPAPELLGGPGTGEIVLTQSPATLSLSPGE
RATLTCSATSSVSYIHWYQQRPGQSPRLLIYSTSNLASGIPARFSGSGSGSDYTLTISSLEPEDFAVYYCQQRSSSPFTF
GSGTKVEIK
Humanized E6 single chain X4 (linker is IgG1 and IgG2 modified hinge region)
(DNA)
(SEQ ID NO: 236)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacaccctgtatctg
caaatgaacagcctgagagccgaggacacggctgtgtattactgtaccagagataactatggccgcaactatgattatggc
atggattattggggccagggcaccctggtgaccgtgagcagcgataaaacccatactaaaccgccaaaaccggcgccggaa
ctgctgggtggtcctggtaccggtactggtggtccgactattaaacctccgaaacctccgaaacctgctccgaacctgctg
ggtggtccggaaattgtgttgacacagtctccagccaccctgtctttgtctccaggggaaagagccaccctcacctgcagc
gccaccagcagtgttagctacatccactggtaccaacagaggcctggccagagccccaggctcctcatctatagcacctcc
aacctggccagcggcatcccagccaggttcagtggcagtgggtctgggagcgactacactctcaccatcagcagcctagag
cctgaagattttgcagtttattactgtcagcagcgtagcagctcccctttcacctttggcagcggcaccaaagtggaaatt
aaa
(amino acids)
(SEQ ID NO: 237)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSDKTHTKPPKPAPELLGGPGTGTGGPTIKPPKPPKPAPN
LLGGPEIVLTQSPATLSLSPGERATLTCSATSSVSYIHWYQQRPGQSPRLLIYSTSNLASGIPARFSGSGSGSDYTLTIS
SLEPEDFAVYYCQQRSSSPFTFGSGTKVEIK
Humanized C2 single chain GS3
(DNA)
(SEQ ID NO: 238)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaact
(amino acids)
(SEQ ID NO: 239)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRT
Humanized C2 single chain IgG (no Cysteine)
(DNA)
(SEQ ID NO: 240)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccgataaaacccatactaaaccgccaaaaccggcgccggaactg
ctgggtggtcctggtaccggtgacattgtgctgacccagtctccagcctccttggccgtgtctccaggacagagggccacc
atcacctgcagagccagtaagagtgtcagtaccagcggatactcctacatgcactggtatcagcagaaaccaggacaacct
cctaaactcctgatttacctggcatccaatctggagagcggggtcccagccaggttcagcggcagtgggtctgggaccgat
ttcaccctcacaattaatcctgtggaagctaatgatactgcaaattattactgtcagcacagtagggagctgcctttcaca
ttcggcggagggaccaaggtggagatcaaacgaact
(amino acids)
(SEQ ID NO: 241)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSDKTHTKPPKPAPELLGGPGTGDIVLTQSPASLAVSPGQR
ATITCRASKSVSTSGYSYMHWYQQKPGQPPKLLIYLASNLESGVPARFSGSGSGTDFTLTINPVEANDTANYYCQHSREL
PFTFGGGTKVEIKRT
Humanized C2 single chain X4 (linker is IgG1 and IgG2 modified hinge region)
(DNA)
(SEQ ID NO: 242)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccgataaaacccatactaaaccgccaaaaccggcgccggaactg
ctgggtggtcctggtaccggtactggtggtccgactattaaacctccgaaacctccgaaacctgctccgaacctgctgggt
ggtccggacattgtgctgacccagtctccagcctccttggccgtgtctccaggacagagggccaccatcacctgcagagcc
agtaagagtgtcagtaccagcggatactcctacatgcactggtatcagcagaaaccaggacaacctcctaaactcctgatt
tacctggcatccaatctggagagcggggtcccagccaggttcagcggcagtgggtctgggaccgatttcaccctcacaatt
aatcctgtggaagctaatgatactgcaaattattactgtcagcacagtagggagctgcctttcacattcggcggagggacc
aaggtggagatcaaacgaact
(amino acids)
(SEQ ID NO: 243)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSDKTHTKPPKPAPELLGGPGTGTGGPTIKPPKPPKPAPNL
LGGPDIVLTQSPASLAVSPGQRATITCRASKSVSTSGYSYMHWYQQKPGQPPKLLIYLASNLESGVPARFSGSGSGTDFT
LTINPVEANDTANYYCQHSRELPFTFGGGTKVEIKRT
Humanized C3 single chain GS3
(DNA)
(SEQ ID NO: 244)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcggcggtggcggatccggcggtggcggatccggcggtggcggatccgat
attgtgatgacccagactccactctctctgtccgtcacccctggacagccggcctccatctcctgcaggtctagtcagacc
attgtccatagtaatggaaacacctatttggagtggtacctgcagaagccaggccagtctccacagctcctgatctataag
gtttccaaccggttctctggagtgccagataggttcagtggcagcgggtcagggacagatttcacactgaaaatcagccgg
gtggaggctgaggatgttggggtttattactgcttccaaggtagccacgtgcctttcaccttcggcggagggaccaaggtg
gagatcaaacgaact
(amino acids)
(SEQ ID NO: 245)
QVQLVQSGAEVKKPGASVKVSCKASGYTFTDYAMNWVRQAPGQGLEWMGVISTFSGNTNFNQKFKGRVTMTTDTSTSTAYM
ELRSLRSDDTAVYYCARSDYYGPYFDYWGQGTTLTVSSGGGGSGGGGSGGGGSDIVMTQTPLSLSVTPGQPASISCRSSQ
TIVHSNGNTYLEWYLQKPGQSPQLLIYKVSNRFSGVPDRFSGSGSGTDFTLKISRVEAEDVGVYYCFQGSHVPFTFGGGT
KVEIKRT
Humanized C3 single chain IgG1 (no Cysteine)
(DNA)
(SEQ ID NO: 246)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcgataaaacccatactaaaccgccaaaaccggcgccggaactgctgggt
ggtcctggtaccggtgatattgtgatgacccagactccactctctctgtccgtcacccctggacagccggcctccatctcc
tgcaggtctagtcagaccattgtccatagtaatggaaacacctatttggagtggtacctgcagaagccaggccagtctcca
cagctcctgatctataaggtttccaaccggttctctggagtgccagataggttcagtggcagcgggtcagggacagatttc
acactgaaaatcagccgggtggaggctgaggatgttggggtttattactgcttccaaggtagccacgtgcctttcaccttc
ggcggagggaccaaggtggagatcaaacgaact
(amino acids)
(SEQ ID NO: 247)
QVQLVQSGAEVKKPGASVKVSCKASGYTFTDYAMNWVRQAPGQGLEWMGVISTFSGNTNFNQKFKGRVTMTTDTSTSTAYM
ELRSLRSDDTAVYYCARSDYYGPYFDYWGQGTTLTVSSDKTHTKPPKPAPELLGGPGTGDIVMTQTPLSLSVTPGQPASI
SCRSSQTIVHSNGNTYLEWYLQKPGQSPQLLIYKVSNRFSGVPDRFSGSGSGTDFTLKISRVEAEDVGVYYCFQGSHVPF
TFGGGTKVEIKRT
Humanized C3 single chain X4 (linker is IgG1 and IgG2 modified hinge region)
(DNA)
(SEQ ID NO: 248)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcgataaaacccatactaaaccgccaaaaccggcgccggaactgctgggt
ggtcctggtaccggtactggtggtccgactattaaacctccgaaacctccgaaacctgctccgaacctgctgggtggtccg
gatattgtgatgacccagactccactctctctgtccgtcacccctggacagccggcctccatctcctgcaggtctagtcag
accattgtccatagtaatggaaacacctatttggagtggtacctgcagaagccaggccagtctccacagctcctgatctat
aaggtttccaaccggttctctggagtgccagataggttcagtggcagcgggtcagggacagatttcacactgaaaatcagc
cgggtggaggctgaggatgttggggtttattactgcttccaaggtagccacgtgcctttcaccttcggcggagggaccaag
gtggagatcaaacgaact
(amino acids)
(SEQ ID NO: 249)
QVQLVQSGAEVKKPGASVKVSCKASGYTFTDYAMNWVRQAPGQGLEWMGVISTFSGNTNFNQKFKGRVTMTTDTSTSTAYM
ELRSLRSDDTAVYYCARSDYYGPYFDYWGQGTTLTVSSDKTHTKPPKPAPELLGGPGTGTGGPTIKPPKPPKPAPNLLGG
PDIVMTQTPLSLSVTPGQPASISCRSSQTIVHSNGNTYLEWYLQKPGQSPQLLIYKVSNRFSGVPDRFSGSGSGTDFTLK
ISRVEAEDVGVYYCFQGSHVPFTFGGGTKVEIKRT
Humanized C8 single chain GS3 (linker is [Gly₄Ser₁]₃)
(DNA)
(SEQ ID NO: 250)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctccggcggtggcggatccggcggtggcggatccggcggtggcggatccgacatc
gtgatgacccagtctccagactccctggctgtgtctctgggcgagagggccaccatcaactgcagggccagcaagagtgtt
agcaccagcggctacagctacatgcactggtaccagcagaaaccaggacagcctcctaagctgctcatttacctggtgtct
aacctggaatccggggtccctgaccgattcagtggcagcgggtctgggacagatttcactctcaccatcagcagcctgcag
gctgaagatgtggcagtttattactgtcaacacattcgggaactgaccaggagtgaattcggcggagggaccaaggtggag
atcaaacgaact
(amino acids)
(SEQ ID NO: 251)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYWGKGTTVTVSSGGGGSGGGGSGGGGSDIVMTQSPDSLAVSLGERATINCRASK
SVSTSGYSYMHWYQQKPGQPPKLLIYLVSNLESGVPDRFSGSGSGTDFTLTISSLQAEDVAVYYCQHIRELTRSEFGGGT
KVEIKRT
Humanized C8 single chain IgG1 (no Cysteine)
(DNA)
(SEQ ID NO: 252)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctccgataaaacccatactaaaccgccaaaaccggcgccggaactgctgggtggt
cctggtaccggtgacatcgtgatgacccagtctccagactccctggctgtgtctctgggcgagagggccaccatcaactgc
agggccagcaagagtgttagcaccagcggctacagctacatgcactggtaccagcagaaaccaggacagcctcctaagctg
ctcatttacctggtgtctaacctggaatccggggtccctgaccgattcagtggcagcgggtctgggacagatttcactctc
accatcagcagcctgcaggctgaagatgtggcagtttattactgtcaacacattcgggaactgaccaggagtgaattcggc
ggagggaccaaggtggagatcaaacgaact
(amino acids)
(SEQ ID NO: 253)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYWGKGTTVTVSSDKTHTKPPKPAPELLGGPGTGDIVMTQSPDSLAVSLGERATI
NCRASKSVSTSGYSYMHWYQQKPGQPPKLLIYLVSNLESGVPDRFSGSGSGTDFTLTISSLQAEDVAVYYCQHIRELTRS
EFGGGTKVEIKRT
Humanized C8 single chain X4 (linker is IgG1 and IgG2 modified hinge region)
(DNA)
(SEQ ID NO: 254)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactacccagactcagtgaagggccgattcaccatctccagagacaacgccaagaactcactgtatctg
caaatgaacagcctgagagccgaggacacggccgtgtattactgtgcgagactgggcggcgacaattactatgagtattgg
ggcaaagggaccacggtcaccgtctcctccgataaaacccatactaaaccgccaaaaccggcgccggaactgctgggtggt
cctggtaccggtactggtggtccgactattaaacctccgaaacctccgaaacctgctccgaacctgctgggtggtccggac
atcgtgatgacccagtctccagactccctggctgtgtctctgggcgagagggccaccatcaactgcagggccagcaagagt
gttagcaccagcggctacagctacatgcactggtaccagcagaaaccaggacagcctcctaagctgctcatttacctggtg
tctaacctggaatccggggtccctgaccgattcagtggcagcgggtctgggacagatttcactctcaccatcagcagcctg
caggctgaagatgtggcagtttattactgtcaacacattcgggaactgaccaggagtgaattcggcggagggaccaaggtg
gagatcaaacgaact
(amino acids)
(SEQ ID NO: 255)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYWGKGTTVTVSSDKTHTKPPKPAPELLGGPGTGTGGPTIKPPKPPKPAPNLLGG
PDIVMTQSPDSLAVSLGERATINCRASKSVSTSGYSYMHWYQQKPGQPPKLLIYLVSNLESGVPDRFSGSGSGTDFTLTI
SSLQAEDVAVYYCQHIRELTRSEFGGGTKVEIKRT
pSECTag2 E6 scFV-FC
(DNA)
(SEQ ID NO: 256)
atggagacagacacactcctgctatgggtactgctgctctgggttccaggttccactggtgacgcggcccagccggccgag
gtgcagctggtggagtctgggggaggcctggtcaagcctggggggtccctgagactctcctgtgcagcctctggattcacc
ttcagtaggtatggcatgagctgggtccgccaggctccagggaagaggctggagtgggtctcaaccattagtggcggaggc
acctacatatactacccagactcagtgaagggccgattcaccatctccagagacaacgccaagaacaccctgtatctgcaa
atgaacagcctgagagccgaggacacggctgtgtattactgtaccagagataactatggccgcaactatgattatggcatg
gattattggggccagggcaccctggtgaccgtgagcagcggcggtggcggatccggcggtggcggatccggcggtggcgga
tccgaaattgtgttgacacagtctccagccaccctgtctttgtctccaggggaaagagccaccctcacctgcagcgccacc
agcagtgttagctacatccactggtaccaacagaggcctggccagagccccaggctcctcatctatagcacctccaacctg
gccagcggcatcccagccaggttcagtggcagtgggtctgggagcgactacactctcaccatcagcagcctagagcctgaa
gattttgcagtttattactgtcagcagcgtagcagctcccctttcacctttggcagcggcaccaaagtggaaattaaagag
cccaaatcttgtgacaaaactcacacatgcccaccgtgcccagcacctgaactcctggggggaccgtcagtcttcctcttc
cccccaaaacccaaggacaccctcatgatctcccggacccctgaggtcacatgcgtggtggtggacgtgagccacgaagac
cctgaggtcaagttcaactggtacgtggacggcgtggaggtgcataatgccaagacaaagccgcgggaggagcagtacaac
agcacgtaccgtgtggtcagcgtcctcaccgtcctgcaccaggactggctgaatggcaaggagtacaagtgcaaggtctcc
aacaaagccctcccagcccccatcgagaaaaccatctccaaagccaaagggcagccccgagaaccacaggtgtacaccctg
cccccatcccgggaggagatgaccaagaaccaggtcagcctgacctgcctggtcaaaggcttctatcccagcgacatcgcc
gtggagtgggagagcaatgggcagccggagaacaactacaagaccacgcctcccgtgctggactccgacggctccttcttc
ctctacagcaagctcaccgtggacaagagcaggtggcagcaggggaacgtcttctcatgctccgtgatgcatgaggctctg
cacaaccactacacgcagaagagcctctccctgtctccgggtaaatgataa
(amino acids)
(SEQ ID NO: 257)
METDTLLLWVLLLWVPGSTGDAAQPAEVQLVESGGGLVKPGGSLRLSCAASGFTFSRYGMSWVRQAPGKRLEWVSTISGG
GTYIYYPDSVKGRFTISRDNAKNTLYLQMNSLRAEDTAVYYCTRDNYGRNYDYGMDYWGQGTLVTVSSGGGGSGGGGSGG
GGSEIVLTQSPATLSLSPGERATLTCSATSSVSYIHWYQQRPGQSPRLLIYSTSNLASGIPARFSGSGSGSDYTLTISSL
EPEDFAVYYCQQRSSSPFTFGSGTKVEIKEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCS
VMHEALHNHYTQKSLSLSPGK**
Human IgG1 Fc sequence:
(DNA)
(SEQ ID NO: 272)
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaatgataa
(amino acids)
(SEQ ID NO: 273)
EPKSCDKTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQKSLSLSPGK**
CAR-T E6 CD8/CD8/CD3z sequence:
N-CD81s-huMNE6scFv-CD8ecd fragment- CD8 transmembrane- CD3zeta-C
(DNA)
(SEQ ID NO: 294)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgccgcgttaagttctcccgatcagccgacgcgcctgcttacaagcagggccagaac
caactgtacaacgagctgaatctcggtagacgggaagagtacgacgtgttggacaaacggagaggccgcgacccagaaatg
ggcggcaagcctcgcaggaaaaacccccaggagggactgtacaatgagttgcagaaagataagatggcagaagcttatagc
gagatcggaatgaagggggaaaggagacgagggaaaggacacgacggcctttatcagggcctgtccacagcaacaaaagat
acgtatgacgccctccatatgcaggcacttccaccacggtgataa
(amino acids)
(SEQ ID NO: 295)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRVKFSRSADAPAYKQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR**
**
CAR-T E6 CD8/CD8/CD28/CD3z sequence:
N-CD81s-huMNE6scFv-CD8ecd fragment- CD8 transmembrane- CD28- CD3zeta-C
(DNA)
(SEQ ID NO: 297)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgcagaagcaagcggtctcggctcctgcattctgattacatgaacatgaccccaaga
agaccaggccccaccaggaaacattaccagccctacgctccgccacgcgacttcgctgcctaccggtcccgcgttaagttc
tcccgatcagccgacgcgcctgcttacaagcagggccagaaccaactgtacaacgagctgaatctcggtagacgggaagag
tacgacgtgttggacaaacggagaggccgcgacccagaaatgggcggcaagcctcgcaggaaaaacccccaggagggactg
tacaatgagttgcagaaagataagatggcagaagcttatagcgagatcggaatgaagggggaaaggagacgagggaaagga
cacgacggcctttatcagggcctgtccacagcaacaaaagatacgtatgacgccctccatatgcaggcacttccaccacgg
tgataa
(amino acids)
(SEQ ID NO: 298)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRSKRSRLLHSDYMNMTPRRPGPTRKHYQPYAPPRDFAAYRSRVKFSRSADAPAYKQGQNQLYNELNL
GRREEYDVLDKRRGRDPEMGGKPRRKNPQEGLYNELQKDKMAEAYSEIGMKGERRRGKGHDGLYQGLSTATKDTYDALHM
QALPPR**
CAR-T E6 CD28/CD3z g BLOCK sequence:
(DNA)
(SEQ ID NO: 299)
tggagctgtgcacacaagaggactggatttcgcctgtgatatctacatttgggccccgctcgcaggcacatgtggagtgct
cctcctctccctggtgattaccctgtactgcagaagcaagcggtctcggctcctgcattctgattacatgaacatgacccc
aagaagaccaggccccaccaggaaacattaccagccctacgctccgccacgcgacttcgctgcctaccggtcccgcgttaa
gttctcccgatcagccgacgcgcctgcttacaagcagggccagaaccaactgtacaacgagctgaatctcggtagacggga
agagtacgacgtgttggacaaacggagaggccgcgacccagaaatgggcggcaagcctcgcaggaaaaacccccaggaggg
actgtacaatgagttgcagaaagataagatggcagaagcttatagcgagatcggaatgaagggggaaaggagacgagggaa
aggacacgacggcctttatcagggcctgtccacagcaacaaaagatacgtatgacgccctccatatgcaggcacttccacc
acggtgataagtttaaacccgctgatcagcctcgactgtgc
CAR-T E6 CD8/CD8/4-1BB/CD3z sequence:
N-CD81s-huMNE6scFv-CD8ecd fragment- CD8 transmembrane- 4-1BB- CD3zeta-C
(DNA)
(SEQ ID NO: 300)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgcaaaaggggccgcaaaaaactcctttacatttttaagcagccttttatgaggcca
gtacagacgactcaagaggaagacgggtgctcatgccgctttcctgaggaggaggaaggagggtgcgaactgcgcgttaag
ttctcccgatcagccgacgcgcctgcttacaagcagggccagaaccaactgtacaacgagctgaatctcggtagacgggaa
gagtacgacgtgttggacaaacggagaggccgcgacccagaaatgggcggcaagcctcgcaggaaaaacccccaggaggga
ctgtacaatgagttgcagaaagataagatggcagaagcttatagcgagatcggaatgaagggggaaaggagacgagggaaa
ggacacgacggcctttatcagggcctgtccacagcaacaaaagatacgtatgacgccctccatatgcaggcacttccacca
cggtgataa
(amino acids)
(SEQ ID NO: 301)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCKRGRKKLLYIFKQPFMRPVQTTQEEDGCSCRFPEEEEGGCELRVKFSRSADAPAYKQGQNQLYNELN
LGRREEYDVLDKRRGRDPEMGGKPRRKNPQEGLYNELQKDKMAEAYSEIGMKGERRRGKGHDGLYQGLSTATKDTYDALH
MQALPPR**
CAR-T E6 CD8/CD8/CD28/4-1BB/CD3z sequence:
N-CD81s-huMNE6scFv-CD8ecd fragment- CD8 transmembrane- CD28- 4-1BB- CD3zeta-C
(DNA)
(SEQ ID NO: 303)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgcagaagcaagcggtctcggctcctgcattctgattacatgaacatgaccccaaga
agaccaggccccaccaggaaacattaccagccctacgctccgccacgcgacttcgctgcctaccggtccaaaaggggccgc
aaaaaactcctttacatttttaagcagccttttatgaggccagtacagacgactcaagaggaagacgggtgctcatgccgc
tttcctgaggaggaggaaggagggtgcgaactgcgcgttaagttctcccgatcagccgacgcgcctgcttacaagcagggc
cagaaccaactgtacaacgagctgaatctcggtagacgggaagagtacgacgtgttggacaaacggagaggccgcgaccca
gaaatgggcggcaagcctcgcaggaaaaacccccaggagggactgtacaatgagttgcagaaagataagatggcagaagct
tatagcgagatcggaatgaagggggaaaggagacgagggaaaggacacgacggcctttatcagggcctgtccacagcaaca
aaagatacgtatgacgccctccatatgcaggcacttccaccacggtgataa
(amino acids)
(SEQ ID NO: 304)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRSKRSRLLHSDYMNMTPRRPGPTRKHYQPYAPPRDFAAYRSKRGRKKLLYIFKQPFMRPVQTTQEED
GCSCRFPEEEEGGCELRVKFSRSADAPAYKQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR**
CAR-T C2 CD8/CD8/CD28/4-1BB/CD3z sequence:
N-CD81s-huMNC2scFv-CD8ecd fragment- CD8 transmembrane- CD28- 4-1BB- CD3zeta-C
(DNA)
(SEQ ID NO: 306)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtccaaaaggggccgcaaaaaactcctttacatttttaagcagccttttatgaggccagtacagacgactcaagaggaa
gacgggtgctcatgccgctttcctgaggaggaggaaggagggtgcgaactgcgcgttaagttctcccgatcagccgacgcg
cctgcttacaagcagggccagaaccaactgtacaacgagctgaatctcggtagacgggaagagtacgacgtgttggacaaa
cggagaggccgcgacccagaaatgggcggcaagcctcgcaggaaaaacccccaggagggactgtacaatgagttgcagaaa
gataagatggcagaagcttatagcgagatcggaatgaagggggaaaggagacgagggaaaggacacgacggcctttatcag
ggcctgtccacagcaacaaaagatacgtatgacgccctccatatgcaggcacttccaccacggtgataa
(amino acids)
(SEQ ID NO: 307)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSSGGGGSGGGGSGGGGSDIVLTQSPASLAVSPGQRATITCRAS
KSVSTSGYSYMHWYQQKPGQPPKLLIYLASNLESGVPARFSGSGSGTDFTLTINPVEANDTANYYCQHSRELPFTFGGGTK
VEIKRTTTTPAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSKRGRKKLLYIFKQPFMRPVQTTQEEDGCSCRFPEEEEGGCELRVKF
SRSADAPAYKQGQNQLYNELNLGRREEYDVLDKRRGRDPEMGGKPRRKNPQEGLYNELQKDKMAEAYSEIGMKGERRRGKG
HDGLYQGLSTATKDTYDALHMQALPPR**
CAR E6 Fc/8/4-1BB/CD3z sequence:
N-CD81s-huMNE6scFv-Human IgG1 Fc- CD8 transmembrane- 4-1BB- CD3zeta-C
(DNA)
(SEQ ID NO: 310)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagcccaaatcttgtgac
aaaactcacacatgcccaccgtgcccagcacctgaactcctggggggaccgtcagtcttcctcttccccccaaaacccaag
gacaccctcatgatctcccggacccctgaggtcacatgcgtggtggtggacgtgagccacgaagaccctgaggtcaagttc
aactggtacgtggacggcgtggaggtgcataatgccaagacaaagccgcgggaggagcagtacaacagcacgtaccgtgtg
gtcagcgtcctcaccgtcctgcaccaggactggctgaatggcaaggagtacaagtgcaaggtctccaacaaagccctccca
gcccccatcgagaaaaccatctccaaagccaaagggcagccccgagaaccacaggtgtacaccctgcccccatcccgggag
gagatgaccaagaaccaggtcagcctgacctgcctggtcaaaggcttctatcccagcgacatcgccgtggagtgggagagc
aatgggcagccggagaacaactacaagaccacgcctcccgtgctggactccgacggctccttcttcctctacagcaagctc
accgtggacaagagcaggtggcagcaggggaacgtcttctcatgctccgtgatgcatgaggctctgcacaaccactacacg
cagaagagcctctccctgtctccgggtaaaatctacatttgggccccgctcgcaggcacatgtggagtgctcctcctctcc
ctggtgattaccctgtactgcaaaaggggccgcaaaaaactcctttacatttttaagcagccttttatgaggccagtacag
acgactcaagaggaagacgggtgctcatgccgctttcctgaggaggaggaaggagggtgcgaactgcgcgttaagttctcc
cgatcagccgacgcgcctgcttacaagcagggccagaaccaactgtacaacgagctgaatctcggtagacgggaagagtac
gacgtgttggacaaacggagaggccgcgacccagaaatgggcggcaagcctcgcaggaaaaacccccaggagggactgtac
aatgagttgcagaaagataagatggcagaagcttatagcgagatcggaatgaagggggaaaggagacgagggaaaggacac
gacggcctttatcagggcctgtccacagcaacaaaagatacgtatgacgccctccatatgcaggcacttccaccacggtga
taa
(amino acids)
(SEQ ID NO: 311)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQGNVFSCSVMHEA
LHNHYTQKSLSLSPGKIYIWAPLAGTCGVLLLSLVITLYCKRGRKKLLYIFKQPFMRPVQTTQEEDGCSCRFPEEEEGGC
ELRVKFSRSADAPAYKQGQNQLYNELNLGRREEYDVLDKRRGRDPEMGGKPRRKNPQEGLYNELQKDKMAEAYSEIGMKG
ERRRGKGHDGLYQGLSTATKDTYDALHMQALPPR**
CAR E6 FcH/8/4-1BB/CD3z sequence:
N-CD81s-huMNE6scFv-Human IgG1 hingeless Fc Y407R- CD8 transmembrane- 4-1BB-
CD3zeta-C
(DNA)
(SEQ ID NO: 315)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagcacctgaactcctgggg
ggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaag
gagtacaagtgcaaggtctccaacaaagccctcccagcccccatcgagaaaaccatctccaaagccaaagggcagccccga
gaaccacaggtgtacaccctgcccccatcccgggaggagatgaccaagaaccaggtcagcctgacctgcctggtcaaaggc
ttctatcccagcgacatcgccgtggagtgggagagcaatgggcagccggagaacaactacaagaccacgcctcccgtgctg
gactccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaatctacatttgggcc
ccgctcgcaggcacatgtggagtgctcctcctctccctggtgattaccctgtactgcaaaaggggccgcaaaaaactcctt
tacatttttaagcagccttttatgaggccagtacagacgactcaagaggaagacgggtgctcatgccgctttcctgaggag
gaggaaggagggtgcgaactgcgcgttaagttctcccgatcagccgacgcgcctgcttacaagcagggccagaaccaactg
tacaacgagctgaatctcggtagacgggaagagtacgacgtgttggacaaacggagaggccgcgacccagaaatgggcggc
aagcctcgcaggaaaaacccccaggagggactgtacaatgagttgcagaaagataagatggcagaagcttatagcgagatc
ggaatgaagggggaaaggagacgagggaaaggacacgacggcctttatcagggcctgtccacagcaacaaaagatacgtat
gacgccctccatatgcaggcacttccaccacggtgataa
(amino acids)
(SEQ ID NO: 316)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSLSLSPG
KIYIWAPLAGTCGVLLLSLVITLYCKRGRKKLLYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNELQKDKMAEAYSEIGMKGERRRGKGHDGLYQGL
STATKDTYDALHMQALPPR**
CAR E6 Fc/4/4-1BB/CD3z sequence:
N-CD81s-huMNE6scFv-Human IgG1 Fc- CD4 transmembrane- CD28- 4-1BB- CD3zeta-C
(DNA)
(SEQ ID NO: 318)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagcccaaatcttgtgac
aaaactcacacatgcccaccgtgcccagcacctgaactcctggggggaccgtcagtcttcctcttccccccaaaacccaag
gacaccctcatgatctcccggacccctgaggtcacatgcgtggtggtggacgtgagccacgaagaccctgaggtcaagttc
aactggtacgtggacggcgtggaggtgcataatgccaagacaaagccgcgggaggagcagtacaacagcacgtaccgtgtg
gtcagcgtcctcaccgtcctgcaccaggactggctgaatggcaaggagtacaagtgcaaggtctccaacaaagccctccca
gcccccatcgagaaaaccatctccaaagccaaagggcagccccgagaaccacaggtgtacaccctgcccccatcccgggag
gagatgaccaagaaccaggtcagcctgacctgcctggtcaaaggcttctatcccagcgacatcgccgtggagtgggagagc
aatgggcagccggagaacaactacaagaccacgcctcccgtgctggactccgacggctccttcttcctctacagcaagctc
accgtggacaagagcaggtggcagcaggggaacgtcttctcatgctccgtgatgcatgaggctctgcacaaccactacacg
cagaagagcctctccctgtctccgggtaaaatggccctgattgtgctggggggcgtcgccggcctcctgcttttcattggg
ctaggcatcttcttcaaaaggggccgcaaaaaactcctttacatttttaagcagccttttatgaggccagtacagacgact
caagaggaagacgggtgctcatgccgctttcctgaggaggaggaaggagggtgcgaactgcgcgttaagttctcccgatca
gccgacgcgcctgcttacaagcagggccagaaccaactgtacaacgagctgaatctcggtagacgggaagagtacgacgtg
ttggacaaacggagaggccgcgacccagaaatgggcggcaagcctcgcaggaaaaacccccaggagggactgtacaatgag
ttgcagaaagataagatggcagaagcttatagcgagatcggaatgaagggggaaaggagacgagggaaaggacacgacggc
ctttatcagggcctgtccacagcaacaaaagatacgtatgacgccctccatatgcaggcacttccaccacggtgataa
(amino acids)
(SEQ ID NO: 319)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQGNVFSCSVMHEA
LHNHYTQKSLSLSPGKMALIVLGGVAGLLLFIGLGIFFKRGRKKLLYIFKQPFMRPVQTTQEEDGCSCRFPEEEEGGCEL
RVKFSRSADAPAYKQGQNQLYNELNLGRREEYDVLDKRRGRDPEMGGKPRRKNPQEGLYNELQKDKMAEAYSEIGMKGER
RRGKGHDGLYQGLSTATKDTYDALHMQALPPR**
CAR E6 FcH/4/4-1BB/CD3z sequence:
N-CD81s-huMNE6scFv-Human IgG1 hingeless Fc Y407R- CD4 transmembrane- CD28-
4-1BB-CD3zeta-C
(DNA)
(SEQ ID NO: 321)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagcacctgaactcctgggg
ggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaag
gagtacaagtgcaaggtctccaacaaagccctcccagcccccatcgagaaaaccatctccaaagccaaagggcagccccga
gaaccacaggtgtacaccctgcccccatcccgggaggagatgaccaagaaccaggtcagcctgacctgcctggtcaaaggc
ttctatcccagcgacatcgccgtggagtgggagagcaatgggcagccggagaacaactacaagaccacgcctcccgtgctg
gactccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaatggccctgattgtg
ctggggggcgtcgccggcctcctgcttttcattgggctaggcatcttcttcaaaaggggccgcaaaaaactcctttacatt
tttaagcagccttttatgaggccagtacagacgactcaagaggaagacgggtgctcatgccgctttcctgaggaggaggaa
ggagggtgcgaactgcgcgttaagttctcccgatcagccgacgcgcctgcttacaagcagggccagaaccaactgtacaac
gagctgaatctcggtagacgggaagagtacgacgtgttggacaaacggagaggccgcgacccagaaatgggcggcaagcct
cgcaggaaaaacccccaggagggactgtacaatgagttgcagaaagataagatggcagaagcttatagcgagatcggaatg
aagggggaaaggagacgagggaaaggacacgacggcctttatcagggcctgtccacagcaacaaaagatacgtatgacgcc
ctccatatgcaggcacttccaccacggtgataa
(amino acids)
(SEQ ID NO: 322)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSLSLSPG
KMALIVLGGVAGLLLFIGLGIFFKRGRKKLLYIFKQPFMRPVQTTQEEDGCSCRFPEEEEGGCELRVKFSRSADAPAYKQ
GQNQLYNELNLGRREEYDVLDKRRGRDPEMGGKPRRKNPQEGLYNELQKDKMAEAYSEIGMKGERRRGKGHDGLYQGLST
ATKDTYDALHMQALPPR**
CAR E6 IgD/8/4-1BB/CD3z sequence:
N-CD81s-huMNE6scFv-IgD hinge region- CD8 transmembrane- 4-1BB- CD3zeta-C
(DNA)
(SEQ ID NO: 323)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagtctccaaaggcaca
ggcctcctcagtgcccactgcacaaccccaagcagagggcagcctcgccaaggcaaccacagccccagccaccacccgta
acacaggaagaggcggcgaagagaagaaaaaggagaaggagaaagaggaacaagaagagagagagacaaagacaccaatc
tacatttgggccccgctcgcaggcacatgtggagtgctcctcctctccctggtgattaccctgtactgcaaaaggggccgc
aaaaaactcctttacatttttaagcagccttttatgaggccagtacagacgactcaagaggaagacgggtgctcatgccgc
tttcctgaggaggaggaaggagggtgcgaactgcgcgttaagttctcccgatcagccgacgcgcctgcttacaagcagggc
cagaaccaactgtacaacgagctgaatctcggtagacgggaagagtacgacgtgttggacaaacggagaggccgcgaccca
gaaatgggcggcaagcctcgcaggaaaaacccccaggagggactgtacaatgagttgcagaaagataagatggcagaagct
tatagcgagatcggaatgaagggggaaaggagacgagggaaaggacacgacggcctttatcagggcctgtccacagcaaca
aaagatacgtatgacgccctccatatgcaggcacttccaccacggtgataa
(amino acids)
(SEQ ID NO: 324)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKESPKAQASSVPTAQPQAEGSLAKATTAPATTRNTGRGGEEKKKEKEKEEQEERETK
TPIYIWAPLAGTCGVLLLSLVITLYCKRGRKKLLYIFKQPFMRPVQTTQEEDGCSCRFPEEEEGGCELRVKFSRSADAPA
YKQGQNQLYNELNLGRREEYDVLDKRRGRDPEMGGKPRRKNPQEGLYNELQKDKMAEAYSEIGMKGERRRGKGHDGLYQG
LSTATKDTYDALHMQALPPR**
CAR E6 IgD/4/4-1BB/CD3z sequence:
N-CD81s-huMNE6scFv-IgD hinge region- CD4 transmembrane- 4-1BB- CD3zeta-C
(DNA)
(SEQ ID NO: 327)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagtctccaaaggcaca
ggcctcctcagtgcccactgcacaaccccaagcagagggcagcctcgccaaggcaaccacagccccagccaccacccgta
acacaggaagaggcggcgaagagaagaaaaaggagaaggagaaagaggaacaagaagagagagagacaaagacaccaatg
gccctgattgtgctggggggcgtcgccggcctcctgcttttcattgggctaggcatcttcttcaaaaggggccgcaaaaaa
ctcctttacatttttaagcagccttttatgaggccagtacagacgactcaagaggaagacgggtgctcatgccgctttcct
gaggaggaggaaggagggtgcgaactgcgcgttaagttctcccgatcagccgacgcgcctgcttacaagcagggccagaac
caactgtacaacgagctgaatctcggtagacgggaagagtacgacgtgttggacaaacggagaggccgcgacccagaaatg
ggcggcaagcctcgcaggaaaaacccccaggagggactgtacaatgagttgcagaaagataagatggcagaagcttatagc
gagatcggaatgaagggggaaaggagacgagggaaaggacacgacggcctttatcagggcctgtccacagcaacaaaagat
acgtatgacgccctccatatgcaggcacttccaccacggtgataa
(amino acids)
(SEQ ID NO: 328)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKESPKAQASSVPTAQPQAEGSLAKATTAPATTRNTGRGGEEKKKEKEKEEQEERETK
TPMALIVLGGVAGLLLFIGLGIFFKRGRKKLLYIFKQPFMRPVQTTQEEDGCSCRFPEEEEGGCELRVKFSRSADAPAYK
QGQNQLYNELNLGRREEYDVLDKRRGRDPEMGGKPRRKNPQEGLYNELQKDKMAEAYSEIGMKGERRRGKGHDGLYQGLS
TATKDTYDALHMQALPPR**
CAR E6 X4/6/4-1BB/CD3z sequence:
N-CD81s-huMNE6scFv- X4 linker- CD8 transmembrane- 4-1BB- CD3zeta-C
(DNA)
(SEQ ID NO: 330)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagacaagacgcacaccaag
ccacctaaaccagctccagaactgctcggaggtcctggcaccggaaccggaggacctaccatcaaaccacctaagccacct
aagcctgctcctaacctgctcggaggacctatctacatttgggccccgctcgcaggcacatgtggagtgctcctcctctcc
ctggtgattaccctgtactgcaaaaggggccgcaaaaaactcctttacatttttaagcagccttttatgaggccagtacag
acgactcaagaggaagacgggtgctcatgccgctttcctgaggaggaggaaggagggtgcgaactgcgcgttaagttctcc
cgatcagccgacgcgcctgcttacaagcagggccagaaccaactgtacaacgagctgaatctcggtagacgggaagagtac
gacgtgttggacaaacggagaggccgcgacccagaaatgggcggcaagcctcgcaggaaaaacccccaggagggactgtac
aatgagttgcagaaagataagatggcagaagcttatagcgagatcggaatgaagggggaaaggagacgagggaaaggacac
gacggcctttatcagggcctgtccacagcaacaaaagatacgtatgacgccctccatatgcaggcacttccaccacggtga
taa
(amino acids)
(SEQ ID NO: 331)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKDKTHTKPPKPAPELLGGPGTGTGGPTIKPPKPPKPAPNLLGGPIYIWAPLAGTCGV
LLLSLVITLYCKRGRKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTATKDTYDALHMQ
ALPPR**
CAR E6 X4/4/4-1BB/CD3z sequence:
N-CD81s-huMNE6scFv-X4 linker- CD4 transmembrane- 4-1BB- CD3zeta-C
(DNA)
(SEQ ID NO: 333)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagacaagacgcacaccaag
ccacctaaaccagctccagaactgctcggaggtcctggcaccggaaccggaggacctaccatcaaaccacctaagccacct
aagcctgctcctaacctgctcggaggacctatggccctgattgtgctggggggcgtcgccggcctcctgcttttcattggg
ctaggcatcttcttcaaaaggggccgcaaaaaactcctttacatttttaagcagccttttatgaggccagtacagacgact
caagaggaagacgggtgctcatgccgctttcctgaggaggaggaaggagggtgcgaactgcgcgttaagttctcccgatca
gccgacgcgcctgcttacaagcagggccagaaccaactgtacaacgagctgaatctcggtagacgggaagagtacgacgtg
ttggacaaacggagaggccgcgacccagaaatgggcggcaagcctcgcaggaaaaacccccaggagggactgtacaatgag
ttgcagaaagataagatggcagaagcttatagcgagatcggaatgaagggggaaaggagacgagggaaaggacacgacggc
ctttatcagggcctgtccacagcaacaaaagatacgtatgacgccctccatatgcaggcacttccaccacggtgataa
(amino acids)
(SEQ ID NO: 334)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKDKTHTKPPKPAPELLGGPGTGTGGPTIKPPKPPKPAPNLLGGPMALIVLGGVAGLL
LFIGLGIFFKRGRKKLLYIFKQPFMRPVQTTQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGERRRGKGHDGLYQGLSTATKDTYDALHMQAL
PPR**
CAR E6 8 + 4/4/4-1BB/CD3z sequence:
N-CD81s-huMNE6scFv- CD8ecd + CD4ecd fragment - CD4 transmembrane- 4-1BB-
CD3zeta-C
(DNA)
(SEQ ID NO: 336)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatggccctgattgtgctggggggcgtcgccggcctcctgcttttc
attgggctaggcatcttcttcaaaaggggccgcaaaaaactcctttacatttttaagcagccttttatgaggccagtacag
acgactcaagaggaagacgggtgctcatgccgctttcctgaggaggaggaaggagggtgcgaactgcgcgttaagttctcc
cgatcagccgacgcgcctgcttacaagcagggccagaaccaactgtacaacgagctgaatctcggtagacgggaagagtac
gacgtgttggacaaacggagaggccgcgacccagaaatgggcggcaagcctcgcaggaaaaacccccaggagggactgtac
aatgagttgcagaaagataagatggcagaagcttatagcgagatcggaatgaagggggaaaggagacgagggaaaggacac
gacggcctttatcagggcctgtccacagcaacaaaagatacgtatgacgccctccatatgcaggcacttccaccacggtga
taa
(amino acids)
(SEQ ID NO: 337)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDMALIVLGGVAG
LLLFIGLGIFFKRGRKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTATKDTYDALHMQ
ALPPR**
Humanized C2 scFV sequence in CAR:
(DNA)
(SEQ ID NO: 339)
gagggccaccatggccttgccagtgacggccctgctgctgccattggctcttctgttgcacgctgccaggcctgaagtgca
gctcgtagagagtggcgggggactggtgaagcccggtggaagcctcagactcagttgcgccgcctcaggtttcactttttc
aggttacgccatgtcctgggtaagacaggcaccggggaaaggactcgagtgggtgtctactatcagctcaggaggcactta
tatatattatcctgactctgtaaaaggccgatttacgatttctcgcgacaatgcaaagaactccctctacctccaaatgaa
cagtcttagggcagaagacactgctgtatactattgtgcacgcctcggcggcgacaactactacgagtactttgacgtgtg
ggggaaagggactaccgtgacagtttcaagcggaggaggtggctcaggtggaggcgggtcaggggggggaggaagtgatat
tgtgctcacacaatccccagcctccctggctgtgtctcccggccaacgcgctacaattacatgtcgggcctccaaaagcgt
gagcaccagcggctacagctacatgcactggtatcaacagaaaccaggacaaccccccaaactgttgatttatctcgcttc
aaacttggagtccggcgtgcctgcgcgcttttcagggagtgggagcggcacagattttacgctgactatcaaccccgtaga
agcaaacgatacagcgaattattattgtcaacattcccgggaactcccctttacgttcggcgggggcacaaaggtcgaaat
taagagaacc
(amino acids)
(SEQ ID NO: 340)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRT
Humanized E6 scFV sequence in CAR:
(DNA)
(SEQ ID NO: 341)
gaggtccagctggttgagagtggcggtgggctggttaagcctggcggctccctgcggctgagctgcgccgcgagtggattt
actttcagccgatatgggatgagttgggtgcggcaagctcccgggaagaggctggaatgggtctcaacaatctccgggggg
ggcacttacatctattaccccgactcagtcaaggggagatttaccatttcacgagacaacgctaagaataccctgtatttg
cagatgaattctctgagagcagaggacacagctgtttactattgtacccgcgacaactatggcaggaactacgactacggt
atggactattggggacaagggacattggttacagtgagcagtggcggcgggggcagcggaggaggaggcagcggtgggggg
ggcagcgagatagtgctcacgcagtcacccgcgactctcagtctctcacctggggaacgagctaccctgacgtgctctgct
acctcctcagtgtcatatattcactggtatcagcaacggcccgggcagtcccctagattgctcatttatagtacctctaat
ctggcctcaggtatccctgcacgattttctggatctggttcaggttctgattacaccctcactatctctagcctggagcct
gaagactttgccgtttattactgccagcagaggtctagctccccattcacctttgggagtgggaccaaggttgaaattaaa
(amino acids)
(SEQ ID NO: 342)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSGGGGSGGGGSGGGGSEIVLTQSPATLSLSPGERATLTC
SATSSVSYIHWYQQRPGQSPRLLIYSTSNLASGIPARFSGSGSGSDYTLTISSLEPEDFAVYYCQQRSSSPFTFGSGTKV
EIK
CD8 leader sequence:
(DNA)
(SEQ ID NO: 343)
atggccctgcccgtgaccgctttgctgctccccctggcgctgctgctgcacgccgccaggcca
(amino acids)
(SEQ ID NO: 344)
MALPVTALLLPLALLLHAARP
CD8 hinge domain sequence:
(DNA)
(SEQ ID NO: 345)
acgacaaccccggcccccagaccaccaacgccagcccccaccatcgccagccaacccctgtctctgagaccagaagcctgt
aggcctgccgccggtggagctgtgcacacaagaggactggatttcgcctgtgat
(amino acids)
(SEQ ID NO: 346)
TTTPAPRPPTPAPTIASQPLSLRPEACRPAAGGAVHTRGLDFACD
CD4 hinge domain sequence:
(DNA)
(SEQ ID NO: 347)
tcgggacaggtcctgctggaatccaacatcaaggttctgcccacatggtccaccccggtgcagcca
(amino acids)
(SEQ ID NO: 348)
SGQVLLESNIKVLPTWSTPVQP
CD28 hinge domain sequence:
(DNA)
(SEQ ID NO: 349)
aaacacctttgtccaagtcccctatttcccggaccttctaagccc
(amino acids)
(SEQ ID NO: 350)
KHLCPSPLFPGPSKP
CD8 + CD4 hinge domain sequence:
(DNA)
(SEQ ID NO: 351)
acgacaaccccggcccccagaccaccaacgccagcccccaccatcgccagccaacccctgtctctgagaccagaagcctgt
aggcctgccgccggtggagctgtgcacacaagaggactggatttcgcctgtgattcgggacaggtcctgctggaatccaac
atcaaggttctgcccacatggtccaccccggtgcagcca
(amino acids)
(SEQ ID NO: 352)
TTTPAPRPPTPAPTIASQPLSLRPEACRPAAGGAVHTRGLDFACDSGQVLLESNIKVLPTWSTPVQP
CD8 + CD28 hinge domain sequence:
(DNA)
(SEQ ID NO: 353)
acgacaaccccggcccccagaccaccaacgccagcccccaccatcgccagccaacccctgtctctgagaccagaagcctgt
aggcctgccgccggtggagctgtgcacacaagaggactggatttcgcctgtgataaacacctttgtccaagtcccctatt
tcccggaccttctaagccc
(amino acids)
(SEQ ID NO: 354)
TTTPAPRPPTPAPTIASQPLSLRPEACRPAAGGAVHTRGLDFACDKHLCPSPLFPGPSKP
CD28 + CD4 hinge domain sequence:
(DNA)
(SEQ ID NO: 355)
aaacacctttgtccaagtcccctatttcccggaccttctaagccctcgggacaggtcctgctggaatccaacatcaaggt
tctgcccacatggtccaccccggtgcagcca
(amino acids)
(SEQ ID NO: 356)
KHLCPSPLFPGPSKPSGQVLLESNIKVLPTWSTPVQP
Human IgD hinge domain sequence:
(DNA)
(SEQ ID NO: 357)
gagtctccaaaggcacaggcctcctcagtgcccactgcacaaccccaagcagagggcagcctcgccaaggcaaccacagc
cccagccaccacccgtaacacaggaagaggcggcgaagagaagaaaaaggagaaggagaaagaggaacaagaagagagag
agacaaagacacca
(amino acids)
(SEQ ID NO: 358)
ESPKAQASSVPTAQPQAEGSLAKATTAPATTRNTGRGGEEKKKEKEKEEQEERETKTP
X4 linker (IgG1 and IgG2 modified hinge region) sequence:
(DNA)
(SEQ ID NO: 359)
gacaagacgcacaccaagccacctaaaccagctccagaactgctcggaggtcctggcaccggaaccggaggacctaccatc
aaaccacctaagccacctaagcctgctcctaacctgctcggaggacct
(amino acids)
(SEQ ID NO: 360)
DKTHTKPPKPAPELLGGPGTGTGGPTIKPPKPPKPAPNLLGGP
CD3 zeta transmembrane domain sequence:
(DNA)
(SEQ ID NO: 361)
ctctgctacctgctggatggaatcctcttcatctatggtgtcattctcactgccttgttcctg
(amino acids)
(SEQ ID NO: 362)
LCYLLDGILFIYGVILTALFL
CD8 transmembrane domain sequence:
(DNA)
(SEQ ID NO: 363)
atctacatttgggccccgctcgcaggcacatgtggagtgctcctcctctccctggtgattaccctgtactgc
(amino acids)
(SEQ ID NO: 364)
IYIWAPLAGTCGVLLLSLVITLYC
CD4 transmembrane domain sequence:
(DNA)
(SEQ ID NO: 365)
atggccctgattgtgctggggggcgtcgccggcctcctgcttttcattgggctaggcatcttcttc
(amino acids)
(SEQ ID NO: 366)
MALIVLGGVAGLLLFIGLGIFF
CD28 transmembrane domain sequence:
(DNA)
(SEQ ID NO: 367)
ttttgggtgctggtggtggttggtggagtcctggcttgctatagcttgctagtaacagtggcctttattattttctgggt
g
(amino acids)
(SEQ ID NO: 368)
FWVLVVVGGVLACYSLLVTVAFIIFWV
4-1BB transmembrane domain sequence:
(DNA)
(SEQ ID NO: 369)
atcatctccttctttcttgcgctgacgtcgactgcgttgctcttcctgctgttcttcctcacgctccgtttctctgttgt
t
(amino acids)
(SEQ ID NO: 370)
IISFFLALTSTALLFLLFFLTLRFSVV
OX40 transmembrane domain sequence:
(DNA)
(SEQ ID NO: 371)
gttgccgccatcctgggcctgggcctggtgctggggctgctgggccccctggccatcctgctggccctgtacctgctc
(amino acids)
(SEQ ID NO: 372)
VAAILGLGLVLGLLGPLAILLALYLL
CD3 zeta domain sequence:
(DNA)
(SEQ ID NO: 373)
cgcgttaagttctcccgatcagccgacgcgcctgcttacaagcagggccagaaccaactgtacaacgagctgaatctcggt
agacgggaagagtacgacgtgttggacaaacggagaggccgcgacccagaaatgggcggcaagcctcgcaggaaaaacccc
caggagggactgtacaatgagttgcagaaagataagatggcagaagcttatagcgagatcggaatgaagggggaaaggaga
cgagggaaaggacacgacggcctttatcagggcctgtccacagcaacaaaagatacgtatgacgccctccatatgcaggca
cttccaccacgg
(amino acids)
(SEQ ID NO: 374)
RVKFSRSADAPAYKQGQNQLYNELNLGRREEYDVLDKRRGRDPEMGGKPRRKNPQEGLYNELQKDKMAEAYSEIGMKGERR
RGKGHDGLYQGLSTATKDTYDALHMQALPPR
CD3 zeta domain variant sequence:
(DNA)
(SEQ ID NO: 375)
agagtgaagttcagcaggagcgcagacgcccccgcgtaccagcagggccagaaccagctctataacgagctcaatctagg
acgaagagaggagtacgatgttttggacaagagacgtggccgggaccctgagatggggggaaagccgagaaggaagaacc
ctcaggaaggcctgtacaatgaactgcagaaagataagatggcggaggcctacagtgagattgggatgaaaggcgagcgc
cggaggggcaaggggcacgatggcctttaccagggtctcagtacagccaccaaggacacctacgacgcccttcacatgca
ggccctgccccctcgc
(amino acids)
(SEQ ID NO: 376)
RVKFSRSADAPAYQQGQNQLYNELNLGRREEYDVLDKRRGRDPEMGGKPRRKNPQEGLYNELQKDKMAEAYSEIGMKGER
RRGKGHDGLYQGLSTATKDTYDALHMQALPPR
CD28 domain sequence:
(DNA)
(SEQ ID NO: 377)
agaagcaagcggtctcggctcctgcattctgattacatgaacatgaccccaagaagaccaggccccaccaggaaacattac
cagccctacgctccgccacgcgacttcgctgcctaccggtcc
(amino acids)
(SEQ ID NO: 378)
RSKRSRLLHSDYMNMTPRRPGPTRKHYQPYAPPRDFAAYRS
4-1BB domain sequence:
(DNA)
(SEQ ID NO: 379)
aaaaggggccgcaaaaaactcctttacatttttaagcagccttttatgaggccagtacagacgactcaagaggaagacggg
tgctcatgccgctttcctgaggaggaggaaggagggtgcgaactg
(amino acids)
(SEQ ID NO: 380)
KRGRKKLLYIFKQPFMRPVQTTQEEDGCSCRFPEEEEGGCEL
OX40 domain sequence:
(DNA)
(SEQ ID NO: 381)
cggagggaccagaggctgccccccgatgcccacaagccccctgggggaggcagtttccggacccccatccaagaggagcag
gccgacgcccactccaccctggccaagatc
(amino acids)
(SEQ ID NO: 382)
RRDQRLPPDAHKPPGGGSFRTPIQEEQADAHSTLAKI
Humanized anti CD3 scFV clone 12F6 (VH-VL) sequence:
(DNA)
(SEQ ID NO: 383)
caggtgcagctggtgcagagcggaggtggagtggtccaacctggaagatctctgagactgagctgtaaggctagcgggta
cacgttcacatcttacacgatgcactgggtgaggcaagcccccggtaagggcctggaatggatcggatatataaacccca
gctcagggtataccaaatataatcagaagttcaaagatcggttcacgatttctgctgataaaagtaagtccaccgctttc
ctgcagatggactcactcaggccagaagatactggtgtttatttctgtgcaaggtggcaggactacgacgtgtactttga
ctattgggggcaggggacgcctgtaacagtatcaagcggcggtggcggatccggcggtggcggatccggcggtggcggat
ccgatattcagatgacccagagcccgagcagcctgagcgcgagcgtgggcgatcgcgtgaccatgacctgccgcgcgagc
agcagcgtgagctatatgcattggtatcagcagaccccgggcaaagcgccgaaaccgtggatttatgcgaccagcaacct
ggcgagcggcgtgccgagccgctttagcggcagcggcagcggcaccgattataccctgaccattagcagcctgcagccgg
aagatattgcgacctattattgccagcagtggagcagcaacccgccgacctttggccagggcaccaaactgcagattacc
cgc
(amino acids)
(SEQ ID NO: 384)
QVQLVQSGGGVVQPGRSLRLSCKASGYTFTSYTMHWVRQAPGKGLEWIGYINPSSGYTKYNQKFKDRFTISADKSKSTAF
LQMDSLRPEDTGVYFCARWQDYDVYFDYWGQGTPVTVSSGGGGSGGGGSGGGGSDIQMTQSPSSLSASVGDRVTMTCRAS
SSVSYMHWYQQTPGKAPKPWIYATSNLASGVPSRFSGSGSGTDYTLTISSLQPEDIATYYCQQWSSNPPTFGQGTKLQIT
R
Humanized anti CD3 scFV clone 12F6 (VL-VH) sequence:
(DNA)
(SEQ ID NO: 385)
gatattcagatgacccagagcccgagcagcctgagcgcgagcgtgggcgatcgcgtgaccatgacctgccgcgcgagcag
cagcgtgagctatatgcattggtatcagcagaccccgggcaaagcgccgaaaccgtggatttatgcgaccagcaacctgg
cgagcggcgtgccgagccgctttagcggcagcggcagcggcaccgattataccctgaccattagcagcctgcagccggaa
gatattgcgacctattattgccagcagtggagcagcaacccgccgacctttggccagggcaccaaactgcagattacccg
cggcggtggcggatccggcggtggcggatccggcggtggcggatcccaggtgcagctggtgcagagcggaggtggagtgg
tccaacctggaagatctctgagactgagctgtaaggctagcgggtacacgttcacatcttacacgatgcactgggtgagg
caagcccccggtaagggcctggaatggatcggatatataaaccccagctcagggtataccaaatataatcagaagttcaa
agatcggttcacgatttctgctgataaaagtaagtccaccgctttcctgcagatggactcactcaggccagaagatactg
gtgtttatttctgtgcaaggtggcaggactacgacgtgtactttgactattgggggcaggggacgcctgtaacagtatca
agc
(amino acids)
(SEQ ID NO: 386)
DIQMTQSPSSLSASVGDRVTMTCRASSSVSYMHWYQQTPGKAPKPWIYATSNLASGVPSRFSGSGSGTDYTLTISSLQPE
DIATYYCQQWSSNPPTFGQGTKLQITRGGGGSGGGGSGGGGSQVQLVQSGGGVVQPGRSLRLSCKASGYTFTSYTMHWVR
QAPGKGLEWIGYINPSSGYTKYNQKFKDRFTISADKSKSTAFLQMDSLRPEDTGVYFCARWQDYDVYFDYWGQGTPVTVS
S
Humanized anti CD3 scFV clone OKT3 (VH-VL) sequence:
(DNA)
(SEQ ID NO: 387)
caggtgcagctggtgcagagcggaggcggagtggtgcagcctggaagaagcctgcgcctgagctgcaaagcgagcggcta
tacctttacccgctataccatgcattgggtgcgccaggcgccgggcaaaggcctggaatggattggctatattaacccga
gccgcggctataccaactataaccagaaagtgaaagatcgctttaccattagcaccgataaaagcaaaagcaccgcgttt
ctgcagatggatagcctgcgcccggaagataccgcggtgtattattgcgcgcgctattatgatgatcattattgcctgga
ttattggggccagggcaccaccctgaccgtgagcagcggcggtggcggatccggcggtggcggatccggcggtggcggat
ccgatattcagatgacccagagcccgagcagcctgagcgcgagcgtgggcgatcgcgtgaccattacctgcagcgcgagc
agcagcgtgagctatatgaactggtatcagcagaccccgggcaaagcgccgaaacgctggatttatgataccagcaaact
ggcgagcggcgtgccgagccgctttagcggcagcggcagcggcaccgattatacctttaccattagcagcctgcagccgg
aagatattgcgacctattattgccagcagtggagcagcaacccgtttacctttggccagggcaccaaactgcagattacc
cgc
(amino acids)
(SEQ ID NO: 388)
QVQLVQSGGGVVQPGRSLRLSCKASGYTFTRYTMHWVRQAPGKGLEWIGYINPSRGYTNYNQKVKDRFTISTDKSKSTAF
LQMDSLRPEDTAVYYCARYYDDHYCLDYWGQGTTLTVSSGGGGSGGGGSGGGGSDIQMTQSPSSLSASVGDRVTITCSAS
SSVSYMNWYQQTPGKAPKRWIYDTSKLASGVPSRFSGSGSGTDYTFTISSLQPEDIATYYCQQWSSNPFTFGQGTKLQIT
R
Humanized anti CD3 scFV clone OKT3 (VH-VL) sequence:
(DNA)
(SEQ ID NO: 389)
gatattcagatgacccagagcccgagcagcctgagcgcgagcgtgggcgatcgcgtgaccattacctgcagcgcgagcag
cagcgtgagctatatgaactggtatcagcagaccccgggcaaagcgccgaaacgctggatttatgataccagcaaactgg
cgagcggcgtgccgagccgctttagcggcagcggcagcggcaccgattatacctttaccattagcagcctgcagccggaa
gatattgcgacctattattgccagcagtggagcagcaacccgtttacctttggccagggcaccaaactgcagattacccg
cggcggtggcggatccggcggtggcggatccggcggtggcggatcccaggtgcagctggtgcagagcggaggcggagtgg
tgcagcctggaagaagcctgcgcctgagctgcaaagcgagcggctatacctttacccgctataccatgcattgggtgcgc
caggcgccgggcaaaggcctggaatggattggctatattaacccgagccgcggctataccaactataaccagaaagtgaa
agatcgctttaccattagcaccgataaaagcaaaagcaccgcgtttctgcagatggatagcctgcgcccggaagataccg
cggtgtattattgcgcgcgctattatgatgatcattattgcctggattattggggccagggcaccaccctgaccgtgagc
agc
(amino acids)
(SEQ ID NO: 390)
DIQMTQSPSSLSASVGDRVTITCSASSSVSYMNWYQQTPGKAPKRWIYDTSKLASGVPSRFSGSGSGTDYTFTISSLQPE
DIATYYCQQWSSNPFTFGQGTKLQITRGGGGSGGGGSGGGGSQVQLVQSGGGVVQPGRSLRLSCKASGYTFTRYTMHWVR
QAPGKGLEWIGYINPSRGYTNYNQKVKDRFTISTDKSKSTAFLQMDSLRPEDTAVYYCARYYDDHYCLDYWGQGTTLTVS
S
HumanizeE6 scFV (VH-VL) sequence:
(DNA)
(SEQ ID NO: 391)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacaccctgtatctg
caaatgaacagcctgagagccgaggacacggctgtgtattactgtaccagagataactatggccgcaactatgattatggc
atggattattggggccagggcaccctggtgaccgtgagcagcggcggtggcggatccggcggtggcggatccggcggtggc
ggatccgaaattgtgttgacacagtctccagccaccctgtctttgtctccaggggaaagagccaccctcacctgcagcgcc
accagcagtgttagctacatccactggtaccaacagaggcctggccagagccccaggctcctcatctatagcacctccaac
ctggccagcggcatcccagccaggttcagtggcagtgggtctgggagcgactacactctcaccatcagcagcctagagcct
gaagattttgcagtttattactgtcagcagcgtagcagctcccctttcacctttggcagcggcaccaaagtggaaattaaa
(amino acids)
(SEQ ID NO: 392)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSGGGGSGGGGSGGGGSEIVLTQSPATLSLSPGERATLTC
SATSSVSYIHWYQQRPGQSPRLLIYSTSNLASGIPARFSGSGSGSDYTLTISSLEPEDFAVYYCQQRSSSPFTFGSGTKV
EIK
HumanizeE6 scFV (VL-VH) sequence:
(DNA)
(SEQ ID NO: 393)
gaaattgtgttgacacagtctccagccaccctgtctttgtctccaggggaaagagccaccctcacctgcagcgccaccagc
agtgttagctacatccactggtaccaacagaggcctggccagagccccaggctcctcatctatagcacctccaacctggcc
agcggcatcccagccaggttcagtggcagtgggtctgggagcgactacactctcaccatcagcagcctagagcctgaagat
tttgcagtttattactgtcagcagcgtagcagctcccctttcacctttggcagcggcaccaaagtggaaattaaaggcggt
ggcggatccggcggtggcggatccggcggtggcggatccgaggtgcagctggtggagtctgggggaggcctggtcaagcct
ggggggtccctgagactctcctgtgcagcctctggattcaccttcagtaggtatggcatgagctgggtccgccaggctcca
gggaagaggctggagtgggtctcaaccattagtggcggaggcacctacatatactacccagactcagtgaagggccgattc
accatctccagagacaacgccaagaacaccctgtatctgcaaatgaacagcctgagagccgaggacacggctgtgtattac
tgtaccagagataactatggccgcaactatgattatggcatggattattggggccagggcaccctggtgaccgtgagcagc
(amino acids)
(SEQ ID NO: 394)
EIVLTQSPATLSLSPGERATLTCSATSSVSYIHWYQQRPGQSPRLLIYSTSNLASGIPARFSGSGSGSDYTLTISSLEPE
DFAVYYCQQRSSSPFTFGSGTKVEIKGGGGSGGGGSGGGGSEVQLVESGGGLVKPGGSLRLSCAASGFTFSRYGMSWVRQ
APGKRLEWVSTISGGGTYIYYPDSVKGRFTISRDNAKNTLYLQMNSLRAEDTAVYYCTRDNYGRNYDYGMDYWGQGTLVT
VSS
HumanizeC2 scFV (VH-VL) sequence:
(DNA)
(SEQ ID NO: 395)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaact
(amino acids)
(SEQ ID NO: 396)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRT
HumanizeE6 scFV (VL-VH) sequence:
(DNA)
(SEQ ID NO: 397)
gacattgtgctgacccagtctccagcctccttggccgtgtctccaggacagagggccaccatcacctgcagagccagtaag
agtgtcagtaccagcggatactcctacatgcactggtatcagcagaaaccaggacaacctcctaaactcctgatttacctg
gcatccaatctggagagcggggtcccagccaggttcagcggcagtgggtctgggaccgatttcaccctcacaattaatcct
gtggaagctaatgatactgcaaattattactgtcagcacagtagggagctgcctttcacattcggcggagggaccaaggtg
gagatcaaacgaactggcggtggcggatccggcggtggcggatccggcggtggcggatccgaggtgcagctggtggagtct
gggggaggcctggtcaagcctggggggtccctgagactctcctgtgcagcctctggattcaccttcagtggctatgccatg
agctgggtccgccaggctccagggaaggggctggagtgggtctcaaccattagtagtggcggaacctacatatactacccc
gactcagtgaagggccgattcaccatctccagagacaacgccaagaactcactgtatctgcaaatgaacagcctgagagcc
gaggacacggccgtgtattactgtgcgagacttgggggggataattactacgaatacttcgatgtctggggcaaagggacc
acggtcaccgtctcctcc
(amino acids)
(SEQ ID NO: 398)
DIVLTQSPASLAVSPGQRATITCRASKSVSTSGYSYMHWYQQKPGQPPKLLIYLASNLESGVPARFSGSGSGTDFTLTIN
PVEANDTANYYCQHSRELPFTFGGGTKVEIKRTGGGGSGGGGSGGGGSEVQLVESGGGLVKPGGSLRLSCAASGFTFSGY
AMSWVRQAPGKGLEWVSTISSGGTYIYYPDSVKGRFTISRDNAKNSLYLQMNSLRAEDTAVYYCARLGGDNYYEYFDVWG
KGTTVTVSS
G₄S₁ linker sequence:
(DNA)
(SEQ ID NO: 399)
ggcggtggcggatcc
(amino acids)
(SEQ ID NO: 400)
GGGGS
[G₄S₁]x3 linker sequence:
(DNA)
(SEQ ID NO: 401)
ggcggtggcggatccggcggtggcggatccggcggtggcggatcc
(amino acids)
(SEQ ID NO: 402)
GGGGSGGGGSGGGGS
8 aa GS linker sequence:
(DNA)
(SEQ ID NO: 403)
ggcggttccggcggtggatccgga
(amino acids)
(SEQ ID NO: 404)
GGSGGGSG
12 aa GS linker sequence:
(DNA)
(SEQ ID NO: 405)
ggcggttccggcggtggatccggcggtggcggatccgga
(amino acids)
(SEQ ID NO: 406)
GGSGGGSGGGSG
13 aa GS linker sequence:
(DNA)
(SEQ ID NO: 407)
ggcggtggatccggcggtggcggatccggcggtggatcc
(amino acids)
(SEQ ID NO: 408)
GGGSGGGGSGGGS
22 aa GS linker sequence:
(DNA)
(SEQ ID NO: 409)
ggcggtggaagcggcggtggcggatccggcagcggcggaagcggcggtggcggatccggcggtgga
(amino acids)
(SEQ ID NO: 4110)
GGGSGGGGSGSGGSGGGGSGGG
24 aa GS linker sequence:
(DNA)
(SEQ ID NO: 411)
ggcggttccggcggtggatccggcggtggcggatccggaggcggttccggcggtggatccggcggtggcggatccgga
(amino acids)
(SEQ ID NO: 412)
GGSGGGSGGGSGGGSGGGSGGGSG
Mouse C3 Heavy chain variable region sequence:
(DNA)
(SEQ ID NO: 413)
caggtccagctgcagcagtctgggcctgagctggtgaggcctggggtctcagtgaagatttcctgcaagggttccggctac
agattcactgattatgctatgaactgggtgaagcagagtcatgcaaagagtctagagtggattggagttattagtactttc
tctggtaatacaaacttcaaccagaagtttaagggcaaggccacaatgactgtagacaaatcctccagcacagcctatatg
gaacttgccagattgacatctgaggattctgccatgtattactgtgcaagatcggattactacggcccatactttgactac
tggggccaaggcaccactctcacagtctcctca
(amino acids)
(SEQ ID NO: 414)
QVQLQQSGPELVRPGVSVKISCKGSGYRFTDYAMNWVKQSHAKSLEWIGVISTFSGNTNFNQKFKGKATMTVDKSSSTAYM
ELARLTSEDSAMYYCARSDYYGPYFDYWGQGTTLTVSS
Mouse C3 heavy chain variable framework region 1 (FWR1) sequence:
(DNA)
(SEQ ID NO: 415)
caggtccagctgcagcagtctgggcctgagctggtgaggcctggggtctcagtgaagatttcctgcaagggttccggcta
cagattcact
(amino acids)
(SEQ ID NO: 416)
QVQLQQSGPELVRPGVSVKISCKGSGYRFT
Mouse C3 heavy chain variable complementarity determining regions 1 (CDR1)
sequence:
(DNA)
(SEQ ID NO: 417)
gattatgctatgaac
(amino acids)
(SEQ ID NO: 418)
DYAMN
Mouse C3 heavy chain variable framework region 2 (FWR2) sequence:
(DNA)
(SEQ ID NO: 419)
tgggtgaagcagagtcatgcaaagagtctagagtggattgga
(amino acids)
(SEQ ID NO: 420)
WVKQSHAKSLEWIG
Mouse C3 heavy chain variable complementarity determining regions 2 (CDR2)
sequence:
(DNA)
(SEQ ID NO: 421)
gttattagtactttctctggtaatacaaacttcaaccagaagtttaagggc
(amino acids)
(SEQ ID NO: 422)
VISTFSGNTNFNQKFKG
Mouse C3 heavy chain variable framework region 3 (FWR3) acid sequence:
(DNA)
(SEQ ID NO: 423)
aaggccacaatgactgtagacaaatcctccagcacagcctatatggaacttgccagattgacatctgaggattctgccat
gtattactgtgcaaga
(amino acids)
(SEQ ID NO: 424)
KATMTVDKSSSTAYMELARLTSEDSAMYYCAR
Mouse C3 heavy chain variable complementarity determining regions 3 (CDR3)
sequence:
(DNA)
(SEQ ID NO: 425)
tcggattactacggcccatactttgactac
(amino acids)
(SEQ ID NO: 426)
SDYYGPYFDY
IGHV1-18*04 heavy chain variable region sequence:
(DNA)
(SEQ ID NO: 427)
caggttcagctggtgcagtctggagctgaggtgaagaagcctggggcctcagtgaaggtctcctgcaaggcttctggttac
acctttaccagctacggtatcagctgggtgcgacaggcccctggacaagggcttgagtggatgggatggatcagcgcttac
aatggtaacacaaactatgcacagaagctccagggcagagtcaccatgaccacagacacatccacgagcacagcctacatg
gagctgaggagcctgagatctgacgacacggccgtgtattactgtgcgagaga
(amino acids)
(SEQ ID NO: 428)
QVQLVQSGAEVKKPGASVKVSCKASGYTFTSYGISWVRQAPGQGLEWMGWISAYNGNTNYAQKLQGRVTMTTDTSTSTAY
MELRSLRSDDTAVYYCAR
IGHV1-18*04 heavy chain variable framework region 1 (FWR1) sequence:
(DNA)
(SEQ ID NO: 429)
caggttcagctggtgcagtctggagctgaggtgaagaagcctggggcctcagtgaaggtctcctgcaaggcttctggtta
cacctttacc
(amino acids)
(SEQ ID NO: 430)
QVQLVQSGAEVKKPGASVKVSCKASGYTFT
IGHV1-18*04 heavy chain variable complementarity determining regions 1 (CDR1)
sequence:
(DNA)
(SEQ ID NO: 431)
agctacggtatcagc
(amino acids)
(SEQ ID NO: 432)
SYGIS
IGHV1-18*04 heavy chain variable framework region 2 (FWR2) sequence:
(DNA)
(SEQ ID NO: 433)
tgggtgcgacaggcccctggacaagggcttgagtggatggga
(amino acids)
(SEQ ID NO: 434)
WVRQAPGQGLEWMG
IGHV1-18*04 heavy chain variable complementarity determining regions 2 (CDR2)
sequence:
(DNA)
(SEQ ID NO: 435)
tggatcagcgcttacaatggtaacacaaactatgcacagaagctccagggc
(amino acids)
(SEQ ID NO: 436)
WISAYNGNTNYAQKLQG
IGHV1-18*04 heavy chain variable framework region 3 (FWR3) sequence:
(DNA)
(SEQ ID NO: 437)
agagtcaccatgaccacagacacatccacgagcacagcctacatggagctgaggagcctgagatctgacgacacggccgt
gtattactgtgcgaga
(amino acids)
(SEQ ID NO: 438)
RVTMTTDTSTSTAYMELRSLRSDDTAVYYCAR
Humanized C3 heavy chain variable region sequence:
(DNA)
(SEQ ID NO: 439)
caggttcagctggtgcagtctggagctgaggtgaagaagcctggggcctcagtgaaggtctcctgcaaggcttctggtta
cacctttaccgactacgccatgaactgggtgcgacaggcccctggacaagggcttgagtggatgggagtgatcagcacct
tcagcggtaacacaaacttcaaccagaagttcaagggcagagtcaccatgaccacagacacatccacgagcacagcctac
atggagctgaggagcctgagatctgacgacacggccgtgtattactgtgcgagaagcgactactacggcccatacttcga
ctactggggccagggcaccaccctgaccgtgtccagc
(amino acids)
(SEQ ID NO: 440)
QVQLVQSGAEVKKPGASVKVSCKASGYTFTDYAMNWVRQAPGQGLEWMGVISTFSGNTNFNQKFKGRVTMTTDTSTSTAY
MELRSLRSDDTAVYYCARSDYYGPYFDYWGQGTTLTVSS
Humanized C3 heavy chain variable framework region 1 (FWR1) acid sequence:
(DNA)
(SEQ ID NO: 441)
caggttcagctggtgcagtctggagctgaggtgaagaagcctggggcctcagtgaaggtctcctgcaaggcttctggtta
cacctttacc
(amino acids)
(SEQ ID NO: 442)
QVQLVQSGAEVKKPGASVKVSCKASGYTFT
Humanized C3 heavy chain variable complementarity determining regions 1 (CDR1)
sequence:
(DNA)
(SEQ ID NO: 443)
gactacgccatgaac
(amino acids)
(SEQ ID NO: 444)
DYAMN
Humanized C3 heavy chain variable framework region 2 (FWR2) acid sequence:
(DNA)
(SEQ ID NO: 445)
tgggtgcgacaggcccctggacaagggcttgagtggatggga
(amino acids)
(SEQ ID NO: 446)
WVRQAPGQGLEWMG
Humanized C3 heavy chain variable complementarity determining regions 2 (CDR2)
sequence:
(DNA)
(SEQ ID NO: 447)
gtgatcagcaccttcagcggtaacacaaacttcaaccagaagttcaagggc
(amino acids)
(SEQ ID NO: 448)
VISTFSGNTNFNQKFKG
Humanized C3 heavy chain variable framework region 3 (FWR3) acid sequence:
(DNA)
(SEQ ID NO: 449)
agagtcaccatgaccacagacacatccacgagcacagcctacatggagctgaggagcctgagatctgacgacacggccgtg
tattactgtgcgaga
(amino acids)
(SEQ ID NO: 450)
RVTMTTDTSTSTAYMELRSLRSDDTAVYYCAR
Humanized C3 heavy chain variable complementarity determining regions 3 (CDR3)
sequence:
(DNA)
(SEQ ID NO: 451)
agcgactactacggcccatacttcgactac
(amino acids)
(SEQ ID NO: 452)
SDYYGPYFDY
Humanized C3 IgG1 heavy chain sequence
(DNA)
(SEQ ID NO: 453)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcgctagcaccaagggcccatcggtcttccccctggcaccctcctccaag
agcacctctgggggcacagcggccctgggctgcctggtcaaggactacttccccgaaccggtgacggtgtcgtggaactca
ggcgccctgaccagcggcgtgcacaccttcccggctgtcctacagtcctcaggactctactccctcagcagcgtggtgaca
gtgccctccagcagcttgggcacccagacctacatctgcaacgtgaatcacaagcccagcaacaccaaggtggacaagaaa
gttgagcccaaatcttgtgacaaaactcacacatgcccaccgtgcccagcacctgaactcctggggggaccgtcagtcttc
ctcttccccccaaaacccaaggacaccctcatgatctcccggacccctgaggtcacatgcgtggtggtggacgtgagccac
gaagaccctgaggtcaagttcaactggtacgtggacggcgtggaggtgcataatgccaagacaaagccgcgggaggagcag
tacaacagcacgtaccgtgtggtcagcgtcctcaccgtcctgcaccaggactggctgaatggcaaggagtacaagtgcaag
gtctccaacaaagccctcccagcccccatcgagaaaaccatctccaaagccaaagggcagccccgagaaccacaggtgtac
accctgcccccatcccgggaggagatgaccaagaaccaggtcagcctgacctgcctggtcaaaggcttctatcccagcgac
atcgccgtggagtgggagagcaatgggcagccggagaacaactacaagaccacgcctcccgtgctggactccgacggctcc
ttcttcctctacagcaagctcaccgtggacaagagcaggtggcagcaggggaacgtcttctcatgctccgtgatgcatgag
gctctgcacaaccactacacgcagaagagcctctccctgtctccgggtaaatgataa
(amino acids)
(SEQ ID NO: 454)
QVQLVQSGAEVKKPGASVKVSCKASGYTFTDYAMNWVRQAPGQGLEWMGVISTFSGNTNFNQKFKGRVTMTTDTSTSTAY
MELRSLRSDDTAVYYCARSDYYGPYFDYWGQGTTLTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK**
Humanized C3 IgG2 heavy chain sequence
(DNA)
(SEQ ID NO: 455)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcgcctccaccaagggcccatcggtcttccccctggcgccctgctccagg
agcacctccgagagcacagccgccctgggctgcctggtcaaggactacttccccgaaccggtgacggtgtcgtggaactca
ggcgctctgaccagcggcgtgcacaccttcccagctgtcctacagtcctcaggactctactccctcagcagcgtggtgacc
gtgccctccagcaacttcggcacccagacctacacctgcaacgtagatcacaagcccagcaacaccaaggtggacaagaca
gttgagcgcaaatgttgtgtcgagtgcccaccgtgcccagcaccacctgtggcaggaccgtcagtcttcctcttcccccca
aaacccaaggacaccctcatgatctcccggacccctgaggtcacgtgcgtggtggtggacgtgagccacgaagaccccgag
gtccagttcaactggtacgtggacggcgtggaggtgcataatgccaagacaaagccacgggaggagcagttcaacagcacg
ttccgtgtggtcagcgtcctcaccgttgtgcaccaggactggctgaacggcaaggagtacaagtgcaaggtctccaacaaa
ggcctcccagcccccatcgagaaaaccatctccaaaaccaaagggcagccccgagaaccacaggtgtacaccctgccccca
tcccgggaggagatgaccaagaaccaggtcagcctgacctgcctggtcaaaggcttctaccccagcgacatcgccgtggag
tgggagagcaatgggcagccggagaacaactacaagaccacacctcccatgctggactccgacggctccttcttcctctac
agcaagctcaccgtggacaagagcaggtggcagcaggggaacgtcttctcatgctccgtgatgcatgaggctctgcacaac
cactacacgcagaagagcctctccctgtctccgggtaaatagtaa
(amino acids)
(SEQ ID NO: 456)
QVQLVQSGAEVKKPGASVKVSCKASGYTFTDYAMNWVRQAPGQGLEWMGVISTFSGNTNFNQKFKGRVTMTTDTSTSTAY
MELRSLRSDDTAVYYCARSDYYGPYFDYWGQGTTLTVSSASTKGPSVFPLAPCSRSTSESTAALGCLVKDYFPEPVTVSW
NSGALTSGVHTFPAVLQSSGLYSLSSVVTVPSSNFGTQTYTCNVDHKPSNTKVDKTVERKCCVECPPCPAPPVAGPSVFL
FPPKPKDTLMISRTPEVTCVVVDVSHEDPEVQFNWYVDGVEVHNAKTKPREEQFNSTFRVVSVLTVVHQDWLNGKEYKCK
VSNKGLPAPIEKTISKTKGQPREPQVYTLPPSREEMTKNQVSLTCLVKGFYPSDIAVEWESNGQPENNYKTTPPMLDSDG
SFFLYSKLTVDKSRWQQGNVFSCSVMHEALHNHYTQKSLSLSPGK**
Mouse C3 Light Chain variable region sequence:
(DNA)
(SEQ ID NO: 458)
gatgttttgatgacccaaactccactctccctgcctgtcagtcttggagatcaagcctccatctcttgcagatctagtcag
accattgtacatagtaatggaaacacctatttagaatggtacctgcagaaaccaggccagtctccaaagctcctgatctac
aaagtttccaaccgattttctggggtcccagacaggttcagtggcagtggatcagggacagatttcacactcaagatcaac
agagtggaggctgaggatctgggagtttattactgctttcaaggttcacatgttccattcacgttcggctcggggacaaag
ttggaaataaaa
(amino acids)
(SEQ ID NO: 459)
DVLMTQTPLSLPVSLGDQASISCRSSQTIVHSNGNTYLEWYLQKPGQSPKLLIYKVSNRFSGVPDRFSGSGSGTDFTLKIN
RVEAEDLGVYYCFQGSHVPFTFGSGTKLEIK
Mouse C3 light chain variable framework region 1 (FWR1) sequence:
(DNA)
(SEQ ID NO: 460)
gatgttttgatgacccaaactccactctccctgcctgtcagtcttggagatcaagcctccatctcttgc
(amino acids)
(SEQ ID NO: 461)
DVLMTQTPLSLPVSLGDQASISC
Mouse C3 light chain variable complementarity determining regions 1 (CDR1)
sequence:
(DNA)
(SEQ ID NO: 462)
agatctagtcagaccattgtacatagtaatggaaacacctatttagaa
(amino acids)
(SEQ ID NO: 463)
RSSQTIVHSNGNTYLE
Mouse C3 light chain variable framework region 2 (FWR2) sequence:
(DNA)
(SEQ ID NO: 464)
tggtacctgcagaaaccaggccagtctccaaagctcctgatctac
(amino acids)
(SEQ ID NO: 465)
WYLQKPGQSPKLLIY
Mouse C3 light chain variable complementarity determining regions 2 (CDR2)
sequence:
(DNA)
(SEQ ID NO: 466)
aaagtttccaaccgattttct
(amino acids)
(SEQ ID NO: 467)
KVSNRFS
Mouse C3 light chain variable framework region 3 (FWR3) sequence:
(DNA)
(SEQ ID NO: 468)
ggggtcccagacaggttcagtggcagtggatcagggacagatttcacactcaagatcaacagagtggaggctgaggatct
gggagtttattactgc
(amino acids)
(SEQ ID NO: 469)
GVPDRFSGSGSGTDFTLKINRVEAEDLGVYYC
Mouse C3 light chain variable complementarity determining regions 3 (CDR3)
sequence:
(DNA)
(SEQ ID NO: 470)
tttcaaggttcacatgttccattcacg
(amino acids)
(SEQ ID NO: 471)
FQGSHVPFT
IGKV2-29*03 light chain variable region sequence:
(DNA)
(SEQ ID NO: 472)
gatattgtgatgacccagactccactctctctgtccgtcacccctggacagccggcctccatctcctgcaagtctagtca
gagcctcctgcatagtgatggaaagacctatttgtattggtacctgcagaagccaggccagtctccacagctcctgatct
atgaagtttccagccggttctctggagtgccagataggttcagtggcagcgggtcagggacagatttcacactgaaaatc
agccgggtggaggctgaggatgttggggtttattactgcatgcaaggtatacaccttcct
(amino acids)
(SEQ ID NO: 473)
DIVMTQTPLSLSVTPGQPASISCKSSQSLLHSDGKTYLYWYLQKPGQSPQLLIYEVSSRFSGVPDRFSGSGSGTDFTLKI
SRVEAEDVGVYYCMQGIHLP
IGKV2-29*03 light chain variable framework region 1 (FWR1) acid sequence:
(DNA)
(SEQ ID NO: 474)
gatattgtgatgacccagactccactctctctgtccgtcacccctggacagccggcctccatctcctgc
(amino acids)
(SEQ ID NO: 475)
DIVMTQTPLSLSVTPGQPASISC
IGKV2-29*03 light chain variable complementarity determining regions 1 (CDR1)
sequence:
(DNA)
(SEQ ID NO: 476)
aagtctagtcagagcctcctgcatagtgatggaaagacctatttgtat
(amino acids)
(SEQ ID NO: 477)
KSSQSLLHSDGKTYLY
IGKV2-29*03 light chain variable framework region 2 (FWR2) sequence:
(DNA)
(SEQ ID NO: 478)
tggtacctgcagaagccaggccagtctccacagctcctgatctat
(amino acids)
(SEQ ID NO: 479)
WYLQKPGQSPQLLIY
IGKV2-29*03 light chain variable complementarity determining regions 2 (CDR2)
sequence:
(DNA)
(SEQ ID NO: 480)
gaagtttccagccggttc
(amino acids)
(SEQ ID NO: 481)
EVSSRFS
IGKV2-29*03 light chain variable framework region 3 (FWR3) sequence:
(DNA)
(SEQ ID NO: 482)
ggagtgccagataggttcagtggcagcgggtcagggacagatttcacactgaaaatcagccgggtggaggctgaggatgt
tggggtttattactgc
(amino acids)
(SEQ ID NO: 483)
GVPDRFSGSGSGTDFTLKISRVEAEDVGVYYC
IGKV2-29*03 light chain variable complementarity determining regions3 (CDR3)
sequence:
(DNA)
(SEQ ID NO: 484)
atgcaaggtatacaccttcct
(amino acids)
(SEQ ID NO: 485)
MQGIHLP
Humanized C3 light chain variable region sequence:
(DNA)
(SEQ ID NO: 486)
gatattgtgatgacccagactccactctctctgtccgtcacccctggacagccggcctccatctcctgcaggtctagtcag
accattgtccatagtaatggaaacacctatttggagtggtacctgcagaagccaggccagtctccacagctcctgatctat
aaggtttccaaccggttctctggagtgccagataggttcagtggcagcgggtcagggacagatttcacactgaaaatcagc
cgggtggaggctgaggatgttggggtttattactgcttccaaggtagccacgtgcctttcaccttcggcggagggaccaag
gtggagatcaaacgaact
(amino acids)
(SEQ ID NO: 487)
DIVMTQTPLSLSVTPGQPASISCRSSQTIVHSNGNTYLEWYLQKPGQSPQLLIYKVSNRFSGVPDRFSGSGSGTDFTLKI
SRVEAEDVGVYYCFQGSHVPFTFGGGTKVEIKRT
Humanized C3 light chain variable framework region 1 (FWR1) acid sequence:
(DNA)
(SEQ ID NO: 488)
gatattgtgatgacccagactccactctctctgtccgtcacccctggacagccggcctccatctcctgc
(amino acids)
(SEQ ID NO: 489)
DIVMTQTPLSLSVTPGQPASISC
Humanized C3 light chain variable complementarity determining regions 1 (CDR1)
sequence:
(DNA)
(SEQ ID NO: 490)
ggtctagtcagaccattgtccatagtaatggaaacacctatttggag
(amino acids)
(SEQ ID NO: 491)
RSSQTIVHSNGNTYLE
Humanized C3 light chain variable framework region 2 (FWR2) acid sequence:
(DNA)
(SEQ ID NO: 492)
tggtacctgcagaagccaggccagtctccacagctcctgatctat
(amino acids)
(SEQ ID NO: 493)
WYLQKPGQSPQLLIY
Humanized C3 light chain variable complementarity determining regions 2 (CDR2)
sequence:
(DNA)
(SEQ ID NO: 494)
aaggtttccaaccggttctct
(amino acids)
(SEQ ID NO: 495)
KVSNRFS
Humanized C3 light chain variable framework region 3 (FWR3) acid sequence:
(DNA)
(SEQ ID NO: 496)
ggagtgccagataggttcagtggcagcgggtcagggacagatttcacactgaaaatcagccgggtggaggctgaggatgtt
ggggtttattactgc
(amino acids)
(SEQ ID NO: 497)
GVPDRFSGSGSGTDFTLKISRVEAEDVGVYYC
Humanized C3 light chain variable complementarity determining regions 3 (CDR3)
sequence:
(DNA)
(SEQ ID NO: 498)
ttccaaggtagccacgtgcctttcacc
(amino acids)
(SEQ ID NO: 499)
FQGSHVPFT
Humanized C3 lambda light chain sequence
(DNA)
(SEQ ID NO: 500)
gatattgtgatgacccagactccactctctctgtccgtcacccctggacagccggcctccatctcctgcaggtctagtcag
accattgtccatagtaatggaaacacctatttggagtggtacctgcagaagccaggccagtctccacagctcctgatctat
aaggtttccaaccggttctctggagtgccagataggttcagtggcagcgggtcagggacagatttcacactgaaaatcagc
cgggtggaggctgaggatgttggggtttattactgcttccaaggtagccacgtgcctttcaccttcggcggagggaccaag
gtggagatcaaacgaactggtcagcccaaggctgccccctcggtcactctgttcccgccctcctctgaggagcttcaagcc
aacaaggccacactggtgtgtctcataagtgacttctacccgggagccgtgacagtggcctggaaggcagatagcagcccc
gtcaaggcgggagtggagaccaccacaccctccaaacaaagcaacaacaagtacgcggccagcagctatctgagcctgacg
cctgagcagtggaagtcccacagaagctacagctgccaggtcacgcatgaagggagcaccgtggagaagacagtggcccct
acagaatgttcatagtaa
(amino acids)
(SEQ ID NO: 501)
DIVMTQTPLSLSVTPGQPASISCRSSQTIVHSNGNTYLEWYLQKPGQSPQLLIYKVSNRFSGVPDRFSGSGSGTDFTLKI
SRVEAEDVGVYYCFQGSHVPFTFGGGTKVEIKRTGQPKAAPSVTLFPPSSEELQANKATLVCLISDFYPGAVTVAWKADS
SPVKAGVETTTPSKQSNNKYAASSYLSLTPEQWKSHRSYSCQVTHEGSTVEKTVAPTECS**
Humanized C3 Kappa light chain
(DNA)
(SEQ ID NO: 502)
gatattgtgatgacccagactccactctctctgtccgtcacccctggacagccggcctccatctcctgcaggtctagtcag
accattgtccatagtaatggaaacacctatttggagtggtacctgcagaagccaggccagtctccacagctcctgatctat
aaggtttccaaccggttctctggagtgccagataggttcagtggcagcgggtcagggacagatttcacactgaaaatcagc
cgggtggaggctgaggatgttggggtttattactgcttccaaggtagccacgtgcctttcaccttcggcggagggaccaag
gtggagatcaaacgaactacggtggctgcaccatctgtcttcatcttcccgccatctgatgagcagttgaaatctggaact
gcctctgttgtgtgcctgctgaataacttctatcccagagaggccaaagtacagtggaaggtggataacgccctccaatcg
ggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaac
aggggagagtgttagtaa
(amino acids)
(SEQ ID NO: 503)
DIVMTQTPLSLSVTPGQPASISCRSSQTIVHSNGNTYLEWYLQKPGQSPQLLIYKVSNRFSGVPDRFSGSGSGTDFTLKI
SRVEAEDVGVYYCFQGSHVPFTFGGGTKVEIKRTTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC**
Mouse C8 heavy chain variable region sequence
(DNA)
(SEQ ID NO: 505)
gaagtgatggtcgtggaaagcggcggtggtctggtaaagccggggggatcccttaagctttcttgcgccgcatccgggttc
acgttctccggctatgccatgtcctgggtccgacagactcccgaaaagcgcttggaatgggtggccactatctcctccggg
gggacgtacatctactaccccgacagtgtgaaaggaagatttacaatatctcgcgacaacgcaaaaaataccttgtatctt
caaatgagctccctgcggtcagaggacactgccatgtactattgcgcccgcctgggcggcgacaattactatgagtat
(amino acids)
(SEQ ID NO: 506)
EVMVVESGGGLVKPGGSLKLSCAASGFTFSGYAMSWVRQTPEKRLEWVATISSGGTYIYYPDSVKGRFTISRDNAKNTLYL
QMSSLRSEDTAMYYCARLGGDNYYEY
Mouse C8 heavy chain variable complementarity determining region 1 (CDR1)
sequence:
(DNA)
(SEQ ID NO: 507)
ggctatgccatgtcc
(amino acids)
(SEQ ID NO: 508)
GYAMS
Mouse C8 heavy chain variable complementarity determining region 2 (CDR2)
sequence:
(DNA)
(SEQ ID NO: 509)
actatctcctccggggggacgtacatctactaccccgacagtgtgaaagga
(amino acids)
(SEQ ID NO: 510)
TISSGGTYIYYPDSVKG
Mouse C8 heavy chain variable complementarity determining region 3 (CDR3)
sequence:
(DNA)
(SEQ ID NO: 511)
ctgggcggcgacaattactatgagtat
(amino acids)
(SEQ ID NO: 512)
LGGDNYYEY
IGHV3-21*04 heavy chain variable region sequence:
(DNA)
(SEQ ID NO: 513)
gaggtgcagctggtggagtctgggggaggcctggtcaagcctggggggtccctgagactctcctgtgcagcctctggatt
caccttcagtagctatagcatgaactgggtccgccaggctccagggaaggggctggagtgggtctcatccattagtagta
gtagtagttacatatactacgcagactcagtgaagggccgattcaccatctccagagacaacgccaagaactcactgtat
ctgcaaatgaacagcctgagagccgaggacacggccgtgtattactgtgcga
(amino acids)
(SEQ ID NO: 514)
EVQLVESGGGLVKPGGSLRLSCAASGFTFSSYSMNWVRQAPGKGLEWVSSISSSSSYIYYADSVKGRFTISRDNAKNSLY
LQMNSLRAEDTAVYYCAR
IGHV3-21*04 heavy chain variable framework region 1 (FWR1) sequence:
(DNA)
(SEQ ID NO: 515)
gaggtgcagctggtggagtctgggggaggcctggtcaagcctggggggtccctgagactctcctgtgcagcctctggatt
caccttcagt
(amino acids)
(SEQ ID NO: 516)
EVQLVESGGGLVKPGGSLRLSCAASGFTFS
IGHV3-21*04 heavy chain variable complementarity determining regions 1 (CDR1)
sequence:
(DNA)
(SEQ ID NO: 517)
agctatagcatgaac
(amino acids)
(SEQ ID NO: 518)
SYSMN
IGHV3-21*04 heavy chain variable framework region 2 (FWR2) sequence:
(DNA)
(SEQ ID NO: 519)
tgggtccgccaggctccagggaaggggctggagtgggtc
(amino acids)
(SEQ ID NO: 520)
WVRQAPGKGLEWV
IGHV3-21*04 heavy chain variable complementarity determining regions 2 (CDR2)
sequence:
(DNA)
(SEQ ID NO: 521)
tcatccattagtagtagtagtagttacatatactacgcagactcagtgaagggc
(amino acids)
(SEQ ID NO: 522)
SSISSSSSYIYYADSVKG
IGHV3-21*04 heavy chain variable framework region 3 (FWR3) sequence:
(DNA)
(SEQ ID NO: 523)
cgattcaccatctccagagacaacgccaagaactcactgtatctgcaaatgaacagcctgagagccgaggacacggccgt
gtattactgtgcga
(amino acids)
(SEQ ID NO: 524)
RFTISRDNAKNSLYLQMNSLRAEDTAVYYCAR
Humanized C8 heavy chain variable region sequence:
(DNA)
(SEQ ID NO: 525)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctcc
(amino acids)
(SEQ ID NO: 526)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYWGKGTTVTVSS
Humanized C8 heavy chain variable framework region 1 (FWR1) sequence:
(DNA)
(SEQ ID NO: 527)
gaggtgcagctggtggagtctgggggaggcctggtcaagcctggggggtccctgagactctcctgtgcagcctctggattc
accttcagt
(amino acids)
(SEQ ID NO: 528)
EVQLVESGGGLVKPGGSLRLSCAASGFTFS
Humanized C8 heavy chain variable complementarity determining region 1 (CDR1)
sequence:
(DNA)
(SEQ ID NO: 529)
ggctatgccatgagc
(amino acids)
(SEQ ID NO: 530)
GYAMS
Humanized C8 heavy chain variable framework region 2 (FWR2) sequence:
(DNA)
(SEQ ID NO: 531)
tgggtccgccaggctccagggaaggggctggagtgggtctca
(amino acids)
(SEQ ID NO: 532)
WVRQAPGKGLEWVS
Humanized C8 heavy chain variable complementarity determining region 2 (CDR2)
sequence:
(DNA)
(SEQ ID NO: 533)
accattagtagtggcggaacctacatatactaccctgactcagtgaagggc
(amino acids)
(SEQ ID NO: 534)
TISSGGTYIYYPDSVKG
Humanized C8 heavy chain variable framework region 3 (FWR3) sequence:
(DNA)
(SEQ ID NO: 535)
cgattcaccatctccagagacaacgccaagaactcactgtatctgcaaatgaacagcctgagagccgaggacacggccgtg
tattactgtgcgaga
(amino acids)
(SEQ ID NO: 536)
RFTISRDNAKNSLYLQMNSLRAEDTAVYYCAR
Humanized C8 heavy chain variable complementarity determining region 3 (CDR3)
sequence:
(DNA)
(SEQ ID NO: 537)
ctgggcggcgataactattatgaatat
(amino acids)
(SEQ ID NO: 538)
LGGDNYYEY
Humanized C8 IgG1 heavy chain sequence
(DNA)
(SEQ ID NO: 539)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctccgctagcaccaagggcccatcggtcttccccctggcaccctcctccaagagc
acctctgggggcacagcggccctgggctgcctggtcaaggactacttccccgaaccggtgacggtgtcgtggaactcaggc
gccctgaccagcggcgtgcacaccttcccggctgtcctacagtcctcaggactctactccctcagcagcgtggtgacagtg
ccctccagcagcttgggcacccagacctacatctgcaacgtgaatcacaagcccagcaacaccaaggtggacaagaaagtt
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaatgataa
(amino acids)
(SEQ ID NO: 540)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYWGKGTTVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK**
Humanized C8 IgG2 heavy chain sequence
(DNA)
(SEQ ID NO: 541)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctccgcctccaccaagggcccatcggtcttccccctggcgccctgctccaggagc
acctccgagagcacagccgccctgggctgcctggtcaaggactacttccccgaaccggtgacggtgtcgtggaactcaggc
gctctgaccagcggcgtgcacaccttcccagctgtcctacagtcctcaggactctactccctcagcagcgtggtgaccgtg
ccctccagcaacttcggcacccagacctacacctgcaacgtagatcacaagcccagcaacaccaaggtggacaagacagtt
gagcgcaaatgttgtgtcgagtgcccaccgtgcccagcaccacctgtggcaggaccgtcagtcttcctcttccccccaaaa
cccaaggacaccctcatgatctcccggacccctgaggtcacgtgcgtggtggtggacgtgagccacgaagaccccgaggtc
cagttcaactggtacgtggacggcgtggaggtgcataatgccaagacaaagccacgggaggagcagttcaacagcacgttc
cgtgtggtcagcgtcctcaccgttgtgcaccaggactggctgaacggcaaggagtacaagtgcaaggtctccaacaaaggc
ctcccagcccccatcgagaaaaccatctccaaaaccaaagggcagccccgagaaccacaggtgtacaccctgcccccatcc
cgggaggagatgaccaagaaccaggtcagcctgacctgcctggtcaaaggcttctaccccagcgacatcgccgtggagtgg
gagagcaatgggcagccggagaacaactacaagaccacacctcccatgctggactccgacggctccttcttcctctacagc
aagctcaccgtggacaagagcaggtggcagcaggggaacgtcttctcatgctccgtgatgcatgaggctctgcacaaccac
tacacgcagaagagcctctccctgtctccgggtaaatagtaa
(amino acids)
(SEQ ID NO: 542)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYWGKGTTVTVSSASTKGPSVFPLAPCSRSTSESTAALGCLVKDYFPEPVTVSWNSG
ALTSGVHTFPAVLQSSGLYSLSSVVTVPSSNFGTQTYTCNVDHKPSNTKVDKTVERKCCVECPPCPAPPVAGPSVFLFPPK
PKDTLMISRTPEVTCVVVDVSHEDPEVQFNWYVDGVEVHNAKTKPREEQFNSTFRVVSVLTVVHQDWLNGKEYKCKVSNKG
LPAPIEKTISKTKGQPREPQVYTLPPSREEMTKNQVSLTCLVKGFYPSDIAVEWESNGQPENNYKTTPPMLDSDGSFFLYS
KLTVDKSRWQQGNVFSCSVMHEALHNHYTQKSLSLSPGK**
Mouse C8 light chain variable region sequence
(DNA)
(SEQ ID NO: 543)
gacatcgtcattacgcagacccctgccagtcttgccgtttctctgggccagagggccactatcagttacagggcgagtaag
tctgtgagtaccagcggctatagttacatgcattggaaccagcagaaaccgggacagccaccacgcctgcttatttatctg
gtgtctaatcttgagtccggggtgcccgccaggttcagcggcagcggctctgggaccgacttcacactcaacattcatcca
gtggaagaagaggacgctgctacatactactgtcaacacattcgggaactgaccaggagtgaa
(amino acids)
(SEQ ID NO: 544)
DIVITQTPASLAVSLGQRATISYRASKSVSTSGYSYMHWNQQKPGQPPRLLIYLVSNLESGVPARFSGSGSGTDFTLNIHP
VEEEDAATYYCQHIRELTRSE
Mouse C8 light chain variable complementarity determining region 1 (CDR1)
sequence:
(DNA)
(SEQ ID NO: 545)
agggcgagtaagtctgtgagtaccagcggctatagttacatgcat
(amino acids)
(SEQ ID NO: 546)
RASKSVSTSGYSYMH
Mouse C8 light chain variable complementarity determining region 2 (CDR2)
sequence:
(DNA)
(SEQ ID NO: 547)
ctggtgtctaatcttgagtcc
(amino acids)
(SEQ ID NO: 548)
LVSNLES
Mouse C8 light chain variable complementarity determining region 3 (CDR3)
sequence:
(DNA)
(SEQ ID NO: 549)
caacacattcgggaactgaccaggagtgaa
(amino acids)
(SEQ ID NO: 550)
QHIRELTRSE
NCBI germline z00023 light chain variable region sequence:
(DNA)
(SEQ ID NO: 551)
gacatcgtgatgacccagtctccagactccctggctgtgtctctgggcgagagggccaccatcaactgcaagtccagccag
agtgttttatacagctccaacaataagaactacttagcttggtaccagcagaaaccaggacagcctcctaagctgctcatt
tactgggcatctacccgggaatccggggtccctgaccgattcagtggcagcgggtctgggacagatttcactctcaccatc
agcagcctgcaggctgaagatgtggcagtttattactgtcagcaatattatagtactcct
(amino acids)
(SEQ ID NO: 552)
DIVMTQSPDSLAVSLGERATINCKSSQSVLYSSNNKNYLAWYQQKPGQPPKLLIYWASTRESGVPDRFSGSGSGTDFTLT
ISSLQAEDVAVYYCQQYYSTP
NCBI germline z00023 light chain variable framework region 1 (FWR1) acid sequence:
(DNA)
(SEQ ID NO: 553)
gacatcgtgatgacccagtctccagactccctggctgtgtctctgggcgagagggccaccatcaactgc
(amino acids)
(SEQ ID NO: 554)
DIVMTQSPDSLAVSLGERATINC
NCBI germline z00023 light chain variable complementarity determining regions 1
(CDR1) sequence:
(DNA)
(SEQ ID NO: 555)
aagtccagccagagtgttttatacagctccaacaataagaactacttagct
(amino acids)
(SEQ ID NO: 556)
KSSQSVLYSSNNKNYLA
NCBI germline z00023 light chain variable framework region 2 (FWR2) sequence:
(DNA)
(SEQ ID NO: 557)
tggtaccagcagaaaccaggacagcctcctaagctgctcatttac
(amino acids)
(SEQ ID NO: 558)
WYQQKPGQPPKLLIY
NCBI germline z00023 light chain variable complementarity determining regions 2
(CDR2) sequence:
(DNA)
(SEQ ID NO: 559)
tgggcatctacccgggaatcc
(amino acids)
(SEQ ID NO: 560)
WASTRES
NCBI germline z00023 light chain variable framework region 3 (FWR3) sequence:
(DNA)
(SEQ ID NO: 561)
ggggtccctgaccgattcagtggcagcgggtctgggacagatttcactctcaccatcagcagcctgcaggctgaagatgt
ggcagtttattactgt
(amino acids)
(SEQ ID NO: 562)
GVPDRFSGSGSGTDFTLTISSLQAEDVAVYYC
NCBI germline z00023 light chain variable complementarity determining regions3
(CDR3) sequence:
(DNA)
(SEQ ID NO: 563)
cagcaatattatagtactcct
(amino acids)
(SEQ ID NO: 564)
QQYYSTP
Humanized C8 light chain variable region sequence
(DNA)
(SEQ ID NO: 565)
gacatcgtgatgacccagtctccagactccctggctgtgtctctgggcgagagggccaccatcaactgcagggccagcaag
agtgttagcaccagcggctacagctacatgcactggtaccagcagaaaccaggacagcctcctaagctgctcatttacctg
gtgtctaacctggaatccggggtccctgaccgattcagtggcagcgggtctgggacagatttcactctcaccatcagcagc
ctgcaggctgaagatgtggcagtttattactgtcaacacattcgggaactgaccaggagtgaattcggcggagggaccaag
gtggagatcaaacgaact
(amino acids)
(SEQ ID NO: 566)
DIVMTQSPDSLAVSLGERATINCRASKSVSTSGYSYMHWYQQKPGQPPKLLIYLVSNLESGVPDRFSGSGSGTDFTLTIS
SLQAEDVAVYYCQHIRELTRSEFGGGTKVEIKRT
Humanized C8 light chain variable framework region 1 (FWR1) sequence:
(DNA)
(SEQ ID NO: 567)
gacatcgtgatgacccagtctccagactccctggctgtgtctctgggcgagagggccaccatcaactgc
(amino acids)
(SEQ ID NO: 568)
DIVMTQSPDSLAVSLGERATINC
Humanized C8 light chain variable complementarity determining region 1 (CDR1)
sequence:
(DNA)
(SEQ ID NO: 569)
agggccagcaagagtgttagcaccagcggctacagctacatg
(amino acids)
(SEQ ID NO: 570)
RASKSVSTSGYSYM
Humanized C8 light chain variable framework region 2 (FWR2) sequence:
(DNA)
(SEQ ID NO: 571)
cactggtaccagcagaaaccaggacagcctcctaagctgctcatttac
(amino acids)
(SEQ ID NO: 572)
HWYQQKPGQPPKLLIY
Humanized C8 light chain variable complementarity determining region 2 (CDR2)
sequence:
(DNA)
(SEQ ID NO: 573)
ctggtgtctaacctggaatcc
(amino acids)
(SEQ ID NO: 574)
LVSNLES
Humanized C8 light chain variable framework region 3 (FWR3) sequence:
(DNA)
(SEQ ID NO: 575)
ggggtccctgaccgattcagtggcagcgggtctgggacagatttcactctcaccatcagcagcctgcaggctgaagatgtg
gcagtttattactgt
(amino acids)
(SEQ ID NO: 576)
GVPDRFSGSGSGTDFTLTISSLQAEDVAVYYC
Humanized C8 light chain variable complementarity determining region 3 (CDR3)
sequence:
(DNA)
(SEQ ID NO: 577)
caacacattcgggaactgaccaggagtgaa
(amino acids)
(SEQ ID NO: 578)
QHIRELTRSE
Humanized C8 Lambda light chain sequence
(DNA)
(SEQ ID NO: 579)
gacatcgtgatgacccagtctccagactccctggctgtgtctctgggcgagagggccaccatcaactgcagggccagcaag
agtgttagcaccagcggctacagctacatgcactggtaccagcagaaaccaggacagcctcctaagctgctcatttacctg
gtgtctaacctggaatccggggtccctgaccgattcagtggcagcgggtctgggacagatttcactctcaccatcagcagc
ctgcaggctgaagatgtggcagtttattactgtcaacacattcgggaactgaccaggagtgaattcggcggagggaccaag
gtggagatcaaacgaactggtcagcccaaggctgccccctcggtcactctgttcccgccctcctctgaggagcttcaagcc
aacaaggccacactggtgtgtctcataagtgacttctacccgggagccgtgacagtggcctggaaggcagatagcagcccc
gtcaaggcgggagtggagaccaccacaccctccaaacaaagcaacaacaagtacgcggccagcagctatctgagcctgacg
cctgagcagtggaagtcccacagaagctacagctgccaggtcacgcatgaagggagcaccgtggagaagacagtggcccct
acagaatgttcatagtaa
(amino acids)
(SEQ ID NO: 580)
DIVMTQSPDSLAVSLGERATINCRASKSVSTSGYSYMHWYQQKPGQPPKLLIYLVSNLESGVPDRFSGSGSGTDFTLTISS
LQAEDVAVYYCQHIRELTRSEFGGGTKVEIKRTGQPKAAPSVTLFPPSSEELQANKATLVCLISDFYPGAVTVAWKADSSP
VKAGVETTTPSKQSNNKYAASSYLSLTPEQWKSHRSYSCQVTHEGSTVEKTVAPTECS**
Humanized C8 Kappa light chain sequence
(DNA)
(SEQ ID NO: 581)
gacatcgtgatgacccagtctccagactccctggctgtgtctctgggcgagagggccaccatcaactgcagggccagcaag
agtgttagcaccagcggctacagctacatgcactggtaccagcagaaaccaggacagcctcctaagctgctcatttacctg
gtgtctaacctggaatccggggtccctgaccgattcagtggcagcgggtctgggacagatttcactctcaccatcagcagc
ctgcaggctgaagatgtggcagtttattactgtcaacacattcgggaactgaccaggagtgaattcggcggagggaccaag
gtggagatcaaacgaactacggtggctgcaccatctgtcttcatcttcccgccatctgatgagcagttgaaatctggaact
gcctctgttgtgtgcctgctgaataacttctatcccagagaggccaaagtacagtggaaggtggataacgccctccaatcg
ggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaac
aggggagagtgttagtaa
(amino acids)
(SEQ ID NO: 582)
DIVMTQSPDSLAVSLGERATINCRASKSVSTSGYSYMHWYQQKPGQPPKLLIYLVSNLESGVPDRFSGSGSGTDFTLTISS
LQAEDVAVYYCQHIRELTRSEFGGGTKVEIKRTTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC**
CAR-T E6 CD8 sequence:
(DNA)
(SEQ ID NO: 584)
gaggtccagctggttgagagtggcggtgggctggttaagcctggcggctccctgcggctgagctgcgccgcgagtggattt
actttcagccgatatgggatgagttgggtgcggcaagctcccgggaagaggctggaatgggtctcaacaatctccgggggg
ggcacttacatctattaccccgactcagtcaaggggagatttaccatttcacgagacaacgctaagaataccctgtatttg
cagatgaattctctgagagcagaggacacagctgtttactattgtacccgcgacaactatggcaggaactacgactacggt
atggactattggggacaagggacattggttacagtgagcagtggcggcgggggcagcggaggaggaggcagcggtgggggg
ggcagcgagatagtgctcacgcagtcacccgcgactctcagtctctcacctggggaacgagctaccctgacgtgctctgct
acctcctcagtgtcatatattcactggtatcagcaacggcccgggcagtcccctagattgctcatttatagtacctctaat
ctggcctcaggtatccctgcacgattttctggatctggttcaggttctgattacaccctcactatctctagcctggagcct
gaagactttgccgtttattactgccagcagaggtctagctccccattcacctttgggagtgggaccaaggttgaaattaaa
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgctgataa
(amino acids)
(SEQ ID NO: 585)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSGGGGSGGGGSGGGGSEIVLTQSPATLSLSPGERATLTC
SATSSVSYIHWYQQRPGQSPRLLIYSTSNLASGIPARFSGSGSGSDYTLTISSLEPEDFAVYYCQQRSSSPFTFGSGTKV
EIKTTTPAPRPPTPAPTIASQPLSLRPEACRPAAGGAVHTRGLDFACDIYIWAPLAGTCGVLLLSLVITLYC**
CAR-T C2 CD8 CD8 sequence:
N-CD81s-huMNC2scFv-CD8ecd fragment- CD8 transmembrane-C
(DNA)
(SEQ ID NO: 586)
gaagtgcagctcgtagagagtggcgggggactggtgaagcccggtggaagcctcagactcagttgcgccgcctcaggtttc
actttttcaggttacgccatgtcctgggtaagacaggcaccggggaaaggactcgagtgggtgtctactatcagctcagga
ggcacttatatatattatcctgactctgtaaaaggccgatttacgatttctcgcgacaatgcaaagaactccctctacctc
caaatgaacagtcttagggcagaagacactgctgtatactattgtgcacgcctcggcggcgacaactactacgagtacttt
gacgtgtgggggaaagggactaccgtgacagtttcaagcggaggaggtggctcaggtggaggcgggtcaggggggggagga
agtgatattgtgctcacacaatccccagcctccctggctgtgtctcccggccaacgcgctacaattacatgtcgggcctcc
aaaagcgtgagcaccagcggctacagctacatgcactggtatcaacagaaaccaggacaaccccccaaactgttgatttat
ctcgcttcaaacttggagtccggcgtgcctgcgcgcttttcagggagtgggagcggcacagattttacgctgactatcaac
cccgtagaagcaaacgatacagcgaattattattgtcaacattcccgggaactcccctttacgttcggcgggggcacaaag
gtcgaaattaagagaaccacgacaaccccggcccccagaccaccaacgccagcccccaccatcgccagccaacccctgtct
ctgagaccagaagcctgtaggcctgccgccggtggagctgtgcacacaagaggactggatttcgcctgtgatatctacatt
tgggccccgctcgcaggcacatgtggagtgctcctcctctccctggtgattaccctgtactgctgataa
(amino acids)
(SEQ ID NO: 587)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTTTTPAPRPPTPAPTIASQPLSLRPEACRPAAGGAVHTRGLDFACDIYIWAPLAGTCGVLLLSLVITLYC**
CD8/4-1BB sequence
N- CD8 transmembrane- 4-1BB-C
(DNA)
(SEQ ID NO: 588)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcaaaaggggccgcaaaaaactcctttacatttttaag
cagccttttatgaggccagtacagacgactcaagaggaagacgggtgctcatgccgctttcctgaggaggaggaaggaggg
tgcgaactgtgataa
(amino acids)
(SEQ ID NO: 589)
TTTPAPRPPTPAPTIASQPLSLRPEACRPAAGGAVHTRGLDFACDIYIWAPLAGTCGVLLLSLVITLYCKRGRKKLLYIF
KQPFMRPVQTTQEEDGCSCRFPEEEEGGCEL**
CD8/CD28 sequence
N- CD8 transmembrane- CD28-C
(DNA)
(SEQ ID NO: 590)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtcctgataa
(amino acids)
(SEQ ID NO: 591)
TTTPAPRPPTPAPTIASQPLSLRPEACRPAAGGAVHTRGLDFACDIYIWAPLAGTCGVLLLSLVITLYCRSKRSRLLHSD
YMNMTPRRPGPTRKHYQPYAPPRDFAAYRS**
CD8/CD3z sequence:
N- CD8 transmembrane- CD3zeta-C
(DNA)
(SEQ ID NO: 592)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgccgcgttaagttctcccgatcagccgacgcgcctgct
tacaagcagggccagaaccaactgtacaacgagctgaatctcggtagacgggaagagtacgacgtgttggacaaacggaga
ggccgcgacccagaaatgggcggcaagcctcgcaggaaaaacccccaggagggactgtacaatgagttgcagaaagataag
atggcagaagcttatagcgagatcggaatgaagggggaaaggagacgagggaaaggacacgacggcctttatcagggcctg
tccacagcaacaaaagatacgtatgacgccctccatatgcaggcacttccaccacggtgataa
(amino acids)
(SEQ ID NO: 593)
TTTPAPRPPTPAPTIASQPLSLRPEACRPAAGGAVHTRGLDFACDIYIWAPLAGTCGVLLLSLVITLYCRVKFSRSADAP
AYKQGQNQLYNELNLGRREEYDVLDKRRGRDPEMGGKPRRKNPQEGLYNELQKDKMAEAYSEIGMKGERRRGKGHDGLYQ
GLSTATKDTYDALHMQALPPR**
CD8/CD28/CD3z sequence:
N- CD8 transmembrane- CD28- CD3zeta-C
(DNA)
(SEQ ID NO: 594)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtcccgcgttaagttctcccgatcagccgacgcgcctgcttacaagcagggccagaaccaactgtacaacgagctgaat
ctcggtagacgggaagagtacgacgtgttggacaaacggagaggccgcgacccagaaatgggcggcaagcctcgcaggaaa
aacccccaggagggactgtacaatgagttgcagaaagataagatggcagaagcttatagcgagatcggaatgaagggggaa
aggagacgagggaaaggacacgacggcctttatcagggcctgtccacagcaacaaaagatacgtatgacgccctccatatg
caggcacttccaccacggtgataa
(amino acids)
(SEQ ID NO: 595)
TTTPAPRPPTPAPTIASQPLSLRPEACRPAAGGAVHTRGLDFACDIYIWAPLAGTCGVLLLSLVITLYCRSKRSRLLHSD
YMNMTPRRPGPTRKHYQPYAPPRDFAAYRSRVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDTYDALHMQALPPR**
CD8/4-1BB/CD3z sequence:
N- CD8 transmembrane- 4-1BB- CD3zeta-C
(DNA)
(SEQ ID NO: 596)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcaaaaggggccgcaaaaaactcctttacatttttaag
cagccttttatgaggccagtacagacgactcaagaggaagacgggtgctcatgccgctttcctgaggaggaggaaggaggg
tgcgaactgcgcgttaagttctcccgatcagccgacgcgcctgcttacaagcagggccagaaccaactgtacaacgagctg
aatctcggtagacgggaagagtacgacgtgttggacaaacggagaggccgcgacccagaaatgggcggcaagcctcgcagg
aaaaacccccaggagggactgtacaatgagttgcagaaagataagatggcagaagcttatagcgagatcggaatgaagggg
gaaaggagacgagggaaaggacacgacggcctttatcagggcctgtccacagcaacaaaagatacgtatgacgccctccat
atgcaggcacttccaccacggtgataa
(amino acids)
(SEQ ID NO: 597)
TTTPAPRPPTPAPTIASQPLSLRPEACRPAAGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR**
CD8/CD28/4-1BB/CD3z sequence:
N- CD8 transmembrane- CD28- 4-1BB- CD3zeta-C
(DNA)
(SEQ ID NO: 598)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtccaaaaggggccgcaaaaaactcctttacatttttaagcagccttttatgaggccagtacagacgactcaagaggaa
gacgggtgctcatgccgctttcctgaggaggaggaaggagggtgcgaactgcgcgttaagttctcccgatcagccgacgcg
cctgcttacaagcagggccagaaccaactgtacaacgagctgaatctcggtagacgggaagagtacgacgtgttggacaaa
cggagaggccgcgacccagaaatgggcggcaagcctcgcaggaaaaacccccaggagggactgtacaatgagttgcagaaa
gataagatggcagaagcttatagcgagatcggaatgaagggggaaaggagacgagggaaaggacacgacggcctttatcag
ggcctgtccacagcaacaaaagatacgtatgacgccctccatatgcaggcacttccaccacggtgataa
(amino acids)
(SEQ ID NO: 599)
TTTPAPRPPTPAPTIASQPLSLRPEACRPAAGGAVHTRGLDFACDIYIWAPLAGTCGVLLLSLVITLYCRSKRSRLLHSD
YMNMTPRRPGPTRKHYQPYAPPRDFAAYRSKRGRKKLLYIFKQPFMRPVQTTQEEDGCSCRFPEEEEGGCELRVKFSRSA
DAPAYKQGQNQLYNELNLGRREEYDVLDKRRGRDPEMGGKPRRKNPQEGLYNELQKDKMAEAYSEIGMKGERRRGKGHDG
LYQGLSTATKDTYDALHMQALPPR**
CAR-T C3 CD8/CD8/4-1BB/CD3z sequence:
N-CD81s-huMNC3scFv-CD8ecd fragment- CD8 transmembrane- 4-1BB- CD3zeta-C
(DNA)
(SEQ ID NO: 600)
atggccctgcccgtgaccgctttgctgctccccctggcgctgctgctgcacgccgccaggccacaggttcagctggtgcag
tctggagctgaggtgaagaagcctggggcctcagtgaaggtctcctgcaaggcttctggttacacctttaccgactacgcc
atgaactgggtgcgacaggcccctggacaagggcttgagtggatgggagtgatcagcaccttcagcggtaacacaaacttc
aaccagaagttcaagggcagagtcaccatgaccacagacacatccacgagcacagcctacatggagctgaggagcctgaga
tctgacgacacggccgtgtattactgtgcgagaagcgactactacggcccatacttcgactactggggccagggcaccacc
ctgaccgtgtccagcggcggtggcggatccggcggtggcggatccggcggtggcggatccgatattgtgatgacccagact
ccactctctctgtccgtcacccctggacagccggcctccatctcctgcaggtctagtcagaccattgtccatagtaatgga
aacacctatttggagtggtacctgcagaagccaggccagtctccacagctcctgatctataaggtttccaaccggttctct
ggagtgccagataggttcagtggcagcgggtcagggacagatttcacactgaaaatcagccgggtggaggctgaggatgtt
ggggtttattactgcttccaaggtagccacgtgcctttcaccttcggcggagggaccaaggtggagatcaaacgaactacg
acaaccccggcccccagaccaccaacgccagcccccaccatcgccagccaacccctgtctctgagaccagaagcctgtagg
cctgccgccggtggagctgtgcacacaagaggactggatttcgcctgtgatatctacatttgggccccgctcgcaggcaca
tgtggagtgctcctcctctccctggtgattaccctgtactgcaaaaggggccgcaaaaaactcctttacatttttaagcag
ccttttatgaggccagtacagacgactcaagaggaagacgggtgctcatgccgctttcctgaggaggaggaaggagggtgc
gaactgcgcgttaagttctcccgatcagccgacgcgcctgcttacaagcagggccagaaccaactgtacaacgagctgaat
ctcggtagacgggaagagtacgacgtgttggacaaacggagaggccgcgacccagaaatgggcggcaagcctcgcaggaaa
aacccccaggagggactgtacaatgagttgcagaaagataagatggcagaagcttatagcgagatcggaatgaagggggaa
aggagacgagggaaaggacacgacggcctttatcagggcctgtccacagcaacaaaagatacgtatgacgccctccatatg
caggcacttccaccacggtgataa
(amino acids)
(SEQ ID NO: 601)
QVQLVQSGAEVKKPGASVKVSCKASGYTFTDYAMNWVRQAPGQGLEWMGVISTFSGNTNFNQKFKGRVTMTTDTSTSTAY
MELRSLRSDDTAVYYCARSDYYGPYFDYWGQGTTLTVSSGGGGSGGGGSGGGGSDIVMTQTPLSLSVTPGQPASISCRSS
QTIVHSNGNTYLEWYLQKPGQSPQLLIYKVSNRFSGVPDRFSGSGSGTDFTLKISRVEAEDVGVYYCFQGSHVPFTFGGG
TKVEIKRTTTTPAPRPPTPAPTIASQPLSLRPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPR**
CAR-T C2 CD8/CD8/CD3z sequence:
N-CD81s-huMNC2scFv-CD8ecd fragment- CD8 transmembrane- CD3zeta-C
(DNA)
(SEQ ID NO: 606)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgccgcgttaagttctcccgatcagccgacgcgcctgct
tacaagcagggccagaaccaactgtacaacgagctgaatctcggtagacgggaagagtacgacgtgttggacaaacggaga
ggccgcgacccagaaatgggcggcaagcctcgcaggaaaaacccccaggagggactgtacaatgagttgcagaaagataag
atggcagaagcttatagcgagatcggaatgaagggggaaaggagacgagggaaaggacacgacggcctttatcagggcctg
tccacagcaacaaaagatacgtatgacgccctccatatgcaggcacttccaccacggtgataa
(amino acids)
(SEQ ID NO: 607)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCRVKFSRSADAPAYKQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR**
CAR-T C2 CD8/CD8/CD28/CD3z sequence:
N-CD81s-huMNC2scFv-CD8ecd fragment- CD8 transmembrane- CD28- CD3zeta-C
(DNA)
(SEQ ID NO: 608)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtcccgcgttaagttctcccgatcagccgacgcgcctgcttacaagcagggccagaaccaactgtacaacgagctgaat
ctcggtagacgggaagagtacgacgtgttggacaaacggagaggccgcgacccagaaatgggcggcaagcctcgcaggaaa
aacccccaggagggactgtacaatgagttgcagaaagataagatggcagaagcttatagcgagatcggaatgaagggggaa
aggagacgagggaaaggacacgacggcctttatcagggcctgtccacagcaacaaaagatacgtatgacgccctccatatg
caggcacttccaccacggtgataa
(amino acids)
(SEQ ID NO: 609)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCRSKRSRLLHSDYMNMTPRRPGPTRKHYQPYAPPRDFAAYRSRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T C2 CD8/CD8/4-1BB/CD3z sequence #13:
N-CD81s-huMNC2scFv-CD8ecd fragment- CD8 transmembrane- 4-1BB- CD3zeta-C
(DNA)
(SEQ ID NO: 610)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcaaaaggggccgcaaaaaactcctttacatttttaag
cagccttttatgaggccagtacagacgactcaagaggaagacgggtgctcatgccgctttcctgaggaggaggaaggaggg
tgcgaactgcgcgttaagttctcccgatcagccgacgcgcctgcttacaagcagggccagaaccaactgtacaacgagctg
aatctcggtagacgggaagagtacgacgtgttggacaaacggagaggccgcgacccagaaatgggcggcaagcctcgcagg
aaaaacccccaggagggactgtacaatgagttgcagaaagataagatggcagaagcttatagcgagatcggaatgaagggg
gaaaggagacgagggaaaggacacgacggcctttatcagggcctgtccacagcaacaaaagatacgtatgacgccctccat
atgcaggcacttccaccacggtgataa
(amino acids)
(SEQ ID NO: 611)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRRGKGHDGLYQGLSTATKD
TYDALHMQALPPR**
CAR-T C2 CD8/CD8/OX40/CD3z sequence:
N-CD81s-huMNC2scFv-CD8ecd fragment- CD8 transmembrane- OX40- CD3zeta-C
(DNA)
(SEQ ID NO: 612)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgccggagggaccagaggctgccccccgatgcccacaag
ccccctgggggaggcagtttccggacccccatccaagaggagcaggccgacgcccactccaccctggccaagatccgcgtt
aagttctcccgatcagccgacgcgcctgcttacaagcagggccagaaccaactgtacaacgagctgaatctcggtagacgg
gaagagtacgacgtgttggacaaacggagaggccgcgacccagaaatgggcggcaagcctcgcaggaaaaacccccaggag
ggactgtacaatgagttgcagaaagataagatggcagaagcttatagcgagatcggaatgaagggggaaaggagacgaggg
aaaggacacgacggcctttatcagggcctgtccacagcaacaaaagatacgtatgacgccctccatatgcaggcacttcca
ccacggtgataa
(amino acids)
(SEQ ID NO: 613)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCRRDQRLPPDAHKPPGGGSFRTPIQEEQADAHSTLAKIRVKFSRSADAPAYKQGQNQLYNEL
NLGRREEYDVLDKRRGRDPEMGGKPRRKNPQEGLYNELQKDKMAEAYSEIGMKGERRRGKGHDGLYQGLSTATKDTYDAL
HMQALPPR**
CAR-T C2 CD8/CD8/CD28/OX40/CD3z sequence:
N-CD81s-huMNC2scFv-CD8ecd fragment- CD8 transmembrane- CD28- OX40- CD3zeta-C
(DNA)
(SEQ ID NO: 614)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtcccggagggaccagaggctgccccccgatgcccacaagccccctgggggaggcagtttccggacccccatccaagag
gagcaggccgacgcccactccaccctggccaagatccgcgttaagttctcccgatcagccgacgcgcctgcttacaagcag
ggccagaaccaactgtacaacgagctgaatctcggtagacgggaagagtacgacgtgttggacaaacggagaggccgcgac
ccagaaatgggcggcaagcctcgcaggaaaaacccccaggagggactgtacaatgagttgcagaaagataagatggcagaa
gcttatagcgagatcggaatgaagggggaaaggagacgagggaaaggacacgacggcctttatcagggcctgtccacagca
acaaaagatacgtatgacgccctccatatgcaggcacttccaccacggtgataa
(amino acids)
(SEQ ID NO: 615)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCRSKRSRLLHSDYMNMTPRRPGPTRKHYQPYAPPRDFAAYRSRRDQRLPPDAHKPPGGGSFR
TPIQEEQADAHSTLAKIRVKFSRSADAPAYKQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR**
CAR-T E6 CD8/CD8/OX40/CD3z sequence:
N-CD81s-huMNE6scFv-CD8ecd fragment- CD8 transmembrane- OX40- CD3zeta-C
(DNA)
(SEQ ID NO: 616)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgccggagggaccagaggctgccccccgatgcccacaagccccctgggggaggcagt
ttccggacccccatccaagaggagcaggccgacgcccactccaccctggccaagatccgcgttaagttctcccgatcagcc
gacgcgcctgcttacaagcagggccagaaccaactgtacaacgagctgaatctcggtagacgggaagagtacgacgtgttg
gacaaacggagaggccgcgacccagaaatgggcggcaagcctcgcaggaaaaacccccaggagggactgtacaatgagttg
cagaaagataagatggcagaagcttatagcgagatcggaatgaagggggaaaggagacgagggaaaggacacgacggcctt
tatcagggcctgtccacagcaacaaaagatacgtatgacgccctccatatgcaggcacttccaccacggtgataa
(amino acids)
(SEQ ID NO: 617)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRRDQRLPPDAHKPPGGGSFRTPIQEEQADAHSTLAKIRVKFSRSADAPAYKQGQNQLYNELNLGRRE
EYDVLDKRRGRDPEMGGKPRRKNPQEGLYNELQKDKMAEAYSEIGMKGERRRGKGHDGLYQGLSTATKDTYDALHMQALP
PR**
CAR-T E6 CD8/CD8/CD28/OX40/CD3z sequence:
N-CD81s-huMNC2scFv-CD8ecd fragment- CD8 transmembrane- CD28- OX40- CD3zeta-C
(DNA)
(SEQ ID NO: 618)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgcagaagcaagcggtctcggctcctgcattctgattacatgaacatgaccccaaga
agaccaggccccaccaggaaacattaccagccctacgctccgccacgcgacttcgctgcctaccggtcccggagggaccag
aggctgccccccgatgcccacaagccccctgggggaggcagtttccggacccccatccaagaggagcaggccgacgcccac
tccaccctggccaagatccgcgttaagttctcccgatcagccgacgcgcctgcttacaagcagggccagaaccaactgtac
aacgagctgaatctcggtagacgggaagagtacgacgtgttggacaaacggagaggccgcgacccagaaatgggcggcaag
cctcgcaggaaaaacccccaggagggactgtacaatgagttgcagaaagataagatggcagaagcttatagcgagatcgga
atgaagggggaaaggagacgagggaaaggacacgacggcctttatcagggcctgtccacagcaacaaaagatacgtatgac
gccctccatatgcaggcacttccaccacggtgataa
(amino acids)
(SEQ ID NO: 619)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRSKRSRLLHSDYMNMTPRRPGPTRKHYQPYAPPRDFAAYRSRRDQRLPPDAHKPPGGGSFRTPIQEE
QADAHSTLAKIRVKFSRSADAPAYKQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR**
MUC1 truncated cytoplasmic sequence
(amino acids)
(SEQ ID NO: 620)
SNIKFRPGSVVVQLTLAFREGTINVHDVETQFNQYKTEAASRY
MUC1 truncated cytoplasmic sequence
(amino acids)
(SEQ ID NO: 621)
SVVVQLTLAFREGTINVHDVETQFNQYKTEAASRY
MUC1 truncated cytoplasmic sequence
(amino acids)
(SEQ ID NO: 622)
VQLTLAFREGTINVHDVETQFNQY
MUC1 truncated cytoplasmic sequence
(amino acids)
(SEQ ID NO: 623)
SNIKFRPGSVVVQLTLAFREGTIN
Primers
(SEQ ID NO: 624)
attctaagcttgggccaccatggaactg
(SEQ ID NO: 625)
tctagagtttaaacttactatttacccggagacagggagag
(SEQ ID NO: 626)
agtatggcccagccggccgaggtgcagctggtggagtctgg
(SEQ ID NO: 627)
tagaaggcacagtcgaggctgatcag
(SEQ ID NO: 628)
attctaagcttgggccaccatggaagc
(SEQ ID NO: 629)
tctagagtttaaacttactaacactctcccctgttgaagc
(SEQ ID NO: 630)
agtatggcccagccggccgaaattgtgttgacacagtctccag
(SEQ ID NO: 631)
tagaaggcacagtcgaggctgatcag
(SEQ ID NO: 632)
actgtcatatggaggtgcagctggtggagtctg
(SEQ ID NO: 633)
actgtctcgagtttaatttccactttggtgccgctgc
(SEQ ID NO: 634)
actgtcatatggaggtgcagctggtggagtctg
(SEQ ID NO: 635)
actgtaccggttttaatttccactttggtgccgctgc
(SEQ ID NO: 636)
cttcttcctcaggagcaagctcaccgtgg
(SEQ ID NO: 637)
gagccgtcggagtccagc
(SEQ ID NO: 638)
gcacctgaactcctgggg
(SEQ ID NO: 639)
tttaatttccactttggtgccg
(SEQ ID NO: 640)
cgcggctagcttaagcttggtaccgagggcca
(SEQ ID NO: 641)
cgcggcggccgcctgatcagcgggtttaaacttatc
MMP9
(DNA)
(SEQ ID NO: 642)
atgagcctctggcagcccctggtcctggtgctcctggtgctgggctgctgctttgctgcccccagacagcgccagtccacc
cttgtgctcttccctggagacctgagaaccaatctcaccgacaggcagctggcagaggaatacctgtaccgctatggttac
actcgggtggcagagatgcgtggagagtcgaaatctctggggcctgcgctgctgcttctccagaagcaactgtccctgccc
gagaccggtgagctggatagcgccacgctgaaggccatgcgaaccccacggtgcggggtcccagacctgggcagattccaa
acctttgagggcgacctcaagtggcaccaccacaacatcacctattggatccaaaactactcggaagacttgccgcgggcg
gtgattgacgacgcctttgcccgcgccttcgcactgtggagcgcggtgacgccgctcaccttcactcgcgtgtacagccgg
gacgcagacatcgtcatccagtttggtgtcgcggagcacggagacgggtatcccttcgacgggaaggacgggctcctggca
cacgcctttcctcctggccccggcattcagggagacgcccatttcgacgatgacgagttgtggtccctgggcaagggcgtc
gtggttccaactcggtttggaaacgcagatggcgcggcctgccacttccccttcatcttcgagggccgctcctactctgcc
tgcaccaccgacggtcgctccgacggcttgccctggtgcagtaccacggccaactacgacaccgacgaccggtttggcttc
tgccccagcgagagactctacacccaggacggcaatgctgatgggaaaccctgccagtttccattcatcttccaaggccaa
tcctactccgcctgcaccacggacggtcgctccgacggctaccgctggtgcgccaccaccgccaactacgaccgggacaag
ctcttcggcttctgcccgacccgagctgactcgacggtgatggggggcaactcggcgggggagctgtgcgtcttccccttc
actttcctgggtaaggagtactcgacctgtaccagcgagggccgcggagatgggcgcctctggtgcgctaccacctcgaac
tttgacagcgacaagaagtggggcttctgcccggaccaaggatacagtttgttcctcgtggcggcgcatgagttcggccac
gcgctgggcttagatcattcctcagtgccggaggcgctcatgtaccctatgtaccgcttcactgaggggccccccttgcat
aaggacgacgtgaatggcatccggcacctctatggtcctcgccctgaacctgagccacggcctccaaccaccaccacaccg
cagcccacggctcccccgacggtctgccccaccggaccccccactgtccacccctcagagcgccccacagctggccccaca
ggtcccccctcagctggccccacaggtccccccactgctggcccttctacggccactactgtgcctttgagtccggtggac
gatgcctgcaacgtgaacatcttcgacgccatcgcggagattgggaaccagctgtatttgttcaaggatgggaagtactgg
cgattctctgagggcagggggagccggccgcagggccccttccttatcgccgacaagtggcccgcgctgccccgcaagctg
gactcggtctttgaggagcggctctccaagaagcttttcttcttctctgggcgccaggtgtgggtgtacacaggcgcgtcg
gtgctgggcccgaggcgtctggacaagctgggcctgggagccgacgtggcccaggtgaccggggccctccggagtggcagg
gggaagatgctgctgttcagcgggcggcgcctctggaggttcgacgtgaaggcgcagatggtggatccccggagcgccagc
gaggtggaccggatgttccccggggtgcctttggacacgcacgacgtcttccagtaccgagagaaagcctatttctgccag
gaccgcttctactggcgcgtgagttcccggagtgagttgaaccaggtggaccaagtgggctacgtgacctatgacatcctg
cagtgccctgaggacgattacaaggatgacgacgataagtgataa
(amino acids)
(SEQ ID NO: 643)
MSLWQPLVLVLLVLGCCFAAPRQRQSTLVLFPGDLRTNLTDRQLAEEYLYRYGYTRVAEMRGESKSLGPALLLLQKQLSL
PETGELDSATLKAMRTPRCGVPDLGRFQTFEGDLKWHHHNITYWIQNYSEDLPRAVIDDAFARAFALWSAVTPLTFTRVY
SRDADIVIQFGVAEHGDGYPFDGKDGLLAHAFPPGPGIQGDAHFDDDELWSLGKGVVVPTRFGNADGAACHFPFIFEGRS
YSACTTDGRSDGLPWCSTTANYDTDDRFGFCPSERLYTQDGNADGKPCQFPFIFQGQSYSACTTDGRSDGYRWCATTANY
DRDKLFGFCPTRADSTVMGGNSAGELCVFPFTFLGKEYSTCTSEGRGDGRLWCATTSNFDSDKKWGFCPDQGYSLFLVAA
HEFGHALGLDHSSVPEALMYPMYRFTEGPPLHKDDVNGIRHLYGPRPEPEPRPPTTTTPQPTAPPTVCPTGPPTVHPSER
PTAGPTGPPSAGPTGPPTAGPSTATTVPLSPVDDACNVNIFDAIAEIGNQLYLFKDGKYWRFSEGRGSRPQGPFLIADKW
PALPRKLDSVFEERLSKKLFFFSGRQVWVYTGASVLGPRRLDKLGLGADVAQVTGALRSGRGKMLLFSGRRLWRFDVKAQ
MVDPRSASEVDRMFPGVPLDTHDVFQYREKAYFCQDRFYWRVSSRSELNQVDQVGYVTYDILQCPEDDYKDDDDK**
MMP9 catalytic domain
(DNA)
(SEQ ID NO: 644)
atgttccaaacctttgagggcgacctcaagtggcaccaccacaacatcacctattggatccaaaactactcggaagacttg
ccgcgggcggtgattgacgacgcctttgcccgcgccttcgcactgtggagcgcggtgacgccgctcaccttcactcgcgtg
tacagccgggacgcagacatcgtcatccagtttggtgtcgcggagcacggagacgggtatcccttcgacgggaaggacggg
ctcctggcacacgcctttcctcctggccccggcattcagggagacgcccatttcgacgatgacgagttgtggtccctgggc
aagggcgtcgtggttccaactcggtttggaaacgcagatggcgcggcctgccacttccccttcatcttcgagggccgctcc
tactctgcctgcaccaccgacggtcgctccgacggcttgccctggtgcagtaccacggccaactacgacaccgacgaccgg
tttggcttctgccccagcgagagactctacacccaggacggcaatgctgatgggaaaccctgccagtttccattcatcttc
caaggccaatcctactccgcctgcaccacggacggtcgctccgacggctaccgctggtgcgccaccaccgccaactacgac
cgggacaagctcttcggcttctgcccgacccgagctgactcgacggtgatggggggcaactcggcgggggagctgtgcgtc
ttccccttcactttcctgggtaaggagtactcgacctgtaccagcgagggccgcggagatgggcgcctctggtgcgctacc
acctcgaactttgacagcgacaagaagtggggcttctgcccggaccaaggatacagtttgttcctcgtggcggcgcatgag
ttcggccacgcgctgggcttagatcattcctcagtgccggaggcgctcatgtaccctatgtaccgcttcactgaggggccc
cccttgcataaggacgacgtgaatggcatccggcacctctatggtcctcgccctgaacctgattacaaggatgacgacgat
aagtgataa
(amino acids)
(SEQ ID NO: 645)
MFQTFEGDLKWHHHNITYWIQNYSEDLPRAVIDDAFARAFALWSAVTPLTFTRVYSRDADIVIQFGVAEHGDGYPFDGKD
GLLAHAFPPGPGIQGDAHFDDDELWSLGKGVVVPTRFGNADGAACHFPFIFEGRSYSACTTDGRSDGLPWCSTTANYDTD
DRFGFCPSERLYTQDGNADGKPCQFPFIFQGQSYSACTTDGRSDGYRWCATTANYDRDKLFGFCPTRADSTVMGGNSAGE
LCVFPFTFLGKEYSTCTSEGRGDGRLWCATTSNFDSDKKWGFCPDQGYSLFLVAAHEFGHALGLDHSSVPEALMYPMYRF
TEGPPLHKDDVNGIRHLYGPRPEPDYKDDDDK**
NFATc1 Promoter (NFATc1P)
(DNA)
(SEQ ID NO: 646)
aggcaggaggaagaggaaaggggcgcagggcgctcggggagcagagccgggggcccgcggtggccgcagaggccgggccgg
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagagg
NFATc1P-MMP9
(DNA)
(SEQ ID NO: 647)
aggcaggaggaagaggaaaggggcgcagggcgctcggggagcagagccgggggcccgcggtggccgcagaggccgggccgg
t
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagaggtctagagccaccatgagcctctggcagcccctggtcctggtgctcctggtgctgg
gctgctgctttgctgcccccagacagcgccagtccacccttgtgctcttccctggagacctgagaaccaatctcaccgaca
ggcagctggcagaggaatacctgtaccgctatggttacactcgggtggcagagatgcgtggagagtcgaaatctctggggc
ctgcgctgctgcttctccagaagcaactgtccctgcccgagaccggtgagctggatagcgccacgctgaaggccatgcgaa
ccccacggtgcggggtcccagacctgggcagattccaaacctttgagggcgacctcaagtggcaccaccacaacatcacct
attggatccaaaactactcggaagacttgccgcgggcggtgattgacgacgcctttgcccgcgccttcgcactgtggagcg
cggtgacgccgctcaccttcactcgcgtgtacagccgggacgcagacatcgtcatccagtttggtgtcgcggagcacggag
acgggtatcccttcgacgggaaggacgggctcctggcacacgcctttcctcctggccccggcattcagggagacgcccatt
tcgacgatgacgagttgtggtccctgggcaagggcgtcgtggttccaactcggtttggaaacgcagatggcgcggcctgcc
acttccccttcatcttcgagggccgctcctactctgcctgcaccaccgacggtcgctccgacggcttgccctggtgcagta
ccacggccaactacgacaccgacgaccggtttggcttctgccccagcgagagactctacacccaggacggcaatgctgatg
ggaaaccctgccagtttccattcatcttccaaggccaatcctactccgcctgcaccacggacggtcgctccgacggctacc
gctggtgcgccaccaccgccaactacgaccgggacaagctcttcggcttctgcccgacccgagctgactcgacggtgatgg
ggggcaactcggcgggggagctgtgcgtcttccccttcactttcctgggtaaggagtactcgacctgtaccagcgagggcc
gcggagatgggcgcctctggtgcgctaccacctcgaactttgacagcgacaagaagtggggcttctgcccggaccaaggat
acagtttgttcctcgtggcggcgcatgagttcggccacgcgctgggcttagatcattcctcagtgccggaggcgctcatgt
accctatgtaccgcttcactgaggggccccccttgcataaggacgacgtgaatggcatccggcacctctatggtcctcgcc
ctgaacctgagccacggcctccaaccaccaccacaccgcagcccacggctcccccgacggtctgccccaccggacccccca
ctgtccacccctcagagcgccccacagctggccccacaggtcccccctcagctggccccacaggtccccccactgctggcc
cttctacggccactactgtgcctttgagtccggtggacgatgcctgcaacgtgaacatcttcgacgccatcgcggagattg
ggaaccagctgtatttgttcaaggatgggaagtactggcgattctctgagggcagggggagccggccgcagggccccttcc
ttatcgccgacaagtggcccgcgctgccccgcaagctggactcggtctttgaggagcggctctccaagaagcttttcttct
tctctgggcgccaggtgtgggtgtacacaggcgcgtcggtgctgggcccgaggcgtctggacaagctgggcctgggagccg
acgtggcccaggtgaccggggccctccggagtggcagggggaagatgctgctgttcagcgggcggcgcctctggaggttcg
acgtgaaggcgcagatggtggatccccggagcgccagcgaggtggaccggatgttccccggggtgcctttggacacgcacg
acgtcttccagtaccgagagaaagcctatttctgccaggaccgcttctactggcgcgtgagttcccggagtgagttgaacc
aggtggaccaagtgggctacgtgacctatgacatcctgcagtgccctgaggacgattacaaggatgacgacgataagtgat
aa
NFATc1P-MMP9cat
(DNA)
(SEQ ID NO: 648)
aggcaggaggaagaggaaaggggcgcagggcgctcggggagcagagccgggggcccgcggtggccgcagaggccgggccgg
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagaggtctagagccaccatgttccaaacctttgagggcgacctcaagtggcaccaccaca
acatcacctattggatccaaaactactcggaagacttgccgcgggcggtgattgacgacgcctttgcccgcgccttcgcac
tgtggagcgcggtgacgccgctcaccttcactcgcgtgtacagccgggacgcagacatcgtcatccagtttggtgtcgcgg
agcacggagacgggtatcccttcgacgggaaggacgggctcctggcacacgcctttcctcctggccccggcattcagggag
acgcccatttcgacgatgacgagttgtggtccctgggcaagggcgtcgtggttccaactcggtttggaaacgcagatggcg
cggcctgccacttccccttcatcttcgagggccgctcctactctgcctgcaccaccgacggtcgctccgacggcttgccct
ggtgcagtaccacggccaactacgacaccgacgaccggtttggcttctgccccagcgagagactctacacccaggacggca
atgctgatgggaaaccctgccagtttccattcatcttccaaggccaatcctactccgcctgcaccacggacggtcgctccg
acggctaccgctggtgcgccaccaccgccaactacgaccgggacaagctcttcggcttctgcccgacccgagctgactcga
cggtgatggggggcaactcggcgggggagctgtgcgtcttccccttcactttcctgggtaaggagtactcgacctgtacca
gcgagggccgcggagatgggcgcctctggtgcgctaccacctcgaactttgacagcgacaagaagtggggcttctgcccgg
accaaggatacagtttgttcctcgtggcggcgcatgagttcggccacgcgctgggcttagatcattcctcagtgccggagg
cgctcatgtaccctatgtaccgcttcactgaggggccccccttgcataaggacgacgtgaatggcatccggcacctctatg
gtcctcgccctgaacctgattacaaggatgacgacgataagtgataa
NFAT response element
(DNA)
(SEQ ID NO: 649)
ggaggaaaaactgtttcatacagaaggcgt
NFAT response element repeats
(DNA)
(SEQ ID NO: 650)
ggaggaaaaactgtttcatacagaaggcgtggaggaaaaactgtttcatacagaaggcgtggaggaaaaactgtttcatac
agaaggcgt
CMV minimal promoter
(DNA)
(SEQ ID NO: 651)
aggtaggcgtgtacggtgggaggtctatataagcagagctggtttagtgaaccgtcagatc
NFATREmCMV-MMP9
(DNA)
(SEQ ID NO: 652)
ggaggaaaaactgtttcatacagaaggcgtggaggaaaaactgtttcatacagaaggcgtggaggaaaaactgtttcatac
agaaggcgtagatctagactcaggtaggcgtgtacggtgggaggtctatataagcagagctggtttagtgaaccgtcagat
ctctagagccaccatgagcctctggcagcccctggtcctggtgctcctggtgctgggctgctgctttgctgcccccagaca
gcgccagtccacccttgtgctcttccctggagacctgagaaccaatctcaccgacaggcagctggcagaggaatacctgta
ccgctatggttacactcgggtggcagagatgcgtggagagtcgaaatctctggggcctgcgctgctgcttctccagaagca
actgtccctgcccgagaccggtgagctggatagcgccacgctgaaggccatgcgaaccccacggtgcggggtcccagacct
gggcagattccaaacctttgagggcgacctcaagtggcaccaccacaacatcacctattggatccaaaactactcggaaga
cttgccgcgggcggtgattgacgacgcctttgcccgcgccttcgcactgtggagcgcggtgacgccgctcaccttcactcg
cgtgtacagccgggacgcagacatcgtcatccagtttggtgtcgcggagcacggagacgggtatcccttcgacgggaagga
cgggctcctggcacacgcctttcctcctggccccggcattcagggagacgcccatttcgacgatgacgagttgtggtccct
gggcaagggcgtcgtggttccaactcggtttggaaacgcagatggcgcggcctgccacttccccttcatcttcgagggccg
ctcctactctgcctgcaccaccgacggtcgctccgacggcttgccctggtgcagtaccacggccaactacgacaccgacga
ccggtttggcttctgccccagcgagagactctacacccaggacggcaatgctgatgggaaaccctgccagtttccattcat
cttccaaggccaatcctactccgcctgcaccacggacggtcgctccgacggctaccgctggtgcgccaccaccgccaacta
cgaccgggacaagctcttcggcttctgcccgacccgagctgactcgacggtgatggggggcaactcggcgggggagctgtg
cgtcttccccttcactttcctgggtaaggagtactcgacctgtaccagcgagggccgcggagatgggcgcctctggtgcgc
taccacctcgaactttgacagcgacaagaagtggggcttctgcccggaccaaggatacagtttgttcctcgtggcggcgca
tgagttcggccacgcgctgggcttagatcattcctcagtgccggaggcgctcatgtaccctatgtaccgcttcactgaggg
gccccccttgcataaggacgacgtgaatggcatccggcacctctatggtcctcgccctgaacctgagccacggcctccaac
caccaccacaccgcagcccacggctcccccgacggtctgccccaccggaccccccactgtccacccctcagagcgccccac
agctggccccacaggtcccccctcagctggccccacaggtccccccactgctggcccttctacggccactactgtgccttt
gagtccggtggacgatgcctgcaacgtgaacatcttcgacgccatcgcggagattgggaaccagctgtatttgttcaagga
tgggaagtactggcgattctctgagggcagggggagccggccgcagggccccttccttatcgccgacaagtggcccgcgct
gccccgcaagctggactcggtctttgaggagcggctctccaagaagcttttcttcttctctgggcgccaggtgtgggtgta
cacaggcgcgtcggtgctgggcccgaggcgtctggacaagctgggcctgggagccgacgtggcccaggtgaccggggccct
ccggagtggcagggggaagatgctgctgttcagcgggcggcgcctctggaggttcgacgtgaaggcgcagatggtggatcc
ccggagcgccagcgaggtggaccggatgttccccggggtgcctttggacacgcacgacgtcttccagtaccgagagaaagc
ctatttctgccaggaccgcttctactggcgcgtgagttcccggagtgagttgaaccaggtggaccaagtgggctacgtgac
ctatgacatcctgcagtgccctgaggacgattacaaggatgacgacgataagtgataa
NFATREmCMV-MMP9cat
(DNA)
(SEQ ID NO: 653)
ggaggaaaaactgtttcatacagaaggcgtggaggaaaaactgtttcatacagaaggcgtggaggaaaaactgtttcatac
agaaggcgtagatctagactcaggtaggcgtgtacggtgggaggtctatataagcagagctggtttagtgaaccgtcagat
ctctagagccaccatgttccaaacctttgagggcgacctcaagtggcaccaccacaacatcacctattggatccaaaacta
ctcggaagacttgccgcgggcggtgattgacgacgcctttgcccgcgccttcgcactgtggagcgcggtgacgccgctcac
cttcactcgcgtgtacagccgggacgcagacatcgtcatccagtttggtgtcgcggagcacggagacgggtatcccttcga
cgggaaggacgggctcctggcacacgcctttcctcctggccccggcattcagggagacgcccatttcgacgatgacgagtt
gtggtccctgggcaagggcgtcgtggttccaactcggtttggaaacgcagatggcgcggcctgccacttccccttcatctt
cgagggccgctcctactctgcctgcaccaccgacggtcgctccgacggcttgccctggtgcagtaccacggccaactacga
caccgacgaccggtttggcttctgccccagcgagagactctacacccaggacggcaatgctgatgggaaaccctgccagtt
tccattcatcttccaaggccaatcctactccgcctgcaccacggacggtcgctccgacggctaccgctggtgcgccaccac
cgccaactacgaccgggacaagctcttcggcttctgcccgacccgagctgactcgacggtgatggggggcaactcggcggg
ggagctgtgcgtcttccccttcactttcctgggtaaggagtactcgacctgtaccagcgagggccgcggagatgggcgcct
ctggtgcgctaccacctcgaactttgacagcgacaagaagtggggcttctgcccggaccaaggatacagtttgttcctcgt
ggcggcgcatgagttcggccacgcgctgggcttagatcattcctcagtgccggaggcgctcatgtaccctatgtaccgctt
cactgaggggccccccttgcataaggacgacgtgaatggcatccggcacctctatggtcctcgccctgaacctgattacaa
ggatgacgacgataagtgataa
C2 scFv
(DNA)
(SEQ ID NO: 654)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaact
(amino acids)
(SEQ ID NO: 655)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRT
CD8 transmembrane domain
(DNA)
(SEQ ID NO: 656)
atctacatctgggcgcccttggccgggacttgtggggtccttctcctgtcactggttatcaccctttactgc
(amino acids)
(SEQ ID NO: 657)
IYIWAPLAGTCGVLLLSLVITLYC
4-1BB domain
(DNA)
(SEQ ID NO: 658)
aaacggggcagaaagaaactcctgtatatattcaaacaaccatttatgagaccagtacaaactactcaagaggaagatggc
tgtagctgccgatttccagaagaagaagaaggaggatgtgaactg
(amino acids)
(SEQ ID NO: 659)
KRGRKKLLYIFKQPFMRPVQTTQEEDGCSCRFPEEEEGGCEL
CD3 zeta domain
(DNA)
(SEQ ID NO: 660)
agagtgaagttcagcaggagcgcagacgcccccgcgtacaagcagggccagaaccagctctataacgagctcaatctagga
cgaagagaggagtacgatgttttggacaagagacgtggccgggaccctgagatggggggaaagccgagaaggaagaaccct
caggaaggcctgtacaatgaactgcagaaagataagatggcggaggcctacagtgagattgggatgaaaggcgagcgccgg
aggggcaaggggcacgatggcctttaccagggtctcagtacagccaccaaggacacctacgacgcccttcacatgcaggcc
ctgccccctcgc
(amino acids)
(SEQ ID NO: 661)
RVKFSRSADAPAYKQGQNQLYNELNLGRREEYDVLDKRRGRDPEMGGKPRRKNPQEGLYNELQKDKMAEAYSEIGMKGER
RRGKGHDGLYQGLSTATKDTYDALHMQALPPR
Human IgG1 Fc linker
(DNA)
(SEQ ID NO: 662)
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaa
(amino acids)
(SEQ ID NO: 663)
EPKSCDKTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQKSLSLSPGK
C2 CAR FC linker
(DNA)
(SEQ ID NO: 664)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagcccaaatcttgtgacaaaactcacacatgcccaccgtgcccagcacctgaactcctgggg
ggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaag
gagtacaagtgcaaggtctccaacaaagccctcccagcccccatcgagaaaaccatctccaaagccaaagggcagccccga
gaaccacaggtgtacaccctgcccccatcccgggaggagatgaccaagaaccaggtcagcctgacctgcctggtcaaaggc
ttctatcccagcgacatcgccgtggagtgggagagcaatgggcagccggagaacaactacaagaccacgcctcccgtgctg
gactccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaatctacatctgggcg
cccttggccgggacttgtggggtccttctcctgtcactggttatcaccctttactgcaaacggggcagaaagaaactcctg
tatatattcaaacaaccatttatgagaccagtacaaactactcaagaggaagatggctgtagctgccgatttccagaagaa
gaagaaggaggatgtgaactgagagtgaagttcagcaggagcgcagacgcccccgcgtacaagcagggccagaaccagctc
tataacgagctcaatctaggacgaagagaggagtacgatgttttggacaagagacgtggccgggaccctgagatgggggga
aagccgagaaggaagaaccctcaggaaggcctgtacaatgaactgcagaaagataagatggcggaggcctacagtgagatt
gggatgaaaggcgagcgccggaggggcaaggggcacgatggcctttaccagggtctcagtacagccaccaaggacacctac
gacgcccttcacatgcaggccctgccccctcgctgataa
(amino acids)
(SEQ ID NO: 665)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTEPKSCDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSLSLSPG
KIYIWAPLAGTCGVLLLSLVITLYCKRGRKKLLYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNELQKDKMAEAYSEIGMKGERRRGKGHDGLYQGL
STATKDTYDALHMQALPPR**
IgD/Fc linker
(DNA)
(SEQ ID NO: 666)
gagtctccaaaggcacaggcctcctcagtgcccactgcacaaccccaagcagagggcagcctcgccaaggcaaccacagcc
ccagccaccacccgtaacacaggaagaggcggcgaagagaagaaaaaggagaaggagaaagaggaacaagaagagagagag
acaaagacaccagagcccaaatcttgtgacaaaactcacacatgcccaccgtgcccagcacctgaactcctggggggaccg
tcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgg
gaggagcagtacaacagcacgtaccgtgtggtcagcgtcctcaccgtcctgcaccaggactggctgaatggcaaggagtac
aagtgcaaggtctccaacaaagccctcccagcccccatcgagaaaaccatctccaaagccaaagggcagccccgagaacca
caggtgtacaccctgcccccatcccgggaggagatgaccaagaaccaggtcagcctgacctgcctggtcaaaggcttctat
cccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtg
atgcatgaggctctgcacaaccactacacgcagaagagcctctccctgtctccgggtaaa
(amino acids)
(SEQ ID NO: 667)
ESPKAQASSVPTAQPQAEGSLAKATTAPATTRNTGRGGEEKKKEKEKEEQEERETKTPEPKSCDKTHTCPPCPAPELLGG
PSVFLFPPKPKDTLMISRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
C2 CAR IgD/FC linker
(DNA)
(SEQ ID NO: 668)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccagagcccaaatcttgtgacaaaactcacacatgcccaccgtgcccagcacct
gaactcctggggggaccgtcagtcttcctcttccccccaaaacccaaggacaccctcatgatctcccggacccctgaggtc
acatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactgg
ctgaatggcaaggagtacaagtgcaaggtctccaacaaagccctcccagcccccatcgagaaaaccatctccaaagccaaa
gggcagccccgagaaccacaggtgtacaccctgcccccatcccgggaggagatgaccaagaaccaggtcagcctgacctgc
ctggtcaaaggcttctatcccagcgacatcgccgtggagtgggagagcaatgggcagccggagaacaactacaagaccacg
cctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaaatc
tacatctgggcgcccttggccgggacttgtggggtccttctcctgtcactggttatcaccctttactgcaaacggggcaga
aagaaactcctgtatatattcaaacaaccatttatgagaccagtacaaactactcaagaggaagatggctgtagctgccga
tttccagaagaagaagaaggaggatgtgaactgagagtgaagttcagcaggagcgcagacgcccccgcgtacaagcagggc
cagaaccagctctataacgagctcaatctaggacgaagagaggagtacgatgttttggacaagagacgtggccgggaccct
gagatggggggaaagccgagaaggaagaaccctcaggaaggcctgtacaatgaactgcagaaagataagatggcggaggcc
tacagtgagattgggatgaaaggcgagcgccggaggggcaaggggcacgatggcctttaccagggtctcagtacagccacc
aaggacacctacgacgcccttcacatgcaggccctgccccctcgctgataa
(amino acids)
(SEQ ID NO: 669)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPEPKSCDKTHTCPP
CPAPELLGGPSVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGKIYIWAPLAGTCGVLLLSLVIT
LYCKRGRKKLLYIFKQPFMRPVQTTQEEDGCSCRFPEEEEGGCELRVKFSRSADAPAYKQGQNQLYNELNLGRREEYDVL
DKRRGRDPEMGGKPRRKNPQEGLYNELQKDKMAEAYSEIGMKGERRRGKGHDGLYQGLSTATKDTYDALHMQALPPR**
Fc hingeless Y407R linker
(DNA)
(SEQ ID NO: 670)
gcacctgaactcctggggggaccgtcagtcttcctcttccccccaaaacccaaggacaccctcatgatctcccggacccct
gaggtcacatgcgtggtggtggacgtgagccacgaagaccctgaggtcaagttcaactggtacgtggacggcgtggaggtg
cataatgccaagacaaagccgcgggaggagcagtacaacagcacgtaccgtgtggtcagcgtcctcaccgtcctgcaccag
gactggctgaatggcaaggagtacaagtgcaaggtctccaacaaagccctcccagcccccatcgagaaaaccatctccaaa
gccaaagggcagccccgagaaccacaggtgtacaccctgcccccatcccgggaggagatgaccaagaaccaggtcagcctg
acctgcctggtcaaaggcttctatcccagcgacatcgccgtggagtgggagagcaatgggcagccggagaacaactacaag
accacgcctcccgtgctggactccgacggctccttcttcctcaggagcaagctcaccgtggacaagagcaggtggcagcag
gggaacgtcttctcatgctccgtgatgcatgaggctctgcacaaccactacacgcagaagagcctctccctgtctccgggt
aaa
(amino acids)
(SEQ ID NO: 671)
APELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLVKGFYPSDIAVEWESNGQPENN
YKTTPPVLDSDGSFFLRSKLTVDKSRWQQGNVFSCSVMHEALHNHYTQKSLSLSPGK
C2 CAR FCHingeless/Y407R linker
(DNA)
(SEQ ID NO: 672)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgcacctgaactcctggggggaccgtcagtcttcctcttccccccaaaacccaaggacaccctc
atgatctcccggacccctgaggtcacatgcgtggtggtggacgtgagccacgaagaccctgaggtcaagttcaactggtac
gtggacggcgtggaggtgcataatgccaagacaaagccgcgggaggagcagtacaacagcacgtaccgtgtggtcagcgtc
ctcaccgtcctgcaccaggactggctgaatggcaaggagtacaagtgcaaggtctccaacaaagccctcccagcccccatc
gagaaaaccatctccaaagccaaagggcagccccgagaaccacaggtgtacaccctgcccccatcccgggaggagatgacc
aagaaccaggtcagcctgacctgcctggtcaaaggcttctatcccagcgacatcgccgtggagtgggagagcaatgggcag
ccggagaacaactacaagaccacgcctcccgtgctggactccgacggctccttcttcctcaggagcaagctcaccgtggac
aagagcaggtggcagcaggggaacgtcttctcatgctccgtgatgcatgaggctctgcacaaccactacacgcagaagagc
ctctccctgtctccgggtaaaatctacatctgggcgcccttggccgggacttgtggggtccttctcctgtcactggttatc
accctttactgcaaacggggcagaaagaaactcctgtatatattcaaacaaccatttatgagaccagtacaaactactcaa
gaggaagatggctgtagctgccgatttccagaagaagaagaaggaggatgtgaactgagagtgaagttcagcaggagcgca
gacgcccccgcgtacaagcagggccagaaccagctctataacgagctcaatctaggacgaagagaggagtacgatgttttg
gacaagagacgtggccgggaccctgagatggggggaaagccgagaaggaagaaccctcaggaaggcctgtacaatgaactg
cagaaagataagatggcggaggcctacagtgagattgggatgaaaggcgagcgccggaggggcaaggggcacgatggcctt
taccagggtctcagtacagccaccaaggacacctacgacgcccttcacatgcaggccctgccccctcgctgataa
(amino acids)
(SEQ ID NO: 673)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTAPELLGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQVSLTCLVKGFYPSDIAVEW
ESNGQPENNYKTTPPVLDSDGSFFLRSKLTVDKSRWQQGNVFSCSVMHEALHNHYTQKSLSLSPGKIYIWAPLAGTCGVL
LLSLVITLYCKRGRKKLLYIFKQPFMRPVQTTQEEDGCSCRFPEEEEGGCELRVKFSRSADAPAYKQGQNQLYNELNLGR
REEYDVLDKRRGRDPEMGGKPRRKNPQEGLYNELQKDKMAEAYSEIGMKGERRRGKGHDGLYQGLSTATKDTYDALHMQA
LPPR**
IgD/FCHingeless/Y407R linker
(DNA)
(SEQ ID NO: 674)
gagtctccaaaggcacaggcctcctcagtgcccactgcacaaccccaagcagagggcagcctcgccaaggcaaccacagcc
ccagccaccacccgtaacacaggaagaggcggcgaagagaagaaaaaggagaaggagaaagaggaacaagaagagagagag
acaaagacaccagcacctgaactcctggggggaccgtcagtcttcctcttccccccaaaacccaaggacaccctcatgatc
tcccggacccctgaggtcacatgcgtggtggtggacgtgagccacgaagaccctgaggtcaagttcaactggtacgtggac
ggcgtggaggtgcataatgccaagacaaagccgcgggaggagcagtacaacagcacgtaccgtgtggtcagcgtcctcacc
gtcctgcaccaggactggctgaatggcaaggagtacaagtgcaaggtctccaacaaagccctcccagcccccatcgagaaa
accatctccaaagccaaagggcagccccgagaaccacaggtgtacaccctgcccccatcccgggaggagatgaccaagaac
caggtcagcctgacctgcctggtcaaaggcttctatcccagcgacatcgccgtggagtgggagagcaatgggcagccggag
aacaactacaagaccacgcctcccgtgctggactccgacggctccttcttcctcaggagcaagctcaccgtggacaagagc
aggtggcagcaggggaacgtcttctcatgctccgtgatgcatgaggctctgcacaaccactacacgcagaagagcctctcc
ctgtctccgggtaaa
(amino acids)
(SEQ ID NO: 675)
ESPKAQASSVPTAQPQAEGSLAKATTAPATTRNTGRGGEEKKKEKEKEEQEERETKTPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSDGSFFLRSKLTV
DKSRWQQGNVFSCSVMHEALHNHYTQKSLSLSPGK
C2 CAR IgD/FCHingeless/Y407R linker
(DNA)
(SEQ ID NO: 676)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccagcacctgaactcctggggggaccgtcagtcttcctcttccccccaaaaccc
aaggacaccctcatgatctcccggacccctgaggtcacatgcgtggtggtggacgtgagccacgaagaccctgaggtcaag
ttcaactggtacgtggacggcgtggaggtgcataatgccaagacaaagccgcgggaggagcagtacaacagcacgtaccgt
gtggtcagcgtcctcaccgtcctgcaccaggactggctgaatggcaaggagtacaagtgcaaggtctccaacaaagccctc
ccagcccccatcgagaaaaccatctccaaagccaaagggcagccccgagaaccacaggtgtacaccctgcccccatcccgg
gaggagatgaccaagaaccaggtcagcctgacctgcctggtcaaaggcttctatcccagcgacatcgccgtggagtgggag
agcaatgggcagccggagaacaactacaagaccacgcctcccgtgctggactccgacggctccttcttcctcaggagcaag
ctcaccgtggacaagagcaggtggcagcaggggaacgtcttctcatgctccgtgatgcatgaggctctgcacaaccactac
acgcagaagagcctctccctgtctccgggtaaaatctacatctgggcgcccttggccgggacttgtggggtccttctcctg
tcactggttatcaccctttactgcaaacggggcagaaagaaactcctgtatatattcaaacaaccatttatgagaccagta
caaactactcaagaggaagatggctgtagctgccgatttccagaagaagaagaaggaggatgtgaactgagagtgaagttc
agcaggagcgcagacgcccccgcgtacaagcagggccagaaccagctctataacgagctcaatctaggacgaagagaggag
tacgatgttttggacaagagacgtggccgggaccctgagatggggggaaagccgagaaggaagaaccctcaggaaggcctg
tacaatgaactgcagaaagataagatggcggaggcctacagtgagattgggatgaaaggcgagcgccggaggggcaagggg
cacgatggcctttaccagggtctcagtacagccaccaaggacacctacgacgcccttcacatgcaggccctgccccctcgc
tgataa
(amino acids)
(SEQ ID NO: 677)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPAPELLGGPSVFLF
PPKPKDTLMISRTPEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAVEWESNGQPENNYKTTPPVLDSDGS
FFLRSKLTVDKSRWQQGNVFSCSVMHEALHNHYTQKSLSLSPGKIYIWAPLAGTCGVLLLSLVITLYCKRGRKKLLYIFK
QPFMRPVQTTQEEDGCSCRFPEEEEGGCELRVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDTYDALHMQALPPR**
IgD linker
(DNA)
(SEQ ID NO: 678)
gagtctccaaaggcacaggcctcctcagtgcccactgcacaaccccaagcagagggcagcctcgccaaggcaaccacagcc
ccagccaccacccgtaacacaggaagaggcggcgaagagaagaaaaaggagaaggagaaagaggaacaagaagagagagag
acaaagacacca
(amino acids)
(SEQ ID NO: 679)
ESPKAQASSVPTAQPQAEGSLAKATTAPATTRNTGRGGEEKKKEKEKEEQEERETKTP
C2 CAR IgD linker
(DNA)
(SEQ ID NO: 680)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccaatctacatctgggcgcccttggccgggacttgtggggtccttctcctgtca
ctggttatcaccctttactgcaaacggggcagaaagaaactcctgtatatattcaaacaaccatttatgagaccagtacaa
actactcaagaggaagatggctgtagctgccgatttccagaagaagaagaaggaggatgtgaactgagagtgaagttcagc
aggagcgcagacgcccccgcgtacaagcagggccagaaccagctctataacgagctcaatctaggacgaagagaggagtac
gatgttttggacaagagacgtggccgggaccctgagatggggggaaagccgagaaggaagaaccctcaggaaggcctgtac
aatgaactgcagaaagataagatggcggaggcctacagtgagattgggatgaaaggcgagcgccggaggggcaaggggcac
gatggcctttaccagggtctcagtacagccaccaaggacacctacgacgcccttcacatgcaggccctgccccctcgctga
taa
(amino acids)
(SEQ ID NO: 681)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPIYIWAPLAGTCGV
LLLSLVITLYCKRGRKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTATKDTYDALHMQ
ALPPR**
X4 linker
(DNA)
(SEQ ID NO: 682)
gacaagacgcacaccaagccacctaaaccagctccagaactgctcggaggtcctggcaccggaaccggaggacctaccatc
aaaccacctaagccacctaagcctgctcctaacctgctcggaggacct
(amino acids)
(SEQ ID NO: 683)
DKTHTKPPKPAPELLGGPGTGTGGPTIKPPKPPKPAPNLLGGP
C2 CAR X4 linker
(DNA)
(SEQ ID NO: 684)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgacaagacgcacaccaagccacctaaaccagctccagaactgctcggaggtcctggcaccgga
accggaggacctaccatcaaaccacctaagccacctaagcctgctcctaacctgctcggaggacctatctacatctgggcg
cccttggccgggacttgtggggtccttctcctgtcactggttatcaccctttactgcaaacggggcagaaagaaactcctg
tatatattcaaacaaccatttatgagaccagtacaaactactcaagaggaagatggctgtagctgccgatttccagaagaa
gaagaaggaggatgtgaactgagagtgaagttcagcaggagcgcagacgcccccgcgtacaagcagggccagaaccagctc
tataacgagctcaatctaggacgaagagaggagtacgatgttttggacaagagacgtggccgggaccctgagatgggggga
aagccgagaaggaagaaccctcaggaaggcctgtacaatgaactgcagaaagataagatggcggaggcctacagtgagatt
gggatgaaaggcgagcgccggaggggcaaggggcacgatggcctttaccagggtctcagtacagccaccaaggacacctac
gacgcccttcacatgcaggccctgccccctcgctgataa
(amino acids)
(SEQ ID NO: 685)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTDKTHTKPPKPAPELLGGPGTGTGGPTIKPPKPPKPAPNLLGGPIYIWAPLAGTCGVLLLSLVITLYCKRGR
KKLLYIFKQPFMRPVQTTQEEDGCSCRFPEEEEGGCELRVKFSRSADAPAYKQGQNQLYNELNLGRREEYDVLDKRRGRD
PEMGGKPRRKNPQEGLYNELQKDKMAEAYSEIGMKGERRRGKGHDGLYQGLSTATKDTYDALHMQALPPR**
OKT3 scFv
(DNA)
(SEQ ID NO: 686)
caggtgcagctggtgcagagcggaggcggagtggtgcagcctggaagaagcctgcgcctgagctgcaaagcgagcggctat
acctttacccgctataccatgcattgggtgcgccaggcgccgggcaaaggcctggaatggattggctatattaacccgagc
cgcggctataccaactataaccagaaagtgaaagatcgctttaccattagcaccgataaaagcaaaagcaccgcgtttctg
cagatggatagcctgcgcccggaagataccgcggtgtattattgcgcgcgctattatgatgatcattattgcctggattat
tggggccagggcaccaccctgaccgtgagcagcggcggtggcggatccggcggtggcggatccggcggtggcggatccgat
attcagatgacccagagcccgagcagcctgagcgcgagcgtgggcgatcgcgtgaccattacctgcagcgcgagcagcagc
gtgagctatatgaactggtatcagcagaccccgggcaaagcgccgaaacgctggatttatgataccagcaaactggcgagc
ggcgtgccgagccgctttagcggcagcggcagcggcaccgattatacctttaccattagcagcctgcagccggaagatatt
gcgacctattattgccagcagtggagcagcaacccgtttacctttggccagggcaccaaactgcagattacccgctgataa
(amino acids)
(SEQ ID NO: 687)
QVQLVQSGGGVVQPGRSLRLSCKASGYTFTRYTMHWVRQAPGKGLEWIGYINPSRGYTNYNQKVKDRFTISTDKSKSTAF
LQMDSLRPEDTAVYYCARYYDDHYCLDYWGQGTTLTVSSGGGGSGGGGSGGGGSDIQMTQSPSSLSASVGDRVTITCSAS
SSVSYMNWYQQTPGKAPKRWIYDTSKLASGVPSRFSGSGSGTDYTFTISSLQPEDIATYYCQQWSSNPFTFGQGTKLQIT
R**
C2-FC-OKT3
(DNA)
(SEQ ID NO: 688)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagcccaaatcttgtgacaaaactcacacatgcccaccgtgcccagcacctgaactcctgggg
ggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaag
gagtacaagtgcaaggtctccaacaaagccctcccagcccccatcgagaaaaccatctccaaagccaaagggcagccccga
gaaccacaggtgtacaccctgcccccatcccgggaggagatgaccaagaaccaggtcagcctgacctgcctggtcaaaggc
ttctatcccagcgacatcgccgtggagtgggagagcaatgggcagccggagaacaactacaagaccacgcctcccgtgctg
gactccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaggcggtggcggatcc
caggtgcagctggtgcagagcggaggcggagtggtgcagcctggaagaagcctgcgcctgagctgcaaagcgagcggctat
acctttacccgctataccatgcattgggtgcgccaggcgccgggcaaaggcctggaatggattggctatattaacccgagc
cgcggctataccaactataaccagaaagtgaaagatcgctttaccattagcaccgataaaagcaaaagcaccgcgtttctg
cagatggatagcctgcgcccggaagataccgcggtgtattattgcgcgcgctattatgatgatcattattgcctggattat
tggggccagggcaccaccctgaccgtgagcagcggcggtggcggatccggcggtggcggatccggcggtggcggatccgat
attcagatgacccagagcccgagcagcctgagcgcgagcgtgggcgatcgcgtgaccattacctgcagcgcgagcagcagc
gtgagctatatgaactggtatcagcagaccccgggcaaagcgccgaaacgctggatttatgataccagcaaactggcgagc
ggcgtgccgagccgctttagcggcagcggcagcggcaccgattatacctttaccattagcagcctgcagccggaagatatt
gcgacctattattgccagcagtggagcagcaacccgtttacctttggccagggcaccaaactgcagattacccgctgataa
(amino acids)
(SEQ ID NO: 689)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTEPKSCDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSLSLSPG
KGGGGSQVQLVQSGGGVVQPGRSLRLSCKASGYTFTRYTMHWVRQAPGKGLEWIGYINPSRGYTNYNQKVKDRFTISTDK
SKSTAFLQMDSLRPEDTAVYYCARYYDDHYCLDYWGQGTTLTVSSGGGGSGGGGSGGGGSDIQMTQSPSSLSASVGDRVT
ITCSASSSVSYMNWYQQTPGKAPKRWIYDTSKLASGVPSRFSGSGSGTDYTFTISSLQPEDIATYYCQQWSSNPFTFGQG
TKLQITR**
C2-IgD/FC-OKT3
(DNA)
(SEQ ID NO: 690)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccagagcccaaatcttgtgacaaaactcacacatgcccaccgtgcccagcacct
gaactcctggggggaccgtcagtcttcctcttccccccaaaacccaaggacaccctcatgatctcccggacccctgaggtc
acatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactgg
ctgaatggcaaggagtacaagtgcaaggtctccaacaaagccctcccagcccccatcgagaaaaccatctccaaagccaaa
gggcagccccgagaaccacaggtgtacaccctgcccccatcccgggaggagatgaccaagaaccaggtcagcctgacctgc
ctggtcaaaggcttctatcccagcgacatcgccgtggagtgggagagcaatgggcagccggagaacaactacaagaccacg
cctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaaggc
ggtggcggatcccaggtgcagctggtgcagagcggaggcggagtggtgcagcctggaagaagcctgcgcctgagctgcaaa
gcgagcggctatacctttacccgctataccatgcattgggtgcgccaggcgccgggcaaaggcctggaatggattggctat
attaacccgagccgcggctataccaactataaccagaaagtgaaagatcgctttaccattagcaccgataaaagcaaaagc
accgcgtttctgcagatggatagcctgcgcccggaagataccgcggtgtattattgcgcgcgctattatgatgatcattat
tgcctggattattggggccagggcaccaccctgaccgtgagcagcggcggtggcggatccggcggtggcggatccggcggt
ggcggatccgatattcagatgacccagagcccgagcagcctgagcgcgagcgtgggcgatcgcgtgaccattacctgcagc
gcgagcagcagcgtgagctatatgaactggtatcagcagaccccgggcaaagcgccgaaacgctggatttatgataccagc
aaactggcgagcggcgtgccgagccgctttagcggcagcggcagcggcaccgattatacctttaccattagcagcctgcag
ccggaagatattgcgacctattattgccagcagtggagcagcaacccgtttacctttggccagggcaccaaactgcagatt
acccgctgataa
(amino acids)
(SEQ ID NO: 691)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPEPKSCDKTHTCPP
CPAPELLGGPSVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGKGGGGSQVQLVQSGGGVVQPGR
SLRLSCKASGYTFTRYTMHWVRQAPGKGLEWIGYINPSRGYTNYNQKVKDRFTISTDKSKSTAFLQMDSLRPEDTAVYYC
ARYYDDHYCLDYWGQGTTLTVSSGGGGSGGGGSGGGGSDIQMTQSPSSLSASVGDRVTITCSASSSVSYMNWYQQTPGKA
PKRWIYDTSKLASGVPSRFSGSGSGTDYTFTISSLQPEDIATYYCQQWSSNPFTFGQGTKLQITR**
C2-FCHingeless/Y407R-OKT3
(DNA)
(SEQ ID NO: 692)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgcacctgaactcctggggggaccgtcagtcttcctcttccccccaaaacccaaggacaccctc
atgatctcccggacccctgaggtcacatgcgtggtggtggacgtgagccacgaagaccctgaggtcaagttcaactggtac
gtggacggcgtggaggtgcataatgccaagacaaagccgcgggaggagcagtacaacagcacgtaccgtgtggtcagcgtc
ctcaccgtcctgcaccaggactggctgaatggcaaggagtacaagtgcaaggtctccaacaaagccctcccagcccccatc
gagaaaaccatctccaaagccaaagggcagccccgagaaccacaggtgtacaccctgcccccatcccgggaggagatgacc
aagaaccaggtcagcctgacctgcctggtcaaaggcttctatcccagcgacatcgccgtggagtgggagagcaatgggcag
ccggagaacaactacaagaccacgcctcccgtgctggactccgacggctccttcttcctcaggagcaagctcaccgtggac
aagagcaggtggcagcaggggaacgtcttctcatgctccgtgatgcatgaggctctgcacaaccactacacgcagaagagc
ctctccctgtctccgggtaaaggcggtggcggatcccaggtgcagctggtgcagagcggaggcggagtggtgcagcctgga
agaagcctgcgcctgagctgcaaagcgagcggctatacctttacccgctataccatgcattgggtgcgccaggcgccgggc
aaaggcctggaatggattggctatattaacccgagccgcggctataccaactataaccagaaagtgaaagatcgctttacc
attagcaccgataaaagcaaaagcaccgcgtttctgcagatggatagcctgcgcccggaagataccgcggtgtattattgc
gcgcgctattatgatgatcattattgcctggattattggggccagggcaccaccctgaccgtgagcagcggcggtggcgga
tccggcggtggcggatccggcggtggcggatccgatattcagatgacccagagcccgagcagcctgagcgcgagcgtgggc
gatcgcgtgaccattacctgcagcgcgagcagcagcgtgagctatatgaactggtatcagcagaccccgggcaaagcgccg
aaacgctggatttatgataccagcaaactggcgagcggcgtgccgagccgctttagcggcagcggcagcggcaccgattat
acctttaccattagcagcctgcagccggaagatattgcgacctattattgccagcagtggagcagcaacccgtttaccttt
ggccagggcaccaaactgcagattacccgctgataa
(amino acids)
(SEQ ID NO: 693)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTAPELLGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQVSLTCLVKGFYPSDIAVEW
ESNGQPENNYKTTPPVLDSDGSFFLRSKLTVDKSRWQQGNVFSCSVMHEALHNHYTQKSLSLSPGKGGGGSQVQLVQSGG
GVVQPGRSLRLSCKASGYTFTRYTMHWVRQAPGKGLEWIGYINPSRGYTNYNQKVKDRFTISTDKSKSTAFLQMDSLRPE
DTAVYYCARYYDDHYCLDYWGQGTTLTVSSGGGGSGGGGSGGGGSDIQMTQSPSSLSASVGDRVTITCSASSSVSYMNWY
QQTPGKAPKRWIYDTSKLASGVPSRFSGSGSGTDYTFTISSLQPEDIATYYCQQWSSNPFTFGQGTKLQITR**
C2-IgD/FCHingeless/Y407R-OKT3
(DNA)
(SEQ ID NO: 694)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccagcacctgaactcctggggggaccgtcagtcttcctcttccccccaaaaccc
aaggacaccctcatgatctcccggacccctgaggtcacatgcgtggtggtggacgtgagccacgaagaccctgaggtcaag
ttcaactggtacgtggacggcgtggaggtgcataatgccaagacaaagccgcgggaggagcagtacaacagcacgtaccgt
gtggtcagcgtcctcaccgtcctgcaccaggactggctgaatggcaaggagtacaagtgcaaggtctccaacaaagccctc
ccagcccccatcgagaaaaccatctccaaagccaaagggcagccccgagaaccacaggtgtacaccctgcccccatcccgg
gaggagatgaccaagaaccaggtcagcctgacctgcctggtcaaaggcttctatcccagcgacatcgccgtggagtgggag
agcaatgggcagccggagaacaactacaagaccacgcctcccgtgctggactccgacggctccttcttcctcaggagcaag
ctcaccgtggacaagagcaggtggcagcaggggaacgtcttctcatgctccgtgatgcatgaggctctgcacaaccactac
acgcagaagagcctctccctgtctccgggtaaaggcggtggcggatcccaggtgcagctggtgcagagcggaggcggagtg
gtgcagcctggaagaagcctgcgcctgagctgcaaagcgagcggctatacctttacccgctataccatgcattgggtgcgc
caggcgccgggcaaaggcctggaatggattggctatattaacccgagccgcggctataccaactataaccagaaagtgaaa
gatcgctttaccattagcaccgataaaagcaaaagcaccgcgtttctgcagatggatagcctgcgcccggaagataccgcg
gtgtattattgcgcgcgctattatgatgatcattattgcctggattattggggccagggcaccaccctgaccgtgagcagc
ggcggtggcggatccggcggtggcggatccggcggtggcggatccgatattcagatgacccagagcccgagcagcctgagc
gcgagcgtgggcgatcgcgtgaccattacctgcagcgcgagcagcagcgtgagctatatgaactggtatcagcagaccccg
ggcaaagcgccgaaacgctggatttatgataccagcaaactggcgagcggcgtgccgagccgctttagcggcagcggcagc
ggcaccgattatacctttaccattagcagcctgcagccggaagatattgcgacctattattgccagcagtggagcagcaac
ccgtttacctttggccagggcaccaaactgcagattacccgctgataa
(amino acids)
(SEQ ID NO: 695)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSSGGGGSGGGGSGGGGSDIVLTQSPASLAVSPGQRATITCRAS
KSVSTSGYSYMHWYQQKPGQPPKLLIYLASNLESGVPARFSGSGSGTDFTLTINPVEANDTANYYCQHSRELPFTFGGGTK
VEIKRTESPKAQASSVPTAQPQAEGSLAKATTAPATTRNTGRGGEEKKKEKEKEEQEERETKTPAPELLGGPSVFLFPPKP
KDTLMISRTPEVTCVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPPVLDSDGSFFLRSK
LTVDKSRWQQGNVFSCSVMHEALHNHYTQKSLSLSPGKGGGGSQVQLVQSGGGVVQPGRSLRLSCKASGYTFTRYTMHWVR
QAPGKGLEWIGYINPSRGYTNYNQKVKDRFTISTDKSKSTAFLQMDSLRPEDTAVYYCARYYDDHYCLDYWGQGTTLTVSS
GGGGSGGGGSGGGGSDIQMTQSPSSLSASVGDRVTITCSASSSVSYMNWYQQTPGKAPKRWIYDTSKLASGVPSRFSGSGS
GTDYTFTISSLQPEDIATYYCQQWSSNPFTFGQGTKLQITR**
C2-IgD-OKT3
(DNA)
(SEQ ID NO: 696)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccaggcggtggcggatcccaggtgcagctggtgcagagcggaggcggagtggtg
cagcctggaagaagcctgcgcctgagctgcaaagcgagcggctatacctttacccgctataccatgcattgggtgcgccag
gcgccgggcaaaggcctggaatggattggctatattaacccgagccgcggctataccaactataaccagaaagtgaaagat
cgctttaccattagcaccgataaaagcaaaagcaccgcgtttctgcagatggatagcctgcgcccggaagataccgcggtg
tattattgcgcgcgctattatgatgatcattattgcctggattattggggccagggcaccaccctgaccgtgagcagcggc
ggtggcggatccggcggtggcggatccggcggtggcggatccgatattcagatgacccagagcccgagcagcctgagcgcg
agcgtgggcgatcgcgtgaccattacctgcagcgcgagcagcagcgtgagctatatgaactggtatcagcagaccccgggc
aaagcgccgaaacgctggatttatgataccagcaaactggcgagcggcgtgccgagccgctttagcggcagcggcagcggc
accgattatacctttaccattagcagcctgcagccggaagatattgcgacctattattgccagcagtggagcagcaacccg
tttacctttggccagggcaccaaactgcagattacccgctgataa
(amino acids)
(SEQ ID NO: 697)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPGGGGSQVQLVQSG
GGVVQPGRSLRLSCKASGYTFTRYTMHWVRQAPGKGLEWIGYINPSRGYTNYNQKVKDRFTISTDKSKSTAFLQMDSLRP
EDTAVYYCARYYDDHYCLDYWGQGTTLTVSSGGGGSGGGGSGGGGSDIQMTQSPSSLSASVGDRVTITCSASSSVSYMNW
YQQTPGKAPKRWIYDTSKLASGVPSRFSGSGSGTDYTFTISSLQPEDIATYYCQQWSSNPFTFGQGTKLQITR**
C2-X4-OKT3
(DNA)
(SEQ ID NO: 698)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgacaagacgcacaccaagccacctaaaccagctccagaactgctcggaggtcctggcaccgga
accggaggacctaccatcaaaccacctaagccacctaagcctgctcctaacctgctcggaggacctggcggtggcggatcc
caggtgcagctggtgcagagcggaggcggagtggtgcagcctggaagaagcctgcgcctgagctgcaaagcgagcggctat
acctttacccgctataccatgcattgggtgcgccaggcgccgggcaaaggcctggaatggattggctatattaacccgagc
cgcggctataccaactataaccagaaagtgaaagatcgctttaccattagcaccgataaaagcaaaagcaccgcgtttctg
cagatggatagcctgcgcccggaagataccgcggtgtattattgcgcgcgctattatgatgatcattattgcctggattat
tggggccagggcaccaccctgaccgtgagcagcggcggtggcggatccggcggtggcggatccggcggtggcggatccgat
attcagatgacccagagcccgagcagcctgagcgcgagcgtgggcgatcgcgtgaccattacctgcagcgcgagcagcagc
gtgagctatatgaactggtatcagcagaccccgggcaaagcgccgaaacgctggatttatgataccagcaaactggcgagc
ggcgtgccgagccgctttagcggcagcggcagcggcaccgattatacctttaccattagcagcctgcagccggaagatatt
gcgacctattattgccagcagtggagcagcaacccgtttacctttggccagggcaccaaactgcagattacccgctgataa
(amino acids)
(SEQ ID NO: 699)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTDKTHTKPPKPAPELLGGPGTGTGGPTIKPPKPPKPAPNLLGGPGGGGSQVQLVQSGGGVVQPGRSLRLSCK
ASGYTFTRYTMHWVRQAPGKGLEWIGYINPSRGYTNYNQKVKDRFTISTDKSKSTAFLQMDSLRPEDTAVYYCARYYDDH
YCLDYWGQGTTLTVSSGGGGSGGGGSGGGGSDIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFGQGTKLQITR**
C2-MMP9
(DNA)
(SEQ ID NO: 700)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactggcggtggcggatccagcctctggcagcccctggtcctggtgctcctggtgctgggctgctgc
tttgctgcccccagacagcgccagtccacccttgtgctcttccctggagacctgagaaccaatctcaccgacaggcagctg
gcagaggaatacctgtaccgctatggttacactcgggtggcagagatgcgtggagagtcgaaatctctggggcctgcgctg
ctgcttctccagaagcaactgtccctgcccgagaccggtgagctggatagcgccacgctgaaggccatgcgaaccccacgg
tgcggggtcccagacctgggcagattccaaacctttgagggcgacctcaagtggcaccaccacaacatcacctattggatc
caaaactactcggaagacttgccgcgggcggtgattgacgacgcctttgcccgcgccttcgcactgtggagcgcggtgacg
ccgctcaccttcactcgcgtgtacagccgggacgcagacatcgtcatccagtttggtgtcgcggagcacggagacgggtat
cccttcgacgggaaggacgggctcctggcacacgcctttcctcctggccccggcattcagggagacgcccatttcgacgat
gacgagttgtggtccctgggcaagggcgtcgtggttccaactcggtttggaaacgcagatggcgcggcctgccacttcccc
ttcatcttcgagggccgctcctactctgcctgcaccaccgacggtcgctccgacggcttgccctggtgcagtaccacggcc
aactacgacaccgacgaccggtttggcttctgccccagcgagagactctacacccaggacggcaatgctgatgggaaaccc
tgccagtttccattcatcttccaaggccaatcctactccgcctgcaccacggacggtcgctccgacggctaccgctggtgc
gccaccaccgccaactacgaccgggacaagctcttcggcttctgcccgacccgagctgactcgacggtgatggggggcaac
tcggcgggggagctgtgcgtcttccccttcactttcctgggtaaggagtactcgacctgtaccagcgagggccgcggagat
gggcgcctctggtgcgctaccacctcgaactttgacagcgacaagaagtggggcttctgcccggaccaaggatacagtttg
ttcctcgtggcggcgcatgagttcggccacgcgctgggcttagatcattcctcagtgccggaggcgctcatgtaccctatg
taccgcttcactgaggggccccccttgcataaggacgacgtgaatggcatccggcacctctatggtcctcgccctgaacct
gagccacggcctccaaccaccaccacaccgcagcccacggctcccccgacggtctgccccaccggaccccccactgtccac
ccctcagagcgccccacagctggccccacaggtcccccctcagctggccccacaggtccccccactgctggcccttctacg
gccactactgtgcctttgagtccggtggacgatgcctgcaacgtgaacatcttcgacgccatcgcggagattgggaaccag
ctgtatttgttcaaggatgggaagtactggcgattctctgagggcagggggagccggccgcagggccccttccttatcgcc
gacaagtggcccgcgctgccccgcaagctggactcggtctttgaggagcggctctccaagaagcttttcttcttctctggg
cgccaggtgtgggtgtacacaggcgcgtcggtgctgggcccgaggcgtctggacaagctgggcctgggagccgacgtggcc
caggtgaccggggccctccggagtggcagggggaagatgctgctgttcagcgggcggcgcctctggaggttcgacgtgaag
gcgcagatggtggatccccggagcgccagcgaggtggaccggatgttccccggggtgcctttggacacgcacgacgtcttc
cagtaccgagagaaagcctatttctgccaggaccgcttctactggcgcgtgagttcccggagtgagttgaaccaggtggac
caagtgggctacgtgacctatgacatcctgcagtgccctgaggacgattacaaggatgacgacgataagtgataa
(amino acids)
(SEQ ID NO: 701)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTGGGGSSLWQPLVLVLLVLGCCFAAPRQRQSTLVLFPGDLRTNLTDRQLAEEYLYRYGYTRVAEMRGESKSL
GPALLLLQKQLSLPETGELDSATLKAMRTPRCGVPDLGRFQTFEGDLKWHHHNITYWIQNYSEDLPRAVIDDAFARAFAL
WSAVTPLTFTRVYSRDADIVIQFGVAEHGDGYPFDGKDGLLAHAFPPGPGIQGDAHFDDDELWSLGKGVVVPTRFGNADG
AACHFPFIFEGRSYSACTTDGRSDGLPWCSTTANYDTDDRFGFCPSERLYTQDGNADGKPCQFPFIFQGQSYSACTTDGR
SDGYRWCATTANYDRDKLFGFCPTRADSTVMGGNSAGELCVFPFTFLGKEYSTCTSEGRGDGRLWCATTSNFDSDKKWGF
CPDQGYSLFLVAAHEFGHALGLDHSSVPEALMYPMYRFTEGPPLHKDDVNGIRHLYGPRPEPEPRPPTTTTPQPTAPPTV
CPTGPPTVHPSERPTAGPTGPPSAGPTGPPTAGPSTATTVPLSPVDDACNVNIFDAIAEIGNQLYLFKDGKYWRFSEGRG
SRPQGPFLIADKWPALPRKLDSVFEERLSKKLFFFSGRQVWVYTGASVLGPRRLDKLGLGADVAQVTGALRSGRGKMLLF
SGRRLWRFDVKAQMVDPRSASEVDRMFPGVPLDTHDVFQYREKAYFCQDRFYWRVSSRSELNQVDQVGYVTYDILQCPED
DYKDDDDK**
C2-MMP9cat
(DNA)
(SEQ ID NO: 702)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactggcggtggcggatccttccaaacctttgagggcgacctcaagtggcaccaccacaacatcacc
tattggatccaaaactactcggaagacttgccgcgggcggtgattgacgacgcctttgcccgcgccttcgcactgtggagc
gcggtgacgccgctcaccttcactcgcgtgtacagccgggacgcagacatcgtcatccagtttggtgtcgcggagcacgga
gacgggtatcccttcgacgggaaggacgggctcctggcacacgcctttcctcctggccccggcattcagggagacgcccat
ttcgacgatgacgagttgtggtccctgggcaagggcgtcgtggttccaactcggtttggaaacgcagatggcgcggcctgc
cacttccccttcatcttcgagggccgctcctactctgcctgcaccaccgacggtcgctccgacggcttgccctggtgcagt
accacggccaactacgacaccgacgaccggtttggcttctgccccagcgagagactctacacccaggacggcaatgctgat
gggaaaccctgccagtttccattcatcttccaaggccaatcctactccgcctgcaccacggacggtcgctccgacggctac
cgctggtgcgccaccaccgccaactacgaccgggacaagctcttcggcttctgcccgacccgagctgactcgacggtgatg
gggggcaactcggcgggggagctgtgcgtcttccccttcactttcctgggtaaggagtactcgacctgtaccagcgagggc
cgcggagatgggcgcctctggtgcgctaccacctcgaactttgacagcgacaagaagtggggcttctgcccggaccaagga
tacagtttgttcctcgtggcggcgcatgagttcggccacgcgctgggcttagatcattcctcagtgccggaggcgctcatg
taccctatgtaccgcttcactgaggggccccccttgcataaggacgacgtgaatggcatccggcacctctatggtcctcgc
cctgaacctgattacaaggatgacgacgataagtgataa
(amino acids)
(SEQ ID NO: 703)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTGGGGSFQTFEGDLKWHHHNITYWIQNYSEDLPRAVIDDAFARAFALWSAVTPLTFTRVYSRDADIVIQFGV
AEHGDGYPFDGKDGLLAHAFPPGPGIQGDAHFDDDELWSLGKGVVVPTRFGNADGAACHFPFIFEGRSYSACTTDGRSDG
LPWCSTTANYDTDDRFGFCPSERLYTQDGNADGKPCQFPFIFQGQSYSACTTDGRSDGYRWCATTANYDRDKLFGFCPTR
ADSTVMGGNSAGELCVFPFTFLGKEYSTCTSEGRGDGRLWCATTSNFDSDKKWGFCPDQGYSLFLVAAHEFGHALGLDHS
SVPEALMYPMYRFTEGPPLHKDDVNGIRHLYGPRPEPDYK
DDDDK**
Other linkers for BITES between two scFvs and between C2 and MMP9 include but
are not limited to those shown as SEQ ID NOS: 705, 707, 709, 711, 713, 715 and
717.
[G₄S₁]x2 linker sequence:
(DNA)
(SEQ ID NO: 704)
ggcggtggcggatccggcggtggcggatcc
(amino acids)
(SEQ ID NO: 705)
GGGGSGGGGS
[G₄S₁]x3 linker sequence:
(DNA)
(SEQ ID NO: 706)
ggcggtggcggatccggcggtggcggatccggcggtggcggatcc
(amino acids)
(SEQ ID NO: 707)
GGGGSGGGGSGGGGS
Long GS linker sequence:
(DNA)
(SEQ ID NO: 708)
ggcggtggaagcggcggtggcggatccggcagcggcggaagcggcggtggcggatccggcggtgga
(amino acids)
(SEQ ID NO: 709)
GGGSGGGGSGSGGSGGGGSGGG
13 aa GS linker sequence:
(DNA)
(SEQ ID NO: 710)
ggcggtggatccggcggtggcggatccggcggtggatcc
(amino acids)
(SEQ ID NO: 711)
GGGSGGGGSGGGS
8 aa GS linker sequence:
(DNA)
(SEQ ID NO: 712)
ggcggttccggcggtggatccgga
(amino acids)
(SEQ ID NO: 713)
GGSGGGSG
12 aa GS linker sequence:
(DNA)
(SEQ ID NO: 714)
ggcggttccggcggtggatccggcggtggcggatccgga
(amino acids)
(SEQ ID NO: 715)
GGSGGGSGGGSG
24 aa GS linker sequence:
(DNA)
(SEQ ID NO: 716)
ggcggttccggcggtggatccggcggtggcggatccggaggcggttccggcggtggatccggcggtggcggatccgga
(amino acids)
(SEQ ID NO: 717)
GGSGGGSGGGSGGGSGGGSGGGSG
CAR-T C2 CD8/CD8/4-1BB/CD3z #44
N-CD81s-huMNC2scFv-CD8ecd fragment- CD8 transmembrane- 4-1BB- CD3zeta-C
(DNA)
(SEQ ID NO: 718)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactacaacaacccctgcccccagacctcctaccccagcccctacaattgccagccagcctctgagcctgaggc
ccgaggcttgtagacctgctgctggcggagccgtgcacaccagaggactggatttcgcctgcgacatctacatctgggcg
cccttggccgggacttgtggggtccttctcctgtcactggttatcaccctttactgcaaacggggcagaaagaaactcct
gtatatattcaaacaaccatttatgagaccagtacaaactactcaagaggaagatggctgtagctgccgatttccagaag
aagaagaaggaggatgtgaactgagagtgaagttcagcaggagcgcagacgcccccgcgtacaagcagggccagaaccag
ctctataacgagctcaatctaggacgaagagaggagtacgatgttttggacaagagacgtggccgggaccctgagatggg
gggaaagccgagaaggaagaaccctcaggaaggcctgtacaatgaactgcagaaagataagatggcggaggcctacagtg
agattgggatgaaaggcgagcgccggaggggcaaggggcacgatggcctttaccagggtctcagtacagccaccaaggac
acctacgacgcccttcacatgcaggccctgccccctcgctgataa
(amino acids)
(SEQ ID NO: 719)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRRGKGHDGLYQGLSTATKD
TYDALHMQALPPR**
CAR-T IgK C2 CD8/CD8/4-1BB/CD3z #45
N-IgK1s-huMNC2scFv-CD8ecd fragment- CD8 transmembrane- 4-1BB- CD3zeta-C
(DNA)
(SEQ ID NO: 720)
atggagacagacacactcctgctatgggtactgctgctctgggttccaggttccactggtgaggtgcagctggtggagtc
tgggggaggcctggtcaagcctggggggtccctgagactctcctgtgcagcctctggattcaccttcagtggctatgcca
tgagctgggtccgccaggctccagggaaggggctggagtgggtctcaaccattagtagtggcggaacctacatatactac
cccgactcagtgaagggccgattcaccatctccagagacaacgccaagaactcactgtatctgcaaatgaacagcctgag
agccgaggacacggccgtgtattactgtgcgagacttgggggggataattactacgaatacttcgatgtctggggcaaag
ggaccacggtcaccgtctcctccggcggtggcggatccggcggtggcggatccggcggtggcggatccgacattgtgctg
acccagtctccagcctccttggccgtgtctccaggacagagggccaccatcacctgcagagccagtaagagtgtcagtac
cagcggatactcctacatgcactggtatcagcagaaaccaggacaacctcctaaactcctgatttacctggcatccaatc
tggagagcggggtcccagccaggttcagcggcagtgggtctgggaccgatttcaccctcacaattaatcctgtggaagct
aatgatactgcaaattattactgtcagcacagtagggagctgcctttcacattcggcggagggaccaaggtggagatcaa
acgaactacaacaacccctgcccccagacctcctaccccagcccctacaattgccagccagcctctgagcctgaggcccg
aggcttgtagacctgctgctggcggagccgtgcacaccagaggactggatttcgcctgcgacatctacatctgggcgccc
ttggccgggacttgtggggtccttctcctgtcactggttatcaccctttactgcaaacggggcagaaagaaactcctgta
tatattcaaacaaccatttatgagaccagtacaaactactcaagaggaagatggctgtagctgccgatttccagaagaag
aagaaggaggatgtgaactgagagtgaagttcagcaggagcgcagacgcccccgcgtacaagcagggccagaaccagctc
tataacgagctcaatctaggacgaagagaggagtacgatgttttggacaagagacgtggccgggaccctgagatgggggg
aaagccgagaaggaagaaccctcaggaaggcctgtacaatgaactgcagaaagataagatggcggaggcctacagtgaga
ttgggatgaaaggcgagcgccggaggggcaaggggcacgatggcctttaccagggtctcagtacagccaccaaggacacc
tacgacgcccttcacatgcaggccctgccccctcgctgataa
(amino acids)
(SEQ ID NO: 721)
METDTLLLWVLLLWVPGSTGEVQLVESGGGLVKPGGSLRLSCAASGFTFSGYAMSWVRQAPGKGLEWVSTISSGGTYIYY
PDSVKGRFTISRDNAKNSLYLQMNSLRAEDTAVYYCARLGGDNYYEYFDVWGKGTTVTVSSGGGGSGGGGSGGGGSDIVL
TQSPASLAVSPGQRATITCRASKSVSTSGYSYMHWYQQKPGQPPKLLIYLASNLESGVPARFSGSGSGTDFTLTINPVEA
NDTANYYCQHSRELPFTFGGGTKVEIKRTTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T C2 op CD8/CD8/4-1BB/CD3z #46
N-CD81s-huMNC2scFv codon optimized-CD8ecd fragment- CD8 transmembrane- 4-1BB-
CD3zeta-C
(DNA)
(SEQ ID NO: 722)
atggccttaccagtgaccgccttgctcctgccgctggccttgctgctccacgccgccaggccggaagtgcagctggtgga
atctggcggcggactcgtgaagcctggcggctctctgagactgagctgtgccgccagcggcttcacctttagcggctacg
ccatgagctgggtgcgccaggctcctggcaaaggcctggaatgggtgtccaccatctctagcggcggcacctacatctac
taccccgacagcgtgaagggccggttcaccatcagccgggacaacgccaagaacagcctgtacctgcagatgaactccct
gcgggccgaggacaccgccgtgtactattgtgctagactgggcggcgacaactactacgagtacttcgacgtgtggggca
agggcaccaccgtgacagtgtctagcggaggcggaggatcaggcggcggaggaagtggcggagggggatctgatatcgtg
ctgacccagagccctgccagcctggctgtgtctcctggacagagggccaccatcacctgtcgggccagcaagagcgtgtc
cacctccggctacagctacatgcactggtatcagcagaagcccggccagccccccaagctgctgatctacctggccagca
acctggaaagcggcgtgcccgctagattttccggctctggcagcggcaccgacttcaccctgaccatcaaccccgtggaa
gccaacgacaccgccaattactactgccagcacagcagagagctgcccttcaccttcggcggaggcaccaaggtggaaat
caagcggaccacaacaacccctgcccccagacctcctaccccagcccctacaattgccagccagcctctgagcctgaggc
ccgaggcttgtagacctgctgctggcggagccgtgcacaccagaggactggatttcgcctgcgacatctacatctgggcg
cccttggccgggacttgtggggtccttctcctgtcactggttatcaccctttactgcaaacggggcagaaagaaactcct
gtatatattcaaacaaccatttatgagaccagtacaaactactcaagaggaagatggctgtagctgccgatttccagaag
aagaagaaggaggatgtgaactgagagtgaagttcagcaggagcgcagacgcccccgcgtacaagcagggccagaaccag
ctctataacgagctcaatctaggacgaagagaggagtacgatgttttggacaagagacgtggccgggaccctgagatggg
gggaaagccgagaaggaagaaccctcaggaaggcctgtacaatgaactgcagaaagataagatggcggaggcctacagtg
agattgggatgaaaggcgagcgccggaggggcaaggggcacgatggcctttaccagggtctcagtacagccaccaaggac
acctacgacgcccttcacatgcaggccctgccccctcgctgataa
(amino acids)
(SEQ ID NO: 723)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRRGKGHDGLYQGLSTATKD
TYDALHMQALPPR**
CAR-T IgK C2 op CD8/CD8/4-1BB/CD3z #47
N-IgK1s-huMNC2scFv codon optimized-CD8ecd fragment- CD8 transmembrane- 4-1BB-
CD3zeta-C
(DNA)
(SEQ ID NO: 724)
atggagacagacacactcctgctatgggtactgctgctctgggttccaggttccactggtgaagtgcagctggtggaatc
tggcggcggactcgtgaagcctggcggctctctgagactgagctgtgccgccagcggcttcacctttagcggctacgcca
tgagctgggtgcgccaggctcctggcaaaggcctggaatgggtgtccaccatctctagcggcggcacctacatctactac
cccgacagcgtgaagggccggttcaccatcagccgggacaacgccaagaacagcctgtacctgcagatgaactccctgcg
ggccgaggacaccgccgtgtactattgtgctagactgggcggcgacaactactacgagtacttcgacgtgtggggcaagg
gcaccaccgtgacagtgtctagcggaggcggaggatcaggcggcggaggaagtggcggagggggatctgatatcgtgctg
acccagagccctgccagcctggctgtgtctcctggacagagggccaccatcacctgtcgggccagcaagagcgtgtccac
ctccggctacagctacatgcactggtatcagcagaagcccggccagccccccaagctgctgatctacctggccagcaacc
tggaaagcggcgtgcccgctagattttccggctctggcagcggcaccgacttcaccctgaccatcaaccccgtggaagcc
aacgacaccgccaattactactgccagcacagcagagagctgcccttcaccttcggcggaggcaccaaggtggaaatcaa
gcggaccacaacaacccctgcccccagacctcctaccccagcccctacaattgccagccagcctctgagcctgaggcccg
aggcttgtagacctgctgctggcggagccgtgcacaccagaggactggatttcgcctgcgacatctacatctgggcgccc
ttggccgggacttgtggggtccttctcctgtcactggttatcaccctttactgcaaacggggcagaaagaaactcctgta
tatattcaaacaaccatttatgagaccagtacaaactactcaagaggaagatggctgtagctgccgatttccagaagaag
aagaaggaggatgtgaactgagagtgaagttcagcaggagcgcagacgcccccgcgtacaagcagggccagaaccagctc
tataacgagctcaatctaggacgaagagaggagtacgatgttttggacaagagacgtggccgggaccctgagatgggggg
aaagccgagaaggaagaaccctcaggaaggcctgtacaatgaactgcagaaagataagatggcggaggcctacagtgaga
ttgggatgaaaggcgagcgccggaggggcaaggggcacgatggcctttaccagggtctcagtacagccaccaaggacacc
tacgacgcccttcacatgcaggccctgccccctcgctgataa
(amino acids)
(SEQ ID NO: 725)
METDTLLLWVLLLWVPGSTGEVQLVESGGGLVKPGGSLRLSCAASGFTFSGYAMSWVRQAPGKGLEWVSTISSGGTYIYY
PDSVKGRFTISRDNAKNSLYLQMNSLRAEDTAVYYCARLGGDNYYEYFDVWGKGTTVTVSSGGGGSGGGGSGGGGSDIVL
TQSPASLAVSPGQRATITCRASKSVSTSGYSYMHWYQQKPGQPPKLLIYLASNLESGVPARFSGSGSGTDFTLTINPVEA
NDTANYYCQHSRELPFTFGGGTKVEIKRTTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T C2 CD8/CD8/4-1BB/CD3z op #48
N-CD81s-huMNC2scFv -CD8ecd fragment- CD8 transmembrane- 4-1BB- CD3zeta (all
domains codon optimized)-C
(DNA)
(SEQ ID NO: 726)
atggccttaccagtgaccgccttgctcctgccgctggccttgctgctccacgccgccaggccggaagtgcagctggtgga
atctggcggcggactcgtgaagcctggcggctctctgagactgagctgtgccgccagcggcttcacctttagcggctacg
ccatgagctgggtgcgccaggctcctggcaaaggcctggaatgggtgtccaccatctctagcggcggcacctacatctac
taccccgacagcgtgaagggccggttcaccatcagccgggacaacgccaagaacagcctgtacctgcagatgaactccct
gcgggccgaggacaccgccgtgtactattgtgctagactgggcggcgacaactactacgagtacttcgacgtgtggggca
agggcaccaccgtgacagtgtctagcggaggcggaggatcaggcggcggaggaagtggcggagggggatctgatatcgtg
ctgacccagagccctgccagcctggctgtgtctcctggacagagggccaccatcacctgtcgggccagcaagagcgtgtc
cacctccggctacagctacatgcactggtatcagcagaagcccggccagccccccaagctgctgatctacctggccagca
acctggaaagcggcgtgcccgctagattttccggctctggcagcggcaccgacttcaccctgaccatcaaccccgtggaa
gccaacgacaccgccaattactactgccagcacagcagagagctgcccttcaccttcggcggaggcaccaaggtggaaat
caagcggaccacaacaacccctgcccccagacctcctaccccagcccctacaattgccagccagcctctgagcctgaggc
ccgaggcttgtagacctgctgctggcggagccgtgcacaccagaggactggatttcgcctgcgacatctacatctgggcc
cctctggccggcacatgtggcgtgctgctgctgagcctcgtgatcaccctgtactgcaagcggggcagaaagaagctgct
gtacatcttcaagcagcccttcatgcggcccgtgcagaccacccaggaagaggacggctgctcctgcagattccccgagg
aagaagaaggcggctgcgagctgagagtgaagttcagcagatccgccgacgcccctgcctacaagcagggccagaaccag
ctgtacaacgagctgaacctgggcagacgggaagagtacgacgtgctggacaagcggagaggcagggaccctgagatggg
cggcaagcccagaagaaagaacccccaggaaggcctgtataacgaactgcagaaagacaagatggccgaggcctacagcg
agatcggaatgaagggcgagcggagaagaggcaagggccacgatggcctgtaccagggcctgagcaccgccaccaaggac
acctatgacgccctgcacatgcaggccctgcctcccagatgataa
(amino acids)
(SEQ ID NO: 727)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRRGKGHDGLYQGLSTATKD
TYDALHMQALPPR**
CAR-T IgK C2 CD8/CD8/4-1BB/CD3z op #49
N-IgK1s-huMNC2scFv -CD8ecd fragment- CD8 transmembrane- 4-1BB- CD3zeta (all
domains codon optimized)-C
(DNA)
(SEQ ID NO: 728)
atggagacagacacactcctgctatgggtactgctgctctgggttccaggttccactggtgaagtgcagctggtggaatc
tggcggcggactcgtgaagcctggcggctctctgagactgagctgtgccgccagcggcttcacctttagcggctacgcca
tgagctgggtgcgccaggctcctggcaaaggcctggaatgggtgtccaccatctctagcggcggcacctacatctactac
cccgacagcgtgaagggccggttcaccatcagccgggacaacgccaagaacagcctgtacctgcagatgaactccctgcg
ggccgaggacaccgccgtgtactattgtgctagactgggcggcgacaactactacgagtacttcgacgtgtggggcaagg
gcaccaccgtgacagtgtctagcggaggcggaggatcaggcggcggaggaagtggcggagggggatctgatatcgtgctg
acccagagccctgccagcctggctgtgtctcctggacagagggccaccatcacctgtcgggccagcaagagcgtgtccac
ctccggctacagctacatgcactggtatcagcagaagcccggccagccccccaagctgctgatctacctggccagcaacc
tggaaagcggcgtgcccgctagattttccggctctggcagcggcaccgacttcaccctgaccatcaaccccgtggaagcc
aacgacaccgccaattactactgccagcacagcagagagctgcccttcaccttcggcggaggcaccaaggtggaaatcaa
gcggaccacaacaacccctgcccccagacctcctaccccagcccctacaattgccagccagcctctgagcctgaggcccg
aggcttgtagacctgctgctggcggagccgtgcacaccagaggactggatttcgcctgcgacatctacatctgggcccct
ctggccggcacatgtggcgtgctgctgctgagcctcgtgatcaccctgtactgcaagcggggcagaaagaagctgctgta
catcttcaagcagcccttcatgcggcccgtgcagaccacccaggaagaggacggctgctcctgcagattccccgaggaag
aagaaggcggctgcgagctgagagtgaagttcagcagatccgccgacgcccctgcctacaagcagggccagaaccagctg
tacaacgagctgaacctgggcagacgggaagagtacgacgtgctggacaagcggagaggcagggaccctgagatgggcgg
caagcccagaagaaagaacccccaggaaggcctgtataacgaactgcagaaagacaagatggccgaggcctacagcgaga
tcggaatgaagggcgagcggagaagaggcaagggccacgatggcctgtaccagggcctgagcaccgccaccaaggacacc
tatgacgccctgcacatgcaggccctgcctcccagatgataa
(amino acids)
(SEQ ID NO: 729)
METDTLLLWVLLLWVPGSTGEVQLVESGGGLVKPGGSLRLSCAASGFTFSGYAMSWVRQAPGKGLEWVSTISSGGTYIYY
PDSVKGRFTISRDNAKNSLYLQMNSLRAEDTAVYYCARLGGDNYYEYFDVWGKGTTVTVSSGGGGSGGGGSGGGGSDIVL
TQSPASLAVSPGQRATITCRASKSVSTSGYSYMHWYQQKPGQPPKLLIYLASNLESGVPARFSGSGSGTDFTLTINPVEA
NDTANYYCQHSRELPFTFGGGTKVEIKRTTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T C2 CD4/CD4/4-1BB/CD3z #50
N-CD81s-huMNC2scFv-CD4ecd fragment- CD4 transmembrane- 4-1BB- CD3zeta-C
(DNA)
(SEQ ID NO: 730)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaacttcgggacaggtcctgctggaatccaacatcaaggttctgcccacatggtccaccccggtgcagccaatgg
ccctgattgtgctggggggcgtcgccggcctcctgcttttcattgggctaggcatcttcttcaaacggggcagaaagaaa
ctcctgtatatattcaaacaaccatttatgagaccagtacaaactactcaagaggaagatggctgtagctgccgatttcc
agaagaagaagaaggaggatgtgaactgagagtgaagttcagcaggagcgcagacgcccccgcgtacaagcagggccaga
accagctctataacgagctcaatctaggacgaagagaggagtacgatgttttggacaagagacgtggccgggaccctgag
atggggggaaagccgagaaggaagaaccctcaggaaggcctgtacaatgaactgcagaaagataagatggcggaggccta
cagtgagattgggatgaaaggcgagcgccggaggggcaaggggcacgatggcctttaccagggtctcagtacagccacca
aggacacctacgacgcccttcacatgcaggccctgccccctcgctgataa
(amino acids)
(SEQ ID NO: 731)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFKRGRKK
LLYIFKQPFMRPVQTTQEEDGCSCRFPEEEEGGCELRVKFSRSADAPAYKQGQNQLYNELNLGRREEYDVLDKRRGRDPE
MGGKPRRKNPQEGLYNELQKDKMAEAYSEIGMKGERRRGKGHDGLYQGLSTATKDTYDALHMQALPPR**
CAR-T C2 FC/CD8/4-1BB/CD3z “Fc” CAR53
N-CD81s-huMNC2scFv-Human IgG1 Fc- CD8 transmembrane- 4-1BB- CD3zeta-C
(DNA)
(SEQ ID NO: 732)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgagcccaaatcttgtgacaaaactcacacatgcccaccgtgcccagcacctgaactcctggggggaccgt
cagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcg
ggaggagcagtacaacagcacgtaccgtgtggtcagcgtcctcaccgtcctgcaccaggactggctgaatggcaaggagt
acaagtgcaaggtctccaacaaagccctcccagcccccatcgagaaaaccatctccaaagccaaagggcagccccgagaa
ccacaggtgtacaccctgcccccatcccgggaggagatgaccaagaaccaggtcagcctgacctgcctggtcaaaggctt
ctatcccagcgacatcgccgtggagtgggagagcaatgggcagccggagaacaactacaagaccacgcctcccgtgctgg
actccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaatctacatctgggc
gcccttggccgggacttgtggggtccttctcctgtcactggttatcaccctttactgcaaacggggcagaaagaaactcc
tgtatatattcaaacaaccatttatgagaccagtacaaactactcaagaggaagatggctgtagctgccgatttccagaa
gaagaagaaggaggatgtgaactgagagtgaagttcagcaggagcgcagacgcccccgcgtacaagcagggccagaacca
gctctataacgagctcaatctaggacgaagagaggagtacgatgttttggacaagagacgtggccgggaccctgagatgg
ggggaaagccgagaaggaagaaccctcaggaaggcctgtacaatgaactgcagaaagataagatggcggaggcctacagt
gagattgggatgaaaggcgagcgccggaggggcaaggggcacgatggcctttaccagggtctcagtacagccaccaagga
cacctacgacgcccttcacatgcaggccctgccccctcgctgataa
(amino acids)
(SEQ ID NO: 733)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGKIYIWAPLAGTCGVLLLSLVITLYCKRGRKKLLYIFKQPFMRPVQTTQEEDGCSCRFPE
EEEGGCELRVKFSRSADAPAYKQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR**
CAR-T C2 IgD/FC/CD8/4-1BB/CD3z “IgD-Fc” CAR54
N-CD81s-huMNC2scFv- IgD hinge- Human IgG1 Fc- CD8 transmembrane- 4-1BB-
CD3zeta-C
(DNA)
(SEQ ID NO: 734)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgagtctccaaaggcacaggcctcctcagtgcccactgcacaaccccaagcagagggcagcctcgccaagg
caaccacagccccagccaccacccgtaacacaggaagaggcggcgaagagaagaaaaaggagaaggagaaagaggaacaa
gaagagagagagacaaagacaccagagcccaaatcttgtgacaaaactcacacatgcccaccgtgcccagcacctgaact
cctggggggaccgtcagtcttcctcttccccccaaaacccaaggacaccctcatgatctcccggacccctgaggtcacat
gcgtggtggtggacgtgagccacgaagaccctgaggtcaagttcaactggtacgtggacggcgtggaggtgcataatgcc
aagacaaagccgcgggaggagcagtacaacagcacgtaccgtgtggtcagcgtcctcaccgtcctgcaccaggactggct
gaatggcaaggagtacaagtgcaaggtctccaacaaagccctcccagcccccatcgagaaaaccatctccaaagccaaag
ggcagccccgagaaccacaggtgtacaccctgcccccatcccgggaggagatgaccaagaaccaggtcagcctgacctgc
ctggtcaaaggcttctatcccagcgacatcgccgtggagtgggagagcaatgggcagccggagaacaactacaagaccac
gcctcccgtgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtaaa
atctacatctgggcgcccttggccgggacttgtggggtccttctcctgtcactggttatcaccctttactgcaaacgggg
cagaaagaaactcctgtatatattcaaacaaccatttatgagaccagtacaaactactcaagaggaagatggctgtagct
gccgatttccagaagaagaagaaggaggatgtgaactgagagtgaagttcagcaggagcgcagacgcccccgcgtacaag
cagggccagaaccagctctataacgagctcaatctaggacgaagagaggagtacgatgttttggacaagagacgtggccg
ggaccctgagatggggggaaagccgagaaggaagaaccctcaggaaggcctgtacaatgaactgcagaaagataagatgg
cggaggcctacagtgagattgggatgaaaggcgagcgccggaggggcaaggggcacgatggcctttaccagggtctcagt
acagccaccaaggacacctacgacgcccttcacatgcaggccctgccccctcgctgataa
(amino acids)
(SEQ ID NO: 735)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTESPKAQASSVPTAQPQAEGSLAKATTAPATTRNTGRGGEEKKKEKEKEEQ
EERETKTPEPKSCDKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMHEALHNHYTQKSLSLSPGK
IYIWAPLAGTCGVLLLSLVITLYCKRGRKKLLYIFKQPFMRPVQTTQEEDGCSCRFPEEEEGGCELRVKFSRSADAPAYK
QGQNQLYNELNLGRREEYDVLDKRRGRDPEMGGKPRRKNPQEGLYNELQKDKMAEAYSEIGMKGERRRGKGHDGLYQGLS
TATKDTYDALHMQALPPR**
CAR-T C2 FCHingeless Y407R/CD8/4-1BB/CD3z “FcH” CAR55
N-CD81s-huMNC2scFv-Human IgG1 hingeless Fc Y407R- CD8 transmembrane- 4-1BB-
CD3zeta-C
(DNA)
(SEQ ID NO: 736)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgcacctgaactcctggggggaccgtcagtcttcctcttccccccaaaacccaaggacaccctcatgatct
cccggacccctgaggtcacatgcgtggtggtggacgtgagccacgaagaccctgaggtcaagttcaactggtacgtggac
ggcgtggaggtgcataatgccaagacaaagccgcgggaggagcagtacaacagcacgtaccgtgtggtcagcgtcctcac
cgtcctgcaccaggactggctgaatggcaaggagtacaagtgcaaggtctccaacaaagccctcccagcccccatcgaga
aaaccatctccaaagccaaagggcagccccgagaaccacaggtgtacaccctgcccccatcccgggaggagatgaccaag
aaccaggtcagcctgacctgcctggtcaaaggcttctatcccagcgacatcgccgtggagtgggagagcaatgggcagcc
ggagaacaactacaagaccacgcctcccgtgctggactccgacggctccttcttcctcaggagcaagctcaccgtggaca
agagcaggtggcagcaggggaacgtcttctcatgctccgtgatgcatgaggctctgcacaaccactacacgcagaagagc
ctctccctgtctccgggtaaaatctacatctgggcgcccttggccgggacttgtggggtccttctcctgtcactggttat
caccctttactgcaaacggggcagaaagaaactcctgtatatattcaaacaaccatttatgagaccagtacaaactactc
aagaggaagatggctgtagctgccgatttccagaagaagaagaaggaggatgtgaactgagagtgaagttcagcaggagc
gcagacgcccccgcgtacaagcagggccagaaccagctctataacgagctcaatctaggacgaagagaggagtacgatgt
tttggacaagagacgtggccgggaccctgagatggggggaaagccgagaaggaagaaccctcaggaaggcctgtacaatg
aactgcagaaagataagatggcggaggcctacagtgagattgggatgaaaggcgagcgccggaggggcaaggggcacgat
ggcctttaccagggtctcagtacagccaccaaggacacctacgacgcccttcacatgcaggccctgccccctcgctgata
a
(amino acids)
(SEQ ID NO: 737)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLRSKLTVDKSRWQQGNVFSCSVMHEALHNHYTQKS
LSLSPGKIYIWAPLAGTCGVLLLSLVITLYCKRGRKKLLYIFKQPFMRPVQTTQEEDGCSCRFPEEEEGGCELRVKFSRS
ADAPAYKQGQNQLYNELNLGRREEYDVLDKRRGRDPEMGGKPRRKNPQEGLYNELQKDKMAEAYSEIGMKGERRRGKGHD
GLYQGLSTATKDTYDALHMQALPPR**
CAR-T C2 IgD/FCHingeless Y407R/CD8/4-1BB/CD3z “IgD FcH” CAR56
N-CD81s- huMNC2scFv- IgD hinge- Human IgG1 hingeless Fc Y407R- CD8
transmembrane- 4-1BB- CD3zeta-C
(DNA)
(SEQ ID NO: 738)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgagtctccaaaggcacaggcctcctcagtgcccactgcacaaccccaagcagagggcagcctcgccaagg
caaccacagccccagccaccacccgtaacacaggaagaggcggcgaagagaagaaaaaggagaaggagaaagaggaacaa
gaagagagagagacaaagacaccagcacctgaactcctggggggaccgtcagtcttcctcttccccccaaaacccaagga
caccctcatgatctcccggacccctgaggtcacatgcgtggtggtggacgtgagccacgaagaccctgaggtcaagttca
actggtacgtggacggcgtggaggtgcataatgccaagacaaagccgcgggaggagcagtacaacagcacgtaccgtgtg
gtcagcgtcctcaccgtcctgcaccaggactggctgaatggcaaggagtacaagtgcaaggtctccaacaaagccctccc
agcccccatcgagaaaaccatctccaaagccaaagggcagccccgagaaccacaggtgtacaccctgcccccatcccggg
aggagatgaccaagaaccaggtcagcctgacctgcctggtcaaaggcttctatcccagcgacatcgccgtggagtgggag
agcaatgggcagccggagaacaactacaagaccacgcctcccgtgctggactccgacggctccttcttcctcaggagcaa
gctcaccgtggacaagagcaggtggcagcaggggaacgtcttctcatgctccgtgatgcatgaggctctgcacaaccact
acacgcagaagagcctctccctgtctccgggtaaaatctacatctgggcgcccttggccgggacttgtggggtccttctc
ctgtcactggttatcaccctttactgcaaacggggcagaaagaaactcctgtatatattcaaacaaccatttatgagacc
agtacaaactactcaagaggaagatggctgtagctgccgatttccagaagaagaagaaggaggatgtgaactgagagtga
agttcagcaggagcgcagacgcccccgcgtacaagcagggccagaaccagctctataacgagctcaatctaggacgaaga
gaggagtacgatgttttggacaagagacgtggccgggaccctgagatggggggaaagccgagaaggaagaaccctcagga
aggcctgtacaatgaactgcagaaagataagatggcggaggcctacagtgagattgggatgaaaggcgagcgccggaggg
gcaaggggcacgatggcctttaccagggtctcagtacagccaccaaggacacctacgacgcccttcacatgcaggccctg
ccccctcgctgataa
(amino acids)
(SEQ ID NO: 739)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTESPKAQASSVPTAQPQAEGSLAKATTAPATTRNTGRGGEEKKKEKEKEEQ
EERETKTPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVEWE
SNGQPENNYKTTPPVLDSDGSFFLRSKLTVDKSRWQQGNVFSCSVMHEALHNHYTQKSLSLSPGKIYIWAPLAGTCGVLL
LSLVITLYCKRGRKKLLYIFKQPFMRPVQTTQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGERRRGKGHDGLYQGLSTATKDTYDALHMQAL
PPR**
CAR-T C2 IgD/CD8/4-1BB/CD3z “IgD” CAR57
N-CD81s-huMNC2scFv- IgD hinge- CD8 transmembrane- 4-1BB- CD3zeta-C
(DNA)
(SEQ ID NO: 740)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgagtctccaaaggcacaggcctcctcagtgcccactgcacaaccccaagcagagggcagcctcgccaagg
caaccacagccccagccaccacccgtaacacaggaagaggcggcgaagagaagaaaaaggagaaggagaaagaggaacaa
gaagagagagagacaaagacaccaatctacatctgggcgcccttggccgggacttgtggggtccttctcctgtcactggt
tatcaccctttactgcaaacggggcagaaagaaactcctgtatatattcaaacaaccatttatgagaccagtacaaacta
ctcaagaggaagatggctgtagctgccgatttccagaagaagaagaaggaggatgtgaactgagagtgaagttcagcagg
agcgcagacgcccccgcgtacaagcagggccagaaccagctctataacgagctcaatctaggacgaagagaggagtacga
tgttttggacaagagacgtggccgggaccctgagatggggggaaagccgagaaggaagaaccctcaggaaggcctgtaca
atgaactgcagaaagataagatggcggaggcctacagtgagattgggatgaaaggcgagcgccggaggggcaaggggcac
gatggcctttaccagggtctcagtacagccaccaaggacacctacgacgcccttcacatgcaggccctgccccctcgctg
ataa
(amino acids)
(SEQ ID NO: 741)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTESPKAQASSVPTAQPQAEGSLAKATTAPATTRNTGRGGEEKKKEKEKEEQ
EERETKTPIYIWAPLAGTCGVLLLSLVITLYCKRGRKKLLYIFKQPFMRPVQTTQEEDGCSCRFPEEEEGGCELRVKFSR
SADAPAYKQGQNQLYNELNLGRREEYDVLDKRRGRDPEMGGKPRRKNPQEGLYNELQKDKMAEAYSEIGMKGERRRGKGH
DGLYQGLSTATKDTYDALHMQALPPR**
CAR-T C2 X4/CD8/4-1BB/CD3z “X4” CAR58
N-CD81s-huMNC2scFv- X4 linker- CD8 transmembrane- 4-1BB- CD3zeta-C
(DNA)
(SEQ ID NO: 742)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgacaagacgcacaccaagccacctaaaccagctccagaactgctcggaggtcctggcaccggaaccggag
gacctaccatcaaaccacctaagccacctaagcctgctcctaacctgctcggaggacctatctacatctgggcgcccttg
gccgggacttgtggggtccttctcctgtcactggttatcaccctttactgcaaacggggcagaaagaaactcctgtatat
attcaaacaaccatttatgagaccagtacaaactactcaagaggaagatggctgtagctgccgatttccagaagaagaag
aaggaggatgtgaactgagagtgaagttcagcaggagcgcagacgcccccgcgtacaagcagggccagaaccagctctat
aacgagctcaatctaggacgaagagaggagtacgatgttttggacaagagacgtggccgggaccctgagatggggggaaa
gccgagaaggaagaaccctcaggaaggcctgtacaatgaactgcagaaagataagatggcggaggcctacagtgagattg
ggatgaaaggcgagcgccggaggggcaaggggcacgatggcctttaccagggtctcagtacagccaccaaggacacctac
gacgcccttcacatgcaggccctgccccctcgctgataa
(amino acids)
(SEQ ID NO: 743)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTDKTHTKPPKPAPELLGGPGTGTGGPTIKPPKPPKPAPNLLGGPIYIWAPL
AGTCGVLLLSLVITLYCKRGRKKLLYIFKQPFMRPVQTTQEEDGCSCRFPEEEEGGCELRVKFSRSADAPAYKQGQNQLY
NELNLGRREEYDVLDKRRGRDPEMGGKPRRKNPQEGLYNELQKDKMAEAYSEIGMKGERRRGKGHDGLYQGLSTATKDTY
DALHMQALPPR**
CAR-T E6 CD8/CD4/41BB/CD3z CAR37
N-CD81s-huMNE6scFv- CD8ecd- CD4 transmembrane- 4-1BB- CD3zeta-C
(DNA)
(SEQ ID NO: 744)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatggccctgattgtgctggggggcgtcgccggcctcctgcttttc
attgggctaggcatcttcttcaaaaggggccgcaaaaaactcctttacatttttaagcagccttttatgaggccagtacag
acgactcaagaggaagacgggtgctcatgccgctttcctgaggaggaggaaggagggtgcgaactgcgcgttaagttctcc
cgatcagccgacgcgcctgcttacaagcagggccagaaccaactgtacaacgagctgaatctcggtagacgggaagagtac
gacgtgttggacaaacggagaggccgcgacccagaaatgggcggcaagcctcgcaggaaaaacccccaggagggactgtac
aatgagttgcagaaagataagatggcagaagcttatagcgagatcggaatgaagggggaaaggagacgagggaaaggacac
gacggcctttatcagggcctgtccacagcaacaaaagatacgtatgacgccctccatatgcaggcacttccaccacggtga
taa
(amino acids)
(SEQ ID NO: 745)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDMALIVLGGVAG
LLLFIGLGIFFKRGRKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTATKDTYDALHMQ
ALPPR**
CAR-T E6 CD4/CD4/CD3z sequence CAR23:
N-CD81s-huMNE6scFv- CD4ecd- CD4 transmembrane- CD3zeta-C
(DNA)
(SEQ ID NO: 746)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaatcgggacaggtcctgctg
gaatccaacatcaaggttctgcccacatggtccaccccggtgcagccaatggccctgattgtgctggggggcgtcgccggc
ctcctgcttttcattgggctaggcatcttcttccgcgttaagttctcccgatcagccgacgcgcctgcttacaagcagggc
cagaaccaactgtacaacgagctgaatctcggtagacgggaagagtacgacgtgttggacaaacggagaggccgcgaccca
gaaatgggcggcaagcctcgcaggaaaaacccccaggagggactgtacaatgagttgcagaaagataagatggcagaagct
tatagcgagatcggaatgaagggggaaaggagacgagggaaaggacacgacggcctttatcagggcctgtccacagcaaca
aaagatacgtatgacgccctccatatgcaggcacttccaccacggtgataa
(amino acids)
(SEQ ID NO: 747)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRVKFSRSADAPA
YKQGQNQLYNELNLGRREEYDVLDKRRGRDPEMGGKPRRKNPQEGLYNELQKDKMAEAYSEIGMKGERRRGKGHDGLYQG
LSTATKDTYDALHMQALPPR**
CAR-T E6 CD4/CD4/CD28/CD3z sequence CAR25:
N-CD81s-huMNE6scFv- CD4ecd- CD4 transmembrane- CD28- CD3zeta-C
(DNA)
(SEQ ID NO: 748)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaatcgggacaggtcctgctg
gaatccaacatcaaggttctgcccacatggtccaccccggtgcagccaatggccctgattgtgctggggggcgtcgccggc
ctcctgcttttcattgggctaggcatcttcttcagaagcaagcggtctcggctcctgcattctgattacatgaacatgacc
ccaagaagaccaggccccaccaggaaacattaccagccctacgctccgccacgcgacttcgctgcctaccggtcccgcgtt
aagttctcccgatcagccgacgcgcctgcttacaagcagggccagaaccaactgtacaacgagctgaatctcggtagacgg
gaagagtacgacgtgttggacaaacggagaggccgcgacccagaaatgggcggcaagcctcgcaggaaaaacccccaggag
ggactgtacaatgagttgcagaaagataagatggcagaagcttatagcgagatcggaatgaagggggaaaggagacgaggg
aaaggacacgacggcctttatcagggcctgtccacagcaacaaaagatacgtatgacgccctccatatgcaggcacttcca
ccacggtgataa
(amino acids)
(SEQ ID NO: 749)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRSKRSRLLHSDY
MNMTPRRPGPTRKHYQPYAPPRDFAAYRSRVKFSRSADAPAYKQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR**
CAR-T E6 CD4/CD4/4-1BB/CD3z sequence CAR31:
N-CD81s-huMNE6scFv- CD4ecd- CD4 transmembrane- 4-1BB- CD3zeta-C
(DNA)
(SEQ ID NO: 750)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaatcgggacaggtcctgctg
gaatccaacatcaaggttctgcccacatggtccaccccggtgcagccaatggccctgattgtgctggggggcgtcgccggc
ctcctgcttttcattgggctaggcatcttcttcaaaaggggccgcaaaaaactcctttacatttttaagcagccttttatg
aggccagtacagacgactcaagaggaagacgggtgctcatgccgctttcctgaggaggaggaaggagggtgcgaactgcgc
gttaagttctcccgatcagccgacgcgcctgcttacaagcagggccagaaccaactgtacaacgagctgaatctcggtaga
cgggaagagtacgacgtgttggacaaacggagaggccgcgacccagaaatgggcggcaagcctcgcaggaaaaacccccag
gagggactgtacaatgagttgcagaaagataagatggcagaagcttatagcgagatcggaatgaagggggaaaggagacga
gggaaaggacacgacggcctttatcagggcctgtccacagcaacaaaagatacgtatgacgccctccatatgcaggcactt
ccaccacggtgataa
(amino acids)
(SEQ ID NO: 751)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFKRGRKKLLYIFK
QPFMRPVQTTQEEDGCSCRFPEEEEGGCELRVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDTYDALHMQALPPR**
CAR-T E6 CD4/CD4/OX40/CD3z sequence:
N-CD81s-huMNE6scFv- CD4ecd- CD4 transmembrane- OX40- CD3zeta-C
(DNA)
(SEQ ID NO: 752)
atggccctgcccgtgaccgctttgctgctccccctggcgctgctgctgcacgccgccaggccagaggtccagctggttga
gagtggcggtgggctggttaagcctggcggctccctgcggctgagctgcgccgcgagtggatttactttcagccgatatg
ggatgagttgggtgcggcaagctcccgggaagaggctggaatgggtctcaacaatctccggggggggcacttacatctat
taccccgactcagtcaaggggagatttaccatttcacgagacaacgctaagaataccctgtatttgcagatgaattctct
gagagcagaggacacagctgtttactattgtacccgcgacaactatggcaggaactacgactacggtatggactattggg
gacaagggacattggttacagtgagcagtggcggcgggggcagcggaggaggaggcagcggtggggggggcagcgagata
gtgctcacgcagtcacccgcgactctcagtctctcacctggggaacgagctaccctgacgtgctctgctacctcctcagt
gtcatatattcactggtatcagcaacggcccgggcagtcccctagattgctcatttatagtacctctaatctggcctcag
gtatccctgcacgattttctggatctggttcaggttctgattacaccctcactatctctagcctggagcctgaagacttt
gccgtttattactgccagcagaggtctagctccccattcacctttgggagtgggaccaaggttgaaattaaatcgggaca
ggtcctgctggaatccaacatcaaggttctgcccacatggtccaccccggtgcagccaatggccctgattgtgctggggg
gcgtcgccggcctcctgcttttcattgggctaggcatcttcttccggagggaccagaggctgccccccgatgcccacaag
ccccctgggggaggcagtttccggacccccatccaagaggagcaggccgacgcccactccaccctggccaagatccgcgt
taagttctcccgatcagccgacgcgcctgcttacaagcagggccagaaccaactgtacaacgagctgaatctcggtagac
gggaagagtacgacgtgttggacaaacggagaggccgcgacccagaaatgggcggcaagcctcgcaggaaaaacccccag
gagggactgtacaatgagttgcagaaagataagatggcagaagcttatagcgagatcggaatgaagggggaaaggagacg
agggaaaggacacgacggcctttatcagggcctgtccacagcaacaaaagatacgtatgacgccctccatatgcaggcac
ttccaccacggtgataa
(amino acids)
(SEQ ID NO: 753)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRRDQRLPPDAHK
PPGGGSFRTPIQEEQADAHSTLAKIRVKFSRSADAPAYKQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR**
CAR-T E6 CD4/CD4/CD28/4-1BB/CD3z sequence CAR38:
N-CD81s-huMNE6scFv- CD4ecd- CD4 transmembrane- CD28- 4-1BB- CD3zeta-C
(DNA)
(SEQ ID NO: 754)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaatcgggacaggtcctgctg
gaatccaacatcaaggttctgcccacatggtccaccccggtgcagccaatggccctgattgtgctggggggcgtcgccggc
ctcctgcttttcattgggctaggcatcttcttcagaagcaagcggtctcggctcctgcattctgattacatgaacatgacc
ccaagaagaccaggccccaccaggaaacattaccagccctacgctccgccacgcgacttcgctgcctaccggtccaaaagg
ggccgcaaaaaactcctttacatttttaagcagccttttatgaggccagtacagacgactcaagaggaagacgggtgctca
tgccgctttcctgaggaggaggaaggagggtgcgaactgcgcgttaagttctcccgatcagccgacgcgcctgcttacaag
cagggccagaaccaactgtacaacgagctgaatctcggtagacgggaagagtacgacgtgttggacaaacggagaggccgc
gacccagaaatgggcggcaagcctcgcaggaaaaacccccaggagggactgtacaatgagttgcagaaagataagatggca
gaagcttatagcgagatcggaatgaagggggaaaggagacgagggaaaggacacgacggcctttatcagggcctgtccaca
gcaacaaaagatacgtatgacgccctccatatgcaggcacttccaccacggtgataa
(amino acids)
(SEQ ID NO: 755)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRSKRSRLLHSDY
MNMTPRRPGPTRKHYQPYAPPRDFAAYRSKRGRKKLLYIFKQPFMRPVQTTQEEDGCSCRFPEEEEGGCELRVKFSRSAD
APAYKQGQNQLYNELNLGRREEYDVLDKRRGRDPEMGGKPRRKNPQEGLYNELQKDKMAEAYSEIGMKGERRRGKGHDGL
YQGLSTATKDTYDALHMQALPPR**
CAR-T E6 CD4/CD4/CD28/OX40/CD3z sequence:
N-CD81s-huMNE6scFv- CD4ecd- CD4 transmembrane- CD28- OX40- CD3zeta-C
(DNA)
(SEQ ID NO: 756)
atggccctgcccgtgaccgctttgctgctccccctggcgctgctgctgcacgccgccaggccagaggtccagctggttga
gagtggcggtgggctggttaagcctggcggctccctgcggctgagctgcgccgcgagtggatttactttcagccgatatg
ggatgagttgggtgcggcaagctcccgggaagaggctggaatgggtctcaacaatctccggggggggcacttacatctat
taccccgactcagtcaaggggagatttaccatttcacgagacaacgctaagaataccctgtatttgcagatgaattctct
gagagcagaggacacagctgtttactattgtacccgcgacaactatggcaggaactacgactacggtatggactattggg
gacaagggacattggttacagtgagcagtggcggcgggggcagcggaggaggaggcagcggtggggggggcagcgagata
gtgctcacgcagtcacccgcgactctcagtctctcacctggggaacgagctaccctgacgtgctctgctacctcctcagt
gtcatatattcactggtatcagcaacggcccgggcagtcccctagattgctcatttatagtacctctaatctggcctcag
gtatccctgcacgattttctggatctggttcaggttctgattacaccctcactatctctagcctggagcctgaagacttt
gccgtttattactgccagcagaggtctagctccccattcacctttgggagtgggaccaaggttgaaattaaatcgggaca
ggtcctgctggaatccaacatcaaggttctgcccacatggtccaccccggtgcagccaatggccctgattgtgctggggg
gcgtcgccggcctcctgcttttcattgggctaggcatcttcttcagaagcaagcggtctcggctcctgcattctgattac
atgaacatgaccccaagaagaccaggccccaccaggaaacattaccagccctacgctccgccacgcgacttcgctgccta
ccggtcccggagggaccagaggctgccccccgatgcccacaagccccctgggggaggcagtttccggacccccatccaag
aggagcaggccgacgcccactccaccctggccaagatccgcgttaagttctcccgatcagccgacgcgcctgcttacaag
cagggccagaaccaactgtacaacgagctgaatctcggtagacgggaagagtacgacgtgttggacaaacggagaggccg
cgacccagaaatgggcggcaagcctcgcaggaaaaacccccaggagggactgtacaatgagttgcagaaagataagatgg
cagaagcttatagcgagatcggaatgaagggggaaaggagacgagggaaaggacacgacggcctttatcagggcctgtcc
acagcaacaaaagatacgtatgacgccctccatatgcaggcacttccaccacggtgataa
(amino acids)
(SEQ ID NO: 757)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRSKRSRLLHSDY
MNMTPRRPGPTRKHYQPYAPPRDFAAYRSRRDQRLPPDAHKPPGGGSFRTPIQEEQADAHSTLAKIRVKFSRSADAPAYK
QGQNQLYNELNLGRREEYDVLDKRRGRDPEMGGKPRRKNPQEGLYNELQKDKMAEAYSEIGMKGERRRGKGHDGLYQGLS
TATKDTYDALHMQALPPR**
CAR-T C2 CD4/CD4/CD3z sequence:
N-CD81s-huMNC2scFv- CD4ecd- CD4 transmembrane- CD3zeta-C
(DNA)
(SEQ ID NO: 758)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
tcgggacaggtcctgctggaatccaacatcaaggttctgcccacatggtccaccccggtgcagccaatggccctgattgtg
ctggggggcgtcgccggcctcctgcttttcattgggctaggcatcttcttccgcgttaagttctcccgatcagccgacgcg
cctgcttacaagcagggccagaaccaactgtacaacgagctgaatctcggtagacgggaagagtacgacgtgttggacaaa
cggagaggccgcgacccagaaatgggcggcaagcctcgcaggaaaaacccccaggagggactgtacaatgagttgcagaaa
gataagatggcagaagcttatagcgagatcggaatgaagggggaaaggagacgagggaaaggacacgacggcctttatcag
ggcctgtccacagcaacaaaagatacgtatgacgccctccatatgcaggcacttccaccacggtgataa
(amino acids)
(SEQ ID NO: 759)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRVKFSR
SADAPAYKQGQNQLYNELNLGRREEYDVLDKRRGRDPEMGGKPRRKNPQEGLYNELQKDKMAEAYSEIGMKGERRRGKGH
DGLYQGLSTATKDTYDALHMQALPPR**
CAR-T C2 CD4/CD4/CD28/CD3z sequence:
N-CD81s-huMNC2scFv- CD4ecd- CD4 transmembrane- CD28- CD3zeta-C
(DNA)
(SEQ ID NO: 760)
atggccttgccagtgacggccctgctgctgccattggctcttctgttgcacgctgccaggcctgaagtgcagctcgtaga
gagtggcgggggactggtgaagcccggtggaagcctcagactcagttgcgccgcctcaggtttcactttttcaggttacg
ccatgtcctgggtaagacaggcaccggggaaaggactcgagtgggtgtctactatcagctcaggaggcacttatatatat
tatcctgactctgtaaaaggccgatttacgatttctcgcgacaatgcaaagaactccctctacctccaaatgaacagtct
tagggcagaagacactgctgtatactattgtgcacgcctcggcggcgacaactactacgagtactttgacgtgtggggga
aagggactaccgtgacagtttcaagcggaggaggtggctcaggtggaggcgggtcaggggggggaggaagtgatattgtg
ctcacacaatccccagcctccctggctgtgtctcccggccaacgcgctacaattacatgtcgggcctccaaaagcgtgag
caccagcggctacagctacatgcactggtatcaacagaaaccaggacaaccccccaaactgttgatttatctcgcttcaa
acttggagtccggcgtgcctgcgcgcttttcagggagtgggagcggcacagattttacgctgactatcaaccccgtagaa
gcaaacgatacagcgaattattattgtcaacattcccgggaactcccctttacgttcggcgggggcacaaaggtcgaaat
taagagaacctcgggacaggtcctgctggaatccaacatcaaggttctgcccacatggtccaccccggtgcagccaatgg
ccctgattgtgctggggggcgtcgccggcctcctgcttttcattgggctaggcatcttcttcagaagcaagcggtctcgg
ctcctgcattctgattacatgaacatgaccccaagaagaccaggccccaccaggaaacattaccagccctacgctccgcc
acgcgacttcgctgcctaccggtcccgcgttaagttctcccgatcagccgacgcgcctgcttacaagcagggccagaacc
aactgtacaacgagctgaatctcggtagacgggaagagtacgacgtgttggacaaacggagaggccgcgacccagaaatg
ggcggcaagcctcgcaggaaaaacccccaggagggactgtacaatgagttgcagaaagataagatggcagaagcttatag
cgagatcggaatgaagggggaaaggagacgagggaaaggacacgacggcctttatcagggcctgtccacagcaacaaaag
atacgtatgacgccctccatatgcaggcacttccaccacggtgataa
(amino acids)
(SEQ ID NO: 761)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRSKRSR
LLHSDYMNMTPRRPGPTRKHYQPYAPPRDFAAYRSRVKFSRSADAPAYKQGQNQLYNELNLGRREEYDVLDKRRGRDPEM
GGKPRRKNPQEGLYNELQKDKMAEAYSEIGMKGERRRGKGHDGLYQGLSTATKDTYDALHMQALPPR**
CAR-T C2 CD4/CD4/4-1BB/CD3z sequence:
N-CD81s-huMNC2scFv- CD4ecd- CD4 transmembrane- 4-1BB- CD3zeta-C
(DNA)
(SEQ ID NO: 762)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
tcgggacaggtcctgctggaatccaacatcaaggttctgcccacatggtccaccccggtgcagccaatggccctgattgtg
ctggggggcgtcgccggcctcctgcttttcattgggctaggcatcttcttcaaaaggggccgcaaaaaactcctttacatt
tttaagcagccttttatgaggccagtacagacgactcaagaggaagacgggtgctcatgccgctttcctgaggaggaggaa
ggagggtgcgaactgcgcgttaagttctcccgatcagccgacgcgcctgcttacaagcagggccagaaccaactgtacaac
gagctgaatctcggtagacgggaagagtacgacgtgttggacaaacggagaggccgcgacccagaaatgggcggcaagcct
cgcaggaaaaacccccaggagggactgtacaatgagttgcagaaagataagatggcagaagcttatagcgagatcggaatg
aagggggaaaggagacgagggaaaggacacgacggcctttatcagggcctgtccacagcaacaaaagatacgtatgacgcc
ctccatatgcaggcacttccaccacggtgataa
(amino acids)
(SEQ ID NO: 763)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFKRGRKK
LLYIFKQPFMRPVQTTQEEDGCSCRFPEEEEGGCELRVKFSRSADAPAYKQGQNQLYNELNLGRREEYDVLDKRRGRDPE
MGGKPRRKNPQEGLYNELQKDKMAEAYSEIGMKGERRRGKGHDGLYQGLSTATKDTYDALHMQALPPR**
CAR-T C2 CD4/CD4/OX40/CD3z sequence:
N-CD81s-huMNC2scFv- CD4ecd- CD4 transmembrane- OX40- CD3zeta-C
(DNA)
(SEQ ID NO: 764)
atggccttgccagtgacggccctgctgctgccattggctcttctgttgcacgctgccaggcctgaagtgcagctcgtaga
gagtggcgggggactggtgaagcccggtggaagcctcagactcagttgcgccgcctcaggtttcactttttcaggttacg
ccatgtcctgggtaagacaggcaccggggaaaggactcgagtgggtgtctactatcagctcaggaggcacttatatatat
tatcctgactctgtaaaaggccgatttacgatttctcgcgacaatgcaaagaactccctctacctccaaatgaacagtct
tagggcagaagacactgctgtatactattgtgcacgcctcggcggcgacaactactacgagtactttgacgtgtggggga
aagggactaccgtgacagtttcaagcggaggaggtggctcaggtggaggcgggtcaggggggggaggaagtgatattgtg
ctcacacaatccccagcctccctggctgtgtctcccggccaacgcgctacaattacatgtcgggcctccaaaagcgtgag
caccagcggctacagctacatgcactggtatcaacagaaaccaggacaaccccccaaactgttgatttatctcgcttcaa
acttggagtccggcgtgcctgcgcgcttttcagggagtgggagcggcacagattttacgctgactatcaaccccgtagaa
gcaaacgatacagcgaattattattgtcaacattcccgggaactcccctttacgttcggcgggggcacaaaggtcgaaat
taagagaacctcgggacaggtcctgctggaatccaacatcaaggttctgcccacatggtccaccccggtgcagccaatgg
ccctgattgtgctggggggcgtcgccggcctcctgcttttcattgggctaggcatcttcttccggagggaccagaggctg
ccccccgatgcccacaagccccctgggggaggcagtttccggacccccatccaagaggagcaggccgacgcccactccac
cctggccaagatccgcgttaagttctcccgatcagccgacgcgcctgcttacaagcagggccagaaccaactgtacaacg
agctgaatctcggtagacgggaagagtacgacgtgttggacaaacggagaggccgcgacccagaaatgggcggcaagcct
cgcaggaaaaacccccaggagggactgtacaatgagttgcagaaagataagatggcagaagcttatagcgagatcggaat
gaagggggaaaggagacgagggaaaggacacgacggcctttatcagggcctgtccacagcaacaaaagatacgtatgacg
ccctccatatgcaggcacttccaccacggtgataa
(amino acids)
(SEQ ID NO: 765)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRRDQRL
PPDAHKPPGGGSFRTPIQEEQADAHSTLAKIRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR**
CAR-T C2 CD4/CD4/CD28/4-1BB/CD3z sequence:
N-CD81s-huMNC2scFv- CD4ecd- CD4 transmembrane- CD28- 4-1BB- CD3zeta-C
(DNA)
(SEQ ID NO: 766)
atggccttgccagtgacggccctgctgctgccattggctcttctgttgcacgctgccaggcctgaagtgcagctcgtaga
gagtggcgggggactggtgaagcccggtggaagcctcagactcagttgcgccgcctcaggtttcactttttcaggttacg
ccatgtcctgggtaagacaggcaccggggaaaggactcgagtgggtgtctactatcagctcaggaggcacttatatatat
tatcctgactctgtaaaaggccgatttacgatttctcgcgacaatgcaaagaactccctctacctccaaatgaacagtct
tagggcagaagacactgctgtatactattgtgcacgcctcggcggcgacaactactacgagtactttgacgtgtggggga
aagggactaccgtgacagtttcaagcggaggaggtggctcaggtggaggcgggtcaggggggggaggaagtgatattgtg
ctcacacaatccccagcctccctggctgtgtctcccggccaacgcgctacaattacatgtcgggcctccaaaagcgtgag
caccagcggctacagctacatgcactggtatcaacagaaaccaggacaaccccccaaactgttgatttatctcgcttcaa
acttggagtccggcgtgcctgcgcgcttttcagggagtgggagcggcacagattttacgctgactatcaaccccgtagaa
gcaaacgatacagcgaattattattgtcaacattcccgggaactcccctttacgttcggcgggggcacaaaggtcgaaat
taagagaacctcgggacaggtcctgctggaatccaacatcaaggttctgcccacatggtccaccccggtgcagccaatgg
ccctgattgtgctggggggcgtcgccggcctcctgcttttcattgggctaggcatcttcttcagaagcaagcggtctcgg
ctcctgcattctgattacatgaacatgaccccaagaagaccaggccccaccaggaaacattaccagccctacgctccgcc
acgcgacttcgctgcctaccggtccaaaaggggccgcaaaaaactcctttacatttttaagcagccttttatgaggccag
tacagacgactcaagaggaagacgggtgctcatgccgctttcctgaggaggaggaaggagggtgcgaactgcgcgttaag
ttctcccgatcagccgacgcgcctgcttacaagcagggccagaaccaactgtacaacgagctgaatctcggtagacggga
agagtacgacgtgttggacaaacggagaggccgcgacccagaaatgggcggcaagcctcgcaggaaaaacccccaggagg
gactgtacaatgagttgcagaaagataagatggcagaagcttatagcgagatcggaatgaagggggaaaggagacgaggg
aaaggacacgacggcctttatcagggcctgtccacagcaacaaaagatacgtatgacgccctccatatgcaggcacttcc
accacggtgataa
(amino acids)
(SEQ ID NO: 767)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRSKRSR
LLHSDYMNMTPRRPGPTRKHYQPYAPPRDFAAYRSKRGRKKLLYIFKQPFMRPVQTTQEEDGCSCRFPEEEEGGCELRVK
FSRSADAPAYKQGQNQLYNELNLGRREEYDVLDKRRGRDPEMGGKPRRKNPQEGLYNELQKDKMAEAYSEIGMKGERRRG
KGHDGLYQGLSTATKDTYDALHMQALPPR**
CAR-T C2 CD4/CD4/CD28/OX40/CD3z sequence:
N-CD81s-huMNC2scFv- CD4ecd- CD4 transmembrane- CD28- OX40- CD3zeta-C
(DNA)
(SEQ ID NO: 768)
atggccttgccagtgacggccctgctgctgccattggctcttctgttgcacgctgccaggcctgaagtgcagctcgtaga
gagtggcgggggactggtgaagcccggtggaagcctcagactcagttgcgccgcctcaggtttcactttttcaggttacg
ccatgtcctgggtaagacaggcaccggggaaaggactcgagtgggtgtctactatcagctcaggaggcacttatatatat
tatcctgactctgtaaaaggccgatttacgatttctcgcgacaatgcaaagaactccctctacctccaaatgaacagtct
tagggcagaagacactgctgtatactattgtgcacgcctcggcggcgacaactactacgagtactttgacgtgtggggga
aagggactaccgtgacagtttcaagcggaggaggtggctcaggtggaggcgggtcaggggggggaggaagtgatattgtg
ctcacacaatccccagcctccctggctgtgtctcccggccaacgcgctacaattacatgtcgggcctccaaaagcgtgag
caccagcggctacagctacatgcactggtatcaacagaaaccaggacaaccccccaaactgttgatttatctcgcttcaa
acttggagtccggcgtgcctgcgcgcttttcagggagtgggagcggcacagattttacgctgactatcaaccccgtagaa
gcaaacgatacagcgaattattattgtcaacattcccgggaactcccctttacgttcggcgggggcacaaaggtcgaaat
taagagaacctcgggacaggtcctgctggaatccaacatcaaggttctgcccacatggtccaccccggtgcagccaatgg
ccctgattgtgctggggggcgtcgccggcctcctgcttttcattgggctaggcatcttcttcagaagcaagcggtctcgg
ctcctgcattctgattacatgaacatgaccccaagaagaccaggccccaccaggaaacattaccagccctacgctccgcc
acgcgacttcgctgcctaccggtcccggagggaccagaggctgccccccgatgcccacaagccccctgggggaggcagtt
tccggacccccatccaagaggagcaggccgacgcccactccaccctggccaagatccgcgttaagttctcccgatcagcc
gacgcgcctgcttacaagcagggccagaaccaactgtacaacgagctgaatctcggtagacgggaagagtacgacgtgtt
ggacaaacggagaggccgcgacccagaaatgggcggcaagcctcgcaggaaaaacccccaggagggactgtacaatgagt
tgcagaaagataagatggcagaagcttatagcgagatcggaatgaagggggaaaggagacgagggaaaggacacgacggc
ctttatcagggcctgtccacagcaacaaaagatacgtatgacgccctccatatgcaggcacttccaccacggtgataa
(amino acids)
(SEQ ID NO: 769)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRSKRSR
LLHSDYMNMTPRRPGPTRKHYQPYAPPRDFAAYRSRRDQRLPPDAHKPPGGGSFRTPIQEEQADAHSTLAKIRVKFSRSA
DAPAYKQGQNQLYNELNLGRREEYDVLDKRRGRDPEMGGKPRRKNPQEGLYNELQKDKMAEAYSEIGMKGERRRGKGHDG
LYQGLSTATKDTYDALHMQALPPR**
CAR-T E6 IgD/FC/CD8/4-1BB/CD3z
N-CD81s-huMNE6scFv- IgD hinge- Human IgG1 Fc- CD8 transmembrane- 4-1BB-
CD3zeta-C
(DNA)
(SEQ ID NO: 770)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagtctccaaaggcacag
gcctcctcagtgcccactgcacaaccccaagcagagggcagcctcgccaaggcaaccacagccccagccaccacccgtaac
acaggaagaggcggcgaagagaagaaaaaggagaaggagaaagaggaacaagaagagagagagacaaagacaccagagccc
aaatcttgtgacaaaactcacacatgcccaccgtgcccagcacctgaactcctggggggaccgtcagtcttcctcttcccc
ccaaaacccaaggacaccctcatgatctcccggacccctgaggtcacatgcgtggtggtggacgtgagccacgaagaccct
gaggtcaagttcaactggtacgtggacggcgtggaggtgcataatgccaagacaaagccgcgggaggagcagtacaacagc
acgtaccgtgtggtcagcgtcctcaccgtcctgcaccaggactggctgaatggcaaggagtacaagtgcaaggtctccaac
aaagccctcccagcccccatcgagaaaaccatctccaaagccaaagggcagccccgagaaccacaggtgtacaccctgccc
ccatcccgggaggagatgaccaagaaccaggtcagcctgacctgcctggtcaaaggcttctatcccagcgacatcgccgtg
gagtgggagagcaatgggcagccggagaacaactacaagaccacgcctcccgtgctggactccgacggctccttcttcctc
tacagcaagctcaccgtggacaagagcaggtggcagcaggggaacgtcttctcatgctccgtgatgcatgaggctctgcac
aaccactacacgcagaagagcctctccctgtctccgggtaaaatctacatctgggcgcccttggccgggacttgtggggtc
cttctcctgtcactggttatcaccctttactgcaaacggggcagaaagaaactcctgtatatattcaaacaaccatttatg
agaccagtacaaactactcaagaggaagatggctgtagctgccgatttccagaagaagaagaaggaggatgtgaactgaga
gtgaagttcagcaggagcgcagacgcccccgcgtacaagcagggccagaaccagctctataacgagctcaatctaggacga
agagaggagtacgatgttttggacaagagacgtggccgggaccctgagatggggggaaagccgagaaggaagaaccctcag
gaaggcctgtacaatgaactgcagaaagataagatggcggaggcctacagtgagattgggatgaaaggcgagcgccggagg
ggcaaggggcacgatggcctttaccagggtctcagtacagccaccaaggacacctacgacgcccttcacatgcaggccctg
ccccctcgctgataa
(amino acids)
(SEQ ID NO: 771)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKESPKAQASSVPTAQPQAEGSLAKATTAPATTRNTGRGGEEKKKEKEKEEQEERETK
TPEPKSCDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHEALHNHYTQKSLSLSPGKIYIWAP
LAGTCGVLLLSLVITLYCKRGRKKLLYIFKQPFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T E6 IgD/FCHingeless Y407R/CD8/4-1BB/CD3z
N-CD81s- huMNE6scFv- IgD hinge- Human IgG1 hingeless Fc Y407R- CD8
transmembrane- 4-1BB- CD3zeta-C
(DNA)
(SEQ ID NO: 772)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagtctccaaaggcacag
gcctcctcagtgcccactgcacaaccccaagcagagggcagcctcgccaaggcaaccacagccccagccaccacccgtaac
acaggaagaggcggcgaagagaagaaaaaggagaaggagaaagaggaacaagaagagagagagacaaagacaccagcacct
gaactcctggggggaccgtcagtcttcctcttccccccaaaacccaaggacaccctcatgatctcccggacccctgaggtc
acatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactgg
ctgaatggcaaggagtacaagtgcaaggtctccaacaaagccctcccagcccccatcgagaaaaccatctccaaagccaaa
gggcagccccgagaaccacaggtgtacaccctgcccccatcccgggaggagatgaccaagaaccaggtcagcctgacctgc
ctggtcaaaggcttctatcccagcgacatcgccgtggagtgggagagcaatgggcagccggagaacaactacaagaccacg
cctcccgtgctggactccgacggctccttcttcctcaggagcaagctcaccgtggacaagagcaggtggcagcaggggaac
gtcttctcatgctccgtgatgcatgaggctctgcacaaccactacacgcagaagagcctctccctgtctccgggtaaaatc
tacatctgggcgcccttggccgggacttgtggggtccttctcctgtcactggttatcaccctttactgcaaacggggcaga
aagaaactcctgtatatattcaaacaaccatttatgagaccagtacaaactactcaagaggaagatggctgtagctgccga
tttccagaagaagaagaaggaggatgtgaactgagagtgaagttcagcaggagcgcagacgcccccgcgtacaagcagggc
cagaaccagctctataacgagctcaatctaggacgaagagaggagtacgatgttttggacaagagacgtggccgggaccct
gagatggggggaaagccgagaaggaagaaccctcaggaaggcctgtacaatgaactgcagaaagataagatggcggaggcc
tacagtgagattgggatgaaaggcgagcgccggaggggcaaggggcacgatggcctttaccagggtctcagtacagccacc
aaggacacctacgacgcccttcacatgcaggccctgccccctcgctgataa
(amino acids)
(SEQ ID NO: 773)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKESPKAQASSVPTAQPQAEGSLAKATTAPATTRNTGRGGEEKKKEKEKEEQEERETK
TPAPELLGGPSVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKGFYPSDIAVEWESNGQPE
NNYKTTPPVLDSDGSFFLRSKLTVDKSRWQQGNVFSCSVMHEALHNHYTQKSLSLSPGKIYIWAPLAGTCGVLLLSLVIT
LYCKRGRKKLLYIFKQPFMRPVQTTQEEDGCSCRFPEEEEGGCELRVKFSRSADAPAYKQGQNQLYNELNLGRREEYDVL
DKRRGRDPEMGGKPRRKNPQEGLYNELQKDKMAEAYSEIGMKGERRRGKGHDGLYQGLSTATKDTYDALHMQALPPR**
NFATc1P2-MMP9
(DNA)
(SEQ ID NO: 774)
caggcctggggacactcgcggcgggaagatttggaggggaggggagggggaggggcgtgggggcgcggcctcgctggagt
ccccctgaccccccgacccccgcccaccggcctgggcgtcctcccgcggcccctcctcccctcccggcgcccggtgctct
ggggcgcgtgccacgcctggctcggcgccgtaggggcccccgcaggtagagacccctggaaatggcctcgacgccgcagg
agcgaggcggccaccaccccgctaatccgggcacgtctctccaggccgaggcctgcggtggaaaagccggggttccattt
gtgctgagtcggggcggccgaatggagccaggcctcgggacgcgggacggacgggctctggccgcgcaccttcgcgggct
ctgcagcgcccgaccgcctcccccggcagggaggaggcgcttgtggggggcacccacggggcacagtgatccctgggggt
ctgcggacctcctgggccccgcagcagacacgagtttagcctttgggtttagtttaaatcacataagggtgtcgtgcaat
cgatttatggtttctacacaccagacactttaacctccaaccccccccatccaaagccaacaagaaaatgcggtgccgtg
ttggcagctgagctgcgcccgaagagacgcagggagacgtaagagaggaaagtgtgagtggccggggggcctccccccgt
cagaagtcgcgcagtcgcgcccataaaacgccccctccgggcggctagggcaggtgagcgcgtccccgggcctccccacg
ccggcccctgccacaggccgtctaggtcgagcagatatttacagaataaaaatgacaataactcgacgtcccgggacggc
cacgcaatctgttagtaatttagcgggatgggaatttcctttctagggcctgccagtgaagcgcttttccaaatttccac
agcgggggaagcctgcgattttacataatgacttcagcatgccgggctttctcgacacccctccccggcccccggccccc
gccccccgccccttttccagcagggccgggctccctccggacacccgcgtggactcaggcgtcccgtctggcccgttcgc
ccccgtttcccccgccagccccagcgcccccctgcccggcccccggattccccgttcccgcccctacgcccccatcccct
ccccgtgcgcccctccccgtgcgcccccctccccgtgcgccccccctccccgtgcgcccccctccccgtgcgccccccct
ccccgggcgcccccctccccgggcgccccccctccccgtgcgcccccccctccccgtgcgccccccctccccgtgcgcgc
cccgcctcttgcgcccctgcccccaggcgagcggctgccgcggcgcggggaggggcgggcgctcggcgactcgtccccgg
ggccccgcgcgggcccgggcagcaggggcgtgatgtcacggcagggagggggcgcgggagccgccgggccggcggggagg
cgggggaggtgttttccagctttaaaaaggcaggaggcagagcgcggccctgcgtcagagcgagactcagaggctccgaa
ctcgccggcggagtcgccgcgccagatcccagcagcagggcgcggaagcttctctcgacattcgtttctagagccaccat
gagcctctggcagcccctggtcctggtgctcctggtgctgggctgctgctttgctgcccccagacagcgccagtccaccct
tgtgctcttccctggagacctgagaaccaatctcaccgacaggcagctggcagaggaatacctgtaccgctatggttacac
tcgggtggcagagatgcgtggagagtcgaaatctctggggcctgcgctgctgcttctccagaagcaactgtccctgcccga
gaccggtgagctggatagcgccacgctgaaggccatgcgaaccccacggtgcggggtcccagacctgggcagattccaaac
ctttgagggcgacctcaagtggcaccaccacaacatcacctattggatccaaaactactcggaagacttgccgcgggcggt
gattgacgacgcctttgcccgcgccttcgcactgtggagcgcggtgacgccgctcaccttcactcgcgtgtacagccggga
cgcagacatcgtcatccagtttggtgtcgcggagcacggagacgggtatcccttcgacgggaaggacgggctcctggcaca
cgcctttcctcctggccccggcattcagggagacgcccatttcgacgatgacgagttgtggtccctgggcaagggcgtcgt
ggttccaactcggtttggaaacgcagatggcgcggcctgccacttccccttcatcttcgagggccgctcctactctgcctg
caccaccgacggtcgctccgacggcttgccctggtgcagtaccacggccaactacgacaccgacgaccggtttggcttctg
ccccagcgagagactctacacccaggacggcaatgctgatgggaaaccctgccagtttccattcatcttccaaggccaatc
ctactccgcctgcaccacggacggtcgctccgacggctaccgctggtgcgccaccaccgccaactacgaccgggacaagct
cttcggcttctgcccgacccgagctgactcgacggtgatggggggcaactcggcgggggagctgtgcgtcttccccttcac
tttcctgggtaaggagtactcgacctgtaccagcgagggccgcggagatgggcgcctctggtgcgctaccacctcgaactt
tgacagcgacaagaagtggggcttctgcccggaccaaggatacagtttgttcctcgtggcggcgcatgagttcggccacgc
gctgggcttagatcattcctcagtgccggaggcgctcatgtaccctatgtaccgcttcactgaggggccccccttgcataa
ggacgacgtgaatggcatccggcacctctatggtcctcgccctgaacctgagccacggcctccaaccaccaccacaccgca
gcccacggctcccccgacggtctgccccaccggaccccccactgtccacccctcagagcgccccacagctggccccacagg
tcccccctcagctggccccacaggtccccccactgctggcccttctacggccactactgtgcctttgagtccggtggacga
tgcctgcaacgtgaacatcttcgacgccatcgcggagattgggaaccagctgtatttgttcaaggatgggaagtactggcg
attctctgagggcagggggagccggccgcagggccccttccttatcgccgacaagtggcccgcgctgccccgcaagctgga
ctcggtctttgaggagcggctctccaagaagcttttcttcttctctgggcgccaggtgtgggtgtacacaggcgcgtcggt
gctgggcccgaggcgtctggacaagctgggcctgggagccgacgtggcccaggtgaccggggccctccggagtggcagggg
gaagatgctgctgttcagcgggcggcgcctctggaggttcgacgtgaaggcgcagatggtggatccccggagcgccagcga
ggtggaccggatgttccccggggtgcctttggacacgcacgacgtcttccagtaccgagagaaagcctatttctgccagga
ccgcttctactggcgcgtgagttcccggagtgagttgaaccaggtggaccaagtgggctacgtgacctatgacatcctgca
gtgccctgaggacgattacaaggatgacgacgataagtgataa
NFATc1P2-MMP9cat
(DNA)
(SEQ ID NO: 775)
caggcctggggacactcgcggcgggaagatttggaggggaggggagggggaggggcgtgggggcgcggcctcgctggagt
ccccctgaccccccgacccccgcccaccggcctgggcgtcctcccgcggcccctcctcccctcccggcgcccggtgctct
ggggcgcgtgccacgcctggctcggcgccgtaggggcccccgcaggtagagacccctggaaatggcctcgacgccgcagg
agcgaggcggccaccaccccgctaatccgggcacgtctctccaggccgaggcctgcggtggaaaagccggggttccattt
gtgctgagtcggggcggccgaatggagccaggcctcgggacgcgggacggacgggctctggccgcgcaccttcgcgggct
ctgcagcgcccgaccgcctcccccggcagggaggaggcgcttgtggggggcacccacggggcacagtgatccctgggggt
ctgcggacctcctgggccccgcagcagacacgagtttagcctttgggtttagtttaaatcacataagggtgtcgtgcaat
cgatttatggtttctacacaccagacactttaacctccaaccccccccatccaaagccaacaagaaaatgcggtgccgtg
ttggcagctgagctgcgcccgaagagacgcagggagacgtaagagaggaaagtgtgagtggccggggggcctccccccgt
cagaagtcgcgcagtcgcgcccataaaacgccccctccgggcggctagggcaggtgagcgcgtccccgggcctccccacg
ccggcccctgccacaggccgtctaggtcgagcagatatttacagaataaaaatgacaataactcgacgtcccgggacggc
cacgcaatctgttagtaatttagcgggatgggaatttcctttctagggcctgccagtgaagcgcttttccaaatttccac
agcgggggaagcctgcgattttacataatgacttcagcatgccgggctttctcgacacccctccccggcccccggccccc
gccccccgccccttttccagcagggccgggctccctccggacacccgcgtggactcaggcgtcccgtctggcccgttcgc
ccccgtttcccccgccagccccagcgcccccctgcccggcccccggattccccgttcccgcccctacgcccccatcccct
ccccgtgcgcccctccccgtgcgcccccctccccgtgcgccccccctccccgtgcgcccccctccccgtgcgccccccct
ccccgggcgcccccctccccgggcgccccccctccccgtgcgcccccccctccccgtgcgccccccctccccgtgcgcgc
cccgcctcttgcgcccctgcccccaggcgagcggctgccgcggcgcggggaggggcgggcgctcggcgactcgtccccgg
ggccccgcgcgggcccgggcagcaggggcgtgatgtcacggcagggagggggcgcgggagccgccgggccggcggggagg
cgggggaggtgttttccagctttaaaaaggcaggaggcagagcgcggccctgcgtcagagcgagactcagaggctccgaa
ctcgccggcggagtcgccgcgccagatcccagcagcagggcgcggaagcttctctcgacattcgtttctagagccaccat
gagcctctggcagcccctggtcctggtgctcctggtgctgggctgctgctttgctttccaaacctttgagggcgacctcaa
gtggcaccaccacaacatcacctattggatccaaaactactcggaagacttgccgcgggcggtgattgacgacgcctttgc
ccgcgccttcgcactgtggagcgcggtgacgccgctcaccttcactcgcgtgtacagccgggacgcagacatcgtcatcca
gtttggtgtcgcggagcacggagacgggtatcccttcgacgggaaggacgggctcctggcacacgcctttcctcctggccc
cggcattcagggagacgcccatttcgacgatgacgagttgtggtccctgggcaagggcgtcgtggttccaactcggtttgg
aaacgcagatggcgcggcctgccacttccccttcatcttcgagggccgctcctactctgcctgcaccaccgacggtcgctc
cgacggcttgccctggtgcagtaccacggccaactacgacaccgacgaccggtttggcttctgccccagcgagagactcta
cacccaggacggcaatgctgatgggaaaccctgccagtttccattcatcttccaaggccaatcctactccgcctgcaccac
ggacggtcgctccgacggctaccgctggtgcgccaccaccgccaactacgaccgggacaagctcttcggcttctgcccgac
ccgagctgactcgacggtgatggggggcaactcggcgggggagctgtgcgtcttccccttcactttcctgggtaaggagta
ctcgacctgtaccagcgagggccgcggagatgggcgcctctggtgcgctaccacctcgaactttgacagcgacaagaagtg
gggcttctgcccggaccaaggatacagtttgttcctcgtggcggcgcatgagttcggccacgcgctgggcttagatcattc
ctcagtgccggaggcgctcatgtaccctatgtaccgcttcactgaggggccccccttgcataaggacgacgtgaatggcat
ccggcacctctatggtcctcgccctgaacctgattacaaggatgacgacgataagtgataa
NFAT response element 2
(DNA)
(SEQ ID NO: 776)
aagaggaaaatttgtttcatacagaaggcgtt
NFAT response element 2 repeats
(DNA)
(SEQ ID NO: 777)
aagaggaaaatttgtttcatacagaaggcgttaagaggaaaatttgtttcatacagaaggcgttaagaggaaaatttgttt
catacagaaggcgttaagaggaaaatttgtttcatacagaaggcgtt
CMV minimal promoter 2
(DNA)
(SEQ ID NO: 778)
taggcgtgtacggtgggaggcctatataagcagagctcgtttagtgaaccgtcagatcgcctggagacgccatccacgctg
ttttgacctccatagaagacaccgggaccgatccagc
NFATRE2mCMV2-MMP9
(DNA)
(SEQ ID NO: 779)
aagaggaaaatttgtttcatacagaaggcgttaagaggaaaatttgtttcatacagaaggcgttaagaggaaaatttgttt
catacagaaggcgttaagaggaaaatttgtttcatacagaaggcgttactagttaggcgtgtacggtgggaggcctatata
agcagagctcgtttagtgaaccgtcagatcgcctggagacgccatccacgctgttttgacctccatagaagacaccgggac
cgatccagcctctcgacattcgtttctagagccaccatgagcctctggcagcccctggtcctggtgctcctggtgctgggc
tgctgctttgctgcccccagacagcgccagtccacccttgtgctcttccctggagacctgagaaccaatctcaccgacagg
cagctggcagaggaatacctgtaccgctatggttacactcgggtggcagagatgcgtggagagtcgaaatctctggggcct
gcgctgctgcttctccagaagcaactgtccctgcccgagaccggtgagctggatagcgccacgctgaaggccatgcgaacc
ccacggtgcggggtcccagacctgggcagattccaaacctttgagggcgacctcaagtggcaccaccacaacatcacctat
tggatccaaaactactcggaagacttgccgcgggcggtgattgacgacgcctttgcccgcgccttcgcactgtggagcgcg
gtgacgccgctcaccttcactcgcgtgtacagccgggacgcagacatcgtcatccagtttggtgtcgcggagcacggagac
gggtatcccttcgacgggaaggacgggctcctggcacacgcctttcctcctggccccggcattcagggagacgcccatttc
gacgatgacgagttgtggtccctgggcaagggcgtcgtggttccaactcggtttggaaacgcagatggcgcggcctgccac
ttccccttcatcttcgagggccgctcctactctgcctgcaccaccgacggtcgctccgacggcttgccctggtgcagtacc
acggccaactacgacaccgacgaccggtttggcttctgccccagcgagagactctacacccaggacggcaatgctgatggg
aaaccctgccagtttccattcatcttccaaggccaatcctactccgcctgcaccacggacggtcgctccgacggctaccgc
tggtgcgccaccaccgccaactacgaccgggacaagctcttcggcttctgcccgacccgagctgactcgacggtgatgggg
ggcaactcggcgggggagctgtgcgtcttccccttcactttcctgggtaaggagtactcgacctgtaccagcgagggccgc
ggagatgggcgcctctggtgcgctaccacctcgaactttgacagcgacaagaagtggggcttctgcccggaccaaggatac
agtttgttcctcgtggcggcgcatgagttcggccacgcgctgggcttagatcattcctcagtgccggaggcgctcatgtac
cctatgtaccgcttcactgaggggccccccttgcataaggacgacgtgaatggcatccggcacctctatggtcctcgccct
gaacctgagccacggcctccaaccaccaccacaccgcagcccacggctcccccgacggtctgccccaccggaccccccact
gtccacccctcagagcgccccacagctggccccacaggtcccccctcagctggccccacaggtccccccactgctggccct
tctacggccactactgtgcctttgagtccggtggacgatgcctgcaacgtgaacatcttcgacgccatcgcggagattggg
aaccagctgtatttgttcaaggatgggaagtactggcgattctctgagggcagggggagccggccgcagggccccttcctt
atcgccgacaagtggcccgcgctgccccgcaagctggactcggtctttgaggagcggctctccaagaagcttttcttcttc
tctgggcgccaggtgtgggtgtacacaggcgcgtcggtgctgggcccgaggcgtctggacaagctgggcctgggagccgac
gtggcccaggtgaccggggccctccggagtggcagggggaagatgctgctgttcagcgggcggcgcctctggaggttcgac
gtgaaggcgcagatggtggatccccggagcgccagcgaggtggaccggatgttccccggggtgcctttggacacgcacgac
gtcttccagtaccgagagaaagcctatttctgccaggaccgcttctactggcgcgtgagttcccggagtgagttgaaccag
gtggaccaagtgggctacgtgacctatgacatcctgcagtgccctgaggacgattacaaggatgacgacgataagtgataa
NFATRE2mCMV2-MMP9cat
(DNA)
(SEQ ID NO: 780)
aagaggaaaatttgtttcatacagaaggcgttaagaggaaaatttgtttcatacagaaggcgttaagaggaaaatttgttt
catacagaaggcgttaagaggaaaatttgtttcatacagaaggcgttactagttaggcgtgtacggtgggaggcctatata
agcagagctcgtttagtgaaccgtcagatcgcctggagacgccatccacgctgttttgacctccatagaagacaccgggac
cgatccagcctcgagctctcgacattcgtttctagagccaccatgagcctctggcagcccctggtcctggtgctcctggtg
ctgggctgctgctttgctttccaaacctttgagggcgacctcaagtggcaccaccacaacatcacctattggatccaaaac
tactcggaagacttgccgcgggcggtgattgacgacgcctttgcccgcgccttcgcactgtggagcgcggtgacgccgctc
accttcactcgcgtgtacagccgggacgcagacatcgtcatccagtttggtgtcgcggagcacggagacgggtatcccttc
gacgggaaggacgggctcctggcacacgcctttcctcctggccccggcattcagggagacgcccatttcgacgatgacgag
ttgtggtccctgggcaagggcgtcgtggttccaactcggtttggaaacgcagatggcgcggcctgccacttccccttcatc
ttcgagggccgctcctactctgcctgcaccaccgacggtcgctccgacggcttgccctggtgcagtaccacggccaactac
gacaccgacgaccggtttggcttctgccccagcgagagactctacacccaggacggcaatgctgatgggaaaccctgccag
tttccattcatcttccaaggccaatcctactccgcctgcaccacggacggtcgctccgacggctaccgctggtgcgccacc
accgccaactacgaccgggacaagctcttcggcttctgcccgacccgagctgactcgacggtgatggggggcaactcggcg
ggggagctgtgcgtcttccccttcactttcctgggtaaggagtactcgacctgtaccagcgagggccgcggagatgggcgc
ctctggtgcgctaccacctcgaactttgacagcgacaagaagtggggcttctgcccggaccaaggatacagtttgttcctc
gtggcggcgcatgagttcggccacgcgctgggcttagatcattcctcagtgccggaggcgctcatgtaccctatgtaccgc
ttcactgaggggccccccttgcataaggacgacgtgaatggcatccggcacctctatggtcctcgccctgaacctgattac
aaggatgacgacgataagtgataa
NFATc1 Promoter fragment (P1)
(DNA)
(SEQ ID NO: 781)
aggcaggaggaagaggaaaggggcgcagggcgctcggggagcagagccgggggcccgcggtggccgcagaggccgggccgg
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagaggctccgaactcgccggcggagtcgccgcgccagatcccagcagcagggcgcgg
NFATc1 Promoter fragment (P2)
(DNA)
(SEQ ID NO: 782)
aggcaggaggaagaggaaaggggcgcagggcgctcggggagcagagccgggggcccgcggtggccgcagaggccgggccgg
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagagg
NFATc1 Promoter fragment (P3)
(DNA)
(SEQ ID NO: 783)
caggcctggggacactcgcggcgggaagatttggaggggaggggagggggaggggcgtgggggcgcggcctcgctggagt
ccccctgaccccccgacccccgcccaccggcctgggcgtcctcccgcggcccctcctcccctcccggcgcccggtgctct
ggggcgcgtgccacgcctggctcggcgccgtaggggcccccgcaggtagagacccctggaaatggcctcgacgccgcagg
agcgaggcggccaccaccccgctaatccgggcacgtctctccaggccgaggcctgcggtggaaaagccggggttccattt
gtgctgagtcggggcggccgaatggagccaggcctcgggacgcgggacggacgggctctggccgcgcaccttcgcgggct
ctgcagcgcccgaccgcctcccccggcagggaggaggcgcttgtggggggcacccacggggcacagtgatccctgggggt
ctgcggacctcctgggccccgcagcagacacgagtttagcctttgggtttagtttaaatcacataagggtgtcgtgcaat
cgatttatggtttctacacaccagacactttaacctccaaccccccccatccaaagccaacaagaaaatgcggtgccgtg
ttggcagctgagctgcgcccgaagagacgcagggagacgtaagagaggaaagtgtgagtggccggggggcctccccccgt
cagaagtcgcgcagtcgcgcccataaaacgccccctccgggcggctagggcaggtgagcgcgtccccgggcctccccacg
ccggcccctgccacaggccgtctaggtcgagcagatatttacagaataaaaatgacaataactcgacgtcccgggacggc
cacgcaatctgttagtaatttagcgggatgggaatttcctttctagggcctgccagtgaagcgcttttccaaatttccac
agcgggggaagcctgcgattttacataatgacttcagcatgccgggctttctcgacacccctccccggcccccggccccc
gccccccgccccttttccagcagggccgggctccctccggacacccgcgtggactcaggcgtcccgtctggcccgttcgc
ccccgtttcccccgccagccccagcgcccccctgcccggcccccggattccccgttcccgcccctacgcccccatcccct
ccccgtgcgcccctccccgtgcgcccccctccccgtgcgccccccctccccgtgcgcccccctccccgtgcgccccccct
ccccgggcgcccccctccccgggcgccccccctccccgtgcgcccccccctccccgtgcgccccccctccccgtgcgcgc
cccgcctcttgcgcccctgcccccaggcgagcggctgccgcggcgcggggaggggcgggcgctcggcgactcgtccccgg
ggccccgcgcgggcccgggcagcaggggcgtgatgtcacggcagggagggggcgcgggagccgccgggccggcggggagg
cgggggaggtgttttccagctttaaaaaggcaggaggcagagcgcggccctgcgtcagagcgagactcagaggctccgaa
ctcgccggcggagtcgccgcgccagatcccagcagcagggcgcgg
NFAT consensus sequence:
(SEQ ID NO: 804)
(A/T)GGAAA(A/N)(A/T/C)N
Current NFAT RE (Form System Biosciences. The sequence is from the mouse IL2
promoter
(DNA)
(SEQ ID NO: 805)
aagaggaaaatttgtttcatacagaaggcgtt
Mouse IL2 Promoter (highlighted in green the NFAT RE used, highlighted in
yellow is the start codon)
(DNA)
(SEQ ID NO: 806)
aactagagacatataaaataacaccaacatccttagatacaacccttcctgagaatttattggacatcatactcttttta
aaaagcataataaacatcaagacacttacacaaaatatgttaaattaaatttaaaacaacaacgacaaaatagtacctca
agctcaacaagcattttaggtgtccttagcttactatttctctggctaactgtatgaagccatctatcaccctgtgtgca
attagctcattgtgtagataagaaggtaaaaccatcttgaaacaggaaaccaatatccttcctgtctaatcaacaaatct
aaaagatttattcttttcatctatctcctcttgcgtttgtccaccacaacaggctgcttacaggttcaggatggttttga
caaagagaacattttcatgagttacttttgtgtctccaccccaaagaggaaaatttgtttcatacagaaggcgttcattg
tatgaattaaaactgccacctaagtgtgggctaacccgaccaagagggatttcacctaaatccattcagtcagtgtatgg
gggtttaaagaaattccagagagtcatcagaagaggaaaaacaaaaggtaatgctttctgccacacaggtagactctttg
aaaatatgtgtaatatgtaaaacatcgtgacacccccatattatttttccagcattaacagtataaattgcctcccatgc
tgaagagctgcctatcacccttgctaatcactcctcacagtgacctcaagtcctgcaggcatgtacagcatgcagctcgc
atcctgtgtcac
NFAT RE (Form PRomega. The sequence is from the humane IL2 promoter
(DNA)
(SEQ ID NO: 807)
ggaggaaaaactgtttcatacagaaggcgt
Possible NFAT RE from ET-1 promoter
(DNA)
(SEQ ID NO: 808)
tccagggaaaatcggagtagaacaagagggatg
Possible NFAT RE from ET-1 promoter
(DNA)
(SEQ ID NO: 809)
actgttggaaaacgtaaacacgttattaaacggt
Possible NFAT RE from human CD3γ
(DNA)
(SEQ ID NO: 810)
tccttaacggaaaaacaaaa
Possible NFAT RE from human CD3γ
(DNA)
(SEQ ID NO: 811)
aaaggaaaaagtatatgttc
Possible NFAT RE from human IL3 promoter
(DNA)
(SEQ ID NO: 812)
atgccatggaaagggtg
Possible NFAT RE from human GPC6
(DNA)
(SEQ ID NO: 813)
aaggggaaatgttgagtctaga
Possible NFAT RE from human growth hormone-releasing hormone
(DNA)
(SEQ ID NO: 814)
AACTTGGAAAAGCATAG
NFATc1 promoter large
(DNA)
(SEQ ID NO: 815)
ttatgccgtctagaggagacatactttctactcaaagctacacacatagactacaacgatgggaaaagacgacacaccaa
cagcgacttcaggaaagctggagtggctgctaatgttagacaaaataggctttttaaaaaaggttttattaaagaggaat
gtttcgtaatgataaaagcactaatctgtgagaaagatacaacaatgataaacatacgtgcagctaataagagagctcca
aaatctatgaagcaaaaactcacagaatgaggggagaagcagttctacaacagagaatggggacttcgatactccacttt
caataatggatacaacaaccaggcagataacaaggcaacagaaggcctgaacaacagtataaaccaattagacctaccag
atatctatagctagcacactccacccaacgacagcagaatacacattcttctcaagcgcacaagtaacatcctccaggat
gggccatgttctaggccatcaaacaaactcaggtggtttgaggccagaggcctctcttttaaccaccacactagggcctt
cggaggaggcaagcagagagttgtcaaagaggccctcaggactgggtgcagtggctcatgactgtaatcccagcacttta
gaaggctgaggcacaaggatcttttgagctcaggagttcaagaaatgagcacttatccactgggcgcggtggctcacgcc
agtaatccagcactttgggaggcttaggcgggcggatcaagaggtcagaagctcaagaccagcctgaccaacatggtgaa
accccgtctctactaaaagtacaaaaattagccgggcgtggtggcgcacacctgtaatcccagctacttgggaggctgag
gcaggagaatcacttgaacccgggaggtggaggttgcagtgagtggagatcacaccattgcaccccagcctgggcaacag
agcgagactccgtctcaaaaaaaaaaaaaaaaaaaaagaaagaaagaaaaagaaaaaaaaagtgagcatgtattttgcca
gagtctggagattagaattaaattagcaaaccagaattatagaaaaagctatttacttttaagtaaacagctgagatttt
tttttttaagtcagtgtgaatgaagctcacagccatggttggagctgagaaagaaggatttccctttagttatgcacctg
tgtcagcaccttctgactttccttctaaagtctggggtgttcctgaggatccgtaagtttggggttcagggtttctacag
catgctgttacttgtgaaacatctctttaaccatgtcccagagttgcccaggagtttaagaccagcctgagcaacatagc
aagacctcatctcaacaacaacaaaaattagaaataaattagccaggtgtggtgacatgtgcctgtagtcccagctactc
agaaggctgaggcatgaggatcacttgggcccaggaagttggggctgcagggagccctgttcatgccgctgcactccagc
ctgcaagacagagcagaaaaaaagaatcaggatcctgggcagagggaggagaggggaccggggtccagcaagcacttggg
gattgactgaatggcgttggggagagatgactccaaagtcctggagtgggtgagaatgactgcgagtggcttttaggtgg
ggaggttcctgcctggccactccgggaggggacgtggggctgaagggtatcaggtgccgtgctgagcagtttggccttga
tcctaatgccctggacacacgtctagggtaggaaagttgactgatccattggtgatctgagtttttagacatggtggtag
tccatgaggtgggtgttcatgctaagagtttagacagggaaacctatgaagcccttagcaaccctccagggaaggggcgt
ggttaaagagatgtttcataagtaacagcatggtatagaaactctgaaccccaaatgtatgggtcctcaggaacacccca
gactttagaaggaaagtcagaaggtgctgacacgggtgtataactaaagggaaatccttctttctcagctccaaccatgg
ctgtgaggttcattcacactgacttaaaaaaaaaaatctcagtttacttaaaagtaaatagctttttctataattctggt
ttgctaatttaatcctagtctccagaccctggctaaataaatgcccatttctccagatggtctcaagagtctctggacat
cgtgggggcccttccctgttggttggaaggtgcctcaggaagaagggggtggattctgagttgagtcaaaacctcaaaga
cccctgatgggaaaagctctcaagtgaccaccgctgtgggccagaatgcaaaactgcaggaacagaacattcgcaggaac
agaacacagtcgtattaagtgattttcccgagcaggaagtggcatctggcctgcggttcagtagggggaggaaagggtgg
gcgcacctgcccctggctggcgcacctgccaggtagccccacgcggcaccgcgtgtgccgagcgcccctgaggatggaaa
gccccacgcggggcaggtggcacccaccctccgaagacgggacgggatggagcgttgagcttcggggcagctccggcccg
gcccgcgctggagacgcccgcatctgccaggatggcgtctcatagccctggtgctcacacatgacgccaggaagccccag
caacagtgaccgcccaggctctagaaaatattggacggggtggatgaacacccaagtgcgctccaggagaagggatttgg
caccccaaggggcttttaaaacggtaagcttctaggggtgtctttgcccccaataatccatagaaacaacagtcatctaa
aaatagtcttgttttctgtcctaagctccttttaactttgttagtcatcaccaatcctaaaataaaacccgtgtaacgtc
tcccctagtagcggctataaacaaacctacgaggaggcaggaggaagaggaaaggggcgcagggcgctcggggagcagag
ccgggggcccgcggtggccgcagaggccgggccggggcgcagaggccgggcgagctggccgcgctctgggccgccgcctc
cggaactccctgcgcctggcgcgcggccaccgtggtcccggcaacggcattaaacagagggaaacagacccgggattccg
tcacccgggcggggggataaggacggctttgagagcagacaggaaaagggagcttttctgcatggggtgaaaaaattatt
tattgaaggaggaggaggcggcagcggaggaaggggaggggcgggaggaggaggaagagccggccgcccccgccccggcc
ccggctcctcaggagccaagggcagcctcgccaggtcggtcccgggctcgaggaccgcggctggggtcgaggggctcagt
ctcccacgtgaccggctgggcgcgccccgccagacccggcctcgggattccctcctcccggcgagtctccgcccgccccg
tcctggaggtggggagaaggagggcggggcgggggggacggaaactctccccgccaaatcctggccccaggcctggggac
actcgcggcgggaagatttggaggggaggggagggggaggggcgtgggggcgcggcctcgctggagtccccctgaccccc
cgacccccgcccaccggcctgggcgtcctcccgcggcccctcctcccctcccggcgcccggtgctctggggcgcgtgcca
cgcctggctcggcgccgtaggggcccccgcaggtagagacccctggaaatggcctcgacgccgcaggagcgaggcggcca
ccaccccgctaatccgggcacgtctctccaggccgaggcctgcggtggaaaagccggggttccatttgtgctgagtcggg
gcggccgaatggagccaggcctcgggacgcgggacggacgggctctggccgcgcaccttcgcgggctctgcagcgcccga
ccgcctcccccggcagggaggaggcgcttgtggggggcacccacggggcacagtgatccctgggggtctgcggacctcct
gggccccgcagcagacacgagtttagcctttgggtttagtttaaatcacataagggtgtcgtgcaatcgatttatggttt
ctacacaccagacactttaacctccaaccccccccatccaaagccaacaagaaaatgcggtgccgtgttggcagctgagc
tgcgcccgaagagacgcagggagacgtaagagaggaaagtgtgagtggccggggggcctccccccgtcagaagtcgcgca
gtcgcgcccataaaacgccccctccgggcggctagggcaggtgagcgcgtccccgggcctccccacgccggcccctgcca
caggccgtctaggtcgagcagatatttacagaataaaaatgacaataactcgacgtcccgggacggccacgcaatctgtt
agtaatttagcgggatgggaatttcctttctagggcctgccagtgaagcgcttttccaaatttccacagcgggggaagcc
tgcgattttacataatgacttcagcatgccgggctttctcgacacccctccccggcccccggcccccgccccccgcccct
tttccagcagggccgggctccctccggacacccgcgtggactcaggcgtcccgtctggcccgttcgcccccgtttccccc
gccagccccagcgcccccctgcccggcccccggattccccgttcccgcccctacgcccccatcccctccccgtgcgcccc
tccccgtgcgcccccctccccgtgcgccccccctccccgtgcgcccccctccccgtgcgccccccctccccgggcgcccc
cctccccgggcgccccccctccccgtgcgcccccccctccccgtgcgccccccctccccgtgcgcgccccgcctcttgcg
cccctgcccccaggcgagcggctgccgcggcgcggggaggggcgggcgctcggcgactcgtccccggggccccgcgcggg
cccgggcagcaggggcgtgatgtcacggcagggagggggcgcgggagccgccgggccggcggggaggcgggggaggtgtt
ttccagctttaaaaaggcaggaggcagagcgcggccctgcgtcagagcgagactcagaggctccgaactcgccggcggag
tcgccgcgccagatcccagcagcagggcgcgggcaccggggcgcgggcagggctcggagccaccgcgcaggtcctagggc
cgcggccgggccccgccacgcgcgcacacgcccctcgatg
NFATc3 promoter sequence
(DNA)
(SEQ ID NO: 816)
gcagccaggcagggtgggcgcgcgtagggggcggggccgggcgcgcggcagggcgcgagagcgcacccgcggcggcggtg
gcggcgactgtgggggggcggcggggaacattggctaagccgacagtggaggcttaggcaccggtggcgggcggctgcgg
ttcctggtgctgctcggcgcgcggccagctttcggaacggaacgctcggcgtcgcgggccccgcccggaaagtttgccgt
ggagtcgcgacctcttggcccgcgcggcccggcatgaagcggcgttgaggagctgctgccgccgcttgccgctgccgccg
ccgccgcctgaggaggagctgcagcaccctgggccacgccg
NFATc2 promoter sequence 1
(DNA)
(SEQ ID NO: 817)
cagagagaggctgcgttcagactggggcactgccatcccctccgcatca
tggggtctgtggaccaaggtaactgactctcgatcccttccagccttttccgctcgctcctcccggccctttcctgctgc
tcccgtcccgggcagcactttcagctcccggcagaggtcggtgcgggaggcctggggaccccgctcgccctcggcgcaca
ggtagcggggcccgcggaggggcgcccgcgccccggccagggaagggacacttgggaaggcgactttggacaactttacg
cgggggcagggaagtgtcccaggccgggattccctaggccagtctgtcgggaggattttcctctccacgggacaccggga
gggattctcgctactaaccgctggctgtttaaccgtttcagcactcggcttttgacagcaa
NFATc2 promoter sequence 2
(DNA)
(SEQ ID NO: 818)
catcatggggtctgtggaccaaggtaactgactctcgatcccttccagccttttccgctcgctc
NFATc1 response element consensus
(DNA)
(SEQ ID NO: 819)
cattttttccat
NFATc1 response element consensus
(DNA)
(SEQ ID NO: 820)
tttttcca
NFAT response elements contained within the Foxp3 enhancer region
(DNA)
(SEQ ID NO: 821)
acttgaaaatgagataaatgttcacctatgttggcttctagtctcttttatggcttcatt
ttttccatttactatagaggttaagagtgtgggtactggagccagactgtctgggacaa
muE6 IgD/CD8/41BB/CD3z
(DNA)
(SEQ ID NO: 822)
atggccctgcccgtgaccgctttgctgctccccctggcgctgctgctgcacgccgccaggccagaggtgaaggtggtggag
tctgggggagacttagtgaagcctggagggtccctgaaactctcctgtgtagtctctggattcactttcagtagatatggc
atgtcttgggttcgccagactccaggcaagaggctggagtgggtcgcaaccattagtggtggcggtacttacatctactat
ccagacagtgtgaaggggcgattcaccatctccagagacaatgccaagaacaccctgtacctgcaaatgagcagtctgaag
tctgaggacacagccatgtatcactgtacaagggataactacggtaggaactacgactacggtatggactactggggtcaa
ggaacctcagtcaccgtctcctcaggcggtggcggatccggcggtggcggatccggcggtggcggatcccaaattgttctc
acccagtctccagcaatcatgtctgcatctccaggggaggaggtcaccctaacctgcagtgccacctcaagtgtaagttac
atacactggttccagcagaggccaggcacttctcccaaactctggatttatagcacatccaacctggcttctggagtccct
gttcgcttcagtggcagtggatatgggacctcttactctctcacaatcagccgaatggaggctgaagatgctgccacttat
tactgccagcaaaggagtagttccccattcacgttcggctcggggacaaagttggaaataaaagagtctccaaaggcaca
ggcctcctcagtgcccactgcacaaccccaagcagagggcagcctcgccaaggcaaccacagccccagccaccacccgta
acacaggaagaggcggcgaagagaagaaaaaggagaaggagaaagaggaacaagaagagagagagacaaagacaccaatc
tacatttgggccccgctcgcaggcacatgtggagtgctcctcctctccctggtgattaccctgtactgcaaaaggggccgc
aaaaaactcctttacatttttaagcagccttttatgaggccagtacagacgactcaagaggaagacgggtgctcatgccgc
tttcctgaggaggaggaaggagggtgcgaactgcgcgttaagttctcccgatcagccgacgcgcctgcttacaagcagggc
cagaaccaactgtacaacgagctgaatctcggtagacgggaagagtacgacgtgttggacaaacggagaggccgcgaccca
gaaatgggcggcaagcctcgcaggaaaaacccccaggagggactgtacaatgagttgcagaaagataagatggcagaagct
tatagcgagatcggaatgaagggggaaaggagacgagggaaaggacacgacggcctttatcagggcctgtccacagcaaca
aaagatacgtatgacgccctccatatgcaggcacttccaccacggtgataa
(amino acids)
(SEQ ID NO: 823)
MALPVTALLLPLALLLHAARPEVKVVESGGDLVKPGGSLKLSCVVSGFTFSRYGMSWVRQTPGKRLEWVATISGGGTYIY
YPDSVKGRFTISRDNAKNTLYLQMSSLKSEDTAMYHCTRDNYGRNYDYGMDYWGQGTSVTVSSGGGGSGGGGSGGGGSQI
VLTQSPAIMSASPGEEVTLTCSATSSVSYIHWFQQRPGTSPKLWIYSTSNLASGVPVRFSGSGYGTSYSLTISRMEAEDA
ATYYCQQRSSSPFTFGSGTKLEIKESPKAQASSVPTAQPQAEGSLAKATTAPATTRNTGRGGEEKKKEKEKEEQEERETK
TPIYIWAPLAGTCGVLLLSLVITLYCKRGRKKLLYIFKQPFMRPVQTTQEEDGCSCRFPEEEEGGCELRVKFSRSADAPA
YKQGQNQLYNELNLGRREEYDVLDKRRGRDPEMGGKPRRKNPQEGLYNELQKDKMAEAYSEIGMKGERRRGKGHDGLYQG
LSTATKDTYDALHMQALPPR**
muC2 IgD/CD8/41BB/CD3z
(DNA)
(SEQ ID NO: 824)
atggccctgcccgtgaccgctttgctgctccccctggcgctgctgctgcacgccgccaggccagaggtccagctggaggag
tcagggggaggcttagtgaagcctggagggtccctgaaactctcctgtgcagcctctggattcactttcagtggctatgcc
atgtcttgggttcgccagactccggagaagaggctggagtgggtcgcaaccattagtagtggtggtacttatatctactat
ccagacagtgtgaaggggcgattcaccatctccagagacaatgccaagaacaccctgtacctgcaaatgagcagtctgagg
tctgaggacacggccatgtattactgtgcaagacttgggggggataattactacgaatacttcgatgtctggggcgcaggg
accacggtcaccgtctcctccggcggtggcggatccggcggtggcggatccggcggtggcggatccgacattgtgatcaca
cagtctacagcttccttaggtgtatctctggggcagagggccaccatctcatgcagggccagcaaaagtgtcagtacatct
ggctatagttatatgcactggtaccaacagagaccaggacagccacccaaactcctcatctatcttgcatccaacctagaa
tctggggtccctgccaggttcagtggcagtgggtctgggacagacttcaccctcaacatccatcctgtggaggaggaggat
gctgcaacctattactgtcagcacagtagggagcttccgttcacgttcggaggggggaccaagctggagataaaagagtc
tccaaaggcacaggcctcctcagtgcccactgcacaaccccaagcagagggcagcctcgccaaggcaaccacagccccag
ccaccacccgtaacacaggaagaggcggcgaagagaagaaaaaggagaaggagaaagaggaacaagaagagagagagaca
aagacaccaatctacatttgggccccgctcgcaggcacatgtggagtgctcctcctctccctggtgattaccctgtactg
caaaaggggccgcaaaaaactcctttacatttttaagcagccttttatgaggccagtacagacgactcaagaggaagacgg
gtgctcatgccgctttcctgaggaggaggaaggagggtgcgaactgcgcgttaagttctcccgatcagccgacgcgcctgc
ttacaagcagggccagaaccaactgtacaacgagctgaatctcggtagacgggaagagtacgacgtgttggacaaacggag
aggccgcgacccagaaatgggcggcaagcctcgcaggaaaaacccccaggagggactgtacaatgagttgcagaaagataa
gatggcagaagcttatagcgagatcggaatgaagggggaaaggagacgagggaaaggacacgacggcctttatcagggcct
gtccacagcaacaaaagatacgtatgacgccctccatatgcaggcacttccaccacggtgataa
(amino acids)
(SEQ ID NO: 825)
MALPVTALLLPLALLLHAARPEVQLEESGGGLVKPGGSLKLSCAASGFTFSGYAMSWVRQTPEKRLEWVATISSGGTYIY
YPDSVKGRFTISRDNAKNTLYLQMSSLRSEDTAMYYCARLGGDNYYEYFDVWGAGTTVTVSSGGGGSGGGGSGGGGSDIV
ITQSTASLGVSLGQRATISCRASKSVSTSGYSYMHWYQQRPGQPPKLLIYLASNLESGVPARFSGSGSGTDFTLNIHPVE
EEDAATYYCQHSRELPFTFGGGTKLEIKESPKAQASSVPTAQPQAEGSLAKATTAPATTRNTGRGGEEKKKEKEKEEQEE
RETKTPIYIWAPLAGTCGVLLLSLVITLYCKRGRKKLLYIFKQPFMRPVQTTQEEDGCSCRFPEEEEGGCELRVKFSRSA
DAPAYKQGQNQLYNELNLGRREEYDVLDKRRGRDPEMGGKPRRKNPQEGLYNELQKDKMAEAYSEIGMKGERRRGKGHDG
LYQGLSTATKDTYDALHMQALPPR**
muE6 CD28/CD28/CD28/CD3z
(DNA)
(SEQ ID NO: 826)
atggccctgcccgtgaccgctttgctgctccccctggcgctgctgctgcacgccgccaggccagaggtgaaggtggtgga
gtctgggggagacttagtgaagcctggagggtccctgaaactctcctgtgtagtctctggattcactttcagtagatatg
gcatgtcttgggttcgccagactccaggcaagaggctggagtgggtcgcaaccattagtggtggcggtacttacatctac
tatccagacagtgtgaaggggcgattcaccatctccagagacaatgccaagaacaccctgtacctgcaaatgagcagtct
gaagtctgaggacacagccatgtatcactgtacaagggataactacggtaggaactacgactacggtatggactactggg
gtcaaggaacctcagtcaccgtctcctcaggcggtggcggatccggcggtggcggatccggcggtggcggatcccaaatt
gttctcacccagtctccagcaatcatgtctgcatctccaggggaggaggtcaccctaacctgcagtgccacctcaagtgt
aagttacatacactggttccagcagaggccaggcacttctcccaaactctggatttatagcacatccaacctggcttctg
gagtccctgttcgcttcagtggcagtggatatgggacctcttactctctcacaatcagccgaatggaggctgaagatgct
gccacttattactgccagcaaaggagtagttccccattcacgttcggctcggggacaaagttggaaataaaaaaacacct
ttgtccaagtcccctatttcccggaccttctaagcccttttgggtgctggtggtggttggtggagtcctggcttgctata
gcttgctagtaacagtggcctttattattttctgggtgagaagcaagcggtctcggctcctgcattctgattacatgaac
atgaccccaagaagaccaggccccaccaggaaacattaccagccctacgctccgccacgcgacttcgctgcctaccggtc
ccgcgttaagttctcccgatcagccgacgcgcctgcttacaagcagggccagaaccaactgtacaacgagctgaatctcg
gtagacgggaagagtacgacgtgttggacaaacggagaggccgcgacccagaaatgggcggcaagcctcgcaggaaaaac
ccccaggagggactgtacaatgagttgcagaaagataagatggcagaagcttatagcgagatcggaatgaagggggaaag
gagacgagggaaaggacacgacggcctttatcagggcctgtccacagcaacaaaagatacgtatgacgccctccatatgc
aggcacttccaccacggtgataa
(amino acids)
(SEQ ID NO: 827)
MALPVTALLLPLALLLHAARPEVKVVESGGDLVKPGGSLKLSCVVSGFTFSRYGMSWVRQTPGKRLEWVATISGGGTYIY
YPDSVKGRFTISRDNAKNTLYLQMSSLKSEDTAMYHCTRDNYGRNYDYGMDYWGQGTSVTVSSGGGGSGGGGSGGGGSQI
VLTQSPAIMSASPGEEVTLTCSATSSVSYIHWFQQRPGTSPKLWIYSTSNLASGVPVRFSGSGYGTSYSLTISRMEAEDA
ATYYCQQRSSSPFTFGSGTKLEIKKHLCPSPLFPGPSKPFWVLVVVGGVLACYSLLVTVAFIIFWVRSKRSRLLHSDYMN
MTPRRPGPTRKHYQPYAPPRDFAAYRSRVKFSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR**
muC2 CD28/CD28/CD28/CD3z
(DNA)
(SEQ ID NO: 828)
atggccctgcccgtgaccgctttgctgctccccctggcgctgctgctgcacgccgccaggccagaggtccagctggaggag
tcagggggaggcttagtgaagcctggagggtccctgaaactctcctgtgcagcctctggattcactttcagtggctatgcc
atgtcttgggttcgccagactccggagaagaggctggagtgggtcgcaaccattagtagtggtggtacttatatctactat
ccagacagtgtgaaggggcgattcaccatctccagagacaatgccaagaacaccctgtacctgcaaatgagcagtctgagg
tctgaggacacggccatgtattactgtgcaagacttgggggggataattactacgaatacttcgatgtctggggcgcaggg
accacggtcaccgtctcctccggcggtggcggatccggcggtggcggatccggcggtggcggatccgacattgtgatcaca
cagtctacagcttccttaggtgtatctctggggcagagggccaccatctcatgcagggccagcaaaagtgtcagtacatct
ggctatagttatatgcactggtaccaacagagaccaggacagccacccaaactcctcatctatcttgcatccaacctagaa
tctggggtccctgccaggttcagtggcagtgggtctgggacagacttcaccctcaacatccatcctgtggaggaggaggat
gctgcaacctattactgtcagcacagtagggagcttccgttcacgttcggaggggggaccaagctggagataaaaaaacac
ctttgtccaagtcccctatttcccggaccttctaagcccttttgggtgctggtggtggttggtggagtcctggcttgctat
agcttgctagtaacagtggcctttattattttctgggtgagaagcaagcggtctcggctcctgcattctgattacatgaac
atgaccccaagaagaccaggccccaccaggaaacattaccagccctacgctccgccacgcgacttcgctgcctaccggtcc
cgcgttaagttctcccgatcagccgacgcgcctgcttacaagcagggccagaaccaactgtacaacgagctgaatctcggt
agacgggaagagtacgacgtgttggacaaacggagaggccgcgacccagaaatgggcggcaagcctcgcaggaaaaacccc
caggagggactgtacaatgagttgcagaaagataagatggcagaagcttatagcgagatcggaatgaagggggaaaggaga
cgagggaaaggacacgacggcctttatcagggcctgtccacagcaacaaaagatacgtatgacgccctccatatgcaggca
cttccaccacggtgataa
(amino acids)
(SEQ ID NO: 829)
MALPVTALLLPLALLLHAARPEVQLEESGGGLVKPGGSLKLSCAASGFTFSGYAMSWVRQTPEKRLEWVATISSGGTYIY
YPDSVKGRFTISRDNAKNTLYLQMSSLRSEDTAMYYCARLGGDNYYEYFDVWGAGTTVTVSSGGGGSGGGGSGGGGSDIV
ITQSTASLGVSLGQRATISCRASKSVSTSGYSYMHWYQQRPGQPPKLLIYLASNLESGVPARFSGSGSGTDFTLNIHPVE
EEDAATYYCQHSRELPFTFGGGTKLEIKKHLCPSPLFPGPSKPFWVLVVVGGVLACYSLLVTVAFIIFWVRSKRSRLLHS
DYMNMTPRRPGPTRKHYQPYAPPRDFAAYRSRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR**
CD19 IgD/CD8/41BB/CD3z
(DNA)
(SEQ ID NO: 830)
atggccctgcccgtgaccgctttgctgctccccctggcgctgctgctgcacgccgccaggccagacatacagatgacgca
gacgaccagcagcctttccgcttccctgggcgaccgagtaaccattagttgtagagcatctcaggatatttctaagtatc
tgaattggtaccaacagaaacctgatggcactgtcaagctcttgatatatcacaccagtcgactccattcaggcgtccct
tccagattcagtgggagtggcagcgggactgattactccctcactatctctaacctggaacaggaagacatcgctacata
cttctgtcagcagggaaacactctcccctatacctttgggggaggaaccaagttggaaataacaggcggtggcggatccg
gcggtggcggatccggcggtggcggatccgaggtgaaactgcaggagtcaggacctggcctggtggcgccctcacagagc
ctgtccgtcacatgcactgtctcaggggtctcattacccgactatggtgtaagctggattcgccagcctccacgaaaggg
tctggagtggctgggagtaatatggggtagtgaaaccacatactataattcagctctcaaatccagactgaccatcatca
aggacaactccaagagccaagttttcttaaaaatgaacagtctgcaaactgatgacacagccatttactactgtgccaaa
cattattactacggtggtagctatgctatggactactggggccaaggaacctcagtcaccgtctcctcagagtctccaaa
ggcacaggcctcctcagtgcccactgcacaaccccaagcagagggcagcctcgccaaggcaaccacagccccagccacca
cccgtaacacaggaagaggcggcgaagagaagaaaaaggagaaggagaaagaggaacaagaagagagagagacaaagaca
ccaatctacatttgggccccgctcgcaggcacatgtggagtgctcctcctctccctggtgattaccctgtactgcaaaag
gggccgcaaaaaactcctttacatttttaagcagccttttatgaggccagtacagacgactcaagaggaagacgggtgctc
atgccgctttcctgaggaggaggaaggagggtgcgaactgcgcgttaagttctcccgatcagccgacgcgcctgcttacaa
gcagggccagaaccaactgtacaacgagctgaatctcggtagacgggaagagtacgacgtgttggacaaacggagaggccg
cgacccagaaatgggcggcaagcctcgcaggaaaaacccccaggagggactgtacaatgagttgcagaaagataagatggc
agaagcttatagcgagatcggaatgaagggggaaaggagacgagggaaaggacacgacggcctttatcagggcctgtccac
agcaacaaaagatacgtatgacgccctccatatgcaggcacttccaccacggtgataa
(amino acids)
(SEQ ID NO: 831)
MALPVTALLLPLALLLHAARPDIQMTQTTSSLSASLGDRVTISCRASQDISKYLNWYQQKPDGTVKLLIYHTSRLHSGVP
SRFSGSGSGTDYSLTISNLEQEDIATYFCQQGNTLPYTFGGGTKLEITGGGGSGGGGSGGGGSEVKLQESGPGLVAPSQS
LSVTCTVSGVSLPDYGVSWIRQPPRKGLEWLGVIWGSETTYYNSALKSRLTIIKDNSKSQVFLKMNSLQTDDTAIYYCAK
HYYYGGSYAMDYWGQGTSVTVSSESPKAQASSVPTAQPQAEGSLAKATTAPATTRNTGRGGEEKKKEKEKEEQEERETKT
PIYIWAPLAGTCGVLLLSLVITLYCKRGRKKLLYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNELQKDKMAEAYSEIGMKGERRRGKGHDGLYQGL
STATKDTYDALHMQALPPR**

Those skilled in the art will recognize, or be able to ascertain using no more than routine experimentation, many equivalents to the specific embodiments of the invention specifically described herein.

What is claimed is:

1. A composition comprising a MUC1* extra cellular domain peptide attached to a surface and an immune cell that bears a receptor that binds to the peptide.

2. The composition of claim 1, wherein the immune cell has been engineered to express a receptor that binds to the peptide.

3. The composition of claim 1, wherein the MUC1* extra cellular domain peptide is PSMGFR.

4. The composition of claim 1, wherein the MUC1* extra cellular domain peptide is PSMGFR with N-terminus extended by the next 9 amino acids from the MUC1* sequence.

5. The composition of claim 1, wherein the MUC1* extra cellular domain peptide is PSMGFR with N-terminus extended by the next 20 amino acids from the MUC1* sequence.

6. A method of pre-stimulating an immune cell that bears a receptor to a cancer antigen comprising the steps of:
(i) contacting the immune cell with a peptide having a sequence derived from the cancer antigen; and
(ii) administering to a patient, diagnosed with cancer or at risk of developing a cancer, a therapeutic amount of the immune cells.

7. The method of claim 6, wherein the immune cell is separated away from the peptide prior to administering to the patient.

8. The method of claim 6, wherein the peptide is free in solution, attached to a surface, attached to a bead or is expressed on a cell.

9. The method of claim 6, wherein the immune cell is a CAR T cell bearing a chimeric antigen receptor (CAR) that comprises an antibody or antibody fragment that binds to the peptide.

10. The method as in claim 6, wherein the immune cell is in a pool of naturally occurring T cells, that may be obtained from the patient wherein the antigen is an antigen expressed by the patient's tumor, further comprising the step of expanding the patient T cells in the presence of the peptide antigen.

11. A method of activating, stimulating expansion of, or selecting for a population of immune cells that express a receptor that recognizes a specific tumor antigen, involving contacting the immune cell expressing the cognate receptor with the antigen to which it binds.

12. The method of claim 11, wherein the immune cell is a CAR T cell harboring anti-MUC1* antibody or fragment thereof, and the stimulating antigen is a peptide derived from the sequence of a cleaved MUC1 that is devoid of the tandem repeat domain.

13. The method of claim 11, wherein the antibody fragment that targets the CAR binds to a peptide and that peptide is used to activate, stimulate or select for the CAR T cells wherein the peptide has a sequence containing at least 15 contiguous amino acids, but can have up to 4 substitutions or deletions, wherein the peptide is derived from:
(i) PSMGFR region of MUC1;
(ii) PSMGFR peptide;
(iii) a peptide having amino acid sequence of
(SEQ ID NO: 620)
SNIKFRPGSVVVQLTLAFREGTINVHDVETQFNQYKTEAASRY;
(iv) a peptide having amino acid sequence of
(SEQ ID NO: 621)
SVVVQLTLAFREGTINVHDVETQFNQYKTEAASRY;
(v) a peptide having amino acid sequence of
(SEQ ID NO: 622)
VQLTLAFREGINVHDVETQFNQY;
or
(vi) a peptide having amino acid sequence of
(SEQ ID NO: 623)
SNIKFRPGSVVVQLTLAFREGTIN.

14. A method of pre-activating an immune cell transduced or transfected with nucleic acid encoding CAR that is specific for MUC1* and/or MUC1 specific cleavage enzyme by co-culturing the immune cell in vitro on a surface that presents a peptide having the sequence of truncated extracellular domain of MUC1, thus obtaining pre-activated immune cell.

15. The method of claim 14, wherein the immune cell is T cell.

16. The method of claim 14, comprising further administering to a patient a composition comprising the obtained pre-activated immune cell.

17. The method of claim 14, wherein the surface is a bead, a cell culture plate or a cell.

18. The method of claim 14, comprising removing the surface before administering the composition to the patient.

19. A method for training CAR T cell to be activated against a specific molecular target that is on a tumor comprising incubating a CAR T cell transduced with nucleic acid encoding an antibody against the specific molecular target with the specific molecular target.

20. The method of claim 19, wherein the specific molecular target is any antigen specific to a tumor cell.

21. The method of claim 20, wherein the specific molecular target is MUC1*.

22. The method of claim 20, wherein the specific molecular target is in the form of:
a) antigen positive cancer cells;
b) an antigen-negative cancer cell line transfected with the antigen;
c) a substrate to which is attached a synthetic antigen or specifically reactive peptide fragment thereof;
d) free antigen or specifically reactive peptide fragment thereof; or
e) an antigen or specifically reactive peptide fragment thereof coupled to BSA but free in solution.

23. The method of claim 19, wherein the incubation is in vitro.

24. The method of claim 22, wherein the substrate is magnetic bead, flask surface, cell culture dish or coated well.

25. The method of claim 22, wherein the antigen peptide is attached to the substrate via bovine serum albumin or human serum albumin.

26. The method of claim 22, wherein the antigen is MUC1* and specifically reactive peptide fragment thereof is MUC1* extra cellular domain peptide.

27. A method of culturing a population of CAR T cells that produce desired memory phenotype and CD4:CD8 ratio of about 50/50, which results in persistence in vivo and low side effects when administered to a patient.

28. A method of culturing a population of CAR T cells such that a greater portion of the CAR T cells are in the CD62L positive, CD45-RO negative (naïve) state and CD62L positive, CD45-RO positive (central memory) state, than CAR T cells in CD62L negative/CD45-RO positive (effector memory) and CD62L negative/CD45-RO negative effector states.

29. The method of claim 28, comprising culturing cells in vitro for 8-15 days, or 9-12 days, so as to yield a high percentage of the CAR T cells in the central memory and naïve state with far less in the effector memory and effector cell state.

30. A method of treating cancer comprising administering to a cancer patient the CAR T cell prepared according to claim 19.
